(19) United States
(12) Patent Application Publication
(10) Pub. No.: US 2003/0203363 A1
(43) Pub. Date: Oct. 30, 2003

(54) Novel human proteins, polynucleotides encoding them and methods of using the same

(76) Inventors: Kimberly A. Spytek, New Haven, CT (US); Corine A.M. Vernet, Branford, CT (US); Velizar T. Tchernev, Branford, CT (US); Uriel M. Malyankar, Branford, CT (US); Valerie Gerlach, Branford, CT (US); Li Li, Branford, CT (US); Bryan D. Zerhusen, Branford, CT (US); Meera Patturajan, Branford, CT (US); Vladimir Y. Gusev, Madison, CT (US); Ramesh Kekuda, Norwalk, CT (US); Carol E. A. Pena, New Haven, CT (US); Mei Zhong, Branford, CT (US); Esha A. Gangolli, Madison, CT (US); Raymond J. Taupier JR., East Haven, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/094,466

(22) Filed: Mar. 7, 2002

Related U.S. Application Data
(60) Provisional application No. 60/274,281, filed on Mar. 8, 2001.
(60) Provisional application No. 60/288,148, filed on May 2, 2001.
(60) Provisional application No. 60/274,849, filed on Mar. 9, 2001.
(60) Provisional application No. 60/275,235, filed on Mar. 12, 2001.
(60) Provisional application No. 60/338,375, filed on Dec. 4, 2001.
(60) Provisional application No. 60/275,579, filed on Mar. 13, 2001.
(60) Provisional application No. 60/335,302, filed on Oct. 31, 2001.
(60) Provisional application No. 60/275,601, filed on Mar. 13, 2001.
(60) Provisional application No. 60/276,000, filed on Mar. 14, 2001.
(60) Provisional application No. 60/277,338, filed on Mar. 20, 2001.
(60) Provisional application No. 60/277,239, filed on Mar. 20, 2001.
(60) Provisional application No. 60/277,327, filed on Mar. 20, 2001.
(60) Provisional application No. 60/294,821, filed on May 31, 2001.
(60) Provisional application No. 60/277,791, filed on Mar. 21, 2001.
(60) Provisional application No. 60/277,833, filed on Mar. 22, 2001.
(60) Provisional application No. 60/278,152, filed on Mar. 23, 2001.
(60) Provisional application No. 60/278,894, filed on Mar. 26, 2001.
(60) Provisional application No. 60/279,036, filed on Mar. 27, 2001.
(60) Provisional application No. 60/279,344, filed on Mar. 28, 2001.
(60) Provisional application No. 60/280,233, filed on Mar. 30, 2001.
(60) Provisional application No. 60/280,802, filed on Apr. 2, 2001.

(51) Int. Cl. C12Q 1/68; C07H 21/04; C12N 9/00; C12P 21/02; C12N 5/06; C07K 14/435

(52) U.S. Cl. 435/6; 435/69.1; 435/183; 435/320.1; 435/325; 530/350; 536/23.2

(57) ABSTRACT

Disclosed are polypeptides and nucleic acids encoding same. Also disclosed are vectors, host cells, antibodies and recombinant methods for producing the polypeptides and polynucleotides, as well as methods for using same.

### RELATED APPLICATIONS

This application claims priority to provisional patent applications U.S. Ser. No. 60/274,281, filed on Mar. 8, 2001; U.S. Ser. No. 60/288,148, filed on May 2, 2001; U.S. Ser. No. 60/274,849, filed on Mar. 9, 2001; U.S. Ser. No. 60/275,235, filed on Mar. 12, 2001; U.S. Ser. No. 60/338,375, filed on Dec. 4, 2001; U.S. Ser. No. 60/275,579, filed on Mar. 13, 2001; U.S. Ser. No. 60/335,302, filed on Oct. 31, 2001; U.S. Ser. No. 60/275,601; filed on Mar. 13, 2001; U.S. Ser. No. 60/276,000, filed on Mar. 14, 2001; U.S. Ser. No. 60/277,338, filed on Mar. 20, 2001; U.S. Ser. No. 60/277,239, filed on Mar. 20, 2001; U.S. Ser. No. 60/277,327, filed on Mar. 20, 2001; U.S. Ser. No. 60/294,821, filed on May 31, 2001; U.S. Ser. No. 60/277,791, filed on Mar. 21, 2001; U.S. Ser. No. 60/277,833, filed on Mar. 22, 2001; U.S. Ser. No. 60/278,152, filed on Mar. 23, 2001; U.S. Ser. No. 60/278,894, filed on Mar. 26, 2001; U.S. Ser. No. 60/279,036, filed on Mar. 27, 2001; U.S. Ser. No. 60/279,344, filed on Mar. 28, 2001; U.S. Ser. No. 60/280,233, filed on Mar. 30, 2001; U.S. Ser. No. 60/280,802, filed on Apr. 2, 2001; each of which is incorporated herein by reference in its entirety.

### FIELD OF THE INVENTION

The present invention is based in part on nucleic acids encoding proteins that are new members of the following protein families: RET finger-like proteins, RNA Polymerase I transcription factor Rrn3-like proteins, CG122292-like proteins, myosin VIIA-like proteins, cytoplasmic protein-like proteins, ankyrin repeat-containing protein-like proteins, WD40 repeat-containing protein-like proteins, zinc finger-containing protein-like proteins, nuclear protein NOP2-like proteins, intracellular protein-like proteins, HBV PX-associated protein 8-like proteins, SM-20-like proteins, synaptonemal complex protein 3-like proteins, paraneoplastic cancer-testis-brain antigen-like proteins, adenylate cyclase associated protein-like proteins, mitochondrial protein-like proteins, PRO2032-like proteins, Leman coiled-coil protein-like proteins, Pax8-like proteins, GTPase activating protein-like proteins, and F-box leucine rich protein-like proteins.

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The present invention is based in part on nucleic acids encoding proteins that are members of the following protein families: RET finger-like proteins, RNA Polymerase I transcription factor Rrn3-like proteins, CG122292-like proteins, myosin VIIA-like proteins, cytoplasmic protein-like proteins, ankyrin repeat-containing protein-like proteins, WD40 repeat-containing protein-like proteins, zinc finger-containing protein-like proteins, nuclear protein NOP2-like proteins, intracellular protein-like proteins, HBV PX-associated protein 8-like proteins, SM-20-like proteins, synaptonemal complex protein 3-like proteins, paraneoplastic cancer-testis-brain antigen-like proteins, adenylate cyclase associated protein-like proteins, mitochondrial protein-like proteins, PRO2032-like proteins, Leman coiled-coil protein-like proteins, Pax8-like proteins, GTPase activating protein-like proteins, and F-box leucine rich protein-like proteins. The novel polynucleotides and polypeptides are referred to herein as NOV1a, NOV1b, NOV2, NOV3, NOV4, NOV5, NOV6a, NOV6b, NOV7, NOV8, NOV9, NOV10a, NOV10b, NOV11, NOV 12, NOV13a, NOV13b, NOV14, NOV15, NOV16, NOV17a, NOV17b, NOV18, NOV19, NOV20, NOV21, NOV22, NOV23, NOV24, NOV25, NOV26, NOV27 and NOV28. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NO:2n-1, wherein n is an integer between 1 and 33. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NO:2n, wherein n is an integer between 1 and 33. The nucleic acid can be, for example, a genomic DNA fragment or a cDNA molecule that includes the nucleic acid sequence of any of SEQ ID NO:2n-1, wherein n is an integer between 1 and 33.

Also included in the invention is an oligonucleotide, e.g., an oligonucleotide which includes at least 6 contiguous nucleotides of a NOVX nucleic acid (e.g., SEQ ID NO:2n-1, wherein n is an integer between 1 and 33) or a complement of said oligonucleotide. Also included in the invention are substantially purified NOVX polypeptides (SEQ ID NO:2n, wherein n is an integer between 1 and 33). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., adrenoleukodystrophy, congenital adrenal hyperplasia, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, cirrhosis, transplantation, systemic lupus erythematosus, autoimmune disease, asthma, emphysema, scleroderma, allergy, adult respiratory distress syndrome (ARDS), lymphedema, allergies, hemophilia, hypercoagulation, idiopathic thrombocytopenic purpura, autoimmune disease, allergies, immunodeficiencies, transplantation, graft versus host disease (GVHD), lymphedema, fertility, diabetes, pancreatitis, obesity, hemophilia, hypercoagulation, idiopathic thrombocytopenic purpura, immunodeficiencies, graft versus host, hypercalcemia, ulcers, anemia, ataxia-telangiectasia, autoimmune disease, immunodeficiencies, cancer, trauma, regeneration (in vitro and in vivo), viral infections, bacterial infections, parasitic infections and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
Sequences and Corresponding SEQ ID Numbers

| | | SEQ ID NO | | |
|---|---|---|---|---|
| NOVX | Internal | (nucleic | SEQ ID NO | |
| Assignment | Identification | acid) | (polypeptide) | Homology |
| 1a | CG57883-01 | 1 | 2 | RET finger |
| 1b | CG57883-02 | 3 | 4 | RET finger |
| 2 | CG57881-01 | 5 | 6 | RET finger |
| 3 | CG58596-01 | 7 | 8 | RNA Polymerase I transcription factor |
| | | | | Rrn3 |
| 4 | CG57407-01 | 9 | 10 | CG122292; DUF6 domain |
| 5 | CG57770-01 | 11 | 12 | Myosin VIIA |
| 6a | CG59233-01 | 13 | 14 | Predicted cytoplasmic protein |
| 6b | CG59233-02 | 15 | 16 | Predicted cytoplasmic protein |
| 7 | CG58649-01 | 17 | 18 | Ankyrin repeat-containing protein |
| 8 | CG58645-01 | 19 | 20 | WD40 repeat-containing protein |
| 9 | CG58632-01 | 21 | 22 | Zinc finger-containing protein |
| 10a | CG58630-01 | 23 | 24 | Nuclear protein NOP2 |
| 10b | CG58630-02 | 25 | 26 | Nuclear protein NOP2 |
| 11 | CG59373-01 | 27 | 28 | Hypothetical intracellular protein |
| 12 | CG57703-01 | 29 | 30 | HBV PX-associated protein 8 |
| 13a | CG58651-01 | 31 | 32 | SM-20 |
| 13b | CG58651-02 | 33 | 34 | SM-20 |
| 14 | CG59574-01 | 35 | 36 | Synaptonemal complex protein 3 |
| 15 | CG59536-01 | 37 | 38 | paraneoplastic cancer-testis-brain |
| | | | | antigen |
| 16 | CG59299-01 | 39 | 40 | adenylate cyclase associated protein |
| 17a | CG59632-01 | 41 | 42 | expressed mitochondrial protein, RIKEN |
| 17b | CG59632-02 | 43 | 44 | expressed mitochondrial protein, RIKEN |
| 18 | CG59653-01 | 45 | 46 | expressed cytoplasmic protein |
| 19 | CG59303-01 | 47 | 48 | mitochondrial protein |
| 20 | CG59673-01 | 49 | 50 | PRO2032 |
| 21 | CG59636-01 | 51 | 52 | Leman coiled-coil protein |
| 22 | CG59675-01 | 53 | 54 | Pax8 |
| 23 | CG59719-01 | 55 | 56 | GTPase activating protein |
| 24 | CG59777-01 | 57 | 58 | GTPase activating protein |
| 25 | CG59658-01 | 59 | 60 | F-box leucine rich protein |
| 26 | CG59907-01 | 61 | 62 | GTPase activating protein |
| 27 | CG59903-01 | 63 | 64 | nuclear protein; KIAA1509 |
| 28 | CG59985-01 | 65 | 66 | WD40 repeat protein |

Table 1 indicates homology of NOVX nucleic acids to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table 1, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., a variety of cancers.

Additional utilities for NOVX nucleic acids and polypeptides according to the invention are disclosed herein.

NOV1 and NOV2: RET Finger-like

NOV1 and NOV2 are homologous to the Ring finger protein family. Thus, NOV1 and NOV2 will function similarly to other members of the Ring finger protein family. This family has transcriptional regulatory activity and is also found in the cytoplasm. Members of this family (e.g., RET finger proteins) can also regulate protein degradation through the ubiquitin pathway. A number of RET finger proteins are involved in oncogenic transformation as well as normal developmental processes. Thus, NOV1 and NOV2 proteins will play a role in normal development as well as dedifferentiation. Specifically, the NOV 1 and NOV2 nucleic acids and proteins of the invention have applications in the diagnosis and/or treatment of various diseases and disorders. For example, the compositions of the present invention will have efficacy for the treatment of patients suffering from: cancer, trauma, regeneration (in vitro and in vivo), viral infections, bacterial infections, parasitic infections, obesity, diabetes and other metabolic diseases as well as other diseases, disorders and conditions.

NOV3: RNA Polymerase I Transcription Factor Rrn3-like

NOV3 is homologous to the RNA polymerase I transcription factor Rrn3 protein family. Thus, NOV3 will function similarly to other members of the Rrn3 protein family. This family mediates transcription of rDNA by RNA polymerase I (Pol I) and is also found in the nucleus. Members of this family are essential genes. Thus, NOV3 nucleic acids and proteins of the invention have applications in the diagnosis and/or treatment of various diseases and disorders. For example, the compositions of the present invention will have efficacy for the treatment of patients suffering from: neurodegenerative disease, Alzheimer, Hodgkin's disease, perinatal asphyxia, systemic sclerosis (scleroderma), pituitary tumor, hepatocellular carcinoma and other malignancies, systemic lupus erythematosus, rheumatic autoimmune diseases, chronic leukemia as well as other diseases, disorders and conditions.

NOV4: CG122292-like

NOV4 is homologous to IPR000620 CG12292 protein, a member of integral membrane protein DUF6 family. Thus, NOV4 will function similarly to other members of the DUF6 subfamily of CG122229-like proteins. The DUF6 family includes the Erwinia PecM protein, which is involved in pectinase, cellulase and blue pigment regulation. NOV4 nucleic acids and proteins of the invention have applications in the diagnosis and/or treatment of various diseases and disorders such as cancer (e.g., pancreatic adenocarcinoma).

NOV5: Myosin VIIA-like

NOV5 is homologous to the Myosin VII protein family. Thus, NOV5 will function similarly to other members of the Myosin VII protein family. Among the members of the Myosin VII protein family are the microtubule-based kinesin motors and actin-based myosin motors generate motions, associated with intracellular trafficking, cell division, and muscle contraction. Thus, NOV5 proteins will play a role in muscle function. NOV5 nucleic acids and proteins of the invention have applications in the diagnosis and/or treatment of various diseases and disorders. For example, the compositions of the present invention will have efficacy for the treatment of patients suffering from: Duchenne's Muscular Dystrophy, deafness, blindness, Inflammatory bowel disease, Diverticular disease, Hyperthyroidism and Hypothyroidism, Lymphedema, Allergies as well as other diseases, disorders and conditions.

NOV6: Predicted Cytoplasmic Protein

NOV6 is an expressed human cytoplasmic protein with homology to an uncharacterized but expressed mouse protein. This human homolog is expressed in numerous human tissues, and will have implications in human diseases. NOV6 nucleic acids and proteins of the invention have applications in the diagnosis and/or treatment of various diseases and disorders. For example, the compositions of the present invention will have efficacy for the treatment of patients suffering from: adrenoleukodystrophy, congenital adrenal hyperplasia, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, cirrhosis, transplantation, systemic lupus erythematosus, autoimmune disease, asthma, emphysema, scleroderma, allergy, adult respiratory distress syndrome (ARDS), lymphedema, allergies, hemophilia, hypercoagulation, idiopathic thrombocytopenic purpura, autoimmune disease, allergies, immunodeficiencies, transplantation, graft versus host disease (GVHD), lymphedema, fertility, diabetes, pancreatitis, obesity, hemophilia, hypercoagulation, idiopathic thrombocytopenic purpura, immunodeficiencies, graft versus host, hypercalcemia, ulcers, anemia, ataxia-telangiectasia, autoimmune disease, immunodeficiencies, cancer, trauma, regeneration (in vitro and in vivo), viral infections, bacterial infections, parasitic infections, as well as other diseases, disorders and conditions.

NOV7: Ankyrin Repeat-containing Protein-like

NOV7 is homologous to the Ankyrin repeat-containing protein family. Thus, NOV7 will function similarly to other members of the Ankyrin repeat-containing protein family. NOV7 protein of the present invention contains ankyrin repeats involved in protein-protein interaction and, thus, will regulate intracellular signal transduction. This domain can also regulate expression/activity of other proteins and /domains thereby affecting cell growth, proliferation, differentiation and survival. NOV7 nucleic acids and proteins of the invention, therefore, have applications in the diagnosis and/or treatment of various diseases and disorders. For example, the compositions of the present invention will have efficacy for the treatment of patients suffering from: cancer, trauma, tissue regeneration (in vitro and in vivo), viral infections, bacterial infections, parasitic infections, immunological disease, respiratory disease, gastro-intestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders as well as other diseases, disorders and conditions.

NOV8: WD40 Repeat-containing Protein-like

NOV8 is homologous to the WD40 repeat-containing protein family. Thus, NOV8 will function similarly to other members of the WD40 repeat-containing protein family. NOV7 of the present invention contains WD40 repeats, a domain has been shown to be involved in protein-protein interactions and signal transduction. WD-repeat proteins are found in all eukaryotes and are implicated in a variety of regulatory functions as a result of protein-protein interactions (e.g., detoxification). Thus, NOV7 nucleic acids and proteins of the invention have applications in the diagnosis and/or treatment of various diseases and disorders. For example, the compositions of the present invention will have efficacy for the treatment of patients suffering from: cancer, trauma, tissue regeneration (in vitro and in vivo), viral infections, bacterial infections, parasitic infections, immunological disease, respiratory disease, gastro-intestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders as well as other diseases, disorders and conditions.

NOV9: Zinc Finger-containing Protein-like

NOV9 is homologous to the Zinc finger-containing protein family. Thus, NOV9 will function similarly to other members of the Zinc finger-containing protein family. NOV9 polypeptide of the present invention contains multiple zinc finger domains of the C2H2 type. These domains are mostly found in transcription factors that regulate gene expression in specific tissues.

NOV9 nucleic acids and proteins of the invention have applications in the diagnosis and/or treatment of various diseases and disorders. NOV9 nucleic acid and protein can be used for targeting genes expressed in specific tissues because of the specificity of the members of this family. The compositions of the present invention will also have efficacy for the treatment of patients suffering from: cancer, trauma, regeneration (in vitro and in vivo), viral infections, bacterial infections, parasitic infections, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, hemophilia, hypercoagulation, idiopathic thrombocytopenic purpura, autoimmune disease, allergies, immunodeficiencies, transplantation, graft versus host disease, hypercalcemia, ulcers, pancreatitis, Hirschsprung's disease, Crohn's Disease, appendicitis, fertility, systemic lupus erythematosus, autoimmune disease, asthma, emphysema, scleroderma, allergy, adult respiratory distress syndrome (ARDS), adrenoleukodystrophy, congenital adrenal hyperplasia as well as other diseases, disorders and conditions.

NOV10: Nuclear Protein NOP2-like Proteins

NOV10 is homologous to the NOP2 protein family and the p120 protein family. In yeast, NOP2 protein is a nucleolar protein that is plays an important role in maintaining the structure of the nucleolus and is essential for yeast cell proliferation. In humans, protein p120, is a cell proliferation marker that has been used as prognostic marker for stages of cancer. Thus, NOV10 of the present invention will play a role in cell proliferation and/or loss of expression associated with lack of cell integrity and apoptosis. NOV10 nucleic acids and proteins of the invention have applications in the diagnosis and/or treatment of various diseases and disorders. For example, the compositions of the present invention will have efficacy for the treatment of patients suffering from: cancer, trauma, regeneration (in vitro and in vivo), viral infections, bacterial infections, parasitic infections, autoimmune disease, asthma, emphysema, scleroderma, allergy, adult respiratory distress syndrome (ARDS), endocrine dysfunctions, diabetes, obesity, growth and reproductive disorders, Von Hippel-Lindau (VHL) syndrome, pancreatitis as well as other diseases, disorders and conditions.

NOV11: Intracellular Protein-like

NOV11 is homologous to bacterial intracellular proteins. NOV11 nucleic acids and proteins of the invention have applications in the diagnosis and/or treatment of various diseases and disorders. For example, the compositions of the present invention will have efficacy for the treatment of patients suffering from various disorders and conditions.

NOV12: HBV PX-associated Protein 8-like

NOV12 is homologous to the HBV PX-associated protein family. Thus, NOV12 will function similarly to other members of the HBV PX-associated protein family. HBx/pX is implicated in the development of hepatocellular carcinoma (HCC) in chronic HBV-infected patients. NOV12 nucleic acids and proteins of the invention have applications in the diagnosis and/or treatment of various diseases and disorders. For example, measurement of NOV12 expression will be useful in the diagnosis of cancer. Also, the compositions of the present invention will have efficacy for the treatment of patients suffering from: liver diseases such as acute hepatitis, hepatitis B, liver transplantation, liver cirrhosis, Von Hippel-Lindau (VHL) syndrome and hepatocarcinoma as well as other diseases, disorders and conditions.

NOV13: SM-20-like

NOV13 is homologous to the SM-20 protein family. Thus, NOV13 will function similarly to other members of the SM-20 protein family. SM-20 protein is a growth factor responsive protein that was first found expressed in blood vessels. SM-20 is also hypothesized to be a mitochondrial protein that promotes cell death through a caspase-dependent mechanism in neurons. NOV13 nucleic acids and proteins of the invention have applications in the diagnosis and/or treatment of various diseases and disorders. For example, the compositions of the present invention will have efficacy for the treatment of patients suffering from: cancer, trauma, tissue regeneration (in vitro and in vivo), viral infections, bacterial infections, parasitic infections, immunological disease, respiratory disease, gastrointestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders as well as other diseases, disorders and conditions.

NOV14: Synaptonemal Complex Protein 3-like

NOV14 is homologous to the Synaptonemal complex protein 3 (SCP3) family. Thus, NOV14 will function similarly to other members of the SCP3 family. SCP3 is responsible for synapsis of homologous chromosomes during meiosis. NOV14 nucleic acids and proteins of the invention have applications in the diagnosis and/or treatment of various diseases and disorders. For example, measurement of NOV14 expression will be useful in the diagnosis of cancer (e.g., testicular cancer) or infertility. Also, the compositions of the present invention will have efficacy for the treatment of patients suffering from: Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, fertility disorders, Hirschsprung's disease, Crohn's disease, appendicitis, diabetes, autoimmune disease, renal artery stenosis, interstitial nephritis, glomerulonephritis, polycystic kidney disease, systemic lupus erythematosus, renal tubular acidosis, IgA nephropathy, cancer, tissue degeneration, bacterial/viral/parasitic infections as well as other diseases, disorders and conditions.

NOV15: Paraneoplastic Cancer-testis-brain Antigen-like

NOV15 is homologous to the paraneoplastic cancer-testis-brain antigen-like protein family. Thus, NOV15 will function similarly to other members of the paraneoplastic cancer-testis-brain antigen-like protein family. Paraneoplastic syndromes are associated with certain types of cancer, such as cancer of the brain or testis. NOV15 nucleic acids and proteins of the invention have applications in the diagnosis and/or treatment of various diseases and disorders. For example, measurement of NOV115 expression will be useful in the diagnosis of neurological syndromes associated with different cancers, such as cancer of the brain or testis. Also, the compositions of the present invention will have efficacy for the treatment of patients suffering from: cancer, trauma, regeneration, viral/bacterial/parasitic infection, systemic lupis erythematosus, autoimmune disease, asthma, emphysema, scleroderma, allergies, and adult respiratory distress syndrome (ARDS), as well as other diseases, disorders and conditions.

NOV16: Adenylate Cyclase Associated Protein-like

NOV16 is homologous to the adenylate cyclase associated protein family. Thus, NOV16 will function similarly to other members of the adenylate cyclase associated protein family. Adenylate cyclase associated proteins regulate actin cytoskeletal organization and are components of the ras signaling pathway. NOV16 nucleic acids and proteins of the invention have applications in the diagnosis and/or treatment of various diseases and disorders. For example, measurement of NOV16 expression will be useful in the diagnosis of cancer, neurological disorders and other diseases involving cell proliferation and/or differentiation. Also, the compositions of the present invention will have efficacy for the treatment of patients suffering from: brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, emphysema, polycystic kidney disease, cystic fibrosis, and cancer; pancreatic disorders including pancreatic insufficiency and cancer; and prostate disorders including prostate cancer as well as other diseases, disorders and conditions.

NOV17: Expressed Mitochondrial Protein-like

NOV17 is homologous to the expressed mitochondrial protein family. Thus, NOV17 will function similarly to other members of the expressed mitochondrial protein family. Mitochondrial proteins are important in metabolism, aging and apoptosis. NOV17 nucleic acids and proteins of the invention have applications in the diagnosis and/or treatment of various diseases and disorders. For example, measurement of NOV17 expression will be useful in the diagnosis of metabolic diseases and/or mitochondrial storage diseases. Also, the compositions of the present invention will have efficacy for the treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, inflammatory bowel disease, diverticular disease, fertility, systemic lupus erythematosus, autoimmune disease, asthma, emphysema, scleroderma, allergy, adult respiratory distress syndrome (ARDS), Von Hippel-Lindau (VHL) syndrome, cirrhosis, transplantation, myasthenia gravis, leukodystrophies, pain, neuroprotection, cancer, trauma, regeneration (in vitro and in vivo), viral infections, bacterial infections, parasitic infections as well as other diseases, disorders and conditions.

NOV18: Expressed Cytoplasmic Protein-like

NOV18 is homologous to the expressed cytoplasmic protein family. Thus, NOV18 will function similarly to other members of the expressed cytoplasmic protein family. Cytoplasmic proteins are important in cell metabolism, signaling, proliferation and differentiation. NOV18 nucleic acids and proteins of the invention have applications in the diagnosis and/or treatment of various diseases and disorders. For example, measurement of NOV18 expression will be useful in the diagnosis of diseases associated with altered expression of cytoplasmic proteins. Also, the compositions of the present invention will have efficacy for the treatment of patients suffering from: anemia, ataxia-telangiectasia, autoimmune disease, immunodeficiencies, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, cirrhosis, transplantation, systemic lupus erythematosus, autoimmune disease, asthma, emphysema, scleroderma, allergy, adult respiratory distress syndrome (ARDS), endometriosis, fertility, diabetes, pancreatitis, obesity, hypercalcemia, ulcers, tonsillitis, endometriosis, cancer, trauma, regeneration (in vitro and in vivo), viral infections, bacterial infections, parasitic infections as well as other diseases, disorders and conditions.

NOV19: Mitochondrial Protein-like

NOV19 is homologous to the mitochondrial protein family. Thus, NOV19 will function similarly to other members of the mitochondrial protein family. Mitochondrial proteins are important in metabolism, aging and apoptosis. NOV19 nucleic acids and proteins of the invention have applications in the diagnosis and/or treatment of various diseases and disorders. For example, measurement of NOV19 expression will be useful in the diagnosis of mitochondrial storage diseases or metabolic disorders. Also, the compositions of the present invention will have efficacy for the treatment of patients suffering from: brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, emphysema, polycystic kidney disease, cystic fibrosis, and cancer; pancreatic disorders including pancreatic insufficiency and cancer; and prostate disorders including prostate cancer as well as other diseases, disorders and conditions.

NOV20: PRO2032-like

NOV20 is homologous to the PRO2032 family. Thus, NOV20 will function similarly to other members of the PRO2032 family. The gene encoding the PRO2032 protein maps to human chromosome 13q31-32, a locus associated with congenital microcoria. NOV20 nucleic acids and proteins of the invention have applications in the diagnosis and/or treatment of various diseases and disorders. For example, measurement of NOV20 expression will be useful in the diagnosis of congenital microcoria. Also, the compositions of the present invention will have efficacy for the treatment of patients suffering from: adrenoleukodystrophy, congenital adrenal hyperplasia, osteoporosis, hypercalcemia, arthritis, ankylosing spondylitis, scoliosis, hemophilia, hypercoagulation, idiopathic thrombocytopenic purpura, autoimmune disease, allergies, immunodeficiencies, transplantation, graft versus host disease, Von Hippel-Lindau (VHL) syndrome, Alzheimer's disease, stroke, tuberous sclerosis, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, renal artery stenosis, interstitial nephritis, glomerulonephritis, polycystic kidney disease, systemic lupus erythematosus, renal tubular acidosis, IgA nephropathy, cirrhosis, asthma, emphysema, scleroderma, adult respiratory distress syndrome (ARDS), lymphedema, hyperparathyroidism, hypoparathyroidism, xerostomia, endocrine dysfunctions, diabetes, obesity, growth and reproductive disorders, hyperthyroidism, hypothyroidism, tonsillitis, endometriosis, fertility, cancer as well as other diseases, disorders and conditions.

NOV21: Leman Coiled-coil Protein-like

NOV21 is homologous to the Leman coiled-coil protein family. Thus, NOV21 will function similarly to other members of the Leman coiled-coil protein family. Extracellular matrix proteins such as keratins may belong to the superfamily of intermediate filament proteins that form alpha-helical coiled-coil dimers which associate laterally and end-to-end to form multimeric filaments. Mutations that perturb keratin filament assembly in vitro can cause blistering human skin disorders in vivo. NOV21 nucleic acids and proteins of the invention have applications in the diagnosis and/or treatment of various diseases and disorders. For example, measurement of NOV21 expression will be useful in the diagnosis of human skin diseases. Also, the compositions of the present invention will have efficacy for the treatment of patients suffering from: diseases of the musculoskeletal system, cancer, connective tissue disorders, heart diseases, Alzheimer's disease, abnormal wound healing, disorders of the skin as well as other diseases, disorders and conditions.

NOV22: Pax8-like

NOV22 is homologous to the Pax8 family. Thus, NOV22 will function similarly to other members of the Pax8 family. Pax8 is a member of a family of transcription factors that are essentially required for the formation of several tissues from all germ layers in the mammalian embryo. In the thyroid gland, PAX8 is essential for the formation of thyroxine-producing follicular cells. NOV22 nucleic acids and proteins of the invention have applications in the diagnosis and/or treatment of various diseases and disorders. For example, measurement of NOV22 expression will be useful in the diagnosis of hyper/hypothyroidism. Also, the compositions of the present invention will have efficacy for the treatment of patients suffering from: thyroid diseases, developmental defects, cancer, especially thyroid carcinomas as well as other diseases, disorders and conditions.

NOV23 and NOV24: GTPase Activating Protein-like

NOV23 and NOV24 are homologous to the GTPase activating protein (GAP) family. Thus, NOV23 and NOV24 will function similarly to other members of the GAP family. The signaling pathway including the guanine nucleotide-binding proteins and the GAPs regulate a variety of processes, including sensual perception, protein synthesis, various transport processes, and cell growth and differentiation. NOV23 and NOV24 nucleic acids and proteins of the invention have applications in the diagnosis and/or treatment of various diseases and disorders. For example, measurement of NOV23 or NOV24 expression will be useful in the diagnosis of cancer. Also, the compositions of the present invention will have efficacy for the treatment of patients suffering from: adrenoleukodystrophy, congenital adrenal hyperplasia, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, arthritis, tendinitis, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, cirrhosis, transplantation, systemic lupus erythematosus, autoimmune disease, asthma, emphysema, scleroderma, allergy, adult respiratory distress syndrome (ARDS), lymphedema, allergies, hemophilia, hypercoagulation, idiopathic thrombocytopenic purpura, autoimmune disease, allergies, immunodeficiencies, transplantation, graft versus host disease (GVHD), lymphedema, fertility, endometriosis, pancreatitis, obesity, hyperparathyroidism, hypoparathyroidism, endocrine dysfunctions, diabetes, obesity, growth and reproductive disorders, inflammatory bowel disease, diverticular disease, hemophilia, hypercoagulation, idiopathic thrombocytopenic purpura, immunodeficiencies, graft versus host, hypercalcemia, ulcers, tonsillitis, cancer, trauma, regeneration (in vitro and in vivo), viral infections, bacterial infections, parasitic infections as well as other diseases, disorders and conditions.

NOV25: F-box Leucine Rich Protein-like

NOV25 is homologous to the F-box leucine rich protein family. Thus, NOV25 will function similarly to other members of the F-box leucine rich protein family. F-box proteins are critical components of the SCF ubiquitin-protein ligase complex and are involved in substrate recognition and recruitment for ubiquitination and consequent degradation by the proteasome. NOV25 nucleic acids and proteins of the invention have applications in the diagnosis and/or treatment of various diseases and disorders. For example, measurement of NOV25 expression will be useful in the diagnosis of lysosomal and other organelle storage diseases. Also, the compositions of the present invention will have efficacy for the treatment of patients suffering from: Adrenoleukodystrophy, Congenital Adrenal Hyperplasia, Hemophilia, hypercoagulation, idiopathic thrombocytopenic purpura, autoimmune disease, allergies, immunodeficiencies, transplantation, Graft versus host, Von Hippel-Lindau (VHL) syndrome, Alzheimer's disease, Stroke, Tuberous sclerosis, hypercalcemia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Lymphedema, Allergies, Psoriasis, Actinic keratosis, Acne, Hair growth, allopecia, pigmentation disorders, endocrine disorders, Endocrine dysfunctions, Diabetes, obesity, Growth and reproductive disorders, Fertility, Endometriosis, Hemophilia, Hypercoagulation, idiopathic thrombocytopenic purpura, Immunodeficiencies, Graft versus host, Hyperthyroidism and Hypothyroidism as well as other diseases, disorders and conditions.

NOV26: GTPase Activating Protein-like

NOV26 is homologous to the GTPase activating protein (GAP) family. Thus, NOV26 will function similarly to other members of the GAP family. The signaling pathway including the guanine nucleotide-binding proteins and the GAPs regulate a variety of processes, including sensual perception, protein synthesis, various transport processes, and cell growth and differentiation. NOV26 nucleic acids and proteins of the invention have applications in the diagnosis and/or treatment of various diseases and disorders. For example, measurement of NOV26 expression will be useful in the diagnosis of cancer. Also, the compositions of the present invention will have efficacy for the treatment of patients suffering from: adrenoleukodystrophy, congenital adrenal hyperplasia, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, arthritis, tendinitis, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, cirrhosis, transplantation, systemic lupus erythematosus, autoimmune disease, asthma, emphysema, scleroderma, allergy, ARDS, lymphedema, allergies, hemophilia, hypercoagulation, idiopathic thrombocytopenic purpura, autoimmune disease, allergies, immunodeficiencies, transplantation, graft versus host disease (GVHD), lymphedema, fertility, endometriosis, pancreatitis, obesity, hyperparathyroidism, hypoparathyroidism, endocrine dysfunctions, diabetes, obesity, growth and reproductive disorders, inflammatory bowel disease, diverticular disease, hemophilia, hypercoagulation, idiopathic thrombocytopenic purpura, immunodeficiencies, graft versus host, hypercalcemia, ulcers, tonsillitis, cancer, trauma, regeneration (in vitro and in vivo), viral infections, bacterial infections, parasitic infections as well as other diseases, disorders and conditions.

NOV27: Nuclear Protein-like

NOV27 is homologous to the nuclear protein family. Thus, NOV27 will function similarly to other members of the nuclear protein family. Nuclear proteins are important in gene expression, cell cycle regulation, intracellular trafficking and cell shape. NOV27 nucleic acids and proteins of the invention have applications in the diagnosis and/or treatment of various diseases and disorders. The compositions of the present invention will have efficacy for the treatment of patients suffering from: adrenoleukodystrophy, congenital adrenal hyperplasia, anemia, ataxia-telangiectasia, autoimmune disease, immunodeficiencies, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, systemic lupus erythematosus, autoimmune disease, asthma, emphysema, scleroderma, allergy, ARDS, lymphedema, allergies, hemophilia, hypercoagulation, idiopathic thrombocytopenic purpura, autoimmune disease, allergies, immunodeficiencies, transplantation, graft versus host disease (GVHD), lymphedema, fertility, endometriosis, fertility, diabetes, Von Hippel-Lindau (VHL) syndrome, pancreatitis, obesity, endocrine dysfunctions, diabetes, obesity, growth and reproductive disorders, hemophilia, hypercoagulation, idiopathic thrombocytopenic purpura, immunodeficiencies, graft versus host, hemophilia, hypercoagulation, idiopathic thrombocytopenic purpura, immunodeficiencies, hyperthyroidism and hypothyroidism, cystitis, incontinence, endometriosis, cancer, trauma, regeneration (in vitro and in vivo), viral infections, bacterial infections, parasitic infections as well as other diseases, disorders and conditions.

NOV28: WD40 Repeat Protein-like

NOV28 is homologous to the WD40 repeat protein family. Thus, NOV28 will function similarly to other members of the WD40 repeat protein family. The WD40 repeat consists of about 40 residues, each containing a central Trp-Asp motif. The WD40 repeat is found in beta-transducin (G-beta) and G-beta-like peptides, yeast STE4, MSI1, CDC4, CDC20, MAK11, PRP4, PWP1 and TUP1, slime-mould AAC3 and coronin, and Drosophila Groucho protein. WD40 repeat-containing proteins are involved in cell cycle progression and cell differentiation. NOV28 nucleic acids and proteins of the invention have applications in the diagnosis and/or treatment of various diseases and disorders. For example, measurement of NOV28 expression will be useful in the diagnosis of defective cell differentiation such as in cancer. Also, the compositions of the present invention will have efficacy for the treatment of patients suffering from: adrenoleukodystrophy, congenital adrenal hyperplasia, anemia, ataxia-telangiectasia, autoimmune disease, immunodeficiencies, hemophilia, hypercoagulation, idiopathic thrombocytopenic purpura, autoimmune disease, allergies, immunodeficiencies, transplantation, graft versus host, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, fertility, atherosclerosis, aneurysm, hypertension, fibromuscular dysplasia, stroke, scleroderma, obesity, transplantation, myocardial infarction, embolism, cardiovascular disorders, bypass surgery, diabetes, tuberous sclerosis, cirrhosis, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, Von Hippel-Lindau (VHL) syndrome, cirrhosis, transplantation, systemic lupus erythematosus, autoimmune disease, asthma, emphysema, scleroderma, allergy, ARDS, lymphedema, allergies, hemophilia, hypercoagulation, idiopathic thrombocytopenic purpura, autoimmune disease, allergies, immunodeficiencies, transplantation, graft versus host disease (GVHD), lymphedema, muscular dystrophy, Lesch-Nyhan syndrome, myasthenia gravis, endometriosis, diabetes, Von Hippel-Lindau (VHL) syndrome, pancreatitis, obesity, endocrine dysfunctions, diabetes, obesity, growth and reproductive disorders, psoriasis, actinic keratosis, tuberous sclerosis, acne, hair growth, allopecia, pigmentation disorders, endocrine disorders, hemophilia, hypercoagulation, idiopathic thrombocytopenic purpura, immunodeficiencies, graft versus host, hypercalcemia, ulcers, hemophilia, hypercoagulation, idiopathic thrombocytopenic purpura, immunodeficiencies, hyperthyroidism and hypothyroidism, cancer, trauma, regeneration (in vitro and in vivo), viral infections, bacterial infections, parasitic infections as well as other diseases, disorders and conditions.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

In one specific embodiment, the invention includes an isolated polypeptide comprising an amino acid sequence selected from the group consisting of: (a) a mature form of the amino acid sequence selected from the group consisting of SEQ ID NO:2n, wherein n is an integer between 1 and 33; (b) a variant of a mature form of the amino acid sequence selected from the group consisting of SEQ ID NO:2n, wherein n is an integer between 1 and 33, wherein any amino acid in the mature form is changed to a different amino acid, provided that no more than 15% of the amino acid residues in the sequence of the mature form are so changed; (c) an amino acid sequence selected from the group consisting of SEQ ID NO:2n, wherein n is an integer between 1 and 33; (d) a variant of the amino acid sequence selected from the group consisting of SEQ ID NO:2n, wherein n is an integer between 1 and 33, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO:2n, wherein n is an integer between 1 and 33; (b) a variant of a mature form of the amino acid sequence selected from the group consisting of SEQ ID NO:2n, wherein n is an integer between 1 and 33, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO:2n, wherein n is an integer between 1 and 33; (d) a variant of the amino acid sequence selected from the group consisting of SEQ ID NO:2n, wherein n is an integer between 1 and 33, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO:2n, wherein n is an integer between 1 and 33, or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO:2n-1, wherein n is an integer between 1 and 33; (b) a nucleotide sequence wherein one or more nucleotides in the nucleotide sequence selected from the group consisting of SEQ ID NO:2n-1, wherein n is an integer between 1 and 33, is changed from that selected from the group consisting of the chosen sequence to a different nucleotide provided that no more than 15% of the nucleotides are so changed; (c) a nucleic acid fragment of the sequence selected from the group consisting of SEQ ID NO:2n-1, wherein n is an integer between 1 and 33; and (d) a nucleic acid fragment wherein one or more nucleotides in the nucleotide sequence selected from the group consisting of SEQ ID NO:2n-1, wherein n is an integer between 1 and 33, is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NO:2n-1, wherein n is an integer between 1 and 33, or a complement of this aforementioned nucleotide sequence, can be isolated using standard molecular biology techniques and the sequence information provided herein. Using all or a portion of the nucleic acid sequence of SEQ ID NO:2n-1, wherein n is an integer between 1 and 33, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NO:2n-1, wherein n is an integer between 1 and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n-1, wherein n is an integer between 1 and 33, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NO:2n-1, wherein n is an integer between 1 and 33is one that is sufficiently complementary to the nucleotide sequence shown SEQ ID NO:2n-1, wherein n is an integer between 1 and 33, that it can hydrogen bond with little or no mismatches to the nucleotide sequence shown SEQ ID NO:2n-1, wherein n is an integer between 1 and 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n-1, wherein n is an integer between 1 and 33, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NO:2n-1, wherein n is an integer between 1 and 33; or an anti-sense strand nucleotide sequence of SEQ ID NO:2n-1, wherein n is an integer between 1 and 33; or of a naturally occurring mutant of SEQ ID NO:2n-1, wherein n is an integer between 1 and 33.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NO:2n-1, wherein n is an integer between 1 and 33, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NO:2n-1, wherein n is an integer between 1 and 33, due to degeneracy of the genetic code and thus encode the same NOVX proteins as that encoded by the nucleotide sequences shown in SEQ ID NO:2n-1, wherein n is an integer between 1 and 33. In another embodiment, an isolated nucleic acid molecule of the invention has a nucleotide sequence encoding a protein having an amino acid sequence shown in SEQ ID NO:2n, wherein n is an integer between 1 and 33.

In addition to the human NOVX nucleotide sequences shown in SEQ ID NO:2n-1, wherein n is an integer between 1 and 33, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NO:2n-1, wherein n is an integer between 1 and 33, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n-1, wherein n is an integer between 1 and 33. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NO:2n-1, wherein n is an integer between 1 and 33, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n-1, wherein n is an integer between 1 and 33, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NO:2n-1, wherein n is an integer between 1 and 33, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NO:2n-1, wherein n is an integer between 1 and 33, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NO:2n, wherein n is an integer between 1 and 33. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n, wherein n is an integer between 1 and 33, yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NO:2n, wherein n is an integer between 1 and 33. Preferably, the protein encoded by the nucleic acid molecule is at least about 60% homologous to SEQ ID NO:2n, wherein n is an integer between 1 and 33; more preferably at least about 70% homologous SEQ ID NO:2n, wherein n is an integer between 1 and 33; still more preferably at least about 80% homologous to SEQ ID NO:2n, wherein n is an integer between 1 and 33; even more preferably at least about 90% homologous to SEQ ID NO:2n, wherein n is an integer between 1 and 33; and most preferably at least about 95% homologous to SEQ ID NO:2n, wherein n is an integer between 1 and 33.

An isolated nucleic acid molecule encoding an NOVX protein homologous to the protein of SEQ ID NO:2n, wherein n is an integer between 1 and 33, can be created by introducing one or more nucleotide substitutions, additions or deletions into the nucleotide sequence of SEQ ID NO:2n-1, wherein n is an integer between 1 and 33, such that one or more amino acid substitutions, additions or deletions are introduced into the encoded protein.

Mutations can be introduced into SEQ ID NO:2n-1, wherein n is an integer between 1 and 33, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis of SEQ ID NO:2n-1, wherein n is an integer between 1 and 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1, wherein n is an integer between 1 and 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NO:2n, wherein n is an integer between 1 and 33, or antisense nucleic acids complementary to an NOVX nucleic acid sequence of SEQ ID NO:2n-1, wherein n is an integer between 1 and 33, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An uαanomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NO:2n-1, wherein n is an integer between 1 and 33). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660:27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NO:2n, wherein n is an integer between 1 and 33. The invention also includes a mutant or variant protein any of whose residues may be changed from the corresponding residues shown in SEQ ID NO:2n, wherein n is an integer between 1 and 33, while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NO:2n, wherein n is an integer between 1 and 33) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NO:2n, wherein n is an integer between 1 and 33. In other embodiments, the NOVX protein is substantially homologous to SEQ ID NO:2n, wherein n is an integer between 1 and 33, and retains the functional activity of the protein of SEQ ID NO:2n, wherein n is an integer between 1 and 33, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NO:2n, wherein n is an integer between 1 and 33, and retains the functional activity of the NOVX proteins of SEQ ID NO:2n, wherein n is an integer between 1 and 33.

Determining Homology between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:2n-1, wherein n is an integer between 1 and 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NO:2n, wherein n is an integer between 1 and 33), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148:2918-2922 (1992). Homodimeric antibodies with enhanced anti-tumor activity can also be prepared using heterobifunctional cross-linkers as described in Wolff et al. Cancer Research, 53:2560-2565 (1993). Alternatively, an antibody can be engineered that has dual Fc regions and can thereby have enhanced complement lysis and ADCC capabilities. See Stevenson et al., Anti-Cancer Drug Design, 3:219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³³In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1 990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20:2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6:229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NO:2n-1, wherein n is an integer between 1 and 33, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NO:2n-1, wherein n is an integer between 1 and 33), but more preferably, is a non-human homologue of a human NOVX gene. For example, a mouse homologue of human NOVX gene of SEQ ID NO:2n-1, wherein n is an integer between 1 and 33, can be used to construct a homologous recombination vector suitable for altering an endogenous NOVX gene in the mouse genome. In one embodiment, the vector is designed such that, upon homologous recombination, the endogenous NOVX gene is functionally disrupted (ie., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991 . Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37:2678; Cho, et al., 1993. Science 261: 1303; Carrell, et al., 1994. Angew. Chem. Int. Ed. Engl. 33:2059; Carell, et al., 1994. Angew. Chem. Int. Ed. Engl. 33:2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, Triton® X-114, Thesit®, decanoyl-N-methylglucamide, Triton® X-200, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72:223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NO:2n-1, wherein n is an integer between 1 and 33, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO:2n-1, wherein n is an integer between 1 and 33, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n-1, wherein n is an integer between 1 and 33, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7:244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86:2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17:2437-2448) or at the extreme 3′-terminus of one primer where, under appropriate conditions, mismatch can prevent, or reduce polymerase extension (see, e.g., Prossner, 1993. Tibtech. 11:238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A. NOVX Clone Information

### Example A1

The NOV1 clone was analyzed, and the nucleotide and predicted polypeptide sequences are shown in Table 1A.

TABLE 1A
NOV1 Sequence Analysis

| | SEQ ID NO:1 | 1046 bP |
|---|---|---|
| NOV1a, | CCATGCATGTAAAGATAAAAGCCCCAAACACTATCAGCTGTTCATTCAGCTCGTGGAA | |
| CG57883-01 | ATTCTAATTCCGTGTTCATTTTTTTTTCTACAGACATTTGCCATGGCTGAGCACTTCA | |
| DNA Sequence | AACAGATCATTAGATGTCCTGTCTGTCTAAAAGATCTTGAAGAAGCCGTGCAACTGAA | |
| | GAAGGTTTACTGTGCCGTTTCTGCTCTGTGGTCTCTCAGAAGGATGACATCAAGCCCA | |
| | AGTACAAGCTGAGGGCGCTGGTTTCCATCATCAAGGAACTAGAGCCCAAGCTGAAATC | |
| | TGTTCTAACAATGAACCCAAGGATGAGGAAGTTTCAAGTGGATATGACGTTCGATGTG | |
| | GACACAGCCAACAACTATCTCATCATTTCTGAAGACCTGAGGAGTTTCCGAAGTGGGG | |
| | ATTTGAGCCAGAATAGGAAGGAGCAAGCTGAGAGGTTCGACACTACCCTGTGCGTCCT | |
| | GGGCACCCCTCGCTTCACTTCCGGCCGCCATTACTGGGAGGTGGACGTGGGAACCAGC | |
| | CAAGTGTCGGATGTGGGCGTGTGCAAGGAATCTGTCAACCGACAGGGGAAGATTGAGC | |
| | TTTCTTCAGAACACGGCTTCTTGACTGTGGGTTGCAGAGAAGGAAAGGTCTTTGCTGC | |
| | CAGCACTGTGCCTATGACTCCTCTCTGGGTGAGTCCCCAGTTGCACAGAGTGGGGATT | |
| | TTCCTGGATGTACGTATGAGGTCCATTGCCTTTTACAATGTTAGTGATGGGTGCCATA | |
| | TCTACACATTCATCGAGATTCCTGTTTGCGAGCCCTGGCGTCCATTTTTTGCTCATAA | |
| | ACGTGGAAGTCAAGATGATCAGAGCATCCTGAGTATCTGTTCTGTGATCAATCCATCC | |
| | ACTGCCAGTGCCCCAGTTTCTTCTGAGGGAAAGTAAATAAACATTTGAACATAATCAT | |
| | CTTTAGGAAGTTTCAGTGCCCCCATAGCCATAGCTAAGAACTTTTCCGCTAGATACAC | |
| | AT | |
| | ORF Start: ATG at 101 | ORF Stop: TAA at 962 |
| | SEQ ID NO:2 | 287 aa MW at 32354.9 kD |
| NOV1a, | MAEHFKQIIRCPVCLKDLEEAVQLKCGYACCLQCLNSLQREPNGEGLLCRFCSVVSQK | |
| CG57883-01 | DDIKPKYKLRALVSIIKELEPKLKSVLTMNPRMRKFQVDMTFDVDTANNYLIISEDLR | |
| Protein | SFRSGDLSQNRKEQAERFDTTLCVLGTPRFTSGRHYWEVDVGTSQAADVGVCKESAAR | |
| Sequence | QGKIELSSEHGFLTVGCREGKVFAASTVPMTPLWVSPQLHRVGIFLDVGMRSIAFYNV | |
| | SDGCHIYTFIEIPVCEPWRPFFAHKRGSQDDQSILSICSVINPSTASAPVSSEGK | |
| | SEQ ID NO:3 | 927 bp |
| NOV 1b, | TGTTCATTCAGCTCGTGGAAATTCTAATTCCGTGTTCATTTTTTTTCTATAGACATTT | |
| CG57883-02 | GCCATGGCTGAGCACTTCAAACAGATCATTAGATGTCCTCTCTGTCTAAAAGATCTTG | |
| DNA Sequence | AAGAAGCCGTGCAACTGAAATGTGGATATGCCTGCTGCCTCCAGTGCCTCAATTCACT | |
| | CCAGAAGGAGCCCGATGGGGAAGGTTTACTGTGCCGTTTCTGCTCTGTGGTCTCTCAG | |
| | AAGGATGACATCAAGCCCAAGTACAAGCTGAGGGCGCTGGTTTCCATCATCAAGGAAC | |
| | TAGAGCCCAAGCTGAAATCTGTTCTAACAATGAACCCAAGGATGAGGAAGTTTCAAGT | |
| | GGATATGACGTTCGATGTGGACACAGCCAACAACTATCTCATCATTTCTGAAGACCTG | |
| | AGGAGTTTCCGAAGTGGGGATTTGAGCCAGAATAGGAAGGAGCAAGCTGAGAGGTTCC | |
| | ACACTGCCCTGTGCGTCCTGGGCACCCCTCGCTTCGCTTCCGGCCGCCATTACTGGGA | |
| | GGTGGACGTGGGCACCAGCCAAGTGTGGGATGTGGGCGTGTGCAAGGAATCTGTGAAC | |
| | CGACAGGGGAAGATTGTGCTTTCTTCAGAACACGGCTTCTTGACTGTGGGTTGCAGAG | |
| | AAGGAAAGGTCTTTGCTGCCAGCACTGTGCCTATGACTCCTCTCTGGGTGAGTCCCCA | |
| | GTTGCACAGAGTGGGGATTTTCCTGGATGTAGGTATGAGGTCCATTGCCTTTTACAAT | |
| | GTTAGTGATGGGTGCCATATCTACACATCCATCGAGATTCCTGTTTGCGAGCCCTGGC | |
| | GTCCATTTTTTGCTCATAAACGTGGAAGTCAAGATGATCAGAGCATCCTGAGTATCTG | |
| | TTCTGTGATCAATCCATCCGCTGCCAGTGCCCCAGTTTCTTCTGAGGGAAAGTAAAT | |
| | ORF Start: ATG at 62 | ORF Stop: TAA at 923 |
| | SEQ ID NO:4 | 287 aa MW at 32161.7 kD |

| | |
|---|---|
| NOV1b, | MAEHFKQIIRCPLCLKDLEEAVQLKCGYACCLQCLNSLQKEPDGEGLLCRFCSVVSQK |
| CG57883-02 | DDIKPKYKLRLVSIIKELEPKLKSVILTMNPRMRKFQAAMTFDVDTAANYLIISEDLR |
| Protein | SFRSGDLSQNRKEQAERFDTALCVLGTPRFASGRHAAEAAVGTSQAADVGVCKESAAR |
| Sequence | QGKIVLSSEHGFLTVGCREGKVFAASTVPMTPLAASPQLHRVGIFLDVGMRSIAFYNV |
| | SDGCHIYTSIEIPVCEPWRPFFAHKRGSQDDQSILSICSVINPSAASAPVSSEGK |

Sequence comparison of the above protein sequences yields the following sequence relationships shown in Table 1B.

TABLE 1B
Comparison of NOV1a against NOV1b.

| | NOV1a Residues/ | Identities/Similarities |
|---|---|---|
| Protein Sequence | Match Residues | for the Matched Region |
| NOV1b | 1 . . . 287 | 279/287 (97%) |
| | 1 . . . 287 | 282/287 (98%) |

Further analysis of the NOV1a protein yielded the following properties shown in Table 1C.

TABLE 1C
Protein Sequence Properties NOV1a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY84899 | A human proliferation and apoptosis related | 1 . . . 282 | 159/282 (56%) | 7e−91 |
| | protein - Homo sapiens, 288 aa. | 1 . . . 282 | 210/282 (74%) | |
| | [WO200023589-A2, 27-APR-2000] | | | |
| AAU14321 | Human novel protein #192 - Homo sapiens, | 1 . . . 282 | 158/282 (56%) | 2e−90 |
| | 317 aa. [WO200155437-A2, 02-AUG-2001] | 30 . . . 311 | 210/282 (74%) | |
| AAB43498 | Human cancer associated protein sequence | 70 . . . 286 | 85/220 (38%) | 1e−37 |
| | SEQ ID NO: 943 - Homo sapiens, 580 aa. | 354 . . . 573 | 122/220 (54%) | |
| | [WO200055350-A1, 21-SEP-2000] | | | |
| ABB20271 | Protein #2270 encoded by probe for | 97 . . . 209 | 70/113 (61%) | 4e−37 |
| | measuring heart cell gene expression - Homo | 1 . . . 113 | 94/113 (82%) | |
| | sapiens, 116 aa. [WO200157274-A2, | | | |
| | 09-AUG-2001] | | | |
| AAM68043 | Human bone marrow expressed probe | 97 . . . 209 | 70/113 (61%) | 4e−37 |
| | encoded protein SEQ ID NO: 28349 - Homo | 1 . . . 113 | 94/113 (82%) | |
| | sapiens, 116 aa. [WO200157276-A2, 09- | | | |
| | AUG-2001] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities | |
| Accession | | Match | for the | Expect |
| Number | Protein/Organism/Length | Residues | Matched Portion | Value |
| O75677 | RET finger protein-like 1 - Homo | 1 . . . 282 | 165/282 (58%) | 8e−96 |
| | sapiens (Human), 288 aa. | 1 . . . 282 | 214/282 (75%) | |
| O75679 | RET finger protein-like 3 - Homo | 1 . . . 282 | 163/282 (57%) | 1e−93 |
| | sapiens (Human), 288 aa. | 1 . . . 282 | 212/282 (74%) | |
| Q9UJ97 | RFPL1S - Homo sapiens (Human), 287 | 1 . . . 282 | 164/282 (58%) | 2e−93 |
| | aa. | 1 . . . 281 | 212/282 (75%) | |
| O75678 | Ret finger protein-like 2 - Homo | 1 . . . 282 | 158/282 (56%) | 6e−90 |
| | sapiens (Human), 288 aa. | 1 . . . 282 | 210/282 (74%) | |
| AAL55432 | RET FINGER PROTETN-LIKE 4 | 1 . . . 285 | 156/287 (54%) | 3e−83 |
| | PROTEIN - Mus musculus (Mouse), | 1 . . . 287 | 202/287 (70%) | |
| | 287 aa. | | | |

PFam analysis predicts that the NOV1a protein contains the domains shown in the Table 1F.

TABLE 1F
Domain Analysis of NOV1a

| | | Identities/ | |
|---|---|---|---|
| | NOV1a | Similarities | |
| | Match | for the | Expect |
| Pfam Domain | Region | Matched Region | Value |
| zf-C3HC4: domain 1 of 1 | 11 . . . 52 | 14/54 (26%) | 0.0015 |
| | | 32/54 (59%) | |
| PHD: domain 1 of 1 | 10 . . . 55 | 15/52 (29%) | 5.2 |
| | | 26/52 (50%) | |
| SPRY: domain 1 of 1 | 148 . . . 273 | 37/157 (24%) | 1.9e−19 |
| | | 87/157 (55%) | |

### Example A2

The NOV2 clone was analyzed, and the nucleotide and predicted polypeptide sequences are shown in Table 2A.

TABLE 2A
NOV2 Sequence Analysis

| | SEQ ID NO:5 | 1010 bp |
|---|---|---|
| NOV2, | ATGTCTTCATGCAGGTAAAGATGAAAGTGTCCCAAGAAC | |
| CG57881- | TATCAGCCATTCCACTCACGTAAAAGCTAATACCATGCC | |
| 01 DNA | TATTTACTCCCAGACAGTGGCCATGGCTGAACACTTTAA | |
| Sequence | ACAAGCAAGCAGTTGTCCTATCTGCCTGGATTATCTTGA | |
| | AAACCCCACGCACCTGAAATGTGGATACATCTGTTGCCT | |
| | CCGATGCATGAACTCACTGCGAAAGGGGCCCGATGGGAA | |
| | GGGGGTGCTGTGCCCTTTCTGCCCTGTGGTCTCTCAGAA | |
| | AAATGACATCAGGCCCGCTGCCCAGCTGGGGGCGCTGGT | |
| | GTCCAAGATCAAGGAACTAGAGCCCAAGGTGAGAGCTGT | |
| | TCTGCAGATGAATCCAAGGATGAGAAAGTTCCAAGTGGA | |
| | TATGACCTTGGATGTGGACACAGCCAACAACGATCTCAT | |
| | CGTTTCTGAAGACCTGAGGCGTGTCCGATGTGGGAATTT | |
| | CAGACAGAATAGGAAGGAGCAAGCTGAGAGGTTCGACAC | |
| | TGCCCTGTGCGTCCTGGGCACCCCTCGCTTCACTTCCGG | |
| | CCGCCATTACTGGGAGGTGGGCGTGGGCACCAGCCAAGT | |
| | GTGGGATGTGGGCGTGTGCAAGGAATCTGTGAACCGACA | |
| | GGGGAACGTTGTACTCTCTTCAGAACTCGGCTTCTGGAC | |
| | TGTGGGTTTGAGACAAGGACAGATCTACTTTGCCAGCAC | |
| | TAAGCCTGTGACGGGTCTCTGGGTGAGCTCAGGTCTACA | |
| | CCGAGTGGGGATTTACCTGGATATAAAAACGAGGGCCAT | |
| | TTCCTTCTATAATGTCAGTGATAGGTCACATATCTTCAC | |
| | ATTCACGAAAATTTCTGCTACTGAGCCACTGCGCCCATG | |
| | TTTTGCTCATGCAGATACAAGTCGTGATGATCACGGATA | |
| | CTTGAGTGTGTGTGTGTAATTAATAATGGCATTGCCAGT | |
| | TCCCCAATTTATCCTGGGCAAGGCAACTATACACTTGAA | |
| | CACAGAAAACATCCACAGTAAGTGGCTGTGTGCTC | |
| | ORF Start: | ORF Stop: TAA at 914 |
| | ATG at 74 | 280 aa |
| | SEQ ID NO:6 | MW at 31431.9 kD |

| | |
|---|---|
| NOV2, | MPIYSQTVAMAEHFKQASSCPICLDYLENPTHLKCGYIC |
| CG57881- | CLRCMNSLRKGPDGKGVLCPFCPVVSQKNDIRPAAQLGA |
| 01 Protein | LVSKIKELEPKVRAVLQMNPRMRKFQVDMTLDVDTANND |
| Sequence | LIVSEDLRRVRCGNFRONRKEOAERFDTALCVLGTPRFT |
| | SGRHYWEVGVGTSQVWDVGVCKESVNRQGNVVLSSELGF |
| | WTVGLRQGQIYFASTKPVTGLWVSSGLHRVGIYLDIKTR |
| | AISFYNVSDRSHIFTFTKISATEPLRPCFAHADTSRDDH |
| | GYLSVCV |

Further analysis of the NOV2 protein yielded the following properties shown in Table 2B.

TABLE 2B
Protein Sequence Properties NOV2

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV2 protein against the Geneseq database, a proprietary database that contains sequences published in patents and patent publication, yielded several homologous proteins shown in Table 2C.

TABLE 2C
Geneseq Results for NOV2

| | | NOV2 | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY84899 | A human proliferation and apoptosis related | 10 . . . 279 | 154/270 (57%) | 7e−88 |
| | protein - Homo sapiens, 288 aa. | 1 . . . 270 | 198/270 (73%) | |
| | [WO200023589-A2, 27-APR-2000] | | | |
| AAU14321 | Human novel protein #192 - Homo sapiens, | 8 . . . 279 | 154/272 (56%) | 1e−87 |
| | 317 aa. [WO200155437-A2, 02-AUG-2001] | 28 . . . 299 | 199/272 (72%) | |
| AAB43498 | Human cancer associated protein sequence | 79 . . . 279 | 83/204 (40%) | 3e−39 |
| | SEQ ID NO: 943 - Homo sapiens, 580 aa. | 354 . . . 557 | 125/204 (60%) | |
| | [WO200055350-A1, 21-SEP-2000] | | | |
| ABB20271 | Protein #2270 encoded by probe for | 106 . . . 218 | 70/113 (61%) | 1e−36 |
| | measuring heart cell gene expression - Homo | 1 . . . 113 | 90/113 (78%) | |
| | sapiens, 116 aa. [WO200157274-A2, 09- | | | |
| | AUG-2001] | | | |
| AAM68043 | Human bone marrow expressed probe | 106 . . . 218 | 70/113 (61%) | 1e−36 |
| | encoded protein SEQ ID NO: 28349 - Homo | 1 . . . 113 | 90/113 (78%) | |
| | sapiens, 116 aa. [WO200157276-A2, 09- | | | |
| | AUG-2001] | | | |

In a BLAST search of public sequence databases, the NOV2 protein was found to have homology to the proteins shown in the BLASTP data in Table 2D.

TABLE 2D
Public BLASTP Results for NOV2

| | | NOV2 | Identities/ | |
|---|---|---|---|---|
| Protein | | Residues/ | Similarities | |
| Accession | | Match | for the | Expect |
| Number | Protein/Organism/Length | Residues | Matched Portion | Value |
| O75679 | RET finger protein-like 3 - Homo | 10 . . . 279 | 157/270 (58%) | 1e−89 |
| | sapiens (Human), 288 aa. | 1 . . . 270 | 199/270 (73%) | |
| O75677 | RET finger protein-like 1 - Homo | 10 . . . 279 | 154/270 (57%) | 7e−88 |
| | sapiens (Human), 288 aa. | 1 . . . 270 | 196/270 (72%) | |
| O75678 | Ret finger protein-like 2 - Homo | 10 . . . 279 | 153/270 (56%) | 6e−87 |
| | sapiens (Human), 288 aa. | 1 . . . 270 | 198/270 (72%) | |
| Q9UJ97 | RFPL1S - Homo sapiens (Human), 287 | 10 . . . 279 | 154/270 (57%) | 1e−85 |
| | aa. | 1 . . . 269 | 194/270 (71%) | |
| AAL55432 | RET FINGER PROTEIN-LIKE 4 | 10 . . . 279 | 149/272 (54%) | 8e−81 |
| | PROTEIN - Mus musculus (Mouse), | 1 . . . 272 | 195/272 (70%) | |
| | 287 aa. | | | |

PFam analysis predicts that the NOV2 protein contains the domains shown in the Table 2E.

TABLE 2E
Domain Analysis of NOV2

| | | Identities/ | |
|---|---|---|---|
| | | Similarities | |
| | NOV2 | for the | Expect |
| Pfam Domain | Match Region | Matched Region | Value |
| zf-C3HC4: | 20 . . . 61 | 18/54 (33%) | 1.3e−05 |
| domain 1 of 1 | | 31/54 (57%) | |
| SPRY: | 157 . . . 272 | 39/157 (25%) | 6.3e−25 |
| domain 1 of 1 | | 92/157 (59%) | |

### Example A3

The NOV3 clone was analyzed, and the nucleotide and predicted polypeptide sequences are shown in Table 3A.

TABLE 3A
NOV3 Sequence Analysis

| | |
|---|---|
| SEQ ID NO:7 | 1770 bp |

| | |
|---|---|
| NOV3, | GGAGCGGCCGCCCAGGTGCGGTCGCGTTAGTTCGGCCCA |
| CG58596-01 | ATGGCGGCACCGCTGCTTCACACGCGTTTGCCGGGAGAT |
| DNA | GCGGCCGCTTCGTCCTCTGCAGTTAAGAAGCTGGGCGCG |
| Sequence | TCGAGGACTGGGATTTCAAATATGCGTGCATTAGAGAAT |
| | GACTTTTTCAATTCTCCCCCAAGAAAAACTGTTCGGTTT |
| | GGTGGAACTGTGACAGAAGTCTTGCTGAAGTACAAAAAG |
| | GGTGAAACAAATGACTTTGAGTTGTTGAAGAACCAGCTG |
| | TTAGATCCAGACATAAAGGATGACCAGATCATCAACTGG |
| | CTGCTAGAATTCCGTTCTTCTATCATGTACTTGACAAAA |
| | GACTTTGAGCAACTTATCAGTATTATATTAAGATTGCCT |
| | TGGTTGAATAGAAGTCAAACAGTAGTGGAAGAGTATTTG |
| | GCTTTTCTTGGTAATCTTGTATCAGCACAGACTGTTTTC |
| | CTCAGACCGTGTCTCAGCATGATTGCTTCCCATTTTGTG |
| | CCTCCCCGAGTGATCATTAAGGAAGGCGATGTAGATGTT |
| | TCAGATTCTGATGATGAAGATGATAATCTTCCTGCAAAT |
| | TTTGACACATGTCACAGAGCCTTGCAAATAATAGCAAGA |
| | TATGTACCATCGACACCGTGGTTTCTCATGCCAATACTG |
| | GTGGAAAAATTTCCATTTGTTCGAAAATCAGAGAGAACA |
| | CTGGAATGTTACGTTCATAACTTACTAAGGATTAGTGTA |
| | TATTTTCCAACCTTGAGGCATGAAATTCTGGAGCTTATT |
| | ATTGAAAAACTACTCAAGCTGGATGTGAATGCATCCCGG |
| | CAGGGTATTGAAGATGCTGAAGAAACAGCAAATCAAACT |
| | TGTGGTGGGACAGATTCCACGGAAGGATTGTTTAATATG |
| | GGATTCGCAGAGGCATTTTTGGAACATCTTTGGAAAAAC |
| | TTGCAGGATCCAAGTAATCCTGCCATCATCAGGCACGCT |
| | GCTGGAAATTATATTGGAAGCTTTTTGGCAAGAGCTAAA |
| | TTTATTTCTCTTATTACTGTAAAACCATGCCTAGATCTT |
| | TTGGTTAACTGGCTGCACATATACCTTAATAACCAGGAT |
| | TCGGGAACAAAGGCATTCTGCGATGTTGCTCTCCATGGA |
| | CCATTTTACTCAGCCTGCCAAGCTGTGTTCTACACCTTT |
| | GTTTTTAGACACAAGCAGCTTTTGAGCGGAAACCTGAAA |
| | GAAGGTTTGCAGTATCCTCAGAGTCTGAATTTTGAGCGG |
| | ATAGTGATGAGCCAGCTAAATCCCCTGAAGATTTGCCTG |
| | CCCTCAGTGGTTAACTTTTTTGCTGCAATCACAAATAAG |
| | TACCAGCTCGTCTTCTGCTACACCATCATTGAGAGGAAC |
| | AATCGCCAGATGCTGCCAGTCATTAGGAGTACCGCTGGA |
| | GGAGACTCAGTGCAGATCTGCACAAACCCGCTGGACACC |
| | TTCTTCCCCTTTGATCCCTGTGTGCTGAAGAGGTCAAAG |
| | AAATTCATTGATCCTATTTATCAGGTGTGGGAAGACATC |
| | AGTGCTGAAGAGCTACAGOAGTTCAAGAAACCCATGAAA |
| | AAGGACTGATTGGGATCACACCAAGCTCCTTTGACACGC |
| | ATTTCCGAAGTCCTTCAAGTAGTGTGGGCTCCCCACCCG |
| | TGTTGTACATGCAACCCAGTCCCCTCTGACGGCAGAAAT |
| | TTGTGACTGAGATGTGACATTTGGGATTCCCCATC |

| | | |
|---|---|---|
| ORF Start: | | |
| ATG at 40 | ORF Stop: TGA at 1723 | |
| SEQ ID NO:8 | 561 aa | MW at 63593.3 kD |

| | |
|---|---|
| NOV3, | MAAPLLHTRLPGDAAASSSAVKKLGASRTGISNMRALEN |
| CG58596-01 | DFFNSPPRKTVRFGGTVTEVLLKYKKGETNDFELLKNQL |
| Protein | LDPDIKDDQIINWLLEFRSSIMYLTKDFEQLISIILRLP |
| Sequence | WLNRSQTVVEEYLAFLGNLVSAQTVFLRPCLSMIASHFV |
| | PPRVIIKEGDVDVSDSDDEDDNLPANFDTCHRALQIIAR |
| | YVPSTPWFLMPILVEKFPFVRKSERTLECYVHNLLRISV |
| | YFPTLRHEILELIIEKLLKLDVNASRQGIEDAEETANQT |
| | CGGTDSTEGLFNMGFAEAFLEHLWKNLQDPSNPAIIRQA |
| | AGNYIGSFLARAKFISLITVKPCLDLLVNWLHIYLNNQD |
| | SGTKAFCDVALHGPFYSACQAVFYTFVFRHKQLLSGNLK |
| | EGLQYPQSLNFERIVMSQLNPLKICLPSVVNFFAAITNK |
| | YQLVFCYTIIERNNRQMLPVIRSTAGGDSVQICTNPLDT |
| | FFPFDPCVLKRSKKFIDPIYQVWEDMSAEELQEFKKPMK |
| | KDIVEDEDDDFLKGEVPQNDTVIGITPSSFDTHFRSPSS |
| | SVGSPPVLYMQPSPL |

Further analysis of the NOV3 protein yielded the following properties shown in Table 3B.

TABLE 3B
Protein Sequence Properties NOV3

| | |
|---|---|
| PSort | 0.3600 probability located in mitochondrial matrix space; |
| analysis: | 0.1485 probability located in microbody (peroxisome); |
| | 0.1000 probability located in lysosome (lumen); 0.0000 |
| | probability located in endoplasmic reticulum (membrane) |
| SignalP | No Known Signal Sequence Predicted |
| analysis: | |

A search of the NOV3 protein against the Geneseq database, a proprietary database that contains sequences published in patents and patent publication, yielded several homologous proteins shown in Table 3C.

TABLE 3C
Geneseq Results for NOV3

| | | NOV3 | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| AAB10936 | Human RNA polymerase I transcription | 1 . . . 286 | 284/286 (99%) | e−162 |
| | factor TIF-1A protein - Homo sapiens, 651 | 1 . . . 286 | 284/286 (99%) | |
| | aa. [WO200055316-A1, 21-SEP-2000] | | | |
| AAB42728 | Human ORFX ORF2492 polypeptide | 286 . . . 532 | 235/247 (95%) | e−137 |
| | sequence SEQ ID NO:4984 - Homo sapiens, | 1 . . . 247 | 238/247 (96%) | |
| | 256 aa. [WO200058473-A2, 05-OCT-2000] | | | |
| AAU27684 | Human full-length polypeptide sequence #9 | 284 . . . 479 | 187/196 (95%) | e−107 |
| | - Homo sapiens, 329 aa. [WO200164834- | 127 . . . 322 | 188/196 (95%) | |
| | A2, 07-SEP-2001] | | | |
| AAY12885 | Human 5′ EST secreted protein SEQ ID | 34 . . . 160 | 122/127 (96%) | 3e−66 |
| | NO:475 - Homo sapiens, 127 aa. | 1 . . . 127 | 125/127 (98%) | |
| | [WO9906549-A2, 11-FEB-1999] | | | |
| AAB43324 | Human ORFX ORF3088 polypeptide | 34 . . . 153 | 89/120 (74%) | 2e−40 |
| | sequence SEQ ID NO:6176 - Homo sapiens, | 1 . . . 90 | 90/120 (74%) | |
| | 90 aa. [WO200058473-A2, 05-OCT-2000] | | | |

In a BLAST search of public sequence databases, the NOV3 protein was found to have homology to the proteins shown in the BLASTP data in Table 3D.

TABLE 3D
Public BLASTP Results for NOV3

| | | NOV3 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9H4F0 | TRANSCRIPTION INITIATION | 1 . . . 286 | 285/286 (99%) | e−162 |
| | FACTOR IA PROTEIN - Homo sapiens | 1 . . . 286 | 285/286 (99%) | |
| | (Human), 651 aa. | | | |
| Q9NYV6 | RRN3 - Homo sapiens (Human), 651 aa. | 1 . . . 286 | 285/286 (99%) | e−162 |
| | | 1 . . . 286 | 285/286 (99%) | |
| CAC16268 | SEQUENCE 1 FROM PATENT | 1 . . . 286 | 284/286 (99%) | e−162 |
| | WO0055316 - Homo sapiens (Human), | 1 . . . 286 | 284/286 (99%) | |
| | 651 aa. | | | |
| O75704 | HYPOTHETICAL 17.4 KDA PROTEIN | 286 . . . 429 | 143/144 (99%) | 8e−80 |
| | - Homo sapiens (Human), 153 aa. | 1 . . . 144 | 144/144 (99%) | |
| Q9BR79 | SIMILAR TO RNA POLYMERASE I | 286 . . . 427 | 142/142 (100%) | 7e−79 |
| | TRANSCRIPTION FACTOR RRN3 - | 1 . . . 142 | 142/142 (100%) | |
| | Homo sapiens (Human), 152 aa. | | | |

PFam analysis predicts that the NOV3 protein contains the domains shown in the Table 3E.

TABLE 3E
Domain Analysis of NOV3

| | | | |
|---|---|---|---|
| | NOV3 | Identities/Similarities | Expect |
| Pfam Domain | Match Region | for the Matched Region | Value |

No Significant Matches Found To Known Sequences

### Example A4

The NOV4 clone was analyzed, and the nucleotide and predicted polypeptide sequences are shown in Table 4A.

TABLE 4A
NOV4 Sequence Analysis

| | SEQ ID NO:9 | 1136 bp |
|---|---|---|
| NOV4, | GAACATTTTGTTAAATGAGCCGAGGGTGTGGAAAATATG | |
| CG57407-01 | ACTTTTATATTGGTCTGGGATTAGCTATGAGCTCCAGCA | |
| DNA | TTTTCACTGGAGGTAGTTTCATTTGGGGAGGGAAAAAAA | |
| Sequence | AAGGCTTTCTGCGACTTGCCAGAAAAGGTCCTCTGAGAG | |
| | CAGGTCAAGGTGGCCACGCATATCCTAAAGAATGGTTGT | |
| | GGTGGGCTAGACTGCTGTCAATGGCAGCTGGCGAGGTGG | |
| | CCAACTCAGCTGCATATGGGTTTGCACCGGCCACACTGG | |
| | TGACTCCACTAGGAACTCTCAGCGTCCTAGTAAGTGCTA | |
| | TTCTTTCTTCATACTTTCTAAATGAAAGATTTAATCCTT | |
| | ACGGGAAATTTGGATGTTTGCTAAGTATTTTAGGATCTA | |
| | CAGTTACGATCACTCATCCTCCAAAAGAAGAGGATATTG | |
| | AGACTCTAAATAAAATATCTCACAAGCTAGGTGATCCAG | |
| | GTTTTGTGGACTTTGCAACACTTGTGGTCATTATGGCCA | |
| | TGATATTAATCTTCCTGGTGGGTCCCCACCAGGGACAAC | |
| | GATCTTGTGTATGTAOCAATTTGCACATTTGTGTATGTA | |
| | ACAAAATTGGCACATTTTCAGTCTCCTGGGTTAAGAGCT | |
| | TAGGCAGTGCTATCAGAGAGCTGTTTGCTGGAAAGCCTG | |
| | CACTGCCACATCCCCTGGCCTATGTTCTGCTGCTAAGCC | |
| | TCATTGTCTGTGTGAACACACAGATTAATTACCTAAATT | |
| | GCGCCCTGGATATATTTAACACTTCCATCATGACTCCAA | |
| | TACATTACATATTCTTTACGACATCAGTTTTTAAACTTG | |
| | TTCAGCTATTATTTTTAAGGAGTGGCAAGATATGCCCAT | |
| | TGATGATGTCACTGGTACTTTTGACTGGCTTTACAATAA | |
| | TCGTGGGGATATTCTTGTTGCATGCTTTTAAGAGTGTCA | |
| | GCTTTAGTCTAGCAAGTCTGCCTGTGTCTCTTCGAAAAG | |
| | ACAAAAAAGCAATGAATGGCAATCTCTCTAATATGTACG | |
| | AAGTTCTTAATAATAATGAAGAAAGCAAAAGCTTAATCT | |
| | GTGGAATCAAACTACACACTGGTGAAAATATCTCCCGAA | |
| | GAAATGGAATTCTGACAGCTTTTTAAGAAAGATATAATT | |
| | AAAAG | |
| | ORF Start: | |
| | ATG at 15 | ORF Stop: TAA at 1116 |
| | SEQ ID NO:10 | 367 aa MW at 40060.8 kD |

| | |
|---|---|
| NOV4, | MSRGCGKYDFYIGLGLAMSSSIFTGGSFIWGGKKKGFLR |
| CG57407-01 | LARKGPLAAGQGGHAYPKEWLWWARLLSMAAGEVANSAA |
| Protein | YGFAPATLVTPLGTLSVLVSAILSSYFLNERFNPYGKFG |
| Sequence | CLLSILGSTVTITHPPKEEDIETLNKISHKLGDPGFVDF |
| | ATLVVIMAAILIFLVGPHQGQTSCVCSNLHICVCNKIGT |
| | FSVSWVKSLGSAIRELFAGKPALPHPLAYVLLLSLIVCV |
| | NTQINYLNCALDIFNTSIMTPIHYIFFTTSVFKLVQLLF |
| | LRSGKICPLMMSLVLLTGFTIIVGIFLLHAFKSVSFSLA |
| | SLPVSLRKDKKAMNGNLSNMYEVLNNNEESKSLICGIKL |
| | HTGENISRRNGILTAF |

Further analysis of the NOV4 protein yielded the following properties shown in Table 4B.

TABLE 4B
Protein Sequence Properties NOV4

| | |
|---|---|
| PSort | 0.6850 probability located in endoplasmic reticulum |
| analysis: | (membrane); 0.6400 probability located in plasma membrane; |
| | 0.4600 probability located in Golgi body; 0.1000 probability |
| | located in endoplasmic reticulum (lumen) |
| SignalP | Likely cleavage site between residues 27 and 28 |
| analysis: | |

A search of the NOV4 protein against the Geneseq database, a proprietary database that contains sequences published in patents and patent publication, yielded several homologous proteins shown in Table 4C.

TABLE 4C
Geneseq Results for NOV4

| | | NOV4 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAM39990 | Human polypeptide SEQ ID NO 3135 - | 1 . . . 367 | 277/368 (75%) | e−148 |
| | Homo sapiens, 360 aa. [WO200153312-A1, | 1 . . . 360 | 309/368 (83%) | |
| | 26-JUL-2001] | | | |
| AAM38999 | Human polypeptide SEQ ID NO 2144 - | 1 . . . 367 | 277/368 (75%) | e−148 |
| | Homo sapiens, 360 aa. [WO200153312-A1, | 1 . . . 360 | 309/368 (83%) | |
| | 26-JUL-2001] | | | |
| AAB18993 | Amino acid sequence of a human | 1 . . . 367 | 277/368 (75%) | e−148 |
| | transmembrane protein - Homo sapiens, | 1 . . . 360 | 309/368 (83%) | |
| | 360 aa. [WO200056891-A2, 28-SEP-2000] | | | |
| AAU30546 | Novel human secreted protein #1037 - | 1 . . . 297 | 204/301 (67%) | e−102 |
| | Homo sapiens, 314 aa. [WO200179449-A2, | 5 . . . 300 | 230/301 (75%) | |
| | 25-OCT-2001] | | | |
| AAW78128 | Human secreted protein encoded by gene 3 | 144 . . . 367 | 159/225 (70%) | 7e−80 |
| | clone HOSBI96 - Homo sapiens, 220 aa. | 1 . . . 219 | 185/225 (81%) | |
| | [WO9856804-A1, 17-DEC-1998] | | | |

In a BLAST search of public sequence databases, the NOV4 protein was found to have homology to the proteins shown in the BLASTP data in Table 4D.

TABLE 4D
Public BLASTP Results for NOV4

| | | NOV4 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q96F03 | SIMILAR TO RIKEN CDNA 3830408P04 | 1 . . . 367 | 276/368 (75%) | e−147 |
| | GENE - Homo sapiens (Human), 360 aa. | 1 . . . 360 | 308/368 (83%) | |
| Q9JJC8 | BRAIN CDNA, CLONE MNCB-2146, | 1 . . . 367 | 267/368 (72%) | e−141 |
| | SIMILAR TO HUMAN CLONE 23773 | 1 . . . 359 | 302/368 (81%) | |
| | MRNA SEQUENCE - Mus musculus | | | |
| | (Mouse), 359 aa. | | | |
| Q9D8E3 | 3830408P04RIK PROTEIN - Mus musculus | 46 . . . 367 | 231/323 (71%) | e−122 |
| | (Mouse), 316 aa. | 1 . . . 316 | 265/323 (81%) | |
| Q9BVS2 | HYPOTHETICAL 32.4 KDA PROTEIN - | 65 . . . 367 | 224/304 (73%) | e−116 |
| | Homo sapiens (Human), 299 aa (fragment). | 2 . . . 299 | 254/304 (82%) | |
| Q9D0N5 | 2600017P10RIK PROTEIN - Mus musculus | 62 . . . 367 | 217/307 (70%) | e−112 |
| | (Mouse), 308 aa. | 9 . . . 308 | 250/307 (80%) | |

PFam analysis predicts that the NOV4 protein contains the domains shown in the Table 4E.

TABLE 4E
Domain Analysis of NOV4

| | NOV4 | Identities/Similarities | Expect |
|---|---|---|---|
| Pfam Domain | Match Region | for the Matched Region | Value |
| DUF6: domain | 3 . . . 130 | 27/144 (19%) | 0.16 |
| 1 of 1 | | 90/144 (62%) | |

### Example A5

The NOV5 clone was analyzed, and the nucleotide and predicted polypeptide sequences are shown in Table 5A.

TABLE 5A
NOV5 Sequence Analysis

| | SEQ ID NO:11 | 6609 bp |
|---|---|---|
| NOV5, | ATGCTGGCAGAGCCCAATTATAGGCTCCTGAAGAGGACGGGTGACAACGTGTGGCTGG | |
| CG57770-01 | AGCCTCCCTCCACCCACAAGACCGGCGTGGCCATCGGGGGCATCATCAAAGAGGCAAA | |
| DNA Sequence | GCCAGGCAAAGTCTTGGTTGAAGATGACGAGGGCAAGGAACACTGGATCCGAGCAGAG | |
| | GACTTTGGTGTCCTCAGTCCCATGCACCCCAACTCAGTCCAGGGTGTGGACGACATGA | |
| | TCCGCCTGGGGGACCTGAACGAGGCAGGCATGGTGCACAACCTCCTGATCCGCTACCA | |
| | GCAGCACAAGATCTATACATACACAGGCTCCATCCTGGTGGCCGTCAACCCGTTCCAG | |
| | GTGCTGCCGCTCTACACCCTGGAGCAGGTACAGCTCTACTACAGCCGCCATATGGGCG | |
| | AGCTGCCCCCGCATGTCTTTGCCATCGCCAACAACTGCTACTTCAGCATGAAGAGGAA | |
| | CAAGAGGGACCAGTGCTGCATCATCAGCGGCGAGTCTGGGGCTGGCAAGACGGAGACC | |
| | ACCAAGCTCATCCTGCAGTTCCTGGCCACCATCAGTGGCCAGCATTCGTGGATTGAGC | |
| | AGCAGGTCCTGGAAGCCAACCCCATCCTGGAGGCCTTTGGAAATGCCAAGACAATCCG | |
| | CAACGACAACTCAAGCCGCTTTGGGAACGGGGTGATCGAGGGCGCGCGCATCGAGCAA | |
| | TTTCTCCTGGAGAAGTCCCGGGTCTGCCGGCAGGCTCCCGAGGAGCGGAACTACCATA | |
| | TCTTCTACTGCATGCTCATGGGGGTGAGTGCTGAGGACAAGCAGCTGCTGAGCCTGGG | |
| | CACGCCCTCCGAGTACCACTACCTGACCATGGGGAACTGCACTTCCTGTGAGGGGCTC | |
| | AACGACGCCAAGGACTACGCCCACATCCGCTCGGCCATGAAGATCCTCCAGTTCTCCG | |
| | ACTCCGAGAGCTGGGACGTCATCAAGCTGCTGGCTGCCATTCTCCACCTGGGGAATGT | |
| | GGGGTTCATGGCTTCGGTCTTCGAGAACCTGGACGCCTCAGACGTGATGGAGACGCCC | |
| | GCCTTTCCCACCGTGATGAAGTTACTGGAGGTGCAGCACCAGGAGCTCCGGGACTGTC | |
| | TGATCAAGCACACCATCCTCATCCGAGGGGAATTTGTCACCAGGTCCCTGAACATTGC | |
| | CCAGGCTGCTGACCGGAGGGACGCCTTTGTCAAGGGCATCTATGGGCACCTCTTCCTC | |
| | TGGATTGTCAAGAAGATCAATGCCGCCATCTTCACACCACCAGCCCAGGACCCCAAAA | |
| | ATGTGCGGAGGGCCATCGGCCTCCTGGACATATTTGGCTTTGAAAATTTCGAGAACAA | |
| | TAGCTTCGAGCAGCTCTGCATCAACTTCGCCAACGAGCACCTGCAGCAGTTCTTTGTG | |
| | CAGCACGTGTTCACCATGGAGCAAGAGGAGTACCGCTCGGAGAACATCTCCTGGGACT | |
| | ATATCCACTACACCGACAATCGGCCCACCCTGGACCTGCTGGcCCTCAACCCCATGAG | |
| | CATCATCTCCCTCCTGGACGAAGAAAGCCGCTTCCCGCAGGGGACAGATCTCACCATG | |
| | CTGCAAAAGCTGAACAGCGTCCATGCCAACAACAAGGCCTTCCTACAGCCCAAGAACA | |
| | TCCACGATGCCAGATTTGGCATTGCCCATTTTGCCGGCGAGGTGTACTACCAAGCAGA | |
| | AGGCTTCCTGGAGAAGAACCGAGACGTGCTGAGCACAGATATCCTCACCCTGGTTTAC | |
| | TCCTCCAAAAACAAGTTTCTGAGGGAGATATTCAACTTGGAGTTAGCAGAGACCAAGC | |
| | TGGGCGCAGACTCAAATAAACGGCCCTCCACCTTAGGAAGCCAGTTCAAACAGTCTCT | |
| | GGACCAGCTGATGAAAATCCTGACCAACTGCCAGCCTTACTTCATCCGCTGCATCAAA | |
| | CCTAATGAGTACAAGAAGCCGCTGCTGTTCGACCGGGAGCTGTGCCTGCGGCAGCTGC | |
| | GATACTCGGGCATGATGGAGACCGTGCACATCCGCAAGTCGGGCTTCCCCATCCGCTA | |
| | CACGTTCGAGGAGTTCTCGCAGAGGTTCGGCGTGTTGCTGCCCAACGCCATGCGGATG | |
| | CAGCTGCAAGGCAACGTCCGCCAGATGACCCTGGGCATCACTGACGTGTGGCTGCGGA | |
| | CAGACAAAGACTGGAAAGCGGGGAAGACAAAAATTTTCCTGAGGGATCATCAGGACAC | |
| | TCTGCTGGAGGTACAGAGAAGCCAGGTGCTAGACAGAGCGGCGCTCAGCATCCAGAAA | |
| | GTCCTTCGGGGCTACAGATACAGGAAGGAGTTCCTGAGGAAGAGGCGGGCAGCTGTGA | |
| | CCCTGCAGGCCTGGTGGAGAGGCTACTGCAACAGGAGGAATTTCAAGCTGATCCTCGT | |
| | GGGCTTTGAGCGCCTGCAGGCTATTGCCCGGAGCCAGCCGCTGGCGAGGCAGTACCAG | |
| | GCCATGCGGCAGAGGACAGTCCAGCTGCAGGCCCTGTGCAGGGGATACCTGGTGCGCC | |
| | AGCAAGTCCAGGCCAAGAGGAGGGCAGTGGTGGTCATTCAGGCCCATGGCCAGGGCAT | |
| | GGCTGCCCGGCGCAACTTCCAGCAAAGGAAGCCCAATGCGCCGCTGGTAATCCCGGCC | |
| | GAGGGGCAGAAAAGCCAAGGCGCTCTCCCTGCCAAGAAGCGCAGAAAAGAAAAAGAAA | |
| | GAAAGAGAAAAGAAGAAAGGAAGGGAAGGAGGGAAAAAGAAAAGAAAAAAAGAAAAAA | |
| | GAAAAGAAAAAAGGAAAATAAAGAAAAGCAAGCAAACAAGCAAAGCATAGTGAGAGAG | |
| | AGAGGAAGGTCCATCTACGACACCGTCACTGACACGGAGATGGTGGAGAAGGTGTTCG | |
| | GCTTCCTCCCTGCCATGATTGGGGGCCAGGAGGGCCAGGCCTCGCCGCACTTTGAGGA | |
| | TCTGGAATCGAAGACCCAGAAGCTGCTTGAGGTTGACCTGGACACAGTCCCAATGGCG | |
| | GAGGAGCCTGAGGAGGATGTGGATGGCCTGGCCGAGTACACCTTCCCCAAGTTTGCTG | |
| | TGACTTACTTCCAGAAATCAGCCAGCCACACACACATCCGGCGGCCCCTCCGATACCC | |
| | GTTGCTTTACCACGAAGATGACACTGACTGCTTGGCCGCCCTGGTCATATGGAACGTC | |
| | ATCCTGAGGTTCATGGGTGATCTCCCAGAGCCAGTGCTGTATGCCAGGAGCAGCCAGC | |
| | AGGGCAGCTCAGTGATGCGGCAGATCCATGACACGCTGGGCAGGGAGCACGGTGCCCA | |
| | GGTTCCACAGCACAGTAGATCTGCACAGGCAAGTGGGGGGCAGCAGCGGGCAGAGGAG | |
| | GGCGACACCTACCAGAGATCTGGCTGCAAGGACAAGGGGACCAAGGATATCTCCTCCA | |
| | TGAAGCTGAAGCGGTCCTCCCGGATCACAGGCCAGGTGGCCAGCCAGCTGAACATTGG | |
| | AGAGGAGGCATTGGAGCCTGATGGCCTTGGTGCAGACCGGCCCATGTCCAACCTGGAG | |
| | AAGGTGCACTTCATCGTGGGCTACGCCATCCTGCGGCCCAGCCTCAGGGATGAGATTT | |
| | ACTGCCAGATCTGCAAGCAGCTCTCGGAGAACTTCAAAACAAGCAGCCTGGCCCGGGG | |
| | CTGGATCCTGCTCAGCCTCTGCCTCGGCTGCTTCCCACCCTCAGAGAGGTTCATGAAG | |
| | TATCTACTGAACTTCATCGGCCAAGGGCCGGCGACCTACGGCCCCTTCTGTGCCGAGC | |
| | GCCTGAGACGCACCTATGCCAATGGGGTGCGTGCGGAGCCCCCCACCTGGCTGGAGCT | |
| | GCAGGCTGTCAAGTCCAAGAAGCACATCCCCATCCAAGTCATCTTGGCCACTGGAGAG | |
| | AGCCTAACCGTCCCCGTGGACTCAGCCTCCACATCTCGGGAAATGTGCATGCACATCG | |
| | CTCACAAGCAGGGCCTCAGCGACCACCTGGGCTTCTCCCTCCAGGTCGCCGTGTACGA | |
| | CAAGTTCTGGTCCCTGGGCAGCGGGCGCGACCACATGATGGATGCCATCGCCCGGTGT | |
| | GAGCAGATGGCCCAGGAGAGGGGCGAGAGCCAGCGCCAGTCACCCTGGCGCATCTACT | |
| | TCCGGAAGGAATTCTTCACCCCCTGGCACGACTCCCGGGAGGACCCTGTCAGCACCGA | |
| | GCTTATTTACCGCCAAGTCCTCCGAGGAGTCTGGTCTGGCGAGTACAGCTTCGAGAAG | |
| | GAGGAAGAGCTGGTTGAGCTGCTGGCCCGGCACTGCTACGTGCAGCTCGGCGCCTCAG | |
| | CAGAGAGCAAGGCTGTCCAGGAGCTGCTGCCCAGCTGCATCCCCCACAAGCTGTACAG | |
| | GACCAAGCCCCCAGACAGGTGGGCGAGCCTCGTCACTGCCGCCTGCGCCAAGGCCCCA | |
| | TACACTCAGAAGCAAGTCACACCACTGGCCGTGCGAGAGCAGGTGGTGGACGCCGCCC | |
| | GCCTGCAGTGGCCGCTGCTCTTCTCCCGGCTCTTCGAAGTCATCACACTCTCAGGCCC | |
| | CCGCCTGCCCAAGACGCAGCTGATCTTGGCTGTTAACTGGAAGGGGCTTTGCTTCCTG | |
| | GACCAGCAGGAGAAGATGCTGCTGGAACTCTCTTTCCCAGAGGTCATGGGTCTGGCCA | |
| | CCAACAGGGAGGCCCAGGGCGGGCAGAGGCTGCTGCTCTCCACGATGCATGAGOAGTA | |
| | CGAGTTTGTGTCACCCAGCAGTGTGGCCATCGCTGAGCTGGTGGCCCTGTTCCTGGAG | |
| | GGCCTGAAGGAGAGGTCCATTTTCGCCATGGCCCTGCAGGACAGGAAGGCCACAGATG | |
| | ACACCACCCTCCTGGCCTTCAAGAAGGGGGACCTGTTGGTCCTCACAAAGAAGCAGGG | |
| | GCTGCTGGCCTCTGAGAACTGGACCCTCGGCCAGAACGACAGGACAGGCAAAGACGGG | |
| | CTGGTGCCCATGGCCTGCCTCTACACCATCCCCACGGTCACTAAGCCCTCGGCACAGC | |
| | TGCTGAGCTTGCTTGCCATGTCACCAGAGAAGAGGAAGCTGGCGGCTCAGGAGGGGCA | |
| | GTTCACAGAGCCACGTCCTGAGGAGCCACCCAAGGAAAAGCTGCACACCCTGGAGGAG | |
| | TTCTCCTATGAGTTCTTCAGGGCTCCAGAGAAGGACATGGTGAGCATGGCCGTGCTGC | |
| | CCCTGGCCCGTGCCCGTGGCCACCTGTGGGCCTATTCCTGCGAGCCGCTGCGACAGCC | |
| | GCTGCTCAM3CGAGTCCACGCCAACGCCGGCGTCGGGGTCAGCGTCTATCCCCAGTCT | |
| | GTGTCAGCGTGGGCTGCCCCAGCACTGTGCTCCTTGACAGCCACACCCATCCTCCGGT | |
| | ACATGGGCGACTACCCTTCTCGGCAGGCCTGGCCCACCCTGGAGCTCACCGACCAGAT | |
| | CTTCACACTGGCCCTGCAGCACCCGGCCCTCCAGGACGAGGTCTACTGCCAGATCCTG | |
| | AAGCAGCTGACGCACAACTCCAACAGGCACAGCGAAGAGCGGGGCTGGCAGCTGCTGT | |
| | GGCTGTGCACGGGCCTCTTCCCGCCCAGCAAGGGGCTGCTGCCCCATGCCCAGAAGTT | |
| | TATAGACACTCGGACGGGGAAGCTGCTGCCCCCCGACTGCAGCCGCCGAATCCAGAAG | |
| | GTCCTGAGGACGGGGCCCCGGAAGCAGCCCCCGCACCAGGTGGAGGTGGAGGCCGCAG | |
| | AGCAGAACGTCTCCCGCATCTGCCACAAGATCTACTTCCCCAATGACACCAGTGAGAT | |
| | GCTGGAGGTGGTTGCCAACACACGGGTGCGGGATGTGTGTGACAGCATTGCCACCAGG | |
| | CTGCAGCTGGCCTCCTGGGAGGGCTGCAGCCTCTTCATCAAGATTTCAGACAAGGTCA | |
| | TCAGCCAGAAGGAGGGAGACTTCTTCTTTGATTCCTTGAGGGAGGTGTCTGACTGGCT | |
| | GAAGAAGAACAAGCCCCAGAAAGAAGGTGCCCTGGGGGCCCCCGTGACGCTCCCCTAC | |
| | CAGGTGTACTTCATGCGGAAATTGTGGCTCAACATATCTCCAGGGAAGGATGTGAATG | |
| | CAGACACCATACTCCATTACCACCAGGAGCTGCCCAAGTACCTGCGCGGATTCCACAA | |
| | GTGTTCGCGGGAGGATGCCATCCACCTGGCGGGCCTCATCTACAAGGCCCAGTTCAAC | |
| | AACGACCGGTCCCAGCTGGCTAGTGTCCCCAAGATCCTGAGGGAACTGGTGCCTGAGA | |
| | ACCTCACACGCCTGATGTCCTCGGAGGAGTGGAAAAAGAGCATCCTTCTAGCCTATGA | |
| | CAAGCATAAGGACAAGACAGTGGAGGAGGCCAAGGTGGCCTTCCTGAAGTGOATCTGC | |
| | CGGTGGCCCACCTTCGGATCCGCCTTCTTCGAGGTGATGCAAACCTCGGAGCCTTCCT | |
| | ACCCGGACGTCATCCTCATCGCCATCAACCGACATGGGGTTCTGCTCATCCACCCCAA | |
| | GACCAAGGACCTGCTCACCACCTATCCCTTCACCAAGATCTCCAGCTGGAGCAGCGGC | |
| | AGCACCTACTTCCACATGGCGCTGGGGAGCCTGGGCCGTGGCAGCCGCCTGCTGTGCG | |
| | AGACCTCCCTGGGCTATAAGATGGATGACCTGCTGACCTCATATGTGCAGCAGCTCCT | |
| | GAGTGCCATGAACAAGCAGCGGGGCTCCAAGGCCCCAGCCCTGGCCAGCACCTAG | |
| | ORF Start: ATG at 1 | ORF Stop: TAG at 6607 |
| | SEQ ID NO:12 | 2202 aa MW at 251200.3 kD |

| | |
|---|---|
| NOV5, | MLAEPNYRLLKRTGDHVWLEPPSTHKTGVAIGGIIKEAKPGKVLVEDDEGKEHWIRAE |
| CG57770-01 | DFGVLSPMHPNSVQGVDDMIRLGDLNEAGMVHNLLIRYQQHKIYTYTGSILVAVNPFQ |
| Protein | VLPLYTLEQVQLYYSRHMGELPPHVFAIANNCYFSMKRNKRDQCCIISGESGAGKTET |
| Sequence | TKLILQFLATISGQHSWIEQQVLEANPILEAFGNAKTIRNDNSSRFGNGVIEGARIEQ |
| | FLLEKSRVCRQAPEERNYHIFYCMLMGVSAEDKQLLSLGTPSEYHYLTMGNCTSCEGL |
| | NDAKDYAHIRSAMKILQPSDSESWDVIKLLAAILHLGNVGFMASVFENLDASDVMETP |
| | AFPTVMKLLEVQHQELRDCLIKHTILIRGEFVTRSLNIAQAADRRDAFVKGIYGHLFL |
| | WIVKKTNAATFTPPAQDPKNVRRAIGLLDIFGFENFENNSFEQLCTNFANEHLQQFFV |
| | QHVFTMEQEEYRSENISWDYIHYTDNRPTLDLLALKPMSIISLLDEESRFPQGTDLTM |
| | LQKLNSVHANNKAFLQPKNIHDARFGIAHFAGEVYYQAEGFLEKNRDVLSTDILTLVY |
| | SSKNKFLREIFNLELAETKLGADSNKRPSTLGSQFKQSLDQLMKILTNCQFYFIRCIK |
| | PNEYKKPLLFDRELCLRQLRYSGMMEYVHIRKSGFPIRYTFEEFSQRFGVLLPNAMRM |
| | QLQGNVRQMTLGITDVWLRTDKDWKAGKTKIFLRDHQDTLLEVQRSQVLDRAALSIQK |
| | VLRGYRYRKEFLRQRRAAVTLQAWWRGYCNRRNFKLILVGFERLQAIARSQPLARQYQ |
| | AMRQRTVQLQALCRGYLVRQQVQAKRRAVVVIQAHGQGMAARRNFQQRKANAPLVIPA |
| | EGQKSQGALPAKKRRKEKERKRKEERKGRREKEKKKRKKKRKKENKEKQANKQSTVRE |
| | RGRSIYDTVTDTEMVEKVFGFLPAMIGGQEGQASPHFEDLESKTQKLLEVDLDTVPMA |
| | EEPEEDVDGLAEYTFPKFAVTYFQKSASHTHIRRPLRYPLLYHEDDTDCLAALVIWNV |
| | ILRFMGDLPEPVLYARSSQQGSSVMRQIHDTLGREHGAQVPQHSRSAQASGGQQPAEE |
| | GDTYQRSGCKDKGTKDISSMKLKRSSRITGQVASQLNIGEEALEPDGLGADRPMSNLE |
| | KVHPIVGYAILRPSLRDEIYCQICKQLSENFKTSSLARGWILLSLCLGCFPPSERFMK |
| | YLLNFTGQGPATYGPFCAERLRRTYANGVRAEPPTWLELQAVKSKKHIPTQVILATGE |
| | SLTVPVDSASTSREMCMHTAHKQGLSDHLGFSLQVAVYDKFWSLGSGRDHMNDATARC |
| | EQMAQERGESQRQSPWRIYFRKEFFTPWHDSREDPVSTELIYRQVLRGVWSGEYSFEK |
| | EEELVELLARHCYVQLGASAESKAVQELLPSCIPHKLYRTKPPDRWASDVTAACAKAP |
| | YTQKQVTPLAVREQVVDAARLQWPLLFSRLFEVITLSGPRLPKTQLILAVNWKGLCFL |
| | DQQEKMLLELSFPEVMGLATNREAQGGQRLLLSTMHEEYEFVSPSSVAIAELVALFLE |
| | GLKERSIFAMALQDRKATDDTTLLAFKKGDLLVLTKKQGLLASENWTLGQNDRTGKTG |
| | LVPMACLYTIPTVTKPSAQLLSLLANSPEKRKLAAQEGQFTEPRPEEPPKEKLHTLEE |
| | FSYEFFRAPEKDMVSMAVLPLARARGHLWAYSCEPLRQPLLKRVHANAGVGVSVYPQS |
| | VSAWAAPALCSLTATPILRYMGDYPSRQAWPTLELTDQIFTLALQHPALQDEVYCQIL |
| | KQLTHNSNRHSEERGWQLLWLCTGLFPPSKGLLPHAQKFIDTRRGKLLAPDCSRRIQK |
| | VLRTGPRKQPPHQVEVEAAEQNVSRICHKIYFPNDTSEMLEVVANTRVRDVCDSIATR |
| | LQLASWEGCSLFIKISDKVISQKEGDFFFDSLREVSDWVKKNKPQKEGALGAPVTLPY |
| | QVYFMRKLWLNISPGKDVNADTILHYHQELPKYLRGFHKCSREDAIHLAGLIYKAQFN |
| | NDRSQLASVPKILRELVPENLTRLMSSEEWKKSILLAYDKHKDKTVEEAKVAFLKWIC |
| | RWPTFGSAFFEVKQTSEPSYPDVILIAINRHGVLLIHPKTKDLLTTYPFTKISSWSSG |
| | STYFHMALGSLGRGSRLLCETSLGYKNDDLLTSYVQQLLSAMNKQRGSKAPALAST |

Further analysis of the NOV5 protein yielded the following properties shown in Table 5B.

TABLE 5B
Protein Sequence Properties NOV5

| | |
|---|---|
| PSort | 0.9100 probability located in nucleus; 0.8500 probability |
| analysis: | located in endoplasmic reticulum (membrane); 0.4400 |
| | probability located in plasma membrane; 0.3811 probability |
| | located in microbody (peroxisome) |
| SignalP | No Known Signal Sequence Predicted |
| analysis: | |

A search of the NOV5 protein against the Geneseq database, a proprietary database that contains sequences published in patents and patent publication, yielded several homologous proteins shown in Table 5C.

TABLE 5C
Geneseq Results for NOV5

| | | NOV5 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAW00603 | Human non-conventional myosin VIIA - | 103 . . . 789 | 425/696 (61%) | 0.0 |
| | Homo sapiens, 697 aa. [EP725136-A1, 07- | 1 . . . 691 | 535/696 (76%) | |
| | AUG-1996] | | | |
| AAW00604 | Murine myosin VII protein - Mus sp, 631 | 103 . . . 727 | 399/634 (62%) | 0.0 |
| | aa. [EP725136-A1, 07-AUG-1996] | 1 . . . 631 | 503/634 (78%) | |
| AAE11891 | Angiogenesis associated human myosin X2 | 14 . . . 946 | 344/959 (35%) | e−169 |
| | (hMX2) protein variant - Homo sapiens, | 5 . . . 877 | 537/959 (55%) | |
| | 2048 aa. [WO200170808-A2, 27-SEP- | | | |
| | 2001] | | | |
| AAE11890 | Angiogenesis associated human myosin X1 | 14 . . . 946 | 344/959 (35%) | e−169 |
| | (hMX1) protein - Homo sapiens, 2057 aa. | 5 . . . 877 | 537/959 (55%) | |
| | [WO200170808-A2, 27-SEP-2001] | | | |
| AAG48638 | Arabidopsis thaliana protein fragment SEQ | 14 . . . 954 | 369/1019 (36%) | e−160 |
| | ID NO:61443 - Arabidopsis thaliana, 1544 | 13 . . . 980 | 545/1019 (53%) | |
| | aa. [EP1033405-A2, 06-SEP-2000] | | | |

In a BLAST search of public sequence databases, the NOV5 protein was found to have homology to the proteins shown in the BLASTP data in Table 5D.

TABLE 5D
Public BLASTP Results for NOV5

| | | NOV5 | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q99MZ6 | MYOSIN-VIIB - Mus musculus | 12 . . . 2201 | 1698/2215 (76%) | 0.0 |
| | (Mouse), 2113 aa. | 5 . . . 2112 | 1886/2215 (84%) | |
| Q9DGG8 | MYOSIN VIIA - Brachydanio rerio | 9 . . . 2195 | 1161/2214 (52%) | 0.0 |
| | (Zebrafish) (Zebra danio), 2179 aa. | 2 . . . 2174 | 1586/2214 (71%) | |
| Q9DGG9 | MYOSIN VIIA - Brachydanio rerio | 9 . . . 2195 | 1159/2214 (52%) | 0.0 |
| | (Zebrafish) (Zebra danio), 2179 aa. | 2 . . . 2174 | 1584/2214 (71%) | |
| Q13402 | Myosin VIIa - Homo sapiens | 9 . . . 2196 | 1176/2253 (52%) | 0.0 |
| | (Human), 2215 aa. | 2 . . . 2213 | 1582/2253 (70%) | |
| A59255 | myosin VIIa, long form - human, | 9 . . . 2196 | 1169/2215 (52%) | 0.0 |
| | 2175 aa. | 2 . . . 2173 | 1577/2215 (70%) | |

PFam analysis predicts that the NOV5 protein contains the domains shown in the Table 5E.

TABLE 5E
Domain Analysis of NOV5

| | | Identities/ | |
|---|---|---|---|
| | | Similarities | |
| | NOV5 | for the | Expect |
| Pfam Domain | Match Region | Matched Region | Value |
| myosin_head: | 74 . . . 730 | 309/738 (42%) | 2.6e−270 |
| domain 1 of 1 | | 504/738 (68%) | |
| IQ: domain 1 of 4 | 746 . . . 766 | 9/21 (43%) | 0.0029 |
| | | 16/21 (76%) | |
| IQ: domain 2 of 4 | 769 . . . 789 | 11/21 (52%) | 1.4e−05 |
| | | 19/21 (90%) | |
| IQ: domain 3 of 4 | 815 . . . 835 | 8/21 (38%) | 0.052 |
| | | 16/21 (76%) | |
| IQ: domain 4 of 4 | 838 . . . 858 | 8/21 (38%) | 0.034 |
| | | 18/21 (86%) | |
| MyTH4: domain 1 of 2 | 1156 . . . 1261 | 52/118 (44%) | 1.2e−42 |
| | | 87/118 (74%) | |
| SH3: domain 1 of 1 | 1573 . . . 1634 | 20/64 (31%) | 0.0066 |
| | | 42/64 (66%) | |
| MyTH4: domain 2 of 2 | 1770 . . . 1876 | 47/118 (40%) | 1.3e−40 |
| | | 87/118 (74%) | |

### Example A6

The NOV6 clone was analyzed, and the nucleotide and predicted polypeptide sequences are shown in Table 6A.

TABLE 6A
NOV6 Sequence Analysis

| | |
|---|---|
| SEQ ID NO:13 | 1379 bp |

| NOV6a, | CTCTGACTCTGCCCTTCATACAAGTGTGATGAACCCCAGTCCCCAGGATACCTACCCA | |
|---|---|---|
| CG59233-01 | GGCCCCACACCTCCCAGCATCCTGCCCAGCCGACGTGGGGGTGGTATTCTGGATGGTG | |
| DNA Sequence | AAATGGACCCCAAAGTACCTGCTATTGAGGAGAACTTGCTAGATGACAAGCATTTGCT | |
| | GAAGCCATGGGATGCTAAGAAGCTATCCTCATCCTCTTCCCGACCTCGGTCCTGTGAA | |
| | GTCCCTGGAATTAGCATCTTTCCATCTCCTGACCAGCCTGCCAATGTGCCTGTCCTCC | |
| | CACCTGCCATGAACACGGGGGGCTCCCTACCTGACCTCACCAACCTGCACTTTCCCCC | |
| | ACCACTGCCCACCCCCCTGGACCCTGAAGAGACAGCCTACCCTAGCCTGAGTGGGGGC | |
| | AACAGTACCTCCAATTTGACCCACACCATGACTCACCTGGGCATCAGCAGGGGCATGG | |
| | GCCTGGGCCCAGGCTATGATGCACCAGGTCTTCATTCACCTCTCAGCCACCCATCCCT | |
| | GCAGTCCTCCCTAAGCAATCCCAACCTCCAGGCTTCCCTGAGCAGTCCTCAGCCCCAG | |
| | CTTCAGGGCTCCCACAGCCACCCCTCTCTGCCTGCCTCCTCCTTGGCCCGCCATGTAC | |
| | TGCCCACCACCTCCCTGGGCCACCCCTCACTCAGTGCTCCCGCTCTCTCCTCCTCCTC | |
| | TTCCTCCTCCTCCACTTCATCTCCTGTTTTGGGCGCCCCCTCTTACCCTGCTTCTACC | |
| | CCTGGGGCCTCCCCCCACCACCGCCGTGTGCCCCTCAGCCCCCTGAGTTTGCTCGCGG | |
| | GCCCAGCCGACGCCAGAAGGTCCCAACAGCAGCTGCCCAAACAGTTTTCGCCAACAAT | |
| | GTCACCCACCTTGTCTTCCATCACTCAGCTGGAGCAGTTCAGCATGGAGAGCCCATCA | |
| | GCCAGCCTGGTGCTGGATCCCCCTGGCTTTTCTGAAGGGCCTGGATTTTTAGGGGGTG | |
| | AGGGGCCAATGGGTGGCCCCCAGGATCCCCACACCTTCAACCACCAGAACTTGACCCA | |
| | CTGTTCCCGCCATGGCTCAGGGCCTAACATCATCCTAACAGGTGACTCCTCTCCAGGT | |
| | TTCTCTAAGGAGATTGCAGCAGCCCTGGCCGGAGTGCCTGGCTTTGAGGTGTCAGCAG | |
| | CTGGATTGGAGCTAGGGCTTGGGCTAGAAGATGAGCTGCGCATGGAGCCACTGGGCCT | |
| | GGAAGGGCTAAACATGCTGAGTGACCCCTGTGCCCTGCTGCCTGATCCTGCTGTGGAG | |
| | GAGTCATTCCGCAGTGACCGGCTCCAATGAGGGCACCTCATCACCATCCCTCTTCTTG | |
| | GCCCCATCCCCCACCACCATTCCTTTCCTCCCTTCCCCCTGGCAG | |
| | ORF Start: ATG at 29 | ORF Stop: TGA at 1304 |
| | SEQ ID NO:14 | 425 aa MW at 44036.6 kD |
| NOV6a, | MNPSPQDTYPGPTPPSILPSRRGGGILDGEMDPKVPAIEENLLDDKHLLKPWDAKKLS | |
| CG59233-01 | SSSSRPRSCEVPGISIFPSPDQPANVPVLPPAMNTGGSLPDLTNLHFPPPLPTPLDPE | |
| Protein | ETAYPSLSGGNSTSNLTHTMTHLGISRGMGLGPGYDAPGLHSPLSHPSLQSSLSNPNL | |
| Sequence | QASLSSPQPQLQGSHSHPSLPASSLARHVLPTTSLGHPSLSAPALSSSSSSSSTSSPV | |
| | LGAPSYPASTPGASPHHRRVPLSPLSLLAGPADARRSQQQLPKQFSPTMSPTLSSITQ | |
| | LEQFSMESPSASLVLDPPGFSEGPGFLGGEGPMGGPQDPHTFNHQNLTHCSAAGSGPN | |
| | IILTGDSSPGFSKEIAAALAGVPFEVSAAGLELGLGLEDELRAAEPLGLEGLAALSDP | |
| | CALLPDPAVEESFRSDRLQ | |
| | SEQ ID NO:15 | 1649 bp |
| NOV6b, | CTCTGACTCTGCCCTTCATACAAGTGTGATGAACCCCAGTCCCCAGGATACCTACCCA | |
| CG59233-02 | GGCCCCACACCTCCCAGCATCCTGCCCAGCCGACGTGGGGGTGGTATTCTGGATGGTG | |
| DNA | AAATGGACCCCAAAGTACCTGCTATTGAGGAGAACTTGCTAGATGACAAGCATTTGCT | |
| Sequence | GAAGCCATGGGATGCTAAGAAGCTATCCTCATCCTCTTCCCGACCTCGGTCCTGTGAA | |
| | GTCCCTGGAATTAGCATCTTTCCATCTCCTGACCAGCCTGCCAATGTGCCTGTCCTCC | |
| | CACCTGCCATGAACACGGGGGGCTCCCTACCTGACCTCACCAACCTGCACTTTCCCCC | |
| | ACCACTGCCCACCCCCCTGGACCCTGAAGAGACAGCCTACCCTAGCCTGAGTGGGGGC | |
| | AACAGTACCTCCAATTTGACCCACACCATGACTCACCTGGGCATCAGCAGGGGCATGG | |
| | GCCTGGGCCCAGGCTATGATGCACCAGGTCTTCATTCACCTCTCAGCCACCCATCCCT | |
| | GCAGTCCTCCCTAAGCAATCCCAACCTCCAGGCTTCCCTGAGAAGTCCTCAGCCCCAG | |
| | CTTCAGGGCTCCCACAGCCACCCCTCTCTGCCTGCCTCCTCCTTGGCCCGCCATGTAC | |
| | TGCCCACCACCTCCCTGGGCCACCCCTCACTCAGTGCTCCGGCTCTCTCCTCCTCCTC | |
| | TTCCTCCTCCTCCACTTCATCTCCTGTTTTGGGCGCCCCCTCTTACCCTGCTTCTACC | |
| | CCTGGGGCCTCCCCCCACCACCGCCGTGTGCCCCTCAGCCCCCTGAGTTTGCTCGCGG | |
| | GCCCAGCCGACGCCAGAAGGTCCCAACAGCAGCTGCCCAAACAGTTTTCGCCAACAAT | |
| | GTCACCCACCTTGTCTTCCATCACTCAGGGCGTCCCCCTGGATACCAGTAAACTGTCC | |
| | ACTGACCAGCGGTTACCCCCATACCCATACAGCTCCCCAAGTCTGGTTCTGCCTACCC | |
| | AGCCCCACACCCCAAAGTCTCTACAGCAGCCAGGGCTGCCCTCTCAGTCTTGTTCAGT | |
| | GCAGTCCTCAGGTGGGCAGCCCCCAGGCAGGCAGTCTCATTATGGGACACCGTACCCA | |
| | CCTGGGCCCAGTGGGCATGGGCAACAGTCTTACCACCGGCCAATGAGTGACTTCAACC | |
| | TGGGGAATCTGGAGCAGTTCACCATGGAGAGCCCATCAGCCAGCCTGGTGCTGCATCC | |
| | CCCTGGCTTTTCTGAAGGGCCTGGATTTTTAGGGGGTGAGGGGCCAATGCGTGGCCCC | |
| | CAGGATCCCCACACCTTCAACCACCAGAACTTGACCCACTGTTCCCGCCATGGCTCAG | |
| | GGCCTAACATCATCCTCACAGGTGACTCCTCTCCAGGTTTCTCTAAGGAGATTGCAGC | |
| | AGCCCTGGCCGGAGTGCCTGGCTTTGAGGTGTCAGCAGCTGGATTGGAGCTAGGGCTT | |
| | GGGCTAGAAGATGAGCTGCGCATGGAGCCACTGGGCCTGGAAGGGCTAAACATGCTGA | |
| | GTGACCCCTGTGCCCTGCTGCCTGATCCTGCTGTGGAGGAGTCATTCCGCAGTGACCG | |
| | GCTCCAATGAGGGCACCTCATCACCATCCCTCTTCTTGGCCCCATCCCCCACCACCAT | |
| | TCCTTTCCTCCCTTCCCCCTGGCAG | |
| | ORF Start: ATG at 29 | ORF Stop: TGA at 1574 |
| | SEQ ID NO:16 | 1515 aa MW at 53598.1 kD |

| | |
|---|---|
| NOV6b, | MNPSPQDTYPGPTPPSILPSRRGGGILDGEMDPKVPAIEENLLDDKHLLKPAAAKKLS |
| CG59233-02 | SSSSRPRSCEVPGISIFPSPDQPANVPVLPPAMNTGGSLPDLTNLHFPPPLPTPLDPE |
| Protein | ETAYPSLSGGNSTSNLTHTMTHLGISRGMGLGPGYDAPOLHSPLSHPSLQSSLSNPNL |
| Sequence | QASLSSPQPQLQGSHSHPSLPASSLARHVLPTTSLGHPSLSAPALSSSSSSSSTSSPV |
| | LGAPSYPASTPGASPHHRRVPLSPLSLLAGPADARRSQQQLPKQFSPTMSPTLSSITQ |
| | GVPLDTSKLSTDQRLPPYPYSSPSLVLPTQPHTPKSLQQPGLPSQSCSVQSSGGQPPG |
| | RQSHYGTPYPPGPSGHGQQSYHRPMSDFNLGNLEQFSMESPSASLVLDPPGFSEGPGF |
| | LGGEGPMGGPQDPHTFNHQNLTHCSRHGSGPNIILTGDSSPGFSKEIAAALAGVPGFE |
| | VSAAGLELGLGLEDELRMEPLGLEGLNMLSDPCALLPDPAVEESFRSDRLQ |

Sequence comparison of the above protein sequences yields the following sequence relationships shown in Table 6B.

TABLE 6B
Comparison of NOV6a against NOV6b.

| | NOV6a Residues/NOV6b | Identities/Similarities |
|---|---|---|
| Protein Sequence | Match Residues | for the Matched Region |
| NOV6b | 1 . . . 290 | 290/290 (100%) |
| | 1 . . . 290 | 290/290 (100%) |
| | 291-425 | 135/135 (100%) |
| | 381 . . . 515 | 135/135 (100%) |

Further analysis of the NOV6a protein yielded the following properties shown in Table 6C.

TABLE 6C
Protein Sequence Properties NOV6a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB18966 | Amino acid sequence of a human | 34 . . . 290 | 255/257 (99%) | e−148 |
| | transmembrane protein - Homo sapiens, 316 | 46 . . . 302 | 257/257 (99%) | |
| | aa. [WO200056891-A2, 28-SEP-2000] | | | |
| ABB17428 | Human nervous system related polypeptide | 11 . . . 155 | 128/155 (82%) | 2e−67 |
| | SEQ ID NO 6085 - Homo sapiens, 183 aa. | 6 . . . 160 | 131/155 (83%) | |
| | [WO200159063-A2, 16-AUG-2001] | | | |
| AAB41913 | Human ORFX ORF1677 polypeptide | 56 . . . l55 | 199/100 (99%) | 1e−54 |
| | sequence SEQ ID NO: 3354 - Homo | 2 . . . 101 | 100/100 (100%) | |
| | sapiens, 107 aa. [WO200058473-A2, 05- | | | |
| | OCT-2000] | | | |
| AAB42781 | Human ORFX ORF2545 polypeptide | 328 . . . 425 | 98/98 (100%) | 6e−52 |
| | sequence SEQ ID NO: 5090 - Homo | 7 . . . 104 | 98/98 (100%) | |
| | sapiens, 104 aa. [WO200058473-A2, 05- | | | |
| | OCT-2000] | | | |
| AAU28355 | Novel human secretory protein, Seq ID No | 35 . . . 424 | 176/478 (36%) | 2e−51 |
| | 712 - Homo sapiens, 657 aa. | 208 . . . 657 | 223/478 (45%) | |
| | [WO200166689-A2, 13-SEP-2001] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9D682 | 4632407F12RIK PROTEIN - Mus | 1 . . . 425 | 395/425 (92%) | 0.0 |
| | musculus (Mouse), 425 aa. | 1 . . . 425 | 405/425 (94%) | |
| AAL55758 | HYPOTHETICAL 24.6 KDA | 163 . . . 425 | 171/264 (64%) | 2e−77 |
| | PROTEIN - Homo sapiens (Human), | 3 . . . 235 | 180/264 (67%) | |
| | 235 aa. | | | |
| O75114 | KIAA0616 PROTEIN - Homo sapiens | 35 . . . 424 | 176/478 (36%) | 9e−51 |
| | (Human), 634 aa (fragment). | 185 . . . 634 | 223/478 (45%) | |
| Q96AI8 | HYPOTHETICAL 51.6 KDA | 35 . . . 424 | 171/468 (36%) | 1e−50 |
| | PROTEIN - Homo sapiens (Human), | 45 . . . 494 | 219/468 (46%) | |
| | 494 aa (fragment). | | | |
| Q9H801 | HYPOTHETICAL 62.4 KDA | 35 . . . 371 | 143/387 (36%) | 5e−41 |
| | PROTETN - Homo sapiens (Human), | 185 . . . 556 | 180/387 (45%) | |
| | 593 aa. | | | |

PFam analysis predicts that the NOV6a protein contains the domains shown in the Table 6F.

TABLE 6F
Domain Analysis of NOV6a

| | | | |
|---|---|---|---|
| Pfam | | Identities/Similarities | Expect |
| Domain | NOV6a Match Region | for the Matched Region | Value |

No Significant Matches Found To Known Sequences

### Example A7

The NOV7 clone was analyzed, and the nucleotide and predicted polypeptide sequences are shown in Table 7A.

TABLE 7A
NOV7 Sequence Analysis

| | SEQ ID NO:17 | 2272 bp |
|---|---|---|
| NOV7, | CCGCCGCCGCCGCCGCCGNCGCTACTGCTGNGGGGGNCG | |
| CG58649-01 | CGGGGGCGCGCAATGGGGCGCGGCTCGGAGGGGAGGNTA | |
| DNA | GGGGGGNGTGCCAGGNCCGAAGCCCAGGCGGGGCCGGGA | |
| Sequence | TGCGGCGCTGAGGCCCAGCATGGCCGGCCCGGGCCCCAC | |
| | CTTCCCGCTGCACCGGCTCGTCTGGGCGAACCGGCATCG | |
| | CGAACTGGAGGCCGCACTGCACAGCCACCAGCACGACAT | |
| | TGAACAGGAGGACCCCCGCGGGCGGACCCCACTGGAGCT | |
| | GGCCGTGTCTCTGGGAAACCTGGAGTCTGTGAGAGTGCT | |
| | CCTTCGACACAATGCCAACGTGGGCAAAGAGAACCGCCA | |
| | GGGCTGGGCAGTCCTGCAGGAGGCAGTCAGCACTGGAGA | |
| | CCCCGAGATGGTGCAGCTGGTGCTCCAGTATCGGGACTA | |
| | CCAGAGGGCCACGCAGAGGCTGGCGGGCATTCCGGAACT | |
| | GCTCAACAAACTTCGCCAGGCCCCCGATTTCTACGTTGA | |
| | GATGAAGTGGGAGTTCACCAGCTGGGTGCCCCTTGTGTC | |
| | TAAGATGTGCCCAAGCGATGTGTACCGCGTGTGGAAGCG | |
| | GGGTGAGAGCCTGCGAGTAGACACCAGTCTCCTGGGCTT | |
| | CGAGCACATGACCTGGCAGCGGGGCCGGAGGAGCTTCAT | |
| | CTTCAAGGGCCAGGAGGCAGGAGCCCTGGTGATGGAAGT | |
| | GGACCATGACCGGCAGGTGGTGCATGTGGAGAcACTGGG | |
| | GCTGACTCTGCAGGAGCCCGAAACACTGCTGGCCGCCAT | |
| | GCGGCCCAGCGAGGAGCATGTGGCCACTCCCCCAAATGT | |
| | GGTATCTGGGCCTGGCGGTCTGAGAAGATGGAAACTGTT | |
| | AGCGGCTACGAGGCCAAGGTGTACAGTGCCACCAACGTG | |
| | GAGCTGGTGACACGCACACGCACGGAGCACCTCTCTGAT | |
| | CAGGACAAGTCGAGGAGCAAAGCGGGGAAGACTCCATTC | |
| | CAGTCCTTCCTGGGGATGGCGCAGCAGCATTCCTCCCAC | |
| | ACCGGGGCCCCCGTGCAGCAGGCAGCAAGCCCCACCAAC | |
| | CCCACAGCCATCTCCCCTGAGGAGTACTTCGACCCCAAC | |
| | TTCAGCCTGGAGTCACGGAACATTGGCCGCCCCATCGAG | |
| | ATGTCCAGCAAAGTACAGAGGTTCAAGGCAACACTGTGG | |
| | CTGAGTGAAGAGCACCCGCTCTCCCTGGGTGACCAGGTG | |
| | ACCCCCATCATCGACCTAATGGCCATCAGCAACGCTCAC | |
| | TTTGCCAAGCTGCGCGACTTCATCACTCTGCGCCTTCCA | |
| | CCTGGCTTCCCCGTCAAAATTGAGATTCCCCTTTTCCAC | |
| | GTGCTCAATGCCCGCATCACCTTCAGCAACCTGTGTGGC | |
| | TGTGATGAGCCCCTGAGCTCCGTGTGGGTGCCGGCCCCC | |
| | AGCTCTGCTGTCGCCGCATCAGGGAACCCTTTCCCGTGC | |
| | GAGGTGGACCCCACCGTGTTTGAAGTGCCCAACGGGTAC | |
| | AGCGTGCTGGGCATGGAGCGCAACGAGCCCCTCCGGGAC | |
| | GAGGACGATGACCTCCTGCAGTTCGCCATCCAGCAGAGC | |
| | CTGCTTGAAGCGGGCACTGAGGCGCAGCAGCTGACCGTC | |
| | TGGGAAGCCCTGACCAACACCCGGCCCGGTGCCCGCCCT | |
| | CCTCCCCAGGCCACGGTTTATGAGGAACAGCTTCAGCTG | |
| | GAGCGGGCCCTCCAGGAAAGCCTGCAGCTGTCCACAGAG | |
| | CCCAGGGGCCCAGGATCCCCTCCCAGGACACCCCCAGCC | |
| | CCCGGTCCACCCAGCTTTGAAGAGCAGCTGCGCCTGGCC | |
| | CTGGAGTTGTCTTCACGGGAGCAGGAGGAGCGCGAGCGG | |
| | CGCGGGCAGCAGGAGGAGGAGGACTTACAGCGGATCCTG | |
| | CAGCTGTCACTCACTGAGCACTGAGCCATAGCCCCGGGA | |
| | CGGCTGGCCAGGCCACTCCCTGCCCGCTTTTGTAATTTA | |
| | TTTATTTATAAACTCTCTGCTGCTGAGCTTGGGGCCTGG | |
| | AGCCCCAGGAATGAGCAGGCAGGGGAGACTGAGATGGAA | |
| | ATAAAGAGACTGTCGCAGCAAAAAAAAAAAAAAAAAAAA | |
| | AACTCGAGGGGGGGCCCGGTACCCAATTCGCCCTATAGT | |
| | GAGTCGTATTACAATTCACTGGCCGTCGTTTTACAACGT | |
| | CGTGACTGGGAAAACCCTGGCGTTACCCAACTTAATCGC | |
| | CTTGCAGCACATCCCCCTTTCGCCAGCTGG | |
| | ORF Start: | |
| | ATG at 137 | ORF Stop: TGA at 1952 |
| | SEQ ID NO:18 | 605 aa MW at 68171.1 kD |

| | |
|---|---|
| NOV7, | MAGPGPTFPLHRLVWANRHRELEAALHSHQHDIEQEDPR |
| CG58649-01 | GRTPLELAVSLGNLESVRVLLRHNANVGKENRQGWAVLQ |
| Protein | EAVSTGDPEMVQLAAQYRDYQAATQRAAGIPELLNKLRQ |
| Sequence | APDFYVEMKWEFTSWVPLVSKMCPSDVYRVWKRGESLRV |
| | DTSLLGFEHMTWQRGRRSFIFKCQEAGALVMEVDHDRQV |
| | VHVETLGLTLQEPETLLAAARPSEEHVASRLTSPIVSTH |
| | LDTRNVAFERNKCGIWGWRSEKMETVSGYEAKVYSATAA |
| | ELVTRTRTEHLSDQDKSRSKAGKTPFQSFLGMAQQHSSH |
| | TGAPVQQAASPTNPTAISPEEYFDPNFSLESRNIGRPIE |
| | MSSKVQRFKATLWLSEEHPLSLGDQVTPIIDLMAISNAH |
| | FAKLRDFITLRLPPGFPVKIEIPLFHVLNARITFSNLCG |
| | CDEPLSSVWVPAPSSAVAASGNPFPCEAAPTVFEVPNGY |
| | SVLGMERNEPLRDEDDDLLQFAIQQSLLEAGTEAEQVTA |
| | AEAATNTRPGAAPPPQATVYEEQLQLERALQESLQLSTE |
| | PRGPGSPPRTPPAPGPPSFEEQLRLALELSSREQEERER |
| | RGQQEEEDLQRILQLSLTEH |

Further analysis of the NOV7 protein yielded the following properties shown in Table 7B.

TABLE 7B
Protein Sequence Properties NOV7

| | |
|---|---|
| PSort | 0.4500 probability located in cytoplasm; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV7 protein against the Geneseq database, a proprietary database that contains sequences published in patents and patent publication, yielded several homologous proteins shown in Table 7C.

TABLE 7C
Geneseq Results for NOV7

| | | NOV7 | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB64373 | Amino acid sequence of human intracellular | 1 . . . 358 | 358/358 (100%) | 0.0 |
| | signalling molecule INTRA5 - Homo | 1 . . . 358 | 358/358 (100%) | |
| | sapiens, 358 aa. [WO200077040-A2, 21- | | | |
| | DEC-2000] | | | |
| AAB94937 | Human protein sequence SEQ ID NO: | 305 . . . 605 | 301/301 (100%) | e−175 |
| | 16430 - Homo sapiens, 301 aa. | 1 . . . 301 | 301/301 (100%) | |
| | [EP1074617-A2, 07-FEB-2001] | | | |
| AAU20601 | Human secreted protein, Seq ID No 593 - | 1 . . . 229 | 219/234 (93%) | e−125 |
| | Homo sapiens, 256 aa. [WO200155326-A2, | 20 . . . 253 | 221/234 (93%) | |
| | 02-AUG-2001] | | | |
| AAB42305 | Human ORFX ORF2069 polypeptide | 5 . . . 341 | 191/337 (56%) | e−111 |
| | sequence SEQ ID NO: 4138 - Homo sapiens, | 8 . . . 342 | 243/337 (71%) | |
| | 353 aa. [WO200058473-A2, 05-OCT-2000] | | | |
| AAG77783 | Human colon cancer antigen protein SEQ ID | 367 . . . 505 | 139/139 (100%) | 1e−76 |
| | NO: 8549 - Homo sapiens, 165 aa. | 1 . . . 139 | 139/139 (100%) | |
| | [WO200122920-A2, 05-APR-2001] | | | |

In a BLAST search of public sequence databases, the NOV7 protein was found to have homology to the proteins shown in the BLASTP data in Table 7D.

TABLE 7D
Public BLASTP Results for NOV7

| | | NOV7 | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q960W7 | LD31969P - Drosophila melanogaster | 8 . . . 605 | 271/646 (41%) | e−137 |
| | (Fruit fly), 637 aa. | 11 . . . 637 | 396/646 (60%) | |
| Q9V8R1 | CG15118 PROTEIN - Drosophila | 8 . . . 605 | 274/707 (38%) | e−131 |
| | melanogaster (Fruit fly), 701 aa. | 11 . . . 701 | 400/707 (55%) | |
| O60736 | KE03 PROTEIN - Homo sapiens | 5 . . . 342 | 191/338 (56%) | e−110 |
| | (Human), 367 aa (fragment). | 8 . . . 343 | 243/338 (71%) | |
| Q18099 | HYPOTHETICAL 67.0 KDA | 8 . . . 604 | 214/630 (33%) | 3e−94 |
| | PROTEIN - Caenorhabditis elegans, | 11 . . . 581 | 334/630 (52%) | |
| | 582 aa. | | | |
| Q9V8C6 | CG5742 PROTEIN (LD29875P) - | 8 . . . 424 | 150/448 (33%) | 4e−60 |
| | Drosophila melanogaster (Fruit fly), | 85 . . . 513 | 246/448 (54%) | |
| | 543 aa. | | | |

PFam analysis predicts that the NOV7 protein contains the domains shown in the Table 7E.

TABLE 7E
Domain Analysis of NOV7

| | | Identities/ | |
|---|---|---|---|
| | NOV7 | Similarities | |
| | Match | for the | Expect |
| Pfam Domain | Region | Matched Region | Value |
| ank: domain 1 of 2 | 39 . . . 71 | 15/33 (45%) | 2.2e−06 |
| | | 26/33 (79%) | |
| ank: domain 2 of 2 | 72 . . . 104 | 9/33 (27%) | 29 |
| | | 21/33 (64%) | |
| UIM: domain 1 of 4 | 481 . . . 498 | 7/18 (39%) | 6.2 |
| | | 15/18 (83%) | |
| UIM: domain 2 of 4 | 527 . . . 544 | 6/18 (33%) | 1.4e+02 |
| | | 13/18 (72%) | |
| UIM: domain 3 of 4 | 563 . . . 580 | 10/18 (56%) | 13 |
| | | 14/18 (78%) | |
| UIM: domain 4 of 4 | 588 . . . 605 | 11/18 (61%) | 1.8 |
| | | 15/18 (83%) | |

### Example A8

The NOV8 clone was analyzed, and the nucleotide and predicted polypeptide sequences are shown in Table 8A.

TABLE 8A
NOV8 Sequence Analysis

| | SEQ ID NO:19 | 3653 bp |
|---|---|---|
| NOV8, | GTGTGGGGCGCACGGTCCCGGGATACTGGGGACGGCGGGGTGGGAGGGCGCCGTCCTG | |
| CG58645-01 | GGGCCGCGGCGGCCTGGGCGGGGGAGATGGCGGCGCGATGGAGCAGCGAAAACGTGGT | |
| DNA Sequence | TGTAGAGTTCCGTGACTCCCAGGCAACTGCGATGTCTGTGGACTGTCTTGGGCAGCAT | |
| | GCAGTGCTTTCTGGCCGCAGATTCTTATACATCGTCAATCTAGATGCCCCTTTCGAAG | |
| | GTCACCGAAAGATCTCTCGCCAGAGCAAATGGGACATTGGAGCTGTGCAGTGGAATCC | |
| | TCATGACAGCTTTGCACACTATTTTGCGGCTTCGAGTAACCAACGAGTAGACCTTTAC | |
| | AAGTGGAAAGACGGCAGTGGGGAAGTTGGCACAACCTTACAAGGCCACACTCGTGTCA | |
| | TCAGCGACTTGGACTGGGCGGTGTTTGAGCCTGACCTCCTGGTTACCAGCTCTGTGGA | |
| | CACCTACATCTACATTTGGGATATCAAAGACACAAGGAAACCTACTGTTGCACTGTCT | |
| | GCTGTTGCGGGTGCCTCCCAGGTCAAATGGAATAAAAAAAATGCTAACTGCCTTGCCA | |
| | CCAGCCATGACGGCGATGTGCGGATATGGGATAAGAGGAAACCCAGTACAGCAGTGGA | |
| | ATATCTAGCCGCCCACCTCTCCAAAATCCATGGCCTGGACTGGCACCCAGACAGCGAG | |
| | CACATTCTTGCTACCTCCAGTCAAGACAATTCTGTGAAGTTCTGGGATTACCGCCAGC | |
| | CTCGGAAATACCTCAATATTCTTCCTTGCCAGGTGCCTGTCTGGAAGGCCAGATACAC | |
| | ACCTTTCAGCAATGGATTGGTGACTGTGATGGTTCCCCAGCTGCGGAGGGAAAAAAGC | |
| | CTTCTCCTGTGGAATGTCTTTGACTTGAACACCCCAGTCCACACCTTCGTGGGGCATG | |
| | ATGATGTGGTCCTGGAGTTCCAGTGGAGGAAGCAGAAGGAAGGGTCCAAGGACTATCA | |
| | ACTGGTGACGTGGTCCCGGGATCAGACCTTGAGAATGTGGCGGGTGGATTCCCAGATG | |
| | CAGAGGCTTTGTGCAAATGACATATTAGATGGTGTTGATGAGTTCATTGAGAGTATTT | |
| | CCCTTCTGCCGGAACCTGAGAAGACCCTGCACACTGAAGATACAGATCACCAGCACAC | |
| | TGCAAGCCATGGGGAGGAAGAAGCCCTAAAAGAAGATCCCCCTAGAAATCTCCTGGAA | |
| | GAGAGGAAATCAGATCAACTGGGGCTGCCTCAGACCTTGCAGCAGGAATTCTCCCTGA | |
| | TCAATGTGCAAATCCGGAATGTCAATGTGGAGATGGATGCGGCAGACAGGAGCTGCAC | |
| | AGTGTCTGTGCACTGCAGCAACCATCGTGTCAAGATGCTGGTGAAGTTCCCTGCACAG | |
| | TACCCAAACAACGCCGCCCCTTCCTTCCAGTTTATTAACCCCACAACCATCACATCCA | |
| | CCATGAAAGCTAAGCTGCTGAAGATCCTGGAGTACACAGCCCTGCAGAAAGTGAAGCG | |
| | TGGCCAQAGCTGCCTGGAGCCCTGCCTGCGCCAGCTCGTCTCCTGCCTTGAGTCCTTT | |
| | GTGAACCAGGAAGACAGCGCTTCCAGCAACCCGTTTGCACTCCCCAACTCTGTCACTC | |
| | CCCCCTTACCGACGTTTGCGCGGGTGACCACGGCTTACGGGTCGTACCAGGACGCCAA | |
| | CATTCCCTTTCCTAGGACTTCTGGGGCCAGGTTCTGCGGAGCAGGTTACCTGGTATAT | |
| | TTCACAAGGCCCATGACAATGCATCGGGCGGTGTCTCCCACAGAGCCTACTCCGAGAT | |
| | CTCTCTCAGCCTTGTCTGCTTATCACACTGGCTTGATCGCGCCCATGAAGATCCGCAC | |
| | AGAGGCCCCTGGGAACCTTCGTTTATACAGTGGGAGCCCCACTCGCAGCGAGAAAGAG | |
| | CAGGTCTCCATCAGCTCCTTCTACTACAAGGAGCGGAAATCAAGACGATGGAAAAGTA | |
| | AGCGTGAGGGATCAGACTCTGGCAATCGACAGATCAAGGCTGCTGGGAAAGTCATCAT | |
| | CCAGGATATTGCTTGCCTCCTGCCTGTTCACAAATCGCTGGGAGAGCTGTACATATTG | |
| | AATGTGAATGATATTCAGGAAACATGTCAGAAGAATGCCGCCTCTGCCTTGCTCGTTG | |
| | GAAGAAAGGATCTTGTCCAGGTTTGGTCGCTGGCTACGGTAGCTACAGATCTTTGCCT | |
| | TGGTCCGAAATCTGACCCAGATTTGGAAACACCCTGGGCTCGACATCCATTTGGGCGG | |
| | CAGCTGCTGGAGTCCCTGTTGGCTCACTATTGCCGGCTCCGGGATGTTCAGACACTGG | |
| | CGATGCTCTGTAGCGTGTTTGAAGCCCAGTCTCGGCCTCAGGGGCTACCAAACCCCTT | |
| | TGGGCCTTTTCCTAACCGTTCTTCTAATCTTGTGGTGTCCCATAGTCGATATCCTAGC | |
| | TTTACCTCTTCTGGTTCCTGCTCCAGTATGTCAGACCCAGGGCTCAACACTGGCGGCT | |
| | GGAACATAGCGGGAAGAGAGGCAGAGCACTTGTCCTCCCCTTGGGGAGAATCCTCACC | |
| | AGAAGAGCTCCGCTTTGGGAGTCTGACCTACAGTGATCCCCGTGAGCGAGAACGTGAC | |
| | CAGCATGATAAAAATAAAAGGCTCCTGGACCCCGCCAATACCCAGCAATTTGATGACT | |
| | TTAAGAAATGCTATGGGGAAATCCTCTACCGTTGGGGTCTGAGAGAGAAGCGAGCTGA | |
| | AGTGTTGAAGTTTGTCTCCTGTCCTCCTGACCCTCACAAAGGGATCGAGTTCGGCGTG | |
| | TACTGCAGCCACTGCCGGAGTGAGGTCCGTGGCACGCAGTGTGCCATCTGCAAAGGCT | |
| | TCACGTTCCAGTGTGCCATCTGTCACGTGGCTGTGCGGGGATCGTCCAATTTCTGCCT | |
| | GACCTGTGGGCACGGTGGCCACACCAGCCACATGATGGAGTGGTTTCGGACCCAGGAG | |
| | GTGTGTCCCACCGGGTGTGGGTGCCACTGCCTGCTTGAAAGCACTTTCTGAACCTACA | |
| | GAAGTTGGGTATTGTCTGAAATCCCAGAGGACCCATAAGTGCCGGTGACAAGCTGTCT | |
| | GTCAGGGGAGAGGCTCCAGAACCTGGGTTCGTCCCCAGTGAGACCGGAGGATGATCCC | |
| | CCAAGGACTGCGCAGCATCAGCTCTTGGTGGGCCTCTGCCTTCTCTTCTGTTTGGCCA | |
| | CCTGGTGTGGATGTCACTGTGTGAAGATAAGGACAGAAGTGCAGAGCTGCGCTTTGTG | |
| | TGTTGTCTATGTCGGCTGAGCTACCAAGGTGGAAGTTTTCATGGAGAAAAGCACCTGG | |
| | CTCCAGGGCCAGTGTTACAGTGTTACCCTGTAAGGTGTTAGCCTTAAACCACCGAGCA | |
| | GCGTTCTCTTGATGCCAGTGCAGAGACCAGAGTCAGATGCCCGAGGACAGTGGGTAGG | |
| | AATTTCATCAACAAATGGACCTATGGCATCATGGCTTTAGAAGCTGGTACATTTACTG | |
| | AGCTGATGGACAGTGGCCTTCTAAAATATGACACTTAAATTGTAAATATGCACTGTAC | |
| | TTAAGGATTCTTAAGATGTATTTTTTTGTTATTTCTCCTCCAGCTGCTATCCCTTGGC | |
| | TAATAATTCTAGTAATTTGAAAAGAGAGAAGTTAAAAA | |
| | ORF Start: ATG at 85 | ORF Stop: TGA at 3007 |
| | SEQ ID NO:20 | 974 aa MW at 109841.1 kD |

| | |
|---|---|
| NOV8, | MAARWSSENVVVEFRDSQATANSVDCLGQHAVLSGRRFLYIVNLDAPFEGHRKISRQS |
| CG58645-01 | KWDIGAVQWNPHDSFAHYFAASSNQRVDLYKWKDGSGEVGTTLQGHTRVISDLDWAVF |
| Protein | EPDLLVTSSVDTYIYIWDIKDTRKPTVALSAVAGASQVKWNKKNANCLATSHDGDVRI |
| Sequence | WDKRKPSTAVEYLAAHLSKIHGLDWHPDSEHILATSSQDNSVKFWDYRQPRKYLNILP |
| | CQVPVWKARYTPFSNGLVTVMVPQLRRENSLLLWNVFDLNTPVHTFVGHDDAALEFQW |
| | RKQKEGSKDYQLVTWSRDQTLRNWRVDSQMQRDCANDILDGVDEFIESISLLPEPEKT |
| | LHTEDTDHQHTASHGEEEALKEDPPRNLLEERKSDQLGIPQTLQQEFSLINVQIRNVN |
| | VEMDAADRSCTVSVHCSNHRVKNLVKFPAQYPNNAAPSFQFINPTTITSTMKAKLLKI |
| | LEYTALQKVKRGQSCLEPCLRQLVSCLESFVNQEDSASSNPFALPNSVTPPLPTFARV |
| | TTAYGSYQDANIPFPRTSGARFCGAGYLVYFTRPMTMHRAVSPTEPTPRSLSALSAYH |
| | TGLIAPMKIRTEAPGNLRLYSGSPTRSEKEQVSISSFYYKERKSRRWKSKREGSDSGN |
| | RQIKAAGKVIIQDIACLLPVHKSLGELYILNVNDIQETCQKNAASALLVGRKDLVQVW |
| | SLATVATDLCLGPKSDPDLETPWARHPFGRQLLESLLAHYCRLRDVQTLAMLCSVFEA |
| | QSRPQGLPNPFGPFPNRSSNLVVSHSRYPSFTSSGSCSSMSDPGLNTGGWNIAGREAA |
| | HLSSPWGESSPEELRFGSLTYSDPRERERDQHDKNKRLLDPANTQQFDDPKKCYGEIL |
| | YRWGLREKRAEVLKFVSCPPDPHKGIEFGVYCSHCRSEVRGTQCAICKGFTFQCAICH |
| | VAVRGSSNFCLTCGHGGHTSHMMEWFRTQEVCPTGCGCHCLLESTF |

Further analysis of the NOV8 protein yielded the following properties shown in Table 8B.

TABLE 8B
Protein Sequence Properties NOV8

| | |
|---|---|
| PSort | 0.8800 probability located in nucleus; 0.5150 probability |
| analysis: | located in mitochondrial matrix space; 0.4251 probability |
| | located in microbody (peroxisome); 0.2422 probability |
| | located in mitochondrial inner membrane |
| SignalP | No Known Signal Sequence Predicted |
| analysis: | |

A search of the NOV8 protein against the Geneseq database, a proprietary database that contains sequences published in patents and patent publication, yielded several homologous proteins shown in Table 8C.

TABLE 8C
Geneseq Results for NOV8

| | | NOV8 | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB93945 | Human protein sequence SEQ ID | 571 . . . 974 | 404/404 (100%) | 0.0 |
| | NO: 13963 - Homo sapiens, 419 aa. | 16 . . . 419 | 404/404 (100%) | |
| | [EP1074617-A2, 07-FEB-2001] | | | |
| AAB31789 | Amino acid sequence of a human | 587 . . . 974 | 388/388 (100%) | 0.0 |
| | detoxification protein - Homo sapiens, 388 | 1 . . . 388 | 388/388 (100%) | |
| | aa. [WO200104305-A2, 18-JAN-2001] | | | |
| AAB56854 | Human prostate cancer antigen protein | 297 . . . 485 | 186/189 (98%) | e−105 |
| | sequence SEQ ID NO: 1432 - Homo | 4 . . . 192 | 187/189 (98%) | |
| | sapiens, 201 aa. [WO200055174-A1, 21- | | | |
| | SEP-2000] | | | |
| AAO05762 | Human polypeptide SEQ ID NO 19654 - | 95 . . . 207 | 99/113 (87%) | 9e−54 |
| | Homo sapiens, 124 aa. [WO200164835-A2, | 11 . . . 123 | 103/113 (90%) | |
| | 07-SEP-2001] | | | |
| AAM93739 | Human polypeptide, SEQ ID NO: 3709 - | 103 . . . 220 | 36/124 (29%) | 3e−12 |
| | Homo sapiens, 446 aa. [EP1130094-A2, 05- | 259 . . . 382 | 59/124 (47%) | |
| | SEP-2001] | | | |

In a BLAST search of public sequence databases, the NOV8 protein was found to have homology to the proteins shown in the BLASTP data in Table 8D.

TABLE 8D
Public BLASTP Results for NOV8

| | | NOV8 | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q96PW5 | KIAA1923 PROTEIN - Homo sapiens | 158 . . . 974 | 814/817 (99%) | 0.0 |
| | (Human), 832 aa (fragment). | 16 . . . 832 | 816/817 (99%) | |
| Q96C31 | HYPOTHETICAL 81.2 KDA | 253 . . . 974 | 720/722 (99%) | 0.0 |
| | PROTEIN - Homo sapiens (Human), | 1 . . . 722 | 721/722 (99%) | |
| | 722 aa. | | | |
| Q9BSW6 | HYPOTHETICAL 64.8 KDA | 1 . . . 571 | 569/571 (99%) | 0.0 |
| | PROTEIN - Homo sapiens (Human), | 1 . . . 571 | 570/571 (99%) | |
| | 571 aa. | | | |
| Q9HA43 | CDNA FLJ12270 FIS, CLONE | 571 . . . 974 | 404/404 (100%) | 0.0 |
| | MAMMA1001649 - Homo sapiens | 16 . . . 419 | 404/404 (100%) | |
| | (Human), 419 aa. | | | |
| Q9VKK2 | CG4705 PROTEIN - Drosophila | 9 . . . 954 | 332/998 (33%) | e−143 |
| | melanogaster (Fruit fly), 1004 aa. | 33 . . . 964 | 497/998 (49%) | |

PFam analysis predicts that the NOV8 protein contains the domains shown in the Table 8E.

TABLE 8E
Domain Analysis of NOV8

| | | Identities/ | |
|---|---|---|---|
| | | Similarities | |
| | NOV8 | for the | Expect |
| Pfam Domain | Match Region | Matched Region | Value |
| WD40: domain 1 of 5 | 51 . . . 89 | 4/40 (10%) | 6.3e+02 |
| | | 27/40 (68%) | |
| WD40: domain 2 of 5 | 97 . . . 134 | 13/38 (34%) | 0.0098 |
| | | 28/38 (74%) | |
| WD40: domain 3 of 5 | 141 . . . 176 | 10/38 (26%) | 2.8 |
| | | 25/38 (66%) | |
| WD40: domain 4 of 5 | 183 . . . 220 | 14/38 (37%) | 0.0042 |
| | | 26/38 (68%) | |
| WD40: domain 5 of 5 | 274 . . . 315 | 11/42 (26%) | 31 |
| | | 31/42 (74%) | |
| PHD: domain 1 of 1 | 901 . . . 930 | 9/51 (18%) | 8.7 |
| | | 21/51 (41%) | |
| zf-UBP: domain 1 of 1 | 924 . . . 943 | 7/21 (33%) | 8 |
| | | 14/21 (67%) | |

### Example A9

The NOV9 clone was analyzed, and the nucleotide and predicted polypeptide sequences are shown in Table 9A.

TABLE 9A
NOV9 Sequence Analysis

| | SEQ ID NO:21 | 2134 bp |
|---|---|---|
| NOV9, | GGCGGCTGCGACGCCCTGGTGCAGAGCAGCGCCGTCAAG | |
| CG58632-01 | ATGATCGACCTCAGCGCCTTCAGCCGCAAGCCCCGGACG | |
| DNA | CTCCGGCATCTGCCCCGAACCCCGAGGCCGGAGCTGAAC | |
| Sequence | GTGGCCCCATATGACCCTCACTTCCCGGCCCCGGCCCGG | |
| | GATGGCTTCCCCGAGCCCAGCATGGCGCTGCCTGGGCCA | |
| | GAGCCCTTGCCCACAGAGTGTGGGTTCGAGCCACCCCAC | |
| | CTGGCCCCCCTGAGTGACCCCGAGGCCCCCAGCATGGAG | |
| | TCCCCGGAGCCTGTCAAGCCGGAACAGGGCTTCGTGTGG | |
| | CAGGAGGCCAGTGAGTTCGAGGCTGAAACGGCGGGTTCG | |
| | ACCGTGGAACGCCACAAGAAGGCCCAGCTGGATCGGCTG | |
| | GACATCAACGTGCAGATTGACGACTCCTATCTGGTGGAG | |
| | GCGGGCGACCGCCAGAAGCGCTGGCAGTGCCGCATGTGC | |
| | GAGAAGTCCTACACGTCCAAGTACAACCTGGTGACGCAA | |
| | ATCCTGGGCCACAACGGCATCAAGCCACACTCGTGCCCA | |
| | CACTGCAGCAAGCTCTTCAAGCAGCCCAGCCACCTGCAG | |
| | ACGCACCTGCTGACGCACCAGGGCACCCGGCCCCACAAG | |
| | TGCCAGGTATGCCACAAGGCCTTCACGCAGACCAGCCAC | |
| | CTCAAGCGCCACATGCTGCTGCACTCGGAGGTCAAGCCC | |
| | TACAGCTGCCACTTCTGCGGCCGCGGCTTCGCCTACCCC | |
| | AGCGAGCTCAAGGCCCACGAAGTGAAGCATGAGAGTGGC | |
| | CGCTGCCATGTCTGCGTCGAGTGCGGCCTGGACTTCTCC | |
| | ACCCTGACCCAGCTCAAGCGCCACCTGGCCTCCCACCAG | |
| | GGCCCCACCCTCTACCAGTGCCTCGAGTGTGACAAGTCC | |
| | TTCCACTACCGCAGCCAGTTGCAGAACCACATGCTCAAG | |
| | CACCAGAACGTGCGACCCTTCGTGTGCACTGAATGCGGC | |
| | ATGGAGTTCAGCCAGATTCACCACCTCAAGCAGCACTCC | |
| | CTCACCCACAAGGGCGTGAAGGAGTTCAAGTGCGAGGTG | |
| | TGTGGCCGGGAGTTCACCCTACAGGCGAACATGAAGCGG | |
| | CACATGCTGATCCACACCAGCGTCCGGCCCTACCAGTGC | |
| | CACATCTGCTTCAAGACCTTTGTACAGAAGCAGACTCTC | |
| | AAGACCCACATGATTGTACACTCGCCCGTGAAGCCATTC | |
| | AAATGCAAGGTGTGCGGGAAGTCCTTCAACCGCATGTAC | |
| | AACCTGCTGGGCCACATGCACCTGCACGCCGGCAGCAAG | |
| | CCCTTCAAGTGCCCCTACTGCTCCAGCAAGTTTAATCTC | |
| | AAGGGCAACCTGAGCCGGCACATGAAGGTCAAGCATGGC | |
| | GTCATGGACATCGGCCTGGACAGCCAAGACCCCATGAGT | |
| | GGAGCCGACAGCGCAATGGACCCTTCAGAGCACGAAGGA | |
| | CACAAGGACATGGACGCACTTCGAGGAGAACGCCTACCC | |
| | AGCTATGAGGAGCGGGGACAGCAGCGCAGAGGCCAGTGT | |
| | CCTCACTGAACAGGCCATGAAAGAGATGGCCTACTACAA | |
| | CGTGCTATAGCGCAAGCTGGGCCACCCCTAACGGGGGCC | |
| | GGGGGCGAGGGCATGGGGGTGAGACCCATGGGCTGCAGG | |
| | CTGCACCTCCTTGCAGCCGAGACACAGTTTATGGGCCCC | |
| | ATTGTTCTGAGCCCTTCCCTTCCCGAAGTCATTCGCACA | |
| | CTAGGGACCTTTGGACCACATGGAGACTGTACTACTGGG | |
| | CCCGGCTGGTGGGCCAGCCCGGGCCAGGGCTCCAGGCAG | |
| | GGACAGGCAAACACGCCAGGCCCAAATCTGGGTCCCCCG | |
| | GGCTGCTCCGCGGAAAGTCGGGCACAACAATGGCGCCCA | |
| | CGGGTGCAGGGTCACAAGGGCCACGGACCAGAGGGCACT | |
| | ACCACGCACAAGCGAACCCTCTACCGGGCCGTCACGAGT | |
| | GACAAAGAAACCCTGATCCACGTTCTTTCCCAACAACGC | |
| | CAAAGAGAAGAAACCACTACACAGACCACCCACACTATG | |
| | ATGACCCCCCTACGGTACGACACAAGAAGATGCACTACA | |
| | CGGCGCCACCACACAGACGCACTCGTAGGAGTAGACGCA | |
| | CCAGATCAGTGTACACCAGTCAGCGACG | |
| | ORF Start: | |
| | ATG at 40 | ORF Stop: TGA at 1528 |
| | SEQ ID NO:22 | 496 aa MW at 56790.7 kD |

| | |
|---|---|
| NOV9, | MIDLSAFSRKPRTLRHLPRTPRPELNVAPYDPHFPAPAR |
| G58632-01 | DGFPEPSMALPGPEPLPTECGFEPPHLAPLSDPEAPSME |
| Protein | SPEPVKPEQGFVWQEASEFEADTAGSTVERHKKAQLDRL |
| Sequence | DINVQIDDSYLVEAGDRQKRWQCRMCEKSYTSKYNLVTH |
| | ILGHNGIKPHSCPHCSKLFKQPSHLQTHLLTHQGTRPHK |
| | CQVCHKAFTQTSNLKRHMLLHSEVKPYSCHFCGRGFAYP |
| | SELKAHEVKHESGRCHVCVECGLDFSTLTQLKRHLASHQ |
| | GPTLYQCLECDKSFHYRSQLQNHMLKHQNVRPFVCTECG |
| | MEFSQIHHLKQHSLTHKGVKEFKCEVCGREFTLQANMKR |
| | HMLIHTSVRPYQCHICFKTFVQKQTLKTHMTVHSPVKPF |
| | KCKVCGKSFNRMYNLLGHMHLHAGSKPFKCPYCSSKFNL |
| | KGNLSRHMKVKHGVMDTGLDSQDPMSGADSANDPSEHEG |
| | HQDMDALRGERLPSYEERGQQRRGQCPH |

Further analysis of the NOV9 protein yielded the following properties shown in Table 9B.

TABLE 9B
Protein Sequence Properties NOV9

| | |
|---|---|
| PSort | 0.6005 probability located in mitochondrial matrix space; |
| analysis: | 0.4200 probability located in nucleus; 0.3108 probability |
| | located in microbody (peroxisome); 0.3067 probability |
| | located in mitochondrial inner membrane |
| SignalP | No Known Signal Sequence Predicted |
| analysis: | |

A search of the NOV9 protein against the Geneseq database, a proprietary database that contains sequences published in patents and patent publication, yielded several homologous proteins shown in Table 9C.

TABLE 9C
Geneseq Results for NOV9

| | | NOV9 | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU15995 | Human novel secreted protein, Seq ID 948 - | 131 . . . 454 | 324/324 (100%) | 0.0 |
| | Homo sapiens, 338 aa. [WO200155322-A2, | 7 . . . 330 | 324/324 (100%) | |
| | 02-AUG-2001] | | | |
| AAU16432 | Human novel secreted protein, Seq ID 1385 - | 228 . . . 373 | 146/146 (100%) | 1e−87 |
| | Homo sapiens, 147 aa. [WO200155322- | 1 . . . 146 | 146/146 (100%) | |
| | A2, 02-AUG-2001] | | | |
| AAB95278 | Human protein sequence SEQ ID NO: | 135 . . . 442 | 124/308 (40%) | 1e−74 |
| | 17486 - Homo sapiens, 803 aa. | 472 . . . 779 | 169/308 (54%) | |
| | [EP1074617-A2, 07-FEB-2001] | | | |
| AAM40001 | Human polypeptide SEQ ID NO 3146 - | 135 . . . 439 | 124/305 (40%) | 4e−73 |
| | Homo sapiens, 700 aa. [WO200153312-A1, | 369 . . . 673 | 165/305 (53%) | |
| | 26-JUL-2001] | | | |
| ABB20596 | Protein #2595 encoded by probe for | 134 . . . 436 | 117/303 (38%) | 4e−72 |
| | measuring heart cell gene expression - | 27 . . . 329 | 175/303 (57%) | |
| | Homo sapiens, 340 aa. [WO200157274-A2, | | | |
| | 09-AUG-2001] | | | |

In a BLAST search of public sequence databases, the NOV9 protein was found to have homology to the proteins shown in the BLASTP data in Table 9D.

TABLE 9D
Public BLASTP Results for NOV9

| | | NOV9 | Identities/ | |
|---|---|---|---|---|
| Protein | | Residues/ | Similarities | |
| Accession | | Match | for the | Expect |
| Number | Protein/Organism/Length | Residues | Matched Portion | Value |
| O42492 | ZINC FINGER PROTEIN - Fugu rubripes | 112 . . . 451 | 255/340 (75%) | e−166 |
| | (Japanese pufferfish) (Takifugu rubripes), | 139 . . . 478 | 295/340 (86%) | |
| | 490 aa. | | | |
| Q96TE6 | CDNA FLJ13029 FIS, CLONE | 135 . . . 442 | 124/308 (40%) | 5e−74 |
| | NT2RP3001057, MODERATELY SIMILAR | 472 . . . 779 | 169/308 (54%) | |
| | TO ZINC FINGER PROTEIN 91 - Homo | | | |
| | sapiens (Human), 803 aa. | | | |
| Q9NR94 | ZINC FINGER PROTEIN - Homo sapiens | 135 . . . 442 | 124/308 (40%) | 5e−74 |
| | (Human), 686 aa. | 355 . . . 662 | 169/308 (54%) | |
| Q9NYT6 | Zinc finger protein 226 - Homo sapiens | 135 . . . 442 | 124/308 (40%) | 5e−74 |
| | (Human), 803 aa. | 472 . . . 779 | 169/308 (54%) | |
| Q96IR4 | SIMILAR TO ZINC FINGER PROTEIN | 135 . . . 439 | 124/305 (40%) | le−72 |
| | 224 - Homo sapiens (Human), 700 aa. | 369 . . . 673 | 165/305 (53%) | |

PFam analysis predicts that the NOV9 protein contains the domains shown in the Table 9E.

TABLE 9E
Domain Analysis of NOV9

| | | Identities/ | |
|---|---|---|---|
| | | Similarities | |
| | NOV9 | for the | Expect |
| Pfam Domain | Match Region | Matched Region | Value |
| zf-C2H2: domain 1 of 11 | 138 . . . 160 | 9/24 (38%) | 0.0011 |
| | | 16/24 (67%) | |
| zf-C2H2: domain 2 of 11 | 166 . . . 188 | 11/24 (46%) | 0.00018 |
| | | 20/24 (83%) | |
| TFIIS: domain 1 of 1 | 166 . . . 204 | 11/39 (28%) | 0.46 |
| | | 20/39 (51%) | |
| zf-C2H2: domain 3 of 11 | 194 . . . 216 | 11/24 (46%) | 8e−05 |
| | | 20/24 (83%) | |
| zf-BED: domain 1 of 1 | 172 . . . 217 | 12/55 (22%) | 2 |
| | | 30/55 (55%) | |
| zf-C2H2: domain 4 of 11 | 222 . . . 244 | 10/24 (42%) | 0.00015 |
| | | 17/24 (71%) | |
| PHD: domain 1 of 1 | 195 . . . 255 | 14/66 (21%) | 9.1 |
| | | 38/66 (58%) | |
| zf-C2H2: domain 5 of 11 | 250 . . . 272 | 11/24 (46%) | 0.0005 |
| | | 17/24 (71%) | |
| zf-C2H2: domain 6 of 11 | 278 . . . 300 | 10/24 (42%) | 7.8e−05 |
| | | 19/24 (79%) | |
| zf-C2H2: domain 7 of 11 | 306 . . . 328 | 10/24 (42%) | 0.0011 |
| | | 18/24 (75%) | |
| zf-C2H2: domain 8 of 11 | 334 . . . 356 | 10/24 (42%) | 8.4e−06 |
| | | 20/24 (83%) | |
| zf-C3HC4: domain 1 of 1 | 364 . . . 376 | 5/13 (38%) | 0.5 |
| | | 11/13 (85%) | |
| zf-C2H2: domain 9 of 11 | 362 . . . 384 | 10/24 (42%) | 1.6e−05 |
| | | 19/24 (79%) | |
| zf-C2H2: domain 10 of 11 | 390 . . . 412 | 12/24 (50%) | 1.7e−05 |
| | | 18/24 (75%) | |
| zf-C2H2: domain 11 of 11 | 418 . . . 441 | 11/25 (44%) | 0.00014 |
| | | 20/25 (80%) | |

### Example A10

The NOV10 clone was analyzed, and the nucleotide and predicted polypeptide sequences are shown in Table 10A.

TABLE 10A
NOV10 Sequence Analysis

| | SEQ ID NO:23 | 1678 bp |
|---|---|---|
| NOV10a, | GGCACGAGGCCCGGAAGTCCGGGCGGGCGCCGAGCCGGGTGCGGCACGAGGCAGAGCC | |
| CG58630-01 | GTAAAGGCGCGCGGGAACATGGGGCTGTATGCTGCAGCTGCAGGCGTGTTGGCCGGCG | |
| DNA Sequence | TGGAGAGCCGCCAGGGCTCTATCAAGGGGTTGGTGTACTCCAGCAACTTCCAGAACGT | |
| | GAAGCAGCTGTACGCGCTGGTGTGCGAAACGCAGCGCTACTCCGCCGTGCTGGATGCT | |
| | GTGATCGCCAGCGCCGGCCTCCTCCGTGCGGAGAAGAAGCTGCGGCCGCACCTGGCCA | |
| | AGGTGCTAGTGTATGAGTTGTTGTTGGGAAAGGGCTTTCGAGGGGGTGGGGGCCGATG | |
| | GAAGGCTCTGTTGGGCCGGCACCAGGCGAGGCTCAAGGCTGAGTTGGCTCGGCTCAAG | |
| | GTTCATCGGGGTGTGAGCCGGAATGAGGACCTGTTGGAAGTGGGATCCAGGCCTGGTC | |
| | CAGCCTCCCAGCTGCCTCGATTTGTGCGTGTGAACACTCTCAAGACCTGCTCCGATGA | |
| | TGTAGTTGATTATTTCAAGAGACAAGGTTTCTCCTATCAGGGTCGGCCTTCCAGCCTC | |
| | GATGACTTACGAGCCCTCAAGGGGAAGCATTTTCTCCTCGACCCCTTGATGCCGGAGC | |
| | TGCTGGTGTTTCCCGCCCAGACAGATCTGCATGAACACCCACTGTACCGGGCCGGACA | |
| | CCTCATTCTGCAGGACAGGGCCAGCTGTCTCCCAGCCATGCTGCTGGACCCCCCGCCA | |
| | GGCTCCCATGTCATCGATGCCTGTGCCGCCCCAGGCAATAAGACCAGTCACTTGGCTG | |
| | CTCTTCTGAAGAACCAAGGGAAGATCTTTGCCTTTGACCTGGATGCCAAGCGGCTGGC | |
| | ATCCATGGCCACGCTGCTGGCCCGGGCTGGCGTCTCTTGCTGTGAACTGGCTGAGGAG | |
| | GACTTCCTGGCGGTCTCCCCCTCGGATCCACGCTACCATGAGGTCCACTACATCCTGC | |
| | TGGATCCTTCCTGCAGTGGCTCCGGTATGCCGAGCAGACAGCTCGAGGAGCCCGGGGC | |
| | AGGCACACCTAGCCCGGTGCGTCTGCATGCCCTGGCAGGGTTCCAGCAGCGAGCCCTG | |
| | TGCCACGCGCTCACTTTCCCTTCCCTGCAGCGGCTCGTCTACTCCATGTGCTCCCTCT | |
| | GCCAGGAGGAGAATGAAGACATGGTACAAGATGCGCTGCAGCAGAACCCGGGCGCCTT | |
| | CAGGCTAGCTCCCGCCCTGCCTGCCCGGCCCCACCGAGGCCTGAGCACGTTCCCGGGT | |
| | GCCGAGCACTGCCTCCGGGCTTCCCCCAAGACCACGCTTAGCGGTGGCTTCTTCGTTG | |
| | CTGTAATTGAACGGGTCGAGATGCCGACCACCTCACAGGCCAAAGCATCAGCACCAGA | |
| | ACGCACACCCAGCCCAGCCCCAAAGAGAAAGAAGAGAGCAAAAAGCTGCAGCCGGTGC | |
| | TTGCACACCGCCTTGCACATAGCAGAGGCTCCGGGCTCACTCCTTCCTGGTGCAAAAC | |
| | GAAGATGCCTGTCCTCTCCGTGGAAGACCCTGGGCCCTCACCGCAGGCAGCAGTTTGC | |
| | GTTTTGAAAGGTTATTGGGTCCCTTCCTCGGGCTGTGTTCTTGCTGGTGAGCAAAAGT | |
| | GTTGCCTGCAGAAATAAAATGCAGAACGTACTCTACGATAAAAAAAAAAAAAAA | |
| | ORF Start: ATG at 77 | ORF Stop:TGA at 1571 |
| | SEQ ID NO:24 | 498 aa MW at 54070.7 kD |
| NOV10a, | MGLYAAAAGVLAGVESRQGSIKGLVYSSNFQNVKQLYALVCETQRYSAVLDAVIASAG | |
| CG58630-01 | LLRAEKKLRPHLAKVLVYELLLGKGFRGGGGRWKALLGRHQARLKAELAALKAARGVS | |
| Protein | RNEDLLEVGSRPGPASQLPRFVRVNTLKTCSDDVVDYFKRQGFSYQGRASSLDDLRAL | |
| Sequence | KGKHFLLDPLMPELLVFPAQTDLHEHPLYRAGHLILQDRASCLPAMLLDPPPGSHVID | |
| | ACAAPGNKTSHLAALLKNQGKIFAFDLDAKRLASMATLLARAGVSCCELAEEDFLAVS | |
| | PSDPRYHEVHYILLDPSCSGSGMPSRQLESPGAGTPSPVRLHAAAGFQQAALCHAATF | |
| | PSLQRLVYSMCSLCQEENEDMVQDADQQNPGAFRLAPAAPAAPHRGLSTFPGAEHCLR | |
| | ASPKTTLSGGFFVAVIERVEMPTTSQAKASAPERTPSPAPKRKKAAKSCSRCLHTALH | |
| | IAEAPGSLLPGGKGRCLSSPWKTLGPHRRQQFAF | |
| | SEQ ID NO:25 | 11219 bp |
| NOV10b, | AAGGCGCGCGGGAACATGGGGCTGTACGCTGCGGTGGCAGGCGTGCTGGCCGGCGTGG | |
| CG58630-02 | AGAGCCGCCAGGGCTCTATCAAGGGGCTGGTGTACTCCAGCAACTTCCAGCCTCGATG | |
| DNA Sequence | ACTTACGAGCCCTCAAGGGGAAGCATTTTCTCCTGGACCCCTTGATGCCGGAGCTGCT | |
| | GGTGTTTCCCGCCCAGACAGATCTGCATGAACACCCACTGTACCGGGCCGGACACCTC | |
| | ATTCTGCAGGACAGGGCCAGCTGTCTCCCAGCCATGCTGCTGGACCCCCGCCAGGCTC | |
| | CCATGTCATGGATGCCTGTGCCACCCCAGGCAATAAAGACCAGTCACTTGGCTGCTCT | |
| | TCTGAAGAACCAAGGGAAGATCTTTGCCTTTGACCTGGGTGCCAGGCGGCTGGCATCC | |
| | ATGGCCACGCTGCTGGCCTGGGCTGGCGTCTCCTGCTGTGAGCTGGCTGAGGAGGACT | |
| | TCCTGGCGGTCTCCCCCTTAGATCCGCGCTATCGTGAGGTCCACTATGTCCTGCTGGA | |
| | TCCTTCCTGCAGTGGCTCGGGTGAGATGGTATGCCGAGCAGACAGCTGGAGGAGCCCG | |
| | GGGCAGGGACACCTTAGCCCGGTGCGTCTGCATGCCCTGGCAGGGTTCCAGCAGCGAG | |
| | CCCTGTGCCACGCGCTCACTTTCCCTTCCCTGCAGCGGCTCGTCTACTCCATGTCCTC | |
| | CCTCTGCCAGGAGGAGAATGAAGACATGGTACAAGATGCGCTGCAGCAGAACCCGGGC | |
| | GCCTTCAGGCTAGCTCCCGCCCTGCCTGCCCGCCCCAACCCAGGCCTGAGCACGTTCC | |
| | CGGGTGCCGAGCACTGCCTCCGGGCTTCCCCCAAGACCACGCTTAGCGGTGGCTTCTT | |
| | CGTTGCTGTAATTGAACGGGTCGAGATGCCGACCACCTCACAGGCCAAAGCATCAGCA | |
| | CCAGAACGCACACCCAGCCCAGCCCCAAAGAGAAAGAAGAGAGCAAAAAGCTGCAGCC | |
| | GGTGCTTGCACACCGCCTTGCACATAGCAGAGGCTCCGGGCTCACTCCTTCCTGGTGG | |
| | GAAAGGAAGATGCCTGTCCTCTCCGTGGAAGACCCTGGGCCCTCACCGCAGGCAGCAG | |
| | TTTGCGTTTTGAAAGGTTATTGGGTCCCTTCCTCGGGCTGTGTTCTTGCTGGTGAGCA | |
| | AAAGTGTTGCCTGCAGAAATAAAATGCAGAACGTACTCTACGATAAAAAAAAAAAAAA | |
| | A | |
| | ORF Start: ATG at 161 | ORF Stop: TGA at 1112 |
| | SEQ ID NO:26 | 1317 aa MW at 34690.7 kD |

| | |
|---|---|
| NOV10b, | MPELLVFPAQTDLHEHPLYRAGHLILQDRASCLPAALLDPRQAPMSAAPVPPQAIKTS |
| CG58630-02 | HLAALLKNQGKIFAFDLGARRLASMATLLAWAGVSCCELAEEDFLAVSPLDPRYREAA |
| Protein | YVLLDPSCSGSGEMVCRADSWRSPGQGHLSPVRLHALAGFQQIAACHAATFPSLQRLV |
| Sequence | YSMCSLCQEENEDMVQDALQQNPGAFRLAPALPARPHRGLSTFPGAEHCLAASPKTTL |
| | SGGFFVAVIERVEMPTTSQAKASAPERTPSPAPKRKKAAKSCSRCLHTAAHIAEAPGS |
| | LLPGGKGRCLSSPWKTLGPHRRQQFAF |

Sequence comparison of the above protein sequences yields the following sequence relationships shown in Table 10B.

TABLE 10B
Comparison of NOVl0a against NOV10b.

| | NOV10a Residues/ | Identities/Similarities |
|---|---|---|
| | NOV10b | for the Matched |
| Protein Sequence | Match Residues | Region |
| NOV10b | 185 . . . 498 | 284/317 (90%) |
| | 1 . . . 317 | 286/317 (90%) |

Further analysis of the NOV 10a protein yielded the following properties shown in Table 10C.

TABLE 10C
Protein Sequence Properties NOV10a

| | |
|---|---|
| PSort | 0.3700 probability located in outside; 0.1900 probability |
| analysis: | located in plasma membrane; 0.1000 probability located in |
| | endoplasmic reticulum (membrane); 0.1000 probability |
| | located in endoplasmic reticulum (lumen) |
| SignalP | No Known Signal Sequence Predicted |
| analysis: | |

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAG89191 | Human secreted protein, SEQ ID NO:311 - | 1 . . . 451 | 442/452 (97%) | 0.0 |
| | Homo sapiens, 466 aa. [WO200142451- | 1 . . . 452 | 448/452 (98%) | |
| | A2, 14-JUN-2001] | | | |
| AAB92639 | Human protein sequence SEQ ID | 1 . . . 428 | 421/428 (98%) | 0.0 |
| | NO:10961 - Homo sapiens, 429 aa. | 1 . . . 428 | 425/428 (98%) | |
| | [EP1074617-A2, 07-FEB-2001] | | | |
| AAY86441 | Human gene 40-encoded protein fragment, | 19 . . . 451 | 419/434 (96%) | 0.0 |
| | SEQ ID NO:356 - Homo sapiens, 470 aa. | 23 . . . 456 | 424/434 (97%) | |
| | [WO9966041-A1, 23-DEC-1999] | | | |
| AAY86442 | Human gene 40-encoded protein fragment, | 67 . . . 456 | 372/391 (95%) | 0.0 |
| | SEQ ID NO:357 - Homo sapiens, 429 aa. | 30 . . . 420 | 380/391 (97%) | |
| | [WO9966041-A1, 23-DEC-1999] | | | |
| AAG89192 | Human secreted protein, SEQ ID NO:312 - | 1 . . . 335 | 335/335 (100%) | 0.0 |
| | Homo sapiens, 382 aa. [WO200142451- | 1 . . . 335 | 335/335 (100%) | |
| | A2, 14-JUN-2001] | | | |

In a BLAST search of public sequence databases, the NOV10a protein was found to have homology to the proteins shown in the BLASTP data in Table 10E.

TABLE 10E
Public BLASTP Results for NOV10a

| | | NOV10a | | |
|---|---|---|---|---|
| Protein | | Match | Identities/ | Expect |
| Number | Protein/Organism/Length | Residues | Matched Portion | Value |
| Q96HT9 | HYPOTHETICAL 50.4 KDA PROTEIN - | 1 . . . 451 | 442/452 (97%) | 0.0 |
| | Homo sapiens (Human), 466 aa. | 1 . . . 452 | 448/452 (98%) | |
| Q9NW70 | HYPOTHETICAL 46.7 KDA PROTEIN - | 1 . . . 428 | 421/428 (98%) | 0.0 |
| | Homo sapiens (Human), 429 aa. | 1 . . . 428 | 425/428 (98%) | |
| Q96P11 | NOL1R - Homo sapiens (Human), 429 aa. | 1 . . . 428 | 419/428 (97%) | 0.0 |
| | | 1 . . . 428 | 423/428 (97%) | |
| Q9HAH4 | HYPOTHETICAL 34.5 KDA PROTEIN | 185 . . . 498 | 290/315 (92%) | e−166 |
| | (NOL1R2) - Homo sapiens (Human), 315 | 1 . . . 315 | 294/315 (93%) | |
| | aa. | | | |
| O14039 | HYPOTHETICAL 51.3 KDA PROTEIN | 1 . . . 451 | 169/463 (36%) | 1e−64 |
| | C2C4.06C IN CHROMOSOME I - | 1 . . . 443 | 248/463 (53%) | |
| | Schizosaccharomyces pombe (Fission | | | |
| | yeast), 455 aa. | | | |

PFam analysis predicts that the NOV10a protein contains the domains shown in the Table 10F.

TABLE 10F
Domain Analysis of NOV10a

| | NOV10a | Identities/Similarities | Expect |
|---|---|---|---|
| Pfam Domain | Match Region | for the Matched Region | Value |
| Nol1_Nop2_Sun: | 201 . . . 276 | 31/90 (34%) | 1.9e−15 |
| domain 1 of 2 | | 57/90 (63%) | |
| Nol1_Nop2_Sun: | 353 . . . 378 | 13/36 (36%) | 0.091 |
| domain 2 of 2 | | 20/36 (56%) | |

### Example A11

The NOV11 clone was analyzed, and the nucleotide and predicted polypeptide sequences are shown in Table 11A.

TABLE 11A
NOV11 Sequence Analysis

| | SEQ ID NO:27 | 560 bp |
|---|---|---|
| NOV 11, | GAGCGATGCCGAACTGAATCTGGCGGTAGAGCGACTGGA | |
| CG59373-01 | CGACAGTGCGCTGACCGCCCTCCTTGATCGTTATGACCA | |
| DNA | GGTGATCACCCTCTAATGGCCACCCTGCACATACTTTCC | |
| Sequence | CATTCGCCGTTCGCCGATAGCCGCCTGGCTAGCTGCCTG | |
| | CGTCTGTTGGGTGCCGCTGATGGTCTGTTGCTCAGCGGC | |
| | GATGCGGTCTACGCGCTACAGCCCGGCACAGCCAACCTG | |
| | CAGGCTCTGCAACTGATGTCCGCCAGCGTTGCGCTCTAT | |
| | GCACTGGGCGAAGACCTAAGCGCCCGCGGCCTGCAGGCG | |
| | CCCGAGCGCGCACAGGTCGTGGACTATCCAGAGTTCGTC | |
| | GAGCTATGCATCCGCTACGCCAAGGTCAACAGCTGGCTA | |
| | TGAGCACGCTCAACGTCGCCGGACGTGAAATTGCCCTGG | |
| | ACAAGGACGGCTACCTGCTCGACCTGCAGGATTGGTCAC | |
| | ACCCCGTAGCCGAGGCGCTGGCCGCAGCTGAAGATTTGC | |
| | AGCTGAGTGAAGAACACTGGGAAATTCTCGACCTGCTGC | |
| | GCACTTCTGAGGAT | |
| | ORF Start: | |
| | ATG at 94 | ORF Stop: TGA at 391 |
| | SEQ ID NO:28 | 99 aa MW at 10589.0 kD |

| | |
|---|---|
| NOV11, | MATLHILSHSPFADSRLASCLRLLGAADGLLLSGDAVYA |
| CG59373-01 | LQPGTANLQALQLMSASVALYALGEDLSARGLQAPERAQ |
| Protein | VVDYPEFVELCIRYAKVNSWL |
| Sequence | |

Further analysis of the NOV11 protein yielded the following properties shown in Table 11B.

TABLE 11B
Protein Sequence Properties NOV11

| | |
|---|---|
| PSort | 0.4856 probability located in mitochondrial matrix space; |
| analysis: | 0.3000 probability located in microbody (peroxisome); 0.2654 |
| | probability located in lysosome (lumen); 0.1962 probability |
| | located in mitochondrial inner membrane |
| SignalP | No Known Signal Sequence Predicted |
| analysis: | |

A search of the NOV11 protein against the Geneseq database, a proprietary database that contains sequences published in patents and patent publication, yielded several homologous proteins shown in Table 11C.

TABLE 11C
Geneseq Results for NOV11

| | | NOV11 | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| AAG98264 | Escherichia coli protein sequence SEQ ID | 4 . . . 98 | 38/97 (39%) | 1e−08 |
| | NO:312 - Escherichia coli, 95 aa. | 2 . . . 95 | 53/97 (54%) | |
| | [WO200148209-A2, 05-JUL-2001] | | | |

In a BLAST search of public sequence databases, the NOV11 protein was found to have homology to the proteins shown in the BLASTP data in Table 11D.

TABLE 11D
Public BLASTP Results for NOV11

| | | NOV11 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9I0N1 | HYPOTHETICAL PROTEIN PA2607 - | 3 . . . 99 | 60/98 (61%) | 4e−25 |
| | Pseudomonas aeruginosa, 101 aa. | 4 . . . 101 | 69/98 (70%) | |
| O87898 | DSRH - Chromatium vinosum, 102 aa. | 1 . . . 99 | 38/103 (36%) | 2e−08 |
| | | 1 . . . 102 | 50/103 (47%) | |
| P45530 | Hypothetical protein yheL - Escherichia | 4 . . . 98 | 38/97 (39%) | 4e−08 |
| | coli, 95 aa. | 2 . . . 95 | 53/97 (54%) | |
| AAG58450 | ORF, HYPOTHETICAL PROTEIN - | 4 . . . 98 | 38/97 (39%) | 8e−08 |
| | Escherichia coli O157:H7 EDL933, 95 aa. | 2 . . . 95 | 53/97 (54%) | |
| AAL22312 | PUTATIVE OXIDATION OF | 4 . . . 98 | 38/97 (39%) | 1e−07 |
| | INTRACELLULAR SULFUR - | 2 . . . 95 | 50/97 (51%) | |
| | Salmonella typhimurium LT2, 95 aa. | | | |

PFam analysis predicts that the NOV11 protein contains the domains shown in the Table 11E.

TABLE 11E
Domain Analysis of NOV11

| | | | |
|---|---|---|---|
| | NOV11 | Identities/Similarities | Expect |
| Pfam Domain | Match Region | for the Matched Region | Value |

No Significant Matches Found To Known Sequences

### Example A12

The NOV12 clone was analyzed, and the nucleotide and predicted polypeptide sequences are shown in Table 12A.

TABLE 12A
NOV12 Sequence Analysis

| | SEQ ID NO:29 | 4380 bp |
|---|---|---|
| NOV12, | GCGGCGGCACGGCGGCGGGTGATGGCTCCTCCGGGCTGCCCGGGTTCGTGCCCCAACT | |
| CG57703-01 | TCGCCGTAGTCTGCTCCTTCTTGGAGCGCTACGGGCCGCTGCTAGACCTGCCTGACTT | |
| DNA Sequence | GCCGTCCCTGAGCTGGACGGGTGTGCAGGCGCCGCGCGGACGTCGCAACGGAGAAGTA | |
| | CCAAAAGAATTGGTGGAGCTCCATTTGAAGCTGATGAGGAAAATTGGCAAATCTGTTA | |
| | CTGCAGACAGATGGGAAAAATATTTGATCAAGTACCTCTGTGAGTGTCAGTTTGATGA | |
| | CAATCTCAAATTCAAGAATATTATTAATGAGGAGGATGCCGATACTATGCGTCTCCAG | |
| | CCAATTGGTCGAGACAAAGATGGCCTCATGTACTGGTACCAATTGGATCAAGATCACA | |
| | ATGTCAGAATGTACATAGAAGAACAAGATGATCAAGATGGCTCTTCATGGAAATGCAT | |
| | TGTCAGAAATCGAAACGAGTTGGCTGAGACTCTTGCACTCCTGAAAGCACAAATTGAT | |
| | CCTGTACTATTGAAAAACTCTAGCCAACAAGACAACTCTTCTCGGGAAAGTCCCAGCT | |
| | TAGAGGATGAGGAGACTAAAAAAGAGGAAGAAACACCTAAACAAGAGGAACAGAAAGA | |
| | AAGTGAAAAGATGAAAAGTGAGGAGCAGCCTATGGATTTAGAAAACCGTTCTACAGCC | |
| | AATGTTCTAGAAGAGACTACTGTGAAAAAAGAAAAAGAAGATGAAAAGGAACTTGTGA | |
| | AACTGCCAGTCATAGTGAAGCTAGAAAAACCTTTGCCAGAAAATGAAGAAAAAAAGAT | |
| | TATCAAAGAAGAAAGTGATTCCTTCAAGGAAAATGTCAAACCCATTAAAGTTGAGGTG | |
| | AAGGAATGTAGAGCAGATCCTAAAGATACCAAAAGTAGCATGGAGAAGCCAGTGGCAC | |
| | AGGAGCCTGAAAGGATCGAATTTGGTGGCAATATTAAATCTTCTCACGAAATTACTGA | |
| | GAAATCTACTGAAGAAACTGAGAAACTTAAAAATGACCAGCAGGCCAAGATACCACTA | |
| | AAAAAACGAGAAATTAAACTGAGTGATGATTTTGACAGTCCAGTCAAGGGACCTTTGT | |
| | GTAAATCAGTTACTCCAACAAAAGAGTTTTTGAAAGATGAAATAAAACAAGAGGAAGA | |
| | GACTTGTAAAAGGATCTCTACAATCACTGCTTTGGGTCATGAAGGGAAACAGCTGGTA | |
| | AATGGAGAAGTTAGTGATGAAAGGGTAGCTCCAAATTTTAAGACAGAACCAATAGAGA | |
| | CAAAGTTTTATGAGACAAAGGAAGAGAGCTATAGCCCCTCTAAGGACAGAAATATCAT | |
| | CACGGAGGGAAATGGAACAGAGTCCTTAAATTCTGTCATAACAAGTATGAAAACAGGT | |
| | GAGCTTGAGAAAGAAACAGCCCCTTTGAGGAAAGATGCAGATAGTTCAATATCAGTCT | |
| | TAGAGATCCATAGTCAAAAAGCACAAATAGAGGAACCCGATCCTCCAGAAATGGAAAC | |
| | TTCTCTTGATTCTTCTGAGATGGCAAAAGATCTCTCTTCAAAAACTGCTTTATCTTCC | |
| | ACCGAGTCGTGTACCATGAAAGGTGAAGAGAAGTCTCCCAAAACTAAGAAGGATAAGC | |
| | GCCCACCAATCCTAGAATGTCTTGAAAAGTTAGAGAAGTCCAAAAAGACTTTTCTTGA | |
| | TAAGGACGCACAAAGATTGAGTCCAATACCAGAAGAAGTTCCAAAGAGTACTCTAGAG | |
| | TCAGAAAAGCCTGGCTCTCCTGAGGCAGCTGAAACTTCTCCACCATCTAATATCATTG | |
| | ACCACTGTGAGAAACTAGCCTCAGAAAAAGAAGTGGTAGAATGCCAGAGTACAAGTAC | |
| | TGTTGGTGGCCAGTCTGTGAAAAAAGTAGACCTAGAAACCCTAAAAGAGGATTCTGAG | |
| | TTCACAAAGGTAGAAATGGATAATCTGGACAATGCCCAGACCTCTGGCATAGAGGAGC | |
| | CTTCTGAGACAAAGGGTTCTATGCAAAAAAGCAAATTCAAATATAAGTTGGTTCCTGA | |
| | AGAAGAAACCACTGCCTCAGAAAATACAGAGATAACCTCTGAAAGGCAGAAAGAGGGC | |
| | ATCAAATTAACAATCAGGATATCAAGTCGGAAAAAGAAGCCCGATTCTCCCCCCAAAG | |
| | TTCTAGAACCAGAAAACAAGCAAGAGAAGACAGAAAAGGAAGAGGAGAAAACAAATGT | |
| | GGGTCGTACTTTAAGAAGATCTCCAAGAATATCTAGACCCACTGCAAAAGTGGCTGAG | |
| | ATCAGAGATCAGAAAGCTGATAAAAAAAGAGGGGAAGGAGAAGATGAGGTGGAAGAAG | |
| | AGTCAACAGCTTTGCAAAAAACTGACAAAAAGGAAATTTTGAAAAAATCAGAGAAAGA | |
| | TACAAATTCTAAAGTAAGCAAGGTAAAACCCAAAGGCAAAGTTCGATGGACTGGTTCT | |
| | CGGACACGTGGCAGATGGAAATATTCCAGCAATGATGAAAGTGAAGGGTCTGGCAGTG | |
| | AAAAATCATCTGCAGCTTCAGAAGAGGAGGAAGAAAAGGAAAGTGAAGAAGCCATCCT | |
| | AGCAGATGATGATGAACCATGCAAAAAATGTGGCCTTCCAAACCATCCTGAGCTAATT | |
| | CTTCTGTGTGACTCTTGCGATAGTGGATACCATACTGCCTGCCTTCGCCCTCCTCTGA | |
| | TGATCATCCCAGATGGAGAATGGTTCTGCCCACCTTGCCAACATAAACTGCTCTGTGA | |
| | AAAATTAGAGGAACAGTTGCAGGATTTGGATGTTGCCTTAAAGAAGAAAGAGCGTGCC | |
| | GAACGAAGAAAAGAACGCTTGGTGTATGTTGGTATCAGTATTGAAAACATCATTCCTC | |
| | CACAAGAGCCAGACTTTTCTGAAGATCAAGAAGAAAAGAAAAAAGATTCAAAAAAATC | |
| | CAAAGCAAACTTGCTTGAAAGGAGGTCAACAAGAACAAGGAAATGTATAAGCTACAGA | |
| | TTTGATGAGTTTGATGAAGCAATTGATGAAGCTATTGAAGATGACATCAAAGAAGCCG | |
| | ATGGAGGAGGAGTTGGCCGAGGAAAAGATATCTCCACCATCACAGGTCATCGTGGGAA | |
| | AGACATCTCTACTATTTTGGATGAACAAAGAAAAGAAAATAAACGACCCCAGAGGGCA | |
| | GCTGCTGCTCGAAGGAAGAAACGCCGGCGATTAAATGATCTCGACAGTGATAGCAACC | |
| | TGGATGAAGAAGAGAGCGAGGATGAATTCAAGATCAGTGATGGATCTCAAGATGAGTT | |
| | TGTTGTGTCTGATGAAAACCCAGATGAAAGTGAAGAAGATCCGCCATCTAATGATGAC | |
| | AGTGACACTGACTTTTGTAGCCGTAGACTGAGGCGACACCCCTCTCGGCCAATGAGGC | |
| | AGAGCAGGCGTTTGCGAAGAAAGACCCCAAAGAAAAAATATTCCGATGATGATGAAGA | |
| | GGAGGAATCTGAGGAGAATAGTAGAGACTCTGAAAGTGACTTCAGTGATGATTTTAGT | |
| | GATGATTTTGTAGAAACTCGGCGAAGGCGGTCAAGGAGAAATCAGAAAAGACAAATTA | |
| | ACTACAAAGAAGACTCAGAAAGTGACGGTTCCCAGAAGAGTTTGCGACGTGGTAAAGA | |
| | AATAAGGCGAGTACACAAGCGAAGACTTTCCAGCTCAGAGAGTGAAGAGAGCTATTTG | |
| | TCCAAGAACTCTGAAGATGATGAGCTAGCTAAAGAATCAAAGCGGTCAGTTCGAAAGC | |
| | GGGGCCGAAGCACAGACGAGTATTCAGAAGCAGATGAGGAGGAGGAGGAAGAGGAAGG | |
| | CAAACCATCCCGCAAACGGCTACACCGGATTGAGACGGATGAGGAGGAGAGTTGTGAC | |
| | AATGCTCATGGAGATGCAAATCAGCCTGCCCGTGACAGCCAGCCTAGGGTCCTGCCCT | |
| | CAGAACAAGAGAGCACCAAGAAGCCCTACCGGATAGAAAGTGATGAGGAAGAGGACTT | |
| | TGAAAATGTAGGCAAAGTGGGGAGCCCATTGGACTATAGCTTAGTGGACTTACCTTCA | |
| | ACCAATGGACAGAGCCCTGGCAAAGCCATTGAGAACTTGATTGGCAAGCCTACTGAGA | |
| | AGTCTCAGACCCCCAAGGACAACAGCACAGCCAGTGCAAGCCTAGCCTCCAATGGGAC | |
| | ACTGACCTTGTTGATTATGTCTGTAACAGTGAACAGTTATAAGACTTTTTTTCCATTT | |
| | TTGTGCTAATTTATTCCACGGTAGCTCTCACACCAGCGGGCCAGTTATTAAAAGCTGT | |
| | TTAATTTTTCCTAGAAAACTCCACTACAGAATCACTTTTAGAAGAAAAATTTCAACAA | |
| | ATCCTGAAGTCTTTCTGTGAAGTGACCAGT | |
| | ORF Start: ATG at 22 | ORF Stop: TAA at 4216 |
| | SEQ ID NO:30 | 1398aa MW at 159105.0 kD |

| | |
|---|---|
| NOV12, | MAPPGCPGSCPNFAVVCSFLERYGPLLDLPELPSLSWTGVQAPRGRRNGEVPKELVEL |
| CG57703-01 | HLKLMRKIGKSVTADRWEKYLIKYLCECQFDDNLKFKNIINEEDAFTMRLQPIGRDKD |
| Protein | GLMYWYQLDQDHNVRMYIEEQDDQDGSSWKCIVRNRNELAETLALLKAQIDPVLLKNS |
| Sequence | SQQDNSSRESPSLEDEETKKEEETPKQEEQKESEKMKSEEQPMDLENRSTANVLEETT |
| | VKKEKEDEKELVKLPVIVKLEKPLPENEEKKIIKEESDSFKENVKPIKVEVKECPADP |
| | KDTKSSMEKPVAQEPERIEFGGNIKSSHEITEKSTEETEKLKNDQQAKIPLKKREIKL |
| | SDDFDSPVKGPLCKSVTPTKEFLKDEIKQEEETCKRISTITALGHEGKQLVNGEVSDE |
| | RVAPNFKTEPIETKFYETKEESYSPSKDRNIITEGNGTESLNSVITSMKTGELEKETA |
| | PLRKDADSSISVLEIHSQKAQIEEPDPPEMETSLDSSEMAKDLSSKTALSSTESCTMK |
| | GEEKSPKTKKDKRPPILECLEKLEKSKKTFLDKDAQRLSPIPEEVPKSTLESEKPGSP |
| | EAAETSPPSNIIDHCEKLASEKEVVECQSTSTVGGQSVKKVDLETLKEDSEFTKVEMD |
| | NLDNAQTSGIEEPSETKQSMQKSKFKYKLVPEEETTASENTEITSERQKEGIKLTIRI |
| | SSRKKKPDSPPKVLEPENKQEKTEKEEEKTNVGRTLRRSPRISRPTAKVAEIRDQKAD |
| | KKRGEGEDEVEEESTALQKTDKKEILKKSEKDTNSKVSKVKPKGKVRWTGSRTRGRWK |
| | YSSNDESEQSCSEKSSAASEEEEEKESEEAILADDDEPCKKCGLPNHPELILLCDSCD |
| | SGYHTACLRPPLMIIPDGEWFCPPCQHKLLCEKLEEQLQDLDVALKKKERAERRKERL |
| | VYVGISIENIIPPQEPDFSEDQEEKKKDSKKSKANLLERRSTRTRKCISYRFDEFDEA |
| | IDEAIEDDIKEADQQGVGRQKDISTITQHRQKDISTILDEERKENKRPQRAAAARRKK |
| | RRRLNDLDSDSNLDEEESEDEFKISDGSQDEFVVSDENPDESEEDPPSNDDSDTDFCS |
| | RRLRRHPSRPMRQSRRLRRKTPKKKYSDDDEEEESEENSRDSESDFSDDFSDDFVETR |
| | RRRSRRNQKRQINYKEDSESDGSQKSLRRGKEIRRVHKRRLSSSESEESYLSKNSEDD |
| | ELAKESKRSVRKRGRSTDEYSEADEEEEEEEGKPSRKRLHRIETDEEESCDNAHGDAN |
| | QPARDSQPRVLPSEQESTKKPYRIESDEEEDFENVQKVGSPLDYSLVDLPSTNGQSPG |
| | KAIENLIGKPTEKSQTPKDNSTASASLASNGTSGGQEAGAPEEEEDELLRVTDLVDYV |
| | CNSEQL |

Further analysis of the NOV12 protein yielded the following properties shown in Table 12B.

TABLE 12B
Protein Sequence Properties NOV12

| | |
|---|---|
| PSort | 0.9800 probability located in nucleus; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV12 protein against the Geneseq database, a proprietary database that contains sequences published in patents and patent publication, yielded several homologous proteins shown in Table 12C.

TABLE 12C
Geneseq Results for NOV12

| | | NOV12 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB83348 | AAP-2 protein sequence - Unidentified, | 1 . . . 1398 | 1386/1400 (99%) | 0.0 |
| | 1400 aa. [EP1118662-A2, 25-JUL-2001] | 1 . . . 1400 | 1386/1400 (99%) | |
| AAB42618 | Human ORFX ORF2382 polypeptide | 310 . . . 1026 | 672/718 (93%) | 0.0 |
| | sequence SEQ ID NO:4764 - Homo | 2 . . . 719 | 683/718 (94%) | |
| | sapiens, 719 aa. [WO200058473-A2, 05- | | | |
| | OCT-2000] | | | |
| AAB92788 | Human protein sequence SEQ ID | 454 . . . 1025 | 571/572 (99%) | 0.0 |
| | NO:11282 - Homo sapiens, 572 aa. | 1 . . . 572 | 572/572 (99%) | |
| | [EP1074617-A2, 07-FEB-2001] | | | |
| AAM25397 | Human protein sequence SEQ ID NO:912 | 453 . . . 1028 | 562/583 (96%) | 0.0 |
| | - Homo sapiens, 583 aa. [WO200153455- | 2 . . . 583 | 564/583 (96%) | |
| | A2, 26-JUL-2001] | | | |
| AAU16231 | Human novel secreted protein, Seq ID | 601 . . . 1030 | 421/430 (97%) | 0.0 |
| | 1184 - Homo sapiens, 432 aa. | 3 . . . 432 | 423/430 (97%) | |
| | [WO200155322-A2, 02-AUG-2001] | | | |

In a BLAST search of public sequence databases, the NOV12 protein was found to have homology to the proteins shown in the BLASTP data in Table 12D.

TABLE 12D
Public BLASTP Results for NOV12

| | | NOV12 | | |
|---|---|---|---|---|
| Protein | | Residues/ | Identities/Similarities | |
| Accession | | Match | for the Matched | Expect |
| Number | Protein/Organism/Length | Residues | Portion | Value |
| Q96T23 | HBV PX ASSOCIATED PROTEIN 8 | 1 . . . 1398 | 1385/1431 (96%) | 0.0 |
| | LARGE ISOFORM - Homo sapiens | 1 . . . 1431 | 1386/1431 (96%) | |
| | (Human), 1431 aa. | | | |
| Q9NYU0 | HBV PX ASSOCIATED PROTEIN-8 - | 210 . . . 1398 | 1188/1189 (99%) | 0.0 |
| | Homo sapiens (Human), 1189 aa. | 1 . . . 1189 | 1189/1189 (99%) | |
| Q9NVZ8 | CDNA FLJ10406 FIS, CLONE | 454 . . . 1025 | 571/572 (99%) | 0.0 |
| | NT2RM4000515 - Homo sapiens | 1 . . . 572 | 572/572 (99%) | |
| | (Human), 572 aa (fragment). | | | |
| Q9H3L8 | MY001 PROTEIN - Homo sapiens | 79 . . . 505 | 414/427 (96%) | 0.0 |
| | (Human), 450 aa. | 9 . . . 435 | 416/427 (96%) | |
| CAC42796 | SEQUENCE 19 FROM PATENT | 607 . . . 900 | 294/294 (100%) | e−170 |
| | EP1120463 - Homo sapiens (Human), | 1 . . . 294 | 294/294 (100%) | |
| | 294 aa (fragment). | | | |

PFam analysis predicts that the NOV12 protein contains the domains shown in the Table 12E.

TABLE 12E
Domain Analysis of NOV12

| | | Identities/Similarities | |
|---|---|---|---|
| | NOV12 | for the | Expect |
| Pfam Domain | Match Region | Matched Region | Value |
| PHD: domain 1 of 1 | 850 . . . 898 | 22/51 (43%) | 1.6e−14 |
| | | 38/51 (75%) | |
| Glycos_transf_1: | 887 . . . 924 | 12/47 (26%) | 8 |
| domain 1 of 1 | | 27/47 (57%) | |
| Virus_HS: domain | 1110 . . . 1145 | 15/47 (32%) | 0.82 |
| 1 of 1 | | 29/47 (62%) | |

### Example A13

The NOV13 clone was analyzed, and the nucleotide and predicted polypeptide sequences are shown in Table 13A.

TABLE 13A
NOV13 Sequence Analysis

| | SEQ ID NO:31 | 1663 bp |
|---|---|---|
| NOV13a, | AGGGTGGCACCATGGGCCCGGGCGGTGCCCTCCATGCCCGGGGGATGACCACTGCCAC | |
| CG58651-01 | TGCTGCCATGGACAGCCCGTGCCAGCCGCAGCCCCTAAGTCAGGCTCTCCCTCAGTTA | |
| DNA | CCAGGGTCTTCGTCAGAGCCCTTGGAGCCTGAGCCTGGCCGGGCCAGCATGGGAGTGG | |
| Sequence | AGAGTTACCTGCCCTGTCCCCTGCTCCCCTCCTACCACTGTCCAGGAGTGCCTAGTGA | |
| | GCGCTGCAGCGCTGGTCACCAAGGGGTGCCAGCGATTGGCAGCCCAGGGCGCACGGCC | |
| | CTTCGGGACGGTTTTGGCGGGCAGGATGGTGGTGAGCTGCGGCCGCTGCAGAGTGAAG | |
| | GCGCTGCAGCGCTGGTCACCAAGGGGTGCCAGCGATTGGCAGCCCAGGGCGCACGGCC | |
| | TGAGGCCCCCAAACGGAAATGGGCCGAGGATGGTGGGGATGCCCCTTCACCCAGCAAA | |
| | CGGCCCTGGGCCAGGCAAGAGAACCAGGAGGCAGAGCGGGAGGGTGGCATGAGCTGCA | |
| | GCTGCAGCAGTGGCAGTGGTGAGGCCAGTGCTGGGCTGATGGAGGAGGCGCTGCCCTC | |
| | TGCGCCCGAGCGCCTGGCCCTGGACTATATCGTGCCCTGCATGCGGTACTACGGCATC | |
| | TGCGTCAAGGACAGCTTCCTGGGGGCAGCACTGGGCGGTCGCGTGCTGGCCGAGGTGG | |
| | AGGCCCTCAAACGGGGTGGGCGCCTGCGAGACGGGCAGCTAGTGAGCCAGAGGGCGAT | |
| | CCCGCCGCGCAGCATCCGTGGGGACCAGATTGCCTGGGTGGAAGGCCATGAACCAGGC | |
| | TGTCGAAGCATTGGTGCCCTCATGGCCCATGTGGACGCCGTCATCCGCCACTGCGCAG | |
| | GGCGGCTGGGCAGCTATGTCATCAATGGGCGCTGCATCACCTGTATCTATTACCTGAA | |
| | TCAGAACTGGGACGTTAAGGTGCATGGCGGCCTGCTGCAGATCTTCCCTGAGGGCCGG | |
| | CCCGTGGTAGCCAACATCGAGCCACTCTTTGACCGGTTGCTCATTTTCTGGTCTGACC | |
| | GGCGGAACCCCCACGAGGTGAAGCCAGCCTATGCCACCAGGTACGCCATCACTGTCTG | |
| | GTATTTTGATGCCAAGGAGCGGGCAGCAGCCAAAGACAAGTATCAGCTAGCATCAGGA | |
| | CAGAAAGGTGTCCAAGTACCTGTATCACAGCCGCCTACGCCCACCTAGTGGCCAGTCC | |
| | CAGAGCCGCATGGCAGACAGCTTAAATGACTTCAGGAGAGCCCTGGGCCTGTGCTGGC | |
| | TGCTCCTTCCCTGCCACCGCTGCTGCTTCTGACTTTGCCTCTGTCCTGCCTGGTGTGG | |
| | AGGGCTCTGTCTGTTGCTGAGGACCAAGGAGGAGAAGAGACCTTTGCTGCCCCATCAT | |
| | GGGGGCTGGGTTGTCACCTGGACAGGGGGCAGCCGTGGACGCCACCGTTACCAACTGA | |
| | AGCTGGGGGCCTGGGTCCTACCCTGTCTGGTCATGACCCCATTAGGTATGGAGAGCTG | |
| | GCGAGCGAGGCATTGTTCACTTCCCACCAGGATGCAGCACTTGGGGTTGAACGTGAGT | |
| | CATGGGCCTCTTGCTGGGAATGGGGTGGGCAGGAGTACCCCCAAGTTCTTCTCATCCT | |
| | CCCACCTGGAATGTTGACTCAATTCCCCAAACCTTGGGC | |
| | ORF Start: ATG at 12 | ORF Stop: TAG at 1206 |
| | SEQ ID NO:32 | 398 aa MW at 42435.6 kD |
| NOV13a, | MGPGGALHARGMTTATAAMDSPCQPQPLSGALPQLPGSSSEPLEPEPGRARMGVESYL | |
| CG58651-01 | PCPLLPSYHCPGVPSEASAGSGTPRATATSTTASPLRDGFGGQDQQELRPLGSEGAAA | |
| Protein | LVTKQCGRLAAQGARPEAPKRKWAEDGGDAPSPSKRPWARQENGEAEREGGMSCSCSS | |
| Sequence | GSGEASAGLMEEALPSAPERLALDYIVPCMRYYGICVKDSFLGAALGGRVLAEVEALK | |
| | RGGRLRDGQLVSQRAIPPRSIRGDQIAWVEGHEPGCRSIGALMAHVDAVIRHCAGRLG | |
| | SYVINGRCITCIYYLNQNWDVKVHGGLLQIFPEGRPVVANIEPLFDRLLIFWSDRRNP | |
| | HEVKPAYATRYAITVWYFDAKERAAAKDKYGLASGQKQVGVPVSQPPTPT | |
| | SEQ ID NO:33 | 1589 bp |
| NOV 13b, | AAGTTGAAACAAGACGAGCGCCGGGGCCGGACGAAAAGCCTCGCCCCCCTGAAGGTAC | |
| CG58651-02 | CCTTCCCAAGCCCTTAGGGACCGCAGAGGACTTGGGGACCAGCAAGCAACCCCCAGGG | |
| DNA | CACGAGAAGAGCTCTTGCTGTCTGCCCTGCCTCACCCTGCCCCACGCCAGGCCCGGTG | |
| Sequence | GCCCCCAGCTGCATCAAGTGGAGGCGGAGGAGGAGGCGGAGGAGGGTGGCACCATGGG | |
| | CCCGGGCGGTGCCCTCCATGCCCGGGGGATGAAGACACTGCTGCCATGGACAGCCCGT | |
| | GCCAGCCGCAGCCCCTAAGTCAGGCTCTCCCTCAGTTACCAGGGTCTTCGTCAGAGCC | |
| | CTTGGAGCCTGAGCCTGGCCGGGCCAGGATGGGAGTGGAGAGTTACCTGCCCTGTCCC | |
| | CTGCTCCCCTCCTACCACTGTCCAGGAGTGCCTAGTGAGGCCTCGGCAGGGAGTGGGA | |
| | CCCCCAGAGCCACAGCCACCTCTACCACTGCCAGCCCTCTTCGGGACGGTTTTGGCGG | |
| | GCAGGATGGTGGTGAGCTGCGGCCGCTGCAGAGTGAAGGCGCTGCAGCGCTGGTCACC | |
| | AAGGGGTGCCAGCGATTGGCAGCCCAGGGCGCACGGCCTGAGGCCCCCAAACGGAAAT | |
| | GGGCCGAGGATGGTGGGGATGCCCCTTCACCCAGCAAACGGCCCTGGGCCAGGCAAGA | |
| | GAACCAGGAGGCAGAGCGGGAGGGTGGCATGAGCTGCAGCTGCAGCAGTCGCAGTGGT | |
| | GAGGCCAGTGCTGGGCTGATGGAGGAGGCGCTGCCCTCTGCGCCCGAGCGCCTGGCCC | |
| | TGGACTATATCGTGCCCTGCATGCGGTACTACGGCATCTGCGTCAAGGACAGCTTCCT | |
| | GGGGGCAGCACTGGGCGGTCGCGTGCTGGCCGAGGTGGAGGCCCTCAAACGGGGTGGC | |
| | CGCCTGCGAGACGGGCAGCTAGTGAGCCAGAGGGCGATCCCGCCGCGCAGCATCCGTG | |
| | GGGACCAGATTGCCTGGGTGGAAGGCCATGAACCAGGCTGTCGAAGCATTGGTGCCCT | |
| | CATGGCCCATGTGGACGCCGTCATCCGCCACTGCGCAGGGCGGCTGGGCAGCTATGTC | |
| | ATCAACGGGCGCACCAAGGCCATGGTGGCGTGTTACCCAGGCAACGGGCTCGGGTACG | |
| | TAAGGCACGTTGACAATCCCCACGGCGATGGGCGCTGCATCACCTGTATCTATTACCT | |
| | GAATCAGAACTGGGACGTTAAGGTAGTGCATGGCGGCCTGCTGCAGATCTTCCCTGAG | |
| | GGCCGGCCCGTGGTAGCCAACATCGAGCCACTCTTTGACCGGTTGCTCATTTTCTGGT | |
| | CTGACCGGCGGAACCCCCACGAGGTGAAGCCAGCCTATGCCACCAGGTATGCCATCAC | |
| | TGTCTGGTATTTTGATGCCAAGGAGCGGGCAGCAGCCAAAGACAAGTATCAGCTAGGT | |
| | ACCTGCTTCCCTCCCTTCAGTCCTTCCTATTCTGTGGGCCCTCTTGGGCCTGATGCCA | |
| | CCCCATCCCCCTCATCAGCCTCTTGTTAAATCCCACCACTCATTTTTCTTCATCTCTG | |
| | CCCACCTTCCTTAGCCCACTCTC | |
| | ORF Start: ATG at 278 | ORF Stop: TAA at 1535 |
| | SEQ ID NO:34 | 419aa MW at 44785.1 kD |

| | |
|---|---|
| NOV13b, | MDSPCQPQPLSGALPQLPGSSSEPLEPEPGRARNGVESYLPCPLLPSYHCPGVPSEAS |
| CG58651-02 | AGSGTPRATATSTTASPLRDGFGGQDQQELRPLGSEQAAALVTKQCGRLAAQGARPEA |
| Protein | PKRKWAEDGQDAPSPSKRPWARQENQEAEREGGMSCSCSSGSGEASAGLMEEALPSAP |
| Sequence | ERLALDYIVPCMRYYGICVKDSFlGAALGGRVLAEVEALKRGGRLRDQQLVSQRAIPP |
| | RSIRGDQIANVEQHEPGCRSIGALMAHVDAVIRHCAGRLGSYVINGRTKAMVACYPGN |
| | GLGYVRHVDNPHGDGRCITCIYYLNGNWDVKVVHGGLLQIFPEGRPVVANIEPLFDRL |
| | LIFWSDRRNPHEVKPAYATRYAITVWYFDAKERAAAKDKYQLGTCFPPFSPSYSVGPL |
| | GPDATPSPSSASC |

Sequence comparison of the above protein sequences yields the following sequence relationships shown in Table 13B.

TABLE 13B
Comparison of NOV13a against NOV13b.

| | | Identities/Similarities |
|---|---|---|
| | NOV13a Residues/NOVl3b | for the Matched |
| Protein Sequence | Match Residues | Region |
| NOV13b | 19-295 | 277/277 (100%) |
| | 1 . . . 277 | 277/277 (100%) |
| | 296 . . . 380 | 84/85 (99%) |
| | 305 . . . 419 | 84/85 (99%) |

Further analysis of the NOV13a protein yielded the following properties shown in Table 13C.

TABLE 13C
Protein Sequence Properties NOV13a

| | |
|---|---|
| PSort | 0.3000 probability located in nucleus; 0.1893 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV13a protein against the Geneseq database, a proprietary database that contains sequences published in patents and patent publication, yielded several homologous proteins shown in Table 13D.

TABLE 13D
Geneseq Results for NOV13a

| | | NOV13a | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| AAB10873 | Human tumor-associated antigen 9D7 protein | 193 . . . 379 | 110/215 (51%) | 2e−61 |
| | - Homo sapiens, 239 aa. [DE19909503-A1, | 12 . . . 226 | 144/215 (66%) | |
| | 07-SEP-2000] | | | |
| ABB03740 | Human musculoskeletal system related | 295 . . . 398 | 103/104 (99%) | 8e−58 |
| | polypeptide SEQ ID NO 1687 - Homo | 47 . . . 150 | 104/104 (99%) | |
| | sapiens, 150 aa. [WO200155367-A1, 02- | | | |
| | AUG-2001] | | | |
| AAB63118 | Human secreted protein sequence encoded | 295 . . . 379 | 84/85 (98%) | 3e−46 |
| | by gene 40 SEQ ID NO:128 - Homo sapiens, | 24 . . . 108 | 85/85 (99%) | |
| | 108 aa. [WO200061748-A1, 19-OCT-2000] | | | |
| AAB63117 | Gene 40 human secreted protein homologous | 295 . . . 379 | 63/85 (74%) | 5e−36 |
| | amino acid sequence #127 - Rattus | 24 . . . 108 | 78/85 (91%) | |
| | norvegicus, 108 aa. [WO200061748-A1, 19- | | | |
| | OCT-2000] | | | |

In a BLAST search of public sequence databases, the NOV13a protein was found to have homology to the proteins shown in the BLASTP data in Table 13E.

TABLE 13E
Public BLASTP Results for NOV13a

| | | NOV13a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q96KS0 | EGLN2 PROTEIN - Homo sapiens | 19 . . . 398 | 380/407 (93%) | 0.0 |
| | (Human), 407 aa. | 1 . . . 407 | 380/407 (93%) | |
| AAK82943 | ESTROGEN-INDUCED TAG 6 - | 19 . . . 398 | 379/407 (93%) | 0.0 |
| | Homo sapiens (Human), 407 aa. | 1 . . . 407 | 379/407 (93%) | |
| AAL65166 | EGLN2 - Mus musculus (Mouse), 419 | 19 . . . 398 | 343/421 (81%) | 0.0 |
| | aa. | 1 . . . 419 | 355/421 (83%) | |
| Q99MI0 | CELL GROWTH REGULATOR | 19 . . . 398 | 342/421 (81%) | 0.0 |
| | FALKOR - Mus musculus (Mouse), | 1 . . . 419 | 355/421 (84%) | |
| | 419 aa. | | | |
| Q91YE2 | EGLN2 PROTEIN - Mus musculus | 19 . . . 398 | 336/421 (79%) | 0.0 |
| | (Mouse), 419 aa. | 1 . . . 419 | 347/421 (81%) | |

PFam analysis predicts that the NOV13a protein contains the domains shown in the Table 13F.

TABLE 13F
Domain Analysis of NOV13a

| | | | |
|---|---|---|---|
| | NOV13a Match | Identities/Similarities | Expect |
| Pfam Domain | Region | for the Matched Region | Value |

No Significant Matches Found To Known Sequences

### Example A14

The NOV14 clone was analyzed, and the nucleotide and predicted polypeptide sequences are shown in Table 14A.

TABLE 14A
NOV14 Sequence Analysis

| | SEQ ID NO:35 | 743 bp |
|---|---|---|
| NOV14, | AAGCATCTAAATATGGTGTCCTCCGGAAAAAAGTATTCC | |
| CG59574-01 | AGGAAATCTGGGAAGCCGTCTGTGGAAGATCAGTTTACG | |
| DNA | AGAGCCTATGACTTTGAGACTGAAGATAAGAAAGATCTG | |
| Sequence | AGTGGATCAGAGGAAGATGTTATTGAAGGTAAGACTGCA | |
| | GTCATTGAGAAACGTAGGAAGAAAAGGTCTTCTGCAGGA | |
| | GTAGTTGAAGATATGGGGGGTGAAGTGCAGAATATGCTG | |
| | GAAGGAGTTGGAGGTGACATTAACAAGGCTCTTCTTGCC | |
| | AAGAGAAAGAGACTAGAAATGTATACCAAGGCTTCTCTC | |
| | AAAACTAGTAACCAGAAAATTGAACATGTTTGGAAAACA | |
| | CAACAAGATCAAAGGCAGAAGCTTAACCAAGAATATTCT | |
| | CAGCAGTTTCTGACTTTGTTTCAGCAGTGGGATTTAGAT | |
| | ATGCAGAAAGCTGAGGAACAAGAAGAAAAAATACTTAAT | |
| | ATGTTTCGACAGCAACAAAAGATTCTTCAACAATCTAGA | |
| | ATTGTTCAGAGCCAGAGATTGAAAACAATTAAACAGTTA | |
| | TATGAGCAGTTCATAAAGAGTATGGAAGAGTTGGAGAAG | |
| | AATCATGATAATCTACTTACTGGTGCACAAAATGAATTT | |
| | AAAAAAGAAATGGCTATGTTGCAAAAAAAAATTATGATG | |
| | GAAACTCAGCAGCAAGAGATAGCAAGTGTTCGGAAGTCT | |
| | CTTCAATCCATGTTATTCTGATGACTCTTTGAAGAAAGA | |
| | AC | |
| | ORF Start: | |
| | ATG at 13 | ORF Stop: TGA at 721 |
| | SEQ ID NO:36 | 236 aa MW at 27686.5 kD |

| | |
|---|---|
| NOV14 | MVSSQKKYSRKSQKPSVEDGFTRAYDFETEDKKDLSGSE |
| CG59574-01 | EDVIEGKTAVIEKRRKKRSSAGVVEDMGGEVQNMLEGVG |
| Protein | QDINKALLAKRKRLEMYTKASLKTSNQKIENVWKTQQDQ |
| Sequence | RQKLNQEYSQQFLTLFQQWDLDMQKAEEGEEKILNMFRG |
| | GQKILGGSRIVQSQRLKTIKQLYEQFIKSMEELEKNHDN |
| | LLTGAQNEFKKEMAMLQKKIMMETQQQEIASVRKSLQSM |
| | LF |

Further analysis of the NOV14 protein yielded the following properties shown in Table 14B.

TABLE 14B
Protein Sequence Properties NOV14

| | |
|---|---|
| PSort | 0.7000 probability located in nucleus; 0.1000 probability |
| analysis: | located in mitochondrial matrix endoplasmic reticulum |
| | (membrane) |
| SignalP | No Known Signal Sequence Predicted |
| analysis: | |

A search of the NOV14 protein against the Geneseq database, a proprietary database that contains sequences published in patents and patent publication, yielded several homologous proteins shown in Table 14C.

TABLE 14C
Geneseq Results for NOV14

| | | NOV14 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAM79147 | Human protein SEQ ID NO 1809 - Homo | 80 . . . 236 | 157/157 (100%) | 8e−84 |
| | sapiens, 249 aa. [WO200157190-A2, 09- | 93 . . . 249 | 157/157 (100%) | |
| | AUG2001] | | | |
| AAM80131 | Human protein SEQ ID NO 3777 - Homo | 38 . . . 151 | 111/114 (97%) | 1e−57 |
| | sapiens, 137 aa. [WO200157190-A2, 09- | 1 . . . 114 | 113/114 (98%) | |
| | AUG-2001] | | | |
| AAM73141 | Human bone marrow expressed probe | 119 . . . 151 | 33/33 (100%) | 4e−12 |
| | encoded protein SEQ ID NO:33447 - Homo | 1 . . . 33 | 33/33 (100%) | |
| | sapiens, 33 aa. [WO200157276-A2, 09- | | | |
| | AUG-2001] | | | |
| AAM60494 | Human brain expressed single exon probe | 119 . . . 151 | 33/33 (100%) | 4e−12 |
| | encoded protein SEQ ID NO:32599 - Homo | 1 . . . 33 | 33/33 (100%) | |
| | sapiens, 33 aa. [WO200157275-A2, 09- | | | |
| | AUG-2001] | | | |
| AAM33356 | Peptide #7393 encoded by probe for | 119 . . . 151 | 33/33 (100%) | 4e−12 |
| | measuring placental gene expression - | 1 . . . 33 | 33/33 (100%) | |
| | Homo sapiens, 33 aa. [WO200157272-A2, | | | |
| | 09-AUG-2001] | | | |

In a BLAST search of public sequence databases, the NOV14 protein was found to have homology to the proteins shown in the BLASTP data in Table 14D.

TABLE 14D
Public BLASTP Results for NOV14

| | | NOV14 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q63520 | Synaptonemal complex protein 3 (SCP-3 | 1 . . . 236 | 171/236 (72%) | 3e−93 |
| | protein) - Rattus norvegicus (Rat), 257 aa. | 24 . . . 257 | 203/236 (85%) | |
| Q60547 | Synaptonemal complex protein 3 (SCP-3 | 1 . . . 236 | 173/236 (73%) | 6e−93 |
| | protein) (Synaptonemal complex protein | 1 . . . 234 | 202/236 (85%) | |
| | COR1) (Meiotic chromosome core protein) - | | | |
| | Mesocricetus auratus (Golden hamster), 234 | | | |
| | aa. | | | |
| P70281 | Synaptonemal complex protein 3 (SCP-3 | 1 . . . 236 | 168/236 (71%) | 4e−91 |
| | protein) - Mus musculus (Mouse), 254 aa. | 21 . . . 254 | 201/236 (84%) | |
| Q9DAC5 | 1700013H16RIK PROTEIN - Mus musculus | 55 . . . 224 | 75/170 (44%) | 3e−38 |
| | (Mouse), 291 aa. | 121 . . . 290 | 120/170 (70%) | |
| Q9D6C3 | 3830403N18RIK PROTEIN - Mus musculus | 25 . . . 219 | 74/195 (37%) | 4e−36 |
| | (Mouse), 208 aa. | 4 . . . 197 | 125/195 (63%) | |

PFam analysis predicts that the NOV14 protein contains the domains shown in the Table 14E.

TABLE 14E
Domain Analysis of NOV14

| | NOV14 | Identities/Similarities | Expect |
|---|---|---|---|
| Pfam Domain | Match Region | for the Matched Region | Value |
| PAH: domain 1 of 1 | 149 . . . 199 | 11/51 (22%) | 9.7 |
| | | 37/51 (73%) | |

### Example A15

The NOV15 clone was analyzed, and the nucleotide and predicted polypeptide sequences are shown in Table 15A.

TABLE 15A
NOV15 Sequence Analysis

| | SEQ ID NO:37 | 1409 bp |
|---|---|---|
| NOV15, | GGCACAGCCCAGGAACGTTGCTTTGGAGAATCCTGCAGA | |
| CG59536-01 | TAAGGCTTTTCCAAAAAGCGCGAGCATCTTGTTGTATTC | |
| DNA | AGATACCCTATCGTCGTCAGTCATGGCTAGCATCACTGC | |
| Sequence | GTGTGTGGGTAACAGCAGGCAGCAGAATGCACCTTTGCC | |
| | GCCTTGGGCCCATTCCATGTTGAGGTCTCTGGGGAGGAG | |
| | TCTCTGTCCTTTAGTGGTCAAAATGGCAGAGAGAAACAT | |
| | GAAGTTGTTCTCAGGAAGAGTGGTGCCAGCCCAGGGGAA | |
| | AGAAACCTTTGAAAACTGGCTGATCCAAGTCAATGAGGT | |
| | CCTGCCAGATTGGAGTATGTCTGAGGAGGAAAAACTCAA | |
| | GCGCTTGATGAAAACACTTAGGGGCCCTGCCCGGGAGGT | |
| | CATGCGTTTGCTTCAGGCGGCCAACCCCAACCTAAGTGT | |
| | AGCAGATTTCTTGCGGGCAATGAAATTGGTGTTTGGGGA | |
| | GTCTGAAAGCAGTGTGACTGCCCATGGTAAATTTTTTAA | |
| | CACCCTGCAGGCACAAGGGGAGAAAGCCTCCCTTTATGT | |
| | GATCCGTTTAGAGGTGCAGCTCCAGAATGCTATTCAGGC | |
| | AGGCATCCTAGCTGAGAAAGATGCAAACCAGACTCGCTT | |
| | GCAACAGCTTCTTTTAGGCGCTGAGCTGAATAGGGACCT | |
| | GCGCTTCAGGCTTAAGCATCTTCTCAGGATGTATGCAAA | |
| | TAAGCAGGAGCGGCTTCCCAATTTCCTGGAGTTAATCAA | |
| | GATGATAAGGGAGGAAGAGGATTGGGATGATGCTTTTAT | |
| | TAAACGGAAGCGGCCGAAAAGGTCTGAGCCAATAATGGA | |
| | GAGGGCAGCCAGCCCTGTGGCATTTCAGGGCGCCCAGCC | |
| | AATAGCAATCAGCAGTGCTGACTGTAACTGCAACGTGAT | |
| | AGAAATAGATGATACCCTTGATGACTCTGATGAGGATGT | |
| | GATCCTGGTGGTGTCTCTGTACCCTTCACTGACACCTAC | |
| | AGGTGCCCCTCCCTTCAGAGGAAGACCCAGACCTCTGGA | |
| | TCAAGTGCTGGTTATTGATTCCCCCAACAATTCTGGGGC | |
| | TCAGTCTCTTTCTACCAGTGGTGGTTCTGGGTATAAGAA | |
| | TGATGGTCCTGGGAATATTCGTAGAGCCAGGAAGCGAAA | |
| | ATACACAACCCGCTGTTCATATTGTGGGGAGGAGGGCCA | |
| | CTCAAAAGAAACCTGTGACAATGAGAGCAACAAGGCCCA | |
| | GGTTTTTGAGAATCTGATCATCACCCTGCAGGAGCTGAC | |
| | ACATACAGAGGAGAGGTCAAAAGAGGTCCCTGGAGAACA | |
| | CAGTGATGCTTCTGAGCCACAGTAAGGATCTAGTCCAGC | |
| | CCTAAATGAGTCCTTGACTGTATTCAGAGTCTGGTAATG | |
| | GGAATAACAGGAGAGGGGGGTGGGTTTCTAACTGCATGA | |
| | ATTAA | |
| | ORF Start: | |
| | ATG at 101 | ORF Stop: TAA at 1310 |
| | SEQ ID NO:38 | 403 aa MW at 45159.8 Kd |

| | |
|---|---|
| NOV15, | MASITACVGNSRQQNAPLPPWAHSMLRSLGRSLCPLVVK |
| CG59536-01 | MAERNMKLFSGRVVPAQGKETFENWLIQVNEVLPDWSMS |
| Protein | EEEKLKRLMKTLRGPAREVMRLLQAANPNLSVADFLRAM |
| Sequence | KLVFGESESSVTAHQKFFNTLQAQGEKASLYVIRLEVQL |
| | QNATQAGILAEKDANQTRLQQLLLGAELNRDLRFRLKHL |
| | LRMYANKQERLPNFLELIKMIREEEDWDDAFIKRKRPKR |
| | SEPIMERAASPVAFQGAQPIAISSADCNCNVIEIDDTLD |
| | DSDEDVILVVSLYPSLTPTGAPPFRGRARPLDQVLVIDS |
| | PNNSGAQSLSTSGGSGYKNDGPGNIRRARKRKYTTRCSY |
| | CGEEGHSKETCDNESNKAQVFENLIITLQELTHTEERSK |
| | EVPGEHSDASEPG |

Further analysis of the NOV15 protein yielded the following properties shown in Table 15B.

TABLE 15B
Protein Sequence Properties NOV15

| | |
|---|---|
| PSort | 0.7000 probability located in nucleus; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV15 protein against the Geneseq database, a proprietary database that contains sequences published in patents and patent publication, yielded several homologous proteins shown in Table 15C.

TABLE 15C
Geneseq Results for NOV15

| | | NOV15 | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| AAB60478 | Human cell cycle and proliferation protein | 1 . . . 402 | 339/403 (84%) | 0.0 |
| | 402 aa. [WO200107471-A2, 01-FEB-2001] | 1 . . . 401 | 364/403 (90%) | |
| AAM25693 | Human protein sequence SEQ ID NO:1208 - | 17 . . . 355 | 278/339 (82%) | e−154 |
| | Homo sapiens, 337 aa. [WO200153455-A2, | 1 . . . 337 | 298/339 (87%) | |
| | 26-JUL-2001] | | | |
| AAB12529 | Human Ma5 protein SEQ ID NO:13 - Homo | 1 . . . 367 | 108/370 (29%) | 2e−37 |
| | sapiens, 463 aa. [JP2000146982-A, 26- | 117 . . . 432 | 177/370 (47%) | |
| | MAY-2000] | | | |
| AAB42315 | Human ORFX ORF2079 polypeptide | 1 . . . 367 | 108/370 (29%) | 2e−37 |
| | sequence SEQ ID NO:4158 - Homo sapiens, | 117 . . . 432 | 177/370 (47%) | |
| | 463 aa. [WO200058473-A2, 05-OCT-2000] | | | |
| AAB12528 | Human Ma4 protein SEQ ID NO:11 - Homo | 22 . . . 226 | 84/205 (40%) | 4e−37 |
| | sapiens, 283 aa. [JP2000146982-A, 26- | 62 . . . 260 | 132/205 (63%) | |
| | MAY-2000] | | | |

In a BLAST search of public sequence databases, the NOV15 protein was found to have homology to the proteins shown in the BLASTP data in Table 15D.

TALBE 15D
Public BLASTP Results for NOV15

| | | NOV15 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9CZA5 | 2810028A01RIK PROTEIN - Mus | 1 . . . 403 | 291/404 (72%) | e−161 |
| | musculus (Mouse), 402 aa. | 1 . . . 402 | 332/404 (82%) | |
| AAH17627 | RIKEN CDNA 1500031H04 GENE - Mus | 1 . . . 393 | 268/394 (68%) | e−151 |
| | musculus (Mouse), 393 aa. | 1 . . . 392 | 322/394 (81%) | |
| Q9DB17 | 1500031H04RIK PROTEIN - Mus | 1 . . . 393 | 267/394 (67%) | e−150 |
| | musculus (Mouse), 393 aa. | 1 . . . 392 | 322/394 (80%) | |
| Q9GMU3 | HYPOTHETICAL 41.3 KDA PROTEIN - | 22 . . . 226 | 86/205 (41%) | 4e−37 |
| | Macaca fascicularis (Crab eating macaque) | 143 . . . 341 | 131/205 (62%) | |
| | (Cynomolgus monkey), 364 aa. | | | |
| Q9H0A4 | HYPOTHETICAL 51.5 KDA PROTEIN - | 1 . . . 367 | 108/370 (29%) | 8e−37 |
| | Homo sapiens (Human), 455 aa. | 117 . . . 432 | 177/370 (47%) | |

PFam analysis predicts that the NOV15 protein contains the domains shown in the Table 15E.

TABLE 15E
Domain Analysis of NOV15

| | | for the | Expect |
|---|---|---|---|
| Pfam Domain | NOV15 Match Region | Matched Region | Value |
| zf-CCHC: | 347 . . . 364 | 6/18 (33%) | 0.059 |
| domain 1 of 1 | | 12/18 (67%) | |

### Example A16

The NOV16 clone was analyzed, and the nucleotide and predicted polypeptide sequences are shown in Table 16A.

TABLE 16A
NOV16 Sequence Analysis

| | SEQ ID NO:39 | 1746 bp |
|---|---|---|
| NOV16, | ATGGTGCTGGCGCGGGTGGCGCGCCGGCCGGCGGGGTCT | |
| CG59299-01 | GCGGAGGCCCAGGCGTCCCTGGCGGGGAAGGCGGCGCTT | |
| DNA | CCGGAGCGTGCCAGGAAGATGTCGAGCCCGGGCATCGAC | |
| Sequence | GGCGACCCCAAGCCTCCATGCTTGCCTCGAAACGGTCTG | |
| | GTGAAGCTGCCGGGCCAGCCCAACGGCCTGGGTGCGGCC | |
| | AGCATCACCAAGGGCACGCCAGCCACCAAGAACCGCCCC | |
| | TGCCAGCCACCACCCCCACCCACCCTCCCACCACCCAGC | |
| | CTGGCTGCTCCACTGTCGCACCTGGGCACCCAGCGGAGC | |
| | GGCCGCCGCTGGCCACGGACGAGAAGATCCTCAATGGGC | |
| | TCTTCTGCTATTTCTCGGCCTCCGAGAAGTGTGTGCTGG | |
| | CCCAGGTGTGCAAGGCCTGGCGGCGCGTGCTGTACCAGC | |
| | CCAAGTTCTGGGCAGGCCTCACGCCGGTGCTGCATGCCA | |
| | AGGAGCTCTACAACGTGCTGCCTGGTGGCGAGAAGGAGT | |
| | TCGTGAACCTGCAGGGTTTTGCCGCCAGAGGCTTCGAGG | |
| | GCTTCTGCCTGGTTGGCGTCTCCGACCTGGACATCTGTG | |
| | ACTTCATTGACAACTATGCGCTCTCCAAGAAGGGTGTCA | |
| | AAGCCATGAGCCTCTGGTGCGTCTGGAGCTGTCGGGCTG | |
| | CAACGACTTCACCGAGGCCGGGCTGTGGTCCAGCCTGAG | |
| | CGCGCGCATCACCTCGCTGAGCGTGAGTGACTGCATCAA | |
| | CGTGGCCGACGACGCCATCGCGGCCATCTCGCAGCTGCT | |
| | GCCCAACCTGGCGGAGCTGAGCCTGCAGGCCTACCACGT | |
| | GACGGACACGGCGCTGGCCTACTTCACGGCGCGCCAGGG | |
| | CCACAGCACGCACACGCTGCGCCTGCTCTCCTGCTGGGA | |
| | GATCACCAACCACGGCGTGGTCAACGTGGTGCACAGCCT | |
| | GCCCAACCTCACCGCGCTCAGCCTCTCGGGCTGCTCCAA | |
| | CGTCACCGACGACGGCGTGGAGCTCGTGGCCGAGAACCT | |
| | GCGCAAGCTGCGCAGCCTTGACCTCTCGTGGTGCCCACG | |
| | CATCACCGACATGGCGCTGGAGTACGTGGCCTGCGACCT | |
| | GCACCGCCTAGAGGAGCTCGTGCTCGACAGGTGTGTACG | |
| | CATCACGGACACTGGCCTCAGCTATCTGTCCACCATGTC | |
| | GTCCCTCCGCAGCCTCTACCTGCGATGGTGCTGCCAGGT | |
| | GCAAGACTTCGGGCTGAAGCACCTCCTGGCCCTGGGGAG | |
| | TTTGCGCCTCCTGTCTCTGGCAGGTGAGACCCCCGTTTC | |
| | TGCTCTGACGCTGGCAGTGACCACCCACCCCCACTTAGT | |
| | CCACCCGCCCAACCTGCCCGGTCCTTGTGCAAACTCACA | |
| | CCCGGCGCGGACACACAGTCCCGGGTCCGAGGCGGAGGA | |
| | GGACGGAGGCGCGGCCCGGCCGTCCCCGCCCGAGCCCTG | |
| | GGCCGCGCGCACTGAGCCGCCCTCTGACCCCGCCGCAGG | |
| | CTGCCCGCTGCTCACCACCACCGGGCTGTCGGGCCTGGT | |
| | GCAGCTGCAGGAGCTGGAGGAGCTGGAGCTGACCAACTG | |
| | CCCCGGGGCCACCCCCGAGCTCTTCAAGTATTTCTCGCA | |
| | GCACCTGCCCCGCTGCCTCGTCATTGAGTAG | |
| | ORF Start: | |
| | ATG at 1 | ORF Stop: TAG at 1744 |
| | SEQ ID NO:40 | 581 aa MW at 62036.5 kD |

| | |
|---|---|
| NOV16 | MVLARVARRPAGSAEAQASLAQKAALPERARKMSSPGID |
| CG59299-01 | QDPKPPCLPRNGLVKLPGQPNGLGAASITKGTPATKNRP |
| Protein | CQPPPPPTLPPPSLAAPLSRAALAGGPCTPAGGPASALA |
| | PGHPAERPPLATDEKILNGLFWYFSACEKCVLAQVCKAW |
| | RRVLYQPKFWAGLTPVLHAKELYNVLPGGEKEFVNLQGF |
| | AARGFEGFCLVGVSDLDICEFIDNYALSKKGVKAMSLKR |
| Sequence | STITDAGLEVMLEQMQGVVRLELSGCNDFTEAGLWSSLS |
| | ARTTSLSVSDCINVADDAIAAISQLLPNLAELSLQAYHV |
| | TDTALAYFTARQGHSTHTLRLLSCWEITNHGVVNVVHSL |
| | PNLTALSLSGCSKVTDDGVELVAENLRKLRSLDLSWCPR |
| | ITDMALEYVACDLHRLEELVLDRCVRITDTGLSYLSTMS |
| | SLRSLYLRWCCQVQDFGLKHLLALGSLRLLSLAGETPVS |
| | ALTLAVTTHPHLVHPPNLPGPCANSHPARTHSPGSEAEE |
| | EGGAARPSPPEPWAARTEPPSDPAAGCPLLTTTGLSGLV |
| | QLQELEELELTNCPGATPELFKYFSQHLPRCLVIE |

Further analysis of the NOV16 protein yielded the following properties shown in Table 16B.

TABLE 16B
Protein Sequence Properties NOV16

| | |
|---|---|
| PSort | 0.6586 probability located in mitochondrial matrix space; |
| analysis: | 0.3512 probability located in mitochondrial inner membrane; |
| | 0.3512 probability located in mitochondrial intermembrane |
| | space; 0.3512 probability located in mitochondrial outer |
| | membrane |
| SignalP | No Known Signal Sequence Predicted |
| analysis: | |

A search of the NOV16 protein against the Geneseq database, a proprietary database that contains sequences published in patents and patent publication, yielded several homologous proteins shown in Table 16C.

TABLE 16C
Geneseq Results for NOV16

| | | NOV16 | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| AAB92791 | Human protein sequence SEQ ID NO:11288 | 131 . . . 450 | 90/334 (26%) | 3e−20 |
| | - Homo sapiens, 423 aa. [EP1074617-A2, | 17 . . . 333 | 146/334 (42%) | |
| | 07-FEB-2001] | | | |
| AAB92961 | Human protein sequence SEQ ID NO:11652 | 131 . . . 450 | 90/334 (26%) | 4e−20 |
| | - Homo sapiens, 423 aa. [EP1074617-A2, | 17 . . . 333 | 146/334 (42%) | |
| | 07-FEB-2001] | | | |
| AAB42628 | Human ORFX ORF2392 polypeptide | 274 . . . 461 | 71/197 (36%) | 6e−20 |
| | sequence SEQ ID NO:4784 - Homo sapiens, | 2 . . . 196 | 104/197 (52%) | |
| | 212 aa. [WO200058473-A2, 05-OCT-2000] | | | |
| AAE08046 | Human full length F-box protein, F1 Alpha - | 131 . . . 450 | 90/334 (26%) | 7e−20 |
| | Homo sapiens, 456 aa. [US6232081-B1, 15- | 19 . . . 335 | 146/334 (42%) | |
| | MAY-2001] | | | |
| AAB48290 | Human ZF1 protein - Homo sapiens, 466 aa. | 131 . . . 450 | 90/334 (26%) | 7e−20 |
| | [WO200075184-A1, 14-DEC-2000] | 17 . . . 333 | 146/334 (42%) | |

In a BLAST search of public sequence databases, the NOV16 protein was found to have homology to the proteins shown in the BLASTP data in Table 16D.

TABLE 16D
Public BLASTP Results for NOV16

| | | NOV16 | | |
|---|---|---|---|---|
| Protein | | Residues/ | Identities/ | |
| Accession | | Match | Similarities for the | |
| Number | Protein/Organism/Length | Residues | Matched Portion | Expect Value |
| Q96S14 | POSSIBLE G-PROTEIN RECEPTOR - | 1 . . . 581 | 581/581 (100%) | 0.0 |
| | Homo sapiens (Human), 581 aa. | 1 . . . 581 | 581/581 (100%) | |
| Q9UJI0 | C380A1.1 (NOVEL PROTEIN) - Homo | 144 . . . 357 | 214/214 (100%) | e−121 |
| | sapiens (Human), 214 aa (fragment). | 1 . . . 214 | 214/214 (100%) | |
| Q9VTL7 | CG14134 PROTEIN - Drosophila | 348 . . . 463 | 85/116 (73%) | 1e−45 |
| | melanogaster (Fruit fly), 176 aa. | 13 . . . 128 | 105/116 (90%) | |
| AAH21329 | HYPOTHETICAL 43.9 KDA | 132 . . . 461 | 98/382 (25%) | 7e−24 |
| | PROTEIN - Mus musculus (Mouse), | 11 . . . 388 | 180/382 (46%) | |
| | 400 aa. | | | |
| Q9W214 | CG9952 PROTEIN (PARTNER OF | 86 . . . 469 | 113/429 (26%) | 1e−23 |
| | PAIRED) - Drosophila melanogaster | 97 . . . 515 | 190/429 (43%) | |
| | (Fruit fly), 538 aa. | | | |

PFam analysis predicts that the NOV16 protein contains the domains shown in the Table 16E.

TABLE 16E
Domain Analysis of NOV16

| | NOV16 | Identities/Similarities | Expect |
|---|---|---|---|
| Pfam Domain | Match Region | for the Matched Region | Value |
| F-box: | 124 . . . 171 | 6/48 (12%) | 1.3 |
| domain 1 of 1 | | 31/48 (65%) | |
| PTS-HPr: | 288 . . . 302 | 5/15 (33%) | 6.4 |
| domain 1 of 1 | | 13/15 (87%) | |

### Example A17

The NOV17 clone was analyzed, and the nucleotide and predicted polypeptide sequences are shown in Table 17A.

TABLE 17A
NOV17 Sequence Analysis

| | SEQ ID NO:41 | 645 bp |
|---|---|---|
| NOV 17a, | GGCTGGTCTGTGTCCCCCGCAGGCCATGGACACCTTCAG | |
| G59632-01 | CACCAAGAGCCTGGCTCTGCAGGCGCAGAAGAAGCTCCT | |
| DNA | GAGTAAGATGGCGTCCAACGCAGTGGTGGCCGTGCTGGT | |
| Sequence | GGATGACACCAGCAGTGAGGTGCTGGATGAGCTGTACCG | |
| | CGCCACCAGGGAGTTCACGCGCAGCCGCAAGGAGGCCCA | |
| | GAAGATGCTCAAGAACCTGGTCAAGGTGGCCCTGAAGCT | |
| | GGGACTGCTGCTGCGTGGGGACCAGCTGGGCGGTGAGGA | |
| | GCTGGCGCTGCTGCGGCGCTTCCGCCACCGGGCGCGCTG | |
| | CCTGGCCATGACGGCCGTCAGCTTCCACCAGGTGGACTT | |
| | CACCTTCGACCGGCGCGTGCTGGCCGCCGGGCTGCTCGA | |
| | GTGCCGCGACCTGCTGCACCAGGCCGTGGGTCCCCACCT | |
| | GACCGCCAAGTCCCACGGCCGCATCAACCACGTGTTCGG | |
| | CCACCTAGCCGACTGCGACTTCCTGGCTGCGCTCTACGG | |
| | CCCCGCCGAGCCCTACCGCTCCCACCTGCGCAGGATCTG | |
| | CGAGGGCCTGGGCCGGATGCTGGACGAGGGCAGCCTCTG | |
| | AACCCCGGCGCCGCCCAACCGCGCCCCTCGCGCCTTTTG | |
| | GGGCTCTCCTGCTGGGCGCGG | |
| | ORF Start: | |
| | ATG at 26 | ORF Stop: TGA at 584 |
| | SEQ ID NO:42 | 186 aa MW at 20827.0 kD |
| NOV17a, | MDTFSTKSLALQAQKKLLSKMASKAVVAVLVDDTSSEVL | |
| CG59632-01 | DELYRATREFYRSRKEAQKMLKNLVKVALKLGLLLRGDQ | |
| Protein | LGGEELALLRRFRHRARCLAMTAVSFHQVDFTFDRRVLA | |
| Sequence | AGLLECRDLLHQAVGPHLTAKSHGRINHVFGHLADCDFL | |
| | AALYGPAEPYRSHLRRICEGLGRMLDEGSL | |
| | SEQ ID NO:43 | 619 bp |
| NOV17a, | GCAGGCCATGGACACCTTCAGCACCAAGAGCCTGGCTCT | |
| CG59632-02 | GCAGGCGCAGAAGAAGCTCCTGAGTAAGATGGCGTCCAA | |
| DNA | GGCAGTGGTGGCCGTGCTGGTGGATGACACCAGCAGTGA | |
| | GGTGCTGGATGAGCTGTACCGCGCCACCAGGGAGTTCAC | |
| | GCGCAGCCGCAAGGAGGCCCAGAAGATGCTCAAGAACCT | |
| | GGTCAAGGTGGCCCTGAAGCTGGGACTGCTGCTGCGTGG | |
| | GGACCAGCTGGGCGGTGAGGAGCTGGCGCTGCTGCGGCG | |
| | CTTCCGCCACCGGGCGCGCTGCCTGGCCATGACGGCCGT | |
| | CAGCTTCCACCAGGTGGACTTCACCTTCGACCGGCGCGT | |
| | GCTGGCCGCCGGGCTGCTCGAGTGCCGCGACCTGCTGCA | |
| | CCAGGCCGTGGGTCCCCACCTGACCGCCAAGTCCCACGG | |
| | CCGCATCAACCACGTGTTCGGCCACCTAGCCGACTGCGA | |
| | CTTCCTGGCTGCGCTCTACGGCCCCGCCGAGCCCTACCG | |
| | CTCCCACCTGCGCAGGATCTGCGAGGGCCTGGGCCGGAT | |
| | GCTGGACGAGGGCAGCCTCTGAACCCCGGCGCCGCCCAA | |
| | CCGCGCCCCTCGCGCCTTTTGGGGCTCTCCTGCT | |
| | ORF Start: | |
| | CAG at 2 | ORF Stop: TGA at 566 |
| | SEQ ID NO:44 | 188 aa MW at 21026.2 kD |

| | |
|---|---|
| NOV17b, | QAMDTFSTKSLALQAQKKLLSKMASKAVVAVLVDDTSSE |
| CG59632-02 | VLDELYRATREFTRSRKEAQKMLKNLVKVALKLGLLLRG |
| Protein | DQLGGEELALLRRFRHRARCLAMTAVSFHQVDFTFDRRV |
| Sequence | LAAGLLECRDLLHQAVGPHLTAKSHGRINHVFQHLADCD |
| | FLAALYGPAEPYRSHLRRICEGLGRMLDEGSL |

Sequence comparison of the above protein sequences yields the following sequence relationships shown in Table 17B.

TABLE 17B
Comparison of NOV17a against NOV17b.

| | | Identities/Similarities |
|---|---|---|
| | NOV17a Residues/NOV17b | for the |
| Protein Sequence | Match Residues | Matched Region |
| NOV17b | 1 . . . 186 | 186/186 (100%) |
| | 3 . . . 188 | |

Further analysis of the NOV17a protein yielded the following properties shown in Table 17C.

TABLE 17C
Protein Sequence Properties NOV17a

| | |
|---|---|
| PSort | 0.4385 probability located in mitochondrial matrix space; |
| analysis: | 0.3000 probability located in microbody (peroxisome); |
| | 0.1789 probability located in lysosome (lumen); 0.1227 |
| | probability located in mitochondrial inner membrane |
| SignalP | No Known Signal Sequence Predicted |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB93474 | Human protein sequence SEQ ID NO:12752 | 2 . . . 186 | 105/185 (56%) | 1e−61 |
| | - Homo sapiens, 188 aa. [EP1074617-A2, | 4 . . . 188 | 148/185 (79%) | |
| | 07-FEB-2001] | | | |
| AAB93396 | Human protein sequence SEQ ID NO:12579 | 2 . . . 186 | 105/185 (56%) | 1e−61 |
| | - Homo sapiens, 198 aa. [EP1074617-A2, | 14 . . . 198 | 148/185 (79%) | |
| | 07-FEB-2001] | | | |
| AAY36722 | Fragment of human secreted protein | 2 . . . 186 | 104/185 (56%) | 6e−61 |
| | encoded by gene 98 - Homo sapiens, 227 aa. | 43 . . . 227 | 147/185 (79%) | |
| | [WO9931117-A1, 24-JUN-1999] | | | |
| AAB60457 | Human cell cycle and proliferation protein | 1 . . . 186 | 106/186 (56%) | 7e−54 |
| | CCYPR-5, SEQ ID NO:5 - Homo sapiens, | 1 . . . 184 | 139/186 (73%) | |
| | 184 aa. [WO200107471-A2, 01-FEB-2001] | | | |
| AAM83841 | Human immune/haematopoietic antigen | 1 . . . 103 | 58/103 (56%) | 4e−26 |
| | SEQ ID NO:11434 - Homo sapiens, 144 aa. | 25 . . . 127 | 81/103 (78%) | |
| | [WO200157182-A2, 09-AUG-2001] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | | |
|---|---|---|---|---|
| Protein | | Residues/ | Identities/ | |
| Accession | | Match | Similarities for the | Expect |
| Number | Protein/Organism/Length | Residues | Matched Portion | Value |
| AAH17672 | SIMILAR TO RIKEN CDNA | 1 . . . 186 | 185/186 (99%) | e−101 |
| | 2600017J23 GENE - Homo sapiens | 1 . . . 186 | 185/186 (99%) | |
| | (Human), 186 aa. | | | |
| Q9DBH7 | 2600017J23RIK PROTEIN - Mus | 1 . . . 186 | 151/186 (81%) | 2e−82 |
| | musculus (Mouse), 186 aa. | 1 . . . 186 | 169/186 (90%) | |
| Q9D0N6 | 2600017J23RIK PROTEIN - Mus | 1 . . . 186 | 151/186 (81%) | 4e−82 |
| | musculus (Mouse), 186 aa. | 1 . . . 186 | 169/186 (90%) | |
| O95379 | MDC-3.13 ISOFORM 2 (TNF- | 2 . . . 186 | 105/185 (56%) | 5e−61 |
| | INDUCED PROTEIN) - Homo sapiens | 14 . . . 198 | 148/185 (79%) | |
| | (Human), 198 aa. | | | |
| Q9UER5 | MDC-3.13 ISOFORM 1 - Homo | 2 . . . 186 | 105/185 (56%) | 5e−61 |
| | sapiens (Human), 190 aa. | 6 . . . 190 | 148/185 (79%) | |

PFam analysis predicts that the NOV17a protein contains the domains shown in the Table 17F.

TABLE 17F
Domain Analysis of NOV17a

| | | | |
|---|---|---|---|
| | | Identities/ | |
| | | Similarities | |
| | NOV17a | for the | |
| Pfam Domain | Match Region | Matched Region | Expect Value |

No Significant Matches Found To Known Sequences

### Example A18

The NOV18 clone was analyzed, and the nucleotide and predicted polypeptide sequences are shown in Table 18A.

TABLE 18A
NOV18 Sequence Analysis

| | SEQ ID NO:45 | 745 bp |
|---|---|---|
| NOV18, | TTGGTGTTGATGGAGGAGCAGGACGCCAGAGTCCCAGCC | |
| CG59653-01 | CTGGAACCGTTCAGAGTGGAGCAGGCACCACCTGTAATC | |
| DNA | TACTATGTCCCTGACTTCATCTCCAAAGAAGAGGAGGAG | |
| Sequence | TATTTGCTTCGACAGGTGACCCCAAAGCCAAAGTGGACC | |
| | CAGCTCTCTGGGAGAAAGTTACAGAACTGGGGTGGGCTT | |
| | CCTCATCCCCGAGGGATGGTTCCTGAGCGGCTGCCCCCA | |
| | TGGCTCCAGCGCTACGTGGACAAAGTGTCAAACCTCAGC | |
| | CTCTTTGGAGGCCTCCCAGCTAACCATGTCCTCGTGAAC | |
| | CAGTATCTGCCTGGGGAGGGCATCATGCCCCACGAGGAC | |
| | GGACCACTGTACTACCCGACTGTCAGCACCATCAGCCTG | |
| | GGCTCCCACACCGTGCTGGACTTCTACGAGCCGCGGCGG | |
| | CCAGAGGACGATGACCCTACAGAACAGGTGGGCCCCCAG | |
| | ACACTGCCCCAGCTACTGCTGGAACCGCGCAGCCTGCTG | |
| | GTGCTCCGCGGCCCCGCCTACACGCGTCTTCTCCACGGC | |
| | ATCGCCGCCGCCCGCGTAGACGCGCTGGACGCCGCCTCC | |
| | TCGCCGCCCAATGCGGCAGCCTGCCCGTCGGCGCGGCCG | |
| | GGAGCCTGCCTGGTGCGCGGCACCCGGGTCTCGCTGACC | |
| | ATCCGCCGCGTGCCCCGCGTGCTGCGCGCCGGCCTCCTG | |
| | CTGGGCAAGTGACCGCCAGGGCCGGGACCCCTCGGATTC | |
| | CCAG | |
| | ORF Start: | |
| | ATG at 10 | ORF Stop: TGA at 712 |
| | SEQ ID NO:46 | 234 aa MW at 25985.6 kD |

| | |
|---|---|
| NOV18, | MEEQDARVPALEPFRVEQAPPVIYYVPDFISKEEEEYLL |
| 59653-01 | RQVTPKPKWTQLSGRKLQNWGGLPHPRGMVPERLPPWLG |
| Protein | RYVDKVSNLSLFGGLPANHVLVNGYLPGEGIMPHEDGPL |
| Sequence | YYPTVSTISLGSHTVLDFYEPRRPEDDDPTEGVGPQTLP |
| | QLLLEPRSLLVLRGPAYTRLLHGIAAARVDALDAASSPP |
| | NAAACPSARPGACLVRGTRVSLTIRRVPRVLRAGLLLGK |

Further analysis of the NOV18 protein yielded the following properties shown in Table 18B.

TABLE 18B
Protein Sequence Properties NOV18

| | |
|---|---|
| PSort | 0.6500 probability located in cytoplasm; 0.3053 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV18 protein against the Geneseq database, a proprietary database that contains sequences published in patents and patent publication, yielded several homologous proteins shown in Table 18C.

TABLE 18C
Geneseq Results for NOV18

| | | NOV18 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAG93258 | Human protein HP10582 - Homo | 15 . . . 222 | 59/226 (26%) | 3e−09 |
| | sapiens, 614 aa. [WO200142302-A1, 14- | 129 . . . 336 | 93/226 (41%) | |
| | JUN-2001] | | | |

In a BLAST search of public sequence databases, the NOV18 protein was found to have homology to the proteins shown in the BLASTP data in Table 18D.

TABLE 18D
Public BLASTP Results for NOV18

| | | NOV18 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q96IE0 | UNKNOWN (PROTEIN FOR | 68 . . . 234 | 161/169 (95%) | 7e−88 |
| | MGC:15677) - Homo sapiens (Human), | 1 . . . 169 | 161/169 (95%) | |
| | 169 aa. | | | |
| Q95TD8 | LD37206P - Drosophila melanogaster | 14 . . . 230 | 112/225 (49%) | 3e−53 |
| | (Fruit fly), 228 aa. | 6 . . . 225 | 147/225 (64%) | |
| Q9VKU5 | CG6144 PROTEIN - Drosophila | 25 . . . 230 | 108/214 (50%) | 4e−50 |
| | melanogaster (Fruit fly), 223 aa. | 12 . . . 220 | 141/214 (65%) | |
| Q9SUP1 | HYPOTHETICAL 27.3 KDA PROTEIN | 11 . . . 225 | 91/233 (39%) | 2e−35 |
| | - Arabidopsis thaliana (Mouse-ear cress), | 3 . . . 234 | 126/233 (54%) | |
| | 241 aa. | | | |
| Q17527 | B0564.2 PROTEIN - Caenorhabditis | 11 . . . 138 | 54/131 (41%) | 3e−24 |
| | elegans, 160 aa. | 11 . . . 140 | 86/131 (65%) | |

PFam analysis predicts that the NOV18 protein contains the domains shown in the Table 18E.

TABLE 18E
Domain Analysis of NOV18

| | | | |
|---|---|---|---|
| | | Identities/ | |
| | | Similarities | |
| | NOV18 | for the | |
| Pfam Domain | Match Region | Matched Region | Expect Value |

No Significant Matches Found To Known Sequences

### Example A19

The NOV19 clone was analyzed, and the nucleotide and predicted polypeptide sequences are shown in Table 19A.

TABLE 19A
NOV19 Sequence Analysis

| | SEQ ID NO:47 | 792 bp |
|---|---|---|
| NOV19, | ATGGCGGCCATCCCCTCCAGCGGCTCGCTCGTGGCCACC | |
| CG59303-01 | CACGACTACTACCGGCGCCGCCTGGGTTCCACTTCCAGC | |
| DNA | AACAGCTCCTGCAGCAGTACCGAGTGCCCCGGGGAAGCC | |
| Sequence | ATTCCCCACCCCCCAGGTCTCCCCAAGGCTGACCCGGGT | |
| | CATTGGTGGGCCAGCTTCTTTTTCGGGAAGTCCACCCTC | |
| | CCGTTCATGGCCACGGTGTTGGAGTCCGCAGAGCACTCG | |
| | GAACCTCCCCAGGCCTCCAGCAGCATGACCGCCTGTGGC | |
| | CTGGCTCGGGACGCCCCGAGGAAGCAGCCCGGCGGCGGC | |
| | TGCAGCAAGCAGACCTTCGCATCAACACAGCAGACACCA | |
| | AAAACCAGTGAGAGCCCCGCTCTCTACCGCCCGGCCCCA | |
| | GCACTCGCTAGCTTTCCTGACACCTGGAACTGTGCACCT | |
| | GGCACCAAGCGGAAAATAAACTCCAAGCAGCCAGTAGCC | |
| | CCGATGGGCACAATTGAAAACATTGGTTATATTACCAAG | |
| | GCTTTGACTGGAATGTCCTATTTTCAGATACGACTAAAG | |
| | GAGTTAGGGTTGACTGCATGGTCCAATAAAGCCCCTTGG | |
| | AAAAAAGCTGGCCTGATGAGTAAAGAATGTCAATTCCTG | |
| | GCAGGTCCCAGAATCTTAAGATACCTTAGGGACCTCAAG | |
| | AAGAGAGGAATTCCCCTAAATCTGTACTTATTGCAGGCA | |
| | AAGTCTGATGGCAAGTCTTTGCGATGGCTTCCTAGCCTC | |
| | AAGAGGCTTTTAAAAGCCCAATCTCAGATTCCTTATAAA | |
| | AAGTTCCAGTAG | |
| | ORF Start: | |
| | ATG at 1 | ORF Stop: TAG at 790 |
| | SEQ ID NO:48 | 263 aa MW at 28761.8 kD |

| | |
|---|---|
| NOV19, | MAAIPSSGSLVATHDYYRRRLGSTSSNSSCSSTECPGEA |
| CG59303-01 | IPHPPGLPKADPGHWWASFFFGKSTLPFMATVLESAEHS |
| Protein | EPPQASSSMTACGLARDAPRKQPGGGCSKQTFASTQQTP |
| Sequence | KTSESPALYRPAPALASFPDTWNCAPGTKRKINSKQPVA |
| | PMGTIENIGYITKALTGMSYFQIRLKELGLTAWSNKAPW |
| | KKAGLMSKECQFLAGPRILRYLRDLKKRGIPLNLYLLQA |
| | KSDQKSLRWLPSLKRLLKAQSQIPYKKFQ |

Further analysis of the NOV19 protein yielded the following properties shown in Table 19B.

TABLE 19B
Protein Sequence Properties NOV19

| | |
|---|---|
| PSort | 0.4558 probability located in mitochondrial matrix space; |
| analysis: | 0.2393 probability located in microbody (peroxisome); 0.1497 |
| | probability located in mitochondrial inner membrane; 0.1497 |
| | probability located in mitochondrial intermembrane space |
| SignalP | Likely cleavage site between residues 12 and 13 |
| analysis: | |

A search of the NOV19 protein against the Geneseq database, a proprietary database that contains sequences published in patents and patent publication, yielded several homologous proteins shown in Table 19C.

TABLE 19C
Geneseq Results for NOV19

| | | NOV19 | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| AAE12624 | Human gene 2 encoded secreted protein | 1 . . . 106 | 104/106 (98%) | 2e−57 |
| | HUSYJ75 - Homo sapiens, 114 aa. | 1 . . . 106 | 104/106 (98%) | |
| | [WO200170804-A1, 27-SEP-2001] | | | |
| AAU04354 | Mammalian toxicological response marker | 1 . . . 106 | 104/106 (98%) | 2e−57 |
| | protein #6 - Homo sapiens, 114 aa. | 1 . . . 106 | 104/106 (98%) | |
| | [WO200136684-A2, 25-MAY-2001] | | | |
| AAG93282 | Human protein HP10052 - Homo sapiens, | 1 . . . 106 | 104/106 (98%) | 2e−57 |
| | 114 aa. [WO200142302-A1, 14-JUN-2001] | 1 . . . 106 | 104/106 (98%) | |
| AAB58965 | Breast and ovarian cancer associated antigen | 20 . . . 106 | 83/87 (95%) | 5e−45 |
| | protein sequence SEQ ID 673 - Homo | 45 . . . 131 | 84/87 (96%) | |
| | sapiens, 139 aa. [WO200055173-A1, 21- | | | |
| | SEP-2000] | | | |

In a BLAST search of public sequence databases, the NOV19 protein was found to have homology to the proteins shown in the BLASTP data in Table 19D.

TABLE 19D
Public BLASTP Results for NOV19

| | | NOV19 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9H3Y8 | DJ697K14.9.1 (NOVEL PROTEIN) | 1 . . . 106 | 104/106 (98%) | 7e−57 |
| | (UNKNOWN) (PROTEIN FOR MGC:2479) | 1 . . . 106 | 104/106 (98%) | |
| | - Homo sapiens (Human), 114 aa. | | | |
| Q9CR37 | 2700038C09RIK PROTEIN | 1 . . . 101 | 74/102 (72%) | 2e−35 |
| | (2610317A05RIK PROTEIN) | 1 . . . 102 | 81/102 (78%) | |
| | (BM401L17.7.1) (NOVEL PROTEIN | | | |
| | (ISOFORM 1)) - Mus musculus (Mouse), | | | |
| | 115 aa. | | | |
| Q9H3Y7 | DJ697K14.9.2 (NOVEL PROTEIN, | 1 . . . 66 | 66/66 (100%) | 2e−34 |
| | ISOFORM 2) - Homo sapiens (Human), 70 | 1 . . . 66 | 66/66 (100%) | |
| | aa. | | | |

PFam analysis predicts that the NOV19 protein contains the domains shown in the Table 19E.

TABLE 19E
Domain Analysis of NOV19

| | | | |
|---|---|---|---|
| | NOV19 | Identities/Similarities | Expect |
| Pfam Domain | Match Region | for the Matched Region | Value |

No Significant Matches Found To Known Sequences

### Example A20

The NOV20 clone was analyzed, and the nucleotide and predicted polypeptide sequences are shown in Table 20A.

TABLE 20A
NOV20 Sequence Analysis

| | SEQ ID NO:49 | 489 bp |
|---|---|---|
| NOV20, | CTCACTATAGGCTTCGAGCGTGACAACACCGTAACCGTT | |
| CG59673-01 | TGTATGGATTACATAAAGGGGCGTTGCATGAGGGAGAAA | |
| DNA | TGCAAATATTTTCACCCTCCTGCACACTTGCAGGCCAAA | |
| Sequence | ATCAAAGCTGCGCAGCACCAAGCCAACCAAGCTGCGGTG | |
| | GCCGCCCAGGCAGCCGCGGCCGCGGCCACAGTCATGGCC | |
| | TTTCCCCCTGGTGCTCTTCATCCTTTACCAAAGAGACAA | |
| | GCACTTGAAAAAAGCAATGGTACCAGCGCGGTCTTTAAC | |
| | CCCAGCGTCTTGCACTACCAGCAGGCTCTCACCAGCGCA | |
| | CAGTTGCACCAACACGCCGCGTTCATTCCAACAGATAAT | |
| | TCTGAAATAATCAGCAGAAACGGAATGGAATGCCAAGAA | |
| | TCTGCATTGAGAATAACTAAACATTGTTACTGTACATAC | |
| | TATCCTGTTTCCTCCTCAATAGAATTGCCACAAACTGCA | |
| | TGCTAAATAAAGATGTAGTTC | |
| | ORF Start: | |
| | ATG at 43 | ORF Stop: TAA at 472 |
| | SEQ ID NO:50 | 143 aa MW at 15597.7 kD |

| | |
|---|---|
| NOV20, | MDYIKGRCMREKCKYFHPPAHLQAKIKAAQHQANQAAVA |
| CG59673-01 | AQAAAAAATVMAFPPGALHPLPKRQALEKSNGTSAVFNP |
| Protein | SVLHYQQALTSAQLQQHAAFIPTDNSEIISRNGMECQES |
| Sequence | ALRITKHCYCTYYPVSSSTELPQTAC |

Further analysis of the NOV20 protein yielded the following properties shown in Table 20B.

TABLE 20B
Protein Sequence Properties NOV20

| | |
|---|---|
| PSort | 0.4500 probability located in cytoplasm; 0.3000 probability |
| analysis: | located in microbody (peroxisome); 0.2185 probability |
| | located in lysosome (lumen); 0.1000 probability located in |
| | mitochondrial matrix space |
| SignalP | No Known Signal Sequence Predicted |
| analysis: | |

A search of the NOV20 protein against the Geneseq database, a proprietary database that contains sequences published in patents and patent publication, yielded several homologous proteins shown in Table 20C.

TABLE 20C
Geneseq Results for NOV20

| | | NOV20 | | |
|---|---|---|---|---|
| | | Residues/ | Identities/ | |
| Geneseq | | Match | Similarities for the | Expect |
| Identifier | Protein/Organism/Length [Patent #, Date] | Residues | Matched Region | Value |
| ABB44614 | Mouse wound healing related polypeptide | 1 . . . 127 | 77/127 (60%) | 6e−35 |
| | SEQ ID NO 103 - Mus musculus, 341 aa. | 221 . . . 337 | 85/127 (66%) | |
| | [CA2325226-A1, 17-MAY-2001] | | | |
| ABB44615 | Human wound healing related polypeptide | 1 . . . 100 | 73/100 (73%) | 8e−35 |
| | SEQ ID NO 104 - Homo sapiens, 370 aa. | 222 . . . 319 | 79/100 (79%) | |
| | [CA2325226-A1, 17-MAY-2001] | | | |
| AAB60764 | Gene 15 related peptide #2 - Homo sapiens, | 1 . . . 100 | 73/100 (73%) | 8e−35 |
| | 205 aa. [WO200076531-A1, 21-DEC-2000] | 45 . . . 142 | 79/100 (79%) | |
| AAB60763 | Gene 15 related peptide #1 - Homo sapiens, | 1 . . . 100 | 73/100 (73%) | 8e−35 |
| | 193 aa. [WO200076531-A1, 21-DEC-2000] | 45 . . . 142 | 79/100 (79%) | |
| AAB75564 | Human secreted protein sequence encoded by | 1 . . . 100 | 73/100 (73%) | 8e−35 |
| | gene 7 SEQ ID NO:118 - Homo sapiens, 205 | 45 . . . 142 | 79/100 (79%) | |
| | aa. [WO200077026-A1, 21-DEC-2000] | | | |

In a BLAST search of public sequence databases, the NOV20 protein was found to have homology to the proteins shown in the BLASTP data in Table 20D.

TABLE 20D
Public BLASTP Results for NOV20

| | | NOV20 | | |
|---|---|---|---|---|
| Protein | | Residues/ | Identities/ | |
| Accession | | Match | Similarities for the | Value |
| Number | Protein/Organism/Length | Residues | Matched Portion | Expect |
| Q9P1F2 | PRO2032 - Homo sapiens (Human), 94 aa. | 50 . . . 143 | 94/94 (100%) | 4e−50 |
| | | 1 . . . 94 | 94/94 (100%) | |
| Q9JKP5 | Muscleblind-like protein (Triplet- | 1 . . . 127 | 77/127 (60%) | 2e−34 |
| | expansion RNA-binding protein) - Mus | 221 . . . 337 | 85/127 (66%) | |
| | musculus (Mouse), 341 aa. | | | |
| Q96P92 | MUSCLEBLIND 41KD ISOFORM - | 1 . . . 100 | 73/100 (73%) | 3e−34 |
| | Homo sapiens (Human), 382 aa. | 222 . . . 319 | 79/100 (79%) | |
| Q96RE3 | 36 KDA MUSCLEBLIND PROTEIN | 1 . . . 100 | 73/100 (73%) | 3e−34 |
| | EXP36 - Homo sapiens (Human), 314 aa. | 154 . . . 251 | 79/100 (79%) | |
| Q9NR56 | Muscleblind-like protein (Triplet- | 1 . . . 100 | 72/117 (61%) | 4e−32 |
| | expansion RNA-binding protein) - Homo | 222 . . . 337 | 80/117 (67%) | |
| | sapiens (Human), 388 aa. | | | |

PFam analysis predicts that the NOV20 protein contains the domains shown in the Table 20E.

TABLE 20E
Domain Analysis of NOV20

| | | | |
|---|---|---|---|
| | NOV20 | Identities/Similarities | Expect |
| Pfam Domain | Match Region | for the Matched Region | Value |

No Significant Matches Found To Known Sequences

### Example A21

The NOV21 clone was analyzed, and the nucleotide and predicted polypeptide sequences are shown in Table 21A.

TABLE 21A
NOV21 Sequence Analysis

| | SEQ ID NO:51 | 4413 bp |
|---|---|---|
| NOV21, | CTCACATTCCTGGCATAGCAGCCGCCTCCGGCGCGGGCCGACCCTGGGGCTGCGCGCT | |
| CG59636-01 | GGGGCGCGAACAGCCAGAGCGTCGGCGCCACGGCCGAGAACACATCTTCGCCGCCGAG | |
| DNA | CTGAGCTGGGCCGAGCCGGAGGTTGTGGTCTGACTGCGCTGGGCACCCTCGGGCCGCA | |
| Sequence | GCGGTGCTCTGGGGCCAGGTGCCACCGGCCATTGTCCAGGCAGCTGTGTGCAAGCCAA | |
| | AGAAGCATGAGGACACTGGAAGACTCCTCGGGGACAGTCCTGCACCGCCTCATCCAGG | |
| | AGCAGCTGCGCTACGGCAACCTGACTGAGACGCGCACGCTGCTAGCCATCCAGCAGCA | |
| | GGCCCTGAGGGGTGGGGCTGGAACTGGGGGTACAGGGAGCCCCCAGGCCTCCCTGGAG | |
| | ATCCTGGCCCCAGAGGACAGTCAGGTGCTGCAGCAGGCCACCAGGCAGGAGCCCCAGG | |
| | GCCACAGCCCAGCAAGGGAGAGGAGCTGCCCACCTATGAGGAGGCCAAAGCCCACTCG | |
| | CCCACAGCCCAGCAAGGGAGAGGAGCTGCCCACCTATGAGGAGGCCAAAGCCCACTCG | |
| | CAGTACTATGCGGCCCAGCAGGCAGGGACCCGGCCACATGCGGGGGACCGAGATCCCC | |
| | GTGGGGCCCCGGGAGGCAGTCGGAGGCAGGACGAGGCCCTGCGGGAGCTGAGGCATGG | |
| | GCACGTGCGCTCGTTGAGTGAACGGCTCCTTCAGTTGTCCCTGGAGAGGAACGGCGCC | |
| | CGGGCCCCCAGCCACATGAGCTCCTCCCACAGCTTCCCACAGCTGGCCCGCAACCAGC | |
| | AGGGCCCCCCACTGAGGGGCCCCCCTGCTGAGGGCCCAGAGTCCCGAGGACCCCCACC | |
| | TCAGTACCCTCATGTTGTACTAGCTCATGAGACCACCACTGCTGTCACTGACCCACGG | |
| | TACCGTGCCCGCGGCAGCCCGCACTTCCAGCATGCTGAAGTCAGGATCCTGCAGGCCC | |
| | AGGTGCCTCCTGTGTTCCTCCAACAGCAGCAGCAGTACCAGTACCTGCAGCAATCTCA | |
| | GGAGCACCCCCCTCCCCCACATCCAGCTGCTCTCGGCCATGGCCCCCTGAGCTCCCTC | |
| | AGTCCACCTGCTGTGGAGGGGCCAGTGAGTGCCCAGGCCTCCTCAGCCACCTCGGGCA | |
| | GTGCCCACCTGGCCCAGNATGGAGGCCGTGCTGAGGGAGAATGCCAGGCTGCAGAGAG | |
| | ACAATGAGCGGCTGCAGAGGGAGCTGGAGAGCTCTGCGGAGAAGGCTGGCCGCATTGA | |
| | GAAGCTGGAAAGCGAAATCCAGCGGCTCTCTGAGGCCCATGAGAGCCTGACCAGAGCC | |
| | TCCTCCAAGCGTGAGGCCCTGGAGAAGACCATGCGGAACAAGATGGACAGTGAAATGA | |
| | GGAGGCTGCAAGACTTCAACCGGGATCTTAGAGACAGATTGGAATCTGCAAATCGCCG | |
| | CCTGGCAAGCAAGACACAGGAGGCCCAGGCCGGCAGTCAGGACATGGTGGCCAAGCTG | |
| | CTTGCTCAGAGCTACGAACAGCAGCAGGAGCAAGAGAAGCTGGAGCGAGAGATGGCAC | |
| | TGCTGCGCGGCGCCATCGAGGACCAGCGGCGGCGTGCCGAGCTGCTGGAGCAGGCTCT | |
| | GGGCAATGCGCAGGGCCGGGCAGCTCGAGCCGAAGAGGAGCTGCGCAAGAAGCAGGCC | |
| | TATGTGGAGAAAGTGGAGCGGCTGCAGCAGGCGCTCGGGCAGCTGCAGGCAGCCTGTG | |
| | AGAAGCGGGAGCAGCTGGAGCTGCGTCTGCGGACTCGCCTGGAGCAGGAACTCAAGGC | |
| | CCTGCGTGCACAGCAGAGACAGGCAGGTGCCCCAGGTGGTAGCAGTGGCAGTGGTGGG | |
| | TCTCCAGAGCTCAGCGCCCTGCGACTGTCAGAACAACTGCGAGAGAAGGAGGAGCAGA | |
| | TCCTGGCGCTGGAGGCCGACATGACCAAGTGGGAGCAGAAGTATTTGGAGGAACGTGC | |
| | CATGAGGCAGTTTGCCATGGATGCGGCTGCCACGGCTGCTGCTCAGCGTGACACCACT | |
| | CTCATCCGACATTCCCCCCAGCCCTCACCCAGCAGCAGCTTCAATGAGGGTCTGCTCA | |
| | CCTGGAGAAGGATGCAGTGATCAAGGTCCTTCAGCAGCGCTCCAGGAGAGACCCTGGC | |
| | AAGGCCATCCAGGGCTCCCTGCGGCCTGCCAAGTCGGTGCCATCTGTTTTCGCGGCTG | |
| | CGGCAGCAGGAACCCAGGGCTGGCAAGGGCTCTCTTCTAGTGAGCGACAAACAGCAGA | |
| | CGCCCCTGCTCGGCTGACTACAGCAGACAGAGCACCCACAGAGGAGCCAGTGGTCACA | |
| | GCTCCCCCTGCTGCCCATGCCAAACACGGGAGCAGAGATGGGAGCACCCAGACTGACG | |
| | GCCCCCCAGACAGCACCTCCACCTGCCTGCCACCGGAGCCTGACAGCCTTCTGGGGTG | |
| | CAGCAGTAGCCAGAGAGCAGCCTCTCTGGACTCTGTAGCTACATCCAGAGTCCAGGAC | |
| | TTGTCAGACATGGTGGAGATACTGATCTGAAGGAGGTGGTGCTTCAGGACTCTGAGCC | |
| | ATTCTCTCCCCTCCTCTGCCCTGTGCCACTCTCAGCCATTTCAGCAGCCCCGTCAACC | |
| | GCTGCTCCGTCCCTTTCCCCAGCCAGACACTCATTCCCATTGACCATCTGGTCCCAGG | |
| | AGCTCAGGAGGAGGACCCCAGGGGAGAGGAGAGCTGTGAGAGCACCGGCACCCCCAGA | |
| | AGACTCTGCTTCTTAGCCCACATTCCTCCGGGCCTTATGGAGAATGAGGATTCAGCCT | |
| | TGACTTCTTGCCCAAGGCCTGCTACTGGGGTAGCAACTGACAGCTCAGAAAGGAGCTG | |
| | AGCTCCCTCTGCCCTGCCAGTTGTCAGTCAGGCAGGGAGGGAGTGGCTGTGTTGGTTT | |
| | GGGGAACTAATTTCCAAGGACGGCTGCCCGTGGACACCAGGTGGACTGGTTCACTAAT | |
| | CAAGTCAGCCATATTGTTCTCTGGCTAAGTTTGGTTCCAGCCAACGTCATCTGCTCTT | |
| | CAGTTCCTCACTGCCTTCTTGGGATACTAAGACTTGAATTTTTTGGGGACTATTAAGG | |
| | GTGTTAGTCTTGGAGAAGACACAGCCTCACCTTCTCACTTGCTGTGGGTGAGGGGCCA | |
| | TTTAAGTGGACTGGGAGACAGTGCGCAGTTTGTATATAATTCCCTTTCTTGTGGAACA | |
| | GAAGACTGAGGCCTGCAGGTTCCGATGTGTCTCCATGGGCTGTGCTCCCCTCTTCCTA | |
| | CTGTCAGTTTCTGAAACTTCTGACTGGCCTCCCAGTTATGCCTCCTCCTCAAGTTCCT | |
| | GGCCCGTGGATGTTAAAGCTGCTCGATTCCCAGGATCTCGGCTGCCTTTTCCTCTATC | |
| | TTGAGCCCTATAAATGCCCACGGGACCCCCACCACCAGCCTCTTGAAGTGGCTCCACA | |
| | GCTCCTGTCCCTGGAACATCCTGTCAGTTTGGTCATAAACCCTGAGCCAGATGAAATG | |
| | AGCCACCGTGAACAGACATCTGCCATGCCCCCAGGTGGGCTTCGGTGGCCCTACCCGG | |
| | TACCAGTTCTCTCTGAGAAACTGGAGATGTCTTGTTAGCATAAGTGTCTTCATTCCCA | |
| | CCTGGAGGGTTTGGGAGAGGAGCAAAGCAGTTGAAAACTAGTTAATGAGCTACAAGAG | |
| | TCAAATAGTCCTCTGAATGGAGCCCCCATCACAAAACAGTGCCCAGGAGGCTGGCTCC | |
| | TCAAGCTACCCATGCCCAGCGCCCTAAAGCAGGACCAGATGCTTTGGAATTGGGGTGA | |
| | AACACCCACATGGCAGCCTGCTAGCAGCAGTGACTTTGACTTCTGGTCTTAAAGAGTC | |
| | CCTCACTTCAGCCCCAGGAGCTATTGGTGGGTTTTAGCAGTTTTGTCTTTACCGTTTT | |
| | TAGTTCTCCTTGATTCTTTGTTTTCTTCCTTTATCGTTTTTAGGTTTGGTATGTGTTG | |
| | TTTTATTTCCATGGTTCCTCAAGTTTCCTTTTTAAACATTTGCATTTGCTGGACAATT | |
| | GCAATTTTTTTTAAAAAATTCCCCTACCCCTGTTTAAAGCTGAAAAATACATTTGGTT | |
| | CATGTGCATTGTTTACAAAGCAAAAAGAAAAAAGAGGAAAAAAAGGCAAAAAATATTG | |
| | TGAAAGAAAAAAAACAACTTAATATATTTTGGATTAATATTTGGTATTTCTTTTAAAG | |
| | TATTTTTTGTGCTGTGAACATTTTCTGCCAAAGACCATGATGTGTGTCTGTATGTTTA | |
| | AGTTATCGTAAATATTTAAAATGTAAACATGGCTGTTTTGTTATGCCACCCTGTACCA | |
| | GGATTGCTGCCGCATTCCACTGGGTATAACAGTATTTTAATTAAAAAATAATAATTAA | |
| | AAGTG | |
| | ORF Start: ATG at 1179 | ORF Stop: TGA at 2580 |
| | SEQ ID NO:52 | 467 aa MW at 51871.4 kD |

| | |
|---|---|
| NOV21, | MEAVLRENARLQRDNERLQRELESSAEKAGRIEKLESEIQRLSEAHESLTRASSKREA |
| CG59636-01 | LEKTMRNKMDSEMRRLQDFNRDLRERLESANRRLASKTQEAQAGSQDMVAKLLAQSYE |
| Protein | QQQEQEKLEREMALLRGAIEDQRRRAELLEQALGNAQGRAARAEEELRKKQAYVEKVE |
| Sequence | RLQQALGQLQAACEKREQLELRLRTRLEQELKALRAQQRQAGAPGGSSGSGGSPELSA |
| | LRLSEQLREKEEQILALEADMTKWEQKYLEERAMRQFAMDAAATAAAQRDTTLIRHSP |
| | QPSPSSSFNEGLLTGGHRHQEMESRLKVLHAQILEKDAVIKVLQQRSRRDPGKAIQGS |
| | LRPAKSVPSVFAAAAAGTQGWQGLSSSERQTADAPARLTTADRAPTEEPVVTAPPAAH |
| | AKHGSRDGSTQTDGPPDSTSTCLPPEPDSLLGCSSSQRAASLDSVATSRVQDLSDMVE |
| | ILI |

Further analysis of the NOV21 protein yielded the following properties shown in Table 21B.

TABLE 21B
Protein Sequence Properties NOV21

| | |
|---|---|
| PSort | 0.4593 probability located in mitochondrial matrix space; |
| analysis: | 0.3000 probability located in microbody (peroxisome); |
| | 0.1552 probability located in mitochondrial inner membrane; |
| | 0.1552 probability located in mitochondrial intermembrane |
| | space |
| SignalP | No Known Signal Sequence Predicted |
| analysis: | |

A search of the NOV21 protein against the Geneseq database, a proprietary database that contains sequences published in patents and patent publication, yielded several homologous proteins shown in Table 21C.

TABLE 21C
Geneseq Results for NOV21

| | | NOV21 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB41861 | Human ORFX ORF1625 polypeptide | 1 . . . 467 | 467/467 (100%) | 0.0 |
| | sequence SEQ ID NO:3250 - Homo | 383 . . . 849 | 467/467 (100%) | |
| | sapiens, 849 aa. [WO200058473-A2, 05- | | | |
| | OCT-2000] | | | |
| AAB92866 | Human protein sequence SEQ ID | 1 . . . 393 | 391/393 (99%) | 0.0 |
| | NO:11448 - Homo sapiens, 706 aa. | 314 . . . 706 | 391/393 (99%) | |
| | [EP1074617-A2, 07-FEB-2001] | | | |
| AAM93770 | Human polypeptide, SEQ ID NO:3774 - | 128 . . . 467 | 339/340 (99%) | 0.0 |
| | Homo sapiens, 340 aa. [EP1130094-A2, 05- | 1 . . . 340 | 340/340 (99%) | |
| | SEP-2001] | | | |
| AAY54052 | An angiogenesis-associated protein which | 11 . . . 427 | 288/440 (51%) | e−110 |
| | binds plasminogen - Homo sapiens, 675 aa. | 32 . . . 466 | 293/440 (65%) | |
| | [WO9966038-A1, 23-DEC-1999] | | | |
| AAY54053 | A variant of an angiogenesis-associated | 11 . . . 427 | 226/440 (51%) | e−108 |
| | protein which binds plasminogen - Homo | 32 . . . 466 | 289/440 (65%) | |
| | sapiens, 675 aa. [WO9966038-A1, 23-DEC- | | | |
| | 1999] | | | |

In a BLAST search of public sequence databases, the NOV21 protein was found to have homology to the proteins shown in the BLASTP data in Table 21D.

TABLE 21D
Public BLASTP Results for NOV21

| | | NOV21 | | |
|---|---|---|---|---|
| Protein | | Residues/ | Identities/Similarities | |
| Accession | | Match | for the Matched | Expect |
| Number | Protein/Organism/Length | Residues | Portion | Value |
| Q9Y2J4 | KIAA0989 PROTEIN - Homo sapiens | 1 . . . 467 | 467/467 (100%) | 0.0 |
| | (Human), 859 aa (fragment). | 393 . . . 859 | 467/467 (100%) | |
| Q96F99 | UNKNOWN (PROTEIN FOR | 1 . . . 467 | 465/467 (99%) | 0.0 |
| | MGC:16955) - Homo sapiens (Human), | 1 . . . 466 | 466/467 (99%) | |
| | 466 aa. | | | |
| Q9UKB4 | LEMAN COILED-COIL PROTEIN - | 1 . . . 467 | 465/467 (99%) | 0.0 |
| | Homo sapiens (Human), 466 aa. | 1 . . . 466 | 466/467 (99%) | |
| Q9QUS0 | LEMAN COILED-COIL PROTEIN | 1 . . . 467 | 409/468 (87%) | 0.0 |
| | VARIANT 2 - Mus musculus (Mouse), | 1 . . . 463 | 420/468 (89%) | |
| | 463 aa. | | | |
| Q9HD27 | ANGIOMOTIN - Homo sapiens | 1 . . . 427 | 228/440 (51%) | e−109 |
| | (Human), 675 aa. | 32 . . . 466 | 293/440 (65%) | |

PFam analysis predicts that the NOV21 protein contains the domains shown in the Table 21E.

TABLE 21E
Domain Analysis of NOV21

| | NOV21 | Identities/Similarities | Expect |
|---|---|---|---|
| Pfam Domain | Match Region | for the Matched Region | Value |
| Adeno_E1B_55K: | 224 . . . 246 | 8/23 (35%) | 4.6 |
| domain 1 of 1 | | 19/23 (83%) | |
| GSPII_IJ: | 265 . . . 362 | 19/136 (14%) | 8.3 |
| domain 1 of 1 | | 62/136 (46%) | |

### Example A22

The NOV22 clone was analyzed, and the nucleotide and predicted polypeptide sequences are shown in Table 22A.

TABLE 22A
NOV22 Sequence Analysis

| | SEQ ID NO:53 | 2717 bp |
|---|---|---|
| NOV22 | TTCAGAAGGAGGAGAGACACCGGGCCCAGGGCACCCTCGCGGGCGGGCGGACCCAAGC | |
| CG59675-01 | AGTGAGGGCCTGCAGCCGGCCGGCCAGGGCAGCGGCAGGCGCGGCCCGGACCTACGGG | |
| DNA | AGGAAGCCCCGAGCCCTCGGCGGGCTGCGAGCGACTCCCCGGCGATGCCTCACAACTC | |
| Sequence | CATCAGATCTGGCCATGGAGGGCTGAACCAGCTGGGAGGGGCCTTTGTGAATGGCAGA | |
| | CCTCTGCCGGAAGTGGTCCGCCAGCGCATCGTAGACCTGGCCCACCAGGGTGTAAGGC | |
| | CCTGCGACATCTCTCGCCAGCTCCGCGTCAGCCATGGCTGCGTCAGCAAGATCCTTGG | |
| | CAGGAGTAGGTACTACGAGACTGGCAGCATCCGGCCTGGAGTGATAGGGGGCTCCAAG | |
| | CCCAAGGTGGCCACCCCCAAGGTGGTGGAGAAGATTGGGGACTACAAACGCCAGAACC | |
| | CTACCATGTTTGCCTGGGAGATCCGAGACCGGCTCCTGGCTGAGGGCGTCTGTGACAA | |
| | TGACACTGTGCCCAGTGTCAGCTCCATTAATAGAATCATCCGGACCAAAGTGCAGCAA | |
| | CCATTCAACCTCCCTATGGACAGCTGCGTGGCCACCAAGTCCCTGAGTCCCGGACACA | |
| | CGCTGATCCCCAGCTCAGCTGTAACTCCCCCGGAGTCACCCCAGTCGGATTCCCTGGG | |
| | CTCCACCTACTCCATCAATGGGCTCCTGGGCATCGCTCAGCCTGGCAGCGACAAGAGG | |
| | AAAATGGATGACAGTGATCAGGATAGCTGCCGACTAAGCATTGACTCACAGAGCAGCA | |
| | GCAGCGGACCCCGAAAGCACCTTCGCACGGATGCCTTCAGCCAGCACCACCTCGAGCC | |
| | GCTCGAGTGCCCATTTGAGCGGCAGCACTACCCAGAGGCCTATGCCTCCCCCAGCCAC | |
| | ACCAAAGGCGAGCAGGGCCTCTACCCGCTGCCCTTGCTCAACAGCACCCTGGACGACG | |
| | GGAAGGCCACCCTGACCCCTTCCAACACGCCACTGGGGCGCAACCTCTCGACTCACCA | |
| | GACCTACCCCGTGGTGGCAGATCCTCACTCACCCTTGGCCATAAAGCAGGAAACCCCC | |
| | GAGGTGTCCAGTTCTAGCTCCACCCCTTGCTCTTTATCTAGCTCCGCCCTTTTGGATC | |
| | TGCAGCAAGTCGGCTCCGGGGTCCCGCCCTTCAATGCCTTTCCCCATGCTGCCTCCGT | |
| | GTACGGGCAGTTCACGGGCCAGGCCCTCCTCTCAGGGCGAGAGATGGTGGGGCCCACG | |
| | CTGCCCGGATACCCACCCCACATCCCCACCAGCGGACAGGGCAGCTATGCCTCCTCTG | |
| | CCATCGCAGGCATGGTGGCAGGAAGTGAATACTCTGGCAATGCCTATGGCCACACCCC | |
| | CTACTCCTCCTACAGCGAGGCCTGGGGCTTCCCCAACTCCAGCTTGCTGAGTTCCCCA | |
| | TATTATTACAGTTCCACATCAAGGCCGAGTGCACCGCCCACCACTGCCACGGCCTTTG | |
| | ACCATCTGTAGTTGCCATGGGGACAGTGGGAGCGACTGAGCAACAGGAGGACTCAGCC | |
| | TGGGACAGGCCCCAGAGAGTCACACAAAGGAATCTTTATTATTACATGAAAAATAACC | |
| | ACAAGTCCAGCATTGCGGCACACTCCCTGTGTGGTTAATTTAATGAACCATGAAAGAC | |
| | AGGATGACCTTGGACAAGGCCAAACTGTCCTCCAAGACTCCTTAATGAGGGGCAGGAG | |
| | TCCCAGGGAAAGAGAACCATGCCATGCTGAAAAAGACAAAATTGAAGAAGAAATGTAG | |
| | CCCCAGCCGGTACCCTCCAAAGGAGAGAAGAAGCAATAGCCGAGGAACTTGGGGGGAT | |
| | GGCGAATGGTTCCTGCCCGGGCCCAAGGGTGCACAGGGCACCTCCATGGCTCCATTAT | |
| | TAACACAACTCTAGCAATTATGGACCATAAGCACTTCCCTCCAGCCCACAAGTCACAG | |
| | CCTGGTGCCGAGGCTCTGCTCACCAGCCACCCAGGGAGTCACCTCCCTCAGCCTCCCG | |
| | CCTGCCCCACACGGAGGCTCTGGCTGTCCTCTTTCCTCCACTCCATTTGCTTGGCTCT | |
| | TTCTACACCTCCCTCTTGGATGGGCTGAGGGCTGGAGCGAGTCCCTCAGAAATTCCAC | |
| | CAGGCTGTCAGCTGACCTCTTTTTCCTGCTGCTGTGAAGGTATAGCACCACCCAGGTC | |
| | CTCCTGCAGTGCGGCATCCCCTTGGCAGCTGCCGTCAGCCAGGCCAGCCCCAGGGAGC | |
| | TTAAAACAGACATTCCACAGGGCCTGGGCCCCTGGGAGGTGAGGTGTGGTGTGCGGCT | |
| | TCACCCAGGGCAGAACAAGGCAGAATCGCAGGAAACCCGCTTCCCCTTCCTGACAGCT | |
| | CCTGCCAAGCCAAATGTGCTTCCTGCAGCTCACGCCCACCAGCTACTGAAGGGACCCA | |
| | AGGCACCCCCTGAAGCCAGCGATAGAGGGTCCCTCTCTGCTCCCCAGCAGCTCCTGCC | |
| | CCCAAGGCCTGACTGTATATACTGTAAATGAAACTTTGTTTGGGTCAAGCTTCCTTCT | |
| | TTCTAACCCCCAGACTTTGGCCTCTGAGTGAAATGTCTCTCTTTGCCCTGTGGGGCTT | |
| | CTCTCCTTGATGCTTCTTTCTTTTTTTAAAGACAACCTGCCATTACCACATGACTCAA | |
| | TAAACCATTGCTCTTCAAAAAAAAAAAAAAAAAAAAAAAAAAAAAAAAA | |
| | ORF Start: ATG at 161 | ORF Stop: TAG at 1517 |
| | SEQ ID NO:54 | 452 aa MW at 48309.3 kD |

| | |
|---|---|
| NOV22, | MPHNSIRSGHGGLNQLGGAFVNGRPLPEVVRQRIVDLAHQGVRPCDISRQLRVSHGCV |
| CG59675-01 | SKILGRSRYYETGSIRPGVIGGSKPKVATPKVVEKIGDYKRQNPTMFAWEIRDRLLAE |
| Protein | GVCDNDTVPSVSSINRIIRTKVQQPFNLPMDSCVATKSLSPGHTLIPSSAVTPPESPQ |
| Sequence | SDSLGSTYSINGLLGIAQPGSDKRKMDDSDQDSCRLSIDSQSSSSGPRKHLRTDAFSQ |
| | HHLEPLECPFERQHYPEAYASPSHTKGEQGLYPLPLLNSTLDDGKATLTPSNTPLGRN |
| | LSTHQTYPVVADPHSPLAIKQETPEVSSSSSTPCSLSSSALLDLQQVGSGVPPFNAFP |
| | HAASVYGQFTGQALLSGREMVGPTLPGYPPHIPTSGQGSYASSAIAGMVAGSEYSGNA |
| | YGHTPYSSYSEAWGFPNSSLLSSPYYYSSTSRPSAPPTTATAFDHL |

Further analysis of the NOV22 protein yielded the following properties shown in Table 22B.

TABLE 22B
Protein Sequence Properties NOV22

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV22 protein against the Geneseq database, a proprietary database that contains sequences published in patents and patent publication, yielded several homologous proteins shown in Table 22C.

TABLE 22C
Geneseq Results for NOV22

| | | NOV22 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB85809 | Human PAX8 protein sequence - Homo | 1 . . . 452 | 450/452 (99%) | 0.0 |
| | sapiens, 450 aa. [WO200152789-A2, 26- | 1 . . . 450 | 450/452 (99%) | |
| | JUL-2001] | | | |
| AAB85799 | Human PAX8 protein sequence - Homo | 1 . . . 452 | 446/452 (98%) | 0.0 |
| | sapiens, 450 aa. [WO200152789-A2, 26- | 1 . . . 450 | 446/452 (98%) | |
| | JUL-2001] | | | |
| AAB85795 | Human PAX8e9-PPARgammae1 protein | 1 . . . 398 | 396/398 (99%) | 0.0 |
| | sequence - Homo sapiens, 874 aa. | 1 . . . 396 | 396/398 (99%) | |
| | [WO200152789-A2, 26-JUL-2001] | | | |
| AAB85794 | Human PAX8e8-PPARgammae1 protein | 1 . . . 383 | 367/383 (95%) | 0.0 |
| | sequence - Homo sapiens, 840 aa. | 1 . . . 381 | 370/383 (95%) | |
| | [WO200152789-A2, 26-JUL-2001] | | | |
| AAB85801 | Human PAX8e9(-exon 8)-PARgammae1 | 1 . . . 398 | 319/398 (80%) | e−176 |
| | protein sequence - Homo sapiens, 811 aa. | 1 . . . 333 | 326/398 (81%) | |
| | [WO200152789-A2, 26-JUL-2001] | | | |

In a BLAST search of public sequence databases, the NOV22 protein was found to have homology to the proteins shown in the BLASTP data in Table 22D.

TABLE 22D
Public BLASTP Results for NOV22

| | | NOV22 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q06710 | Paired box protein PAX-8, isoforms | 1 . . . 452 | 447/452 (98%) | 0.0 |
| | 8A/8B - Homo sapiens (Human), 450 | 1 . . . 450 | 447/452 (98%) | |
| | aa. | | | |
| Q96J49 | PAIRED BOX GENE 8 - Homo | 1 . . . 452 | 446/452 (98%) | 0.0 |
| | sapiens (Human), 450 aa. | 1 . . . 450 | 446/452 (98%) | |
| P47240 | Paired box protein PAX-8, isoform 8A | 1 . . . 452 | 442/461 (95%) | 0.0 |
| | - Canis familiaris (Dog), 459 aa. | 1 . . . 459 | 443/461 (95%) | |
| AAH20526 | PAIRED BOX GENE 8 - Mus | 1 . . . 452 | 438/459 (95%) | 0.0 |
| | musculus (Mouse), 457 aa. | 1 . . . 457 | 441/459 (95%) | |
| Q00288 | Paired box protein PAX-8 - Mus | 1 . . . 452 | 438/459 (95%) | 0.0 |
| | musculus (Mouse), 457 aa. | 1 . . . 457 | 441/459 (95%) | |

PFam analysis predicts that the NOV22 protein contains the domains shown in the Table 22E.

TABLE 22E
Domain Analysis of NOV22

| | | Identities/ | |
|---|---|---|---|
| | | Similarities | |
| | NOV22 | for the | |
| Pfam Domain | Match Region | Matched Region | Expect Value |
| PAX: domain 1 of 1 | 9 . . . 135 | 100/127 (79%) | 1.8e−84 |
| | | 124/127 (98%) | |

### Example A23

The NOV23 clone was analyzed, and the nucleotide and predicted polypeptide sequences are shown in Table 23A.

TABLE 23A
NOV23 Sequence Analysis

| | |
|---|---|
| SEQ ID NO:55 | 5447 bp |

| NOV23, | TGCCCGTGGACTATGACCACCTATCGGGCCATCCCCAGCGATGGTGTGGACCTGGCAG | |
|---|---|---|
| CG59719-01 | CCAGCTGTGGCGCCAGGGTGGGCGATGTCCTCCCTGGGCCACACACAGGGGACTACGC | |
| DNA | TCCCTTGGGATTCTGGGCCCAGAATGGCAGCATGTCCCAGCCTCTTGGCGAGAGCCCG | |
| Sequence | GCCACCGCCACCGCCACCGCCACCGCCACCACCCGCCCCAGCCCCACCACTCCCGCAA | |
| | TGCCCAAGATGGGCGTGCGCGCAAGGGTGGCCGACTGGCCGCCCAAGCGGGAGGCCCT | |
| | GAGAGAGCACAGCAACCCAAGCCCCTCCCAGGACACAGATGGCACAAAGGCCACCAAG | |
| | ATGGCCCATTCCATGAGGAGCATACAGAACGGACAGCCCCCCACCAGCACCCCGGCTT | |
| | CCTCAGGGTCCAAAGCCTTCCACCGACTCTCCAGGAGAAGGTCCAAAGACGTGGAGTT | |
| | CCAGGACGGGTGGCCCCGGTCCCCCGGCAGGGCCTTCCTCCCCCTTCGGCACCGCAGC | |
| | AGCAGCGAGATCACCCTCAGCGAGTGTGACGCGGAGGACGCGGGGGAGCCGCGGGGGG | |
| | CCCGGCACACGGGGGCGCTGCCCCTCTTCCGCGAGTACGGGAGCACCTCGTCCATCGA | |
| | CGTGCAGGGCATGCCCGAGCAGAGCTTCTTCGACATCCTGAACGAGTTCCGCAGCGAG | |
| | CAGCCCGACGCCCGAGGGTGCCAGGCCCTCACCGAGCTCCTCCGGGCAGATCCTGGCC | |
| | CACACCTCATGGGGGGCGGCGGCGGAGCCAAGGGGGACTCCCACAACGGGCAGCCCGC | |
| | CAAGGACAGCCTCCTGCCACTGCAGCCCACGAAGGAGAAGGAGAAGGCCCGGAAGAAA | |
| | GGAGCAGCAAACCCGAGGGGGAGGCTGGGCGTTCCCCGGGGGAGGCCGACGAGGGCCG | |
| | GAGCCCCCCGGAAGCCAGCAGGCCGTGGGTGTGTCAGAAGAGCTTCGCCCACTTCGAC | |
| | GTGCAGAGCATGCTGTTCGACCTCAACGAGGCGGCCGCCAACAGGGTGTCGGTGTCGC | |
| | AGCGGCGGAACACCACCACGGGTGCTTCGGCCGCTTCCGCCGCCTCGGCCATGGCCTC | |
| | CCTCACGGCCTCGCGGGCCCACAGCCTCGGAGGCCTGGACCCGGCCTTCACCAGCACA | |
| | GAGGACCTAAACTGCAAGGAGAACTTGGAGCAGGACCTCGGCGATGACAACAGCAACG | |
| | CGTGAGCTTCTCCCGGGCTTCCGTGGGCTCCCCGAGCAGCGGCGAGGGCCACCTGGCA | |
| | GAGCCCGCCCTGAGCGCCTACCGCACCAACGCCAGCATCTCGGTGTTGGAAGTTCCCA | |
| | AGGAGCAGCAGCGGACGCAGAGTCGGCCCCGGCAGTACAGCATCGAGCATGTGGACCT | |
| | GGGCGCCCGCTACTACCAGGATTACTTCGTGGGCAAAGAACATGCCAATTACTTCGGC | |
| | GTGGATGAGAAGCTGGGGCCAGTGGCTGTGAGCATTAAGCGGGAGAAGCTGGAAGACC | |
| | ACAAGGAGCACGGACCTCAGTACCAGTACAGGATCATCTTCCGGACCCGCGAGCTCAT | |
| | CACCCTGCGGGGCTCCATCCTGGAAGATGCTACGCCCACAGCCACCAAGCATGGGACC | |
| | CGGCGGGGCCTGCCCTTGAAGGATGCCCTGGAGTATGTCATCCCCGAGCTCAACATCC | |
| | ACTGCCTGCGGCTGGCCCTCAACACCCCCAAGGTGACGGAGCAACTGCTGAAGCTCGA | |
| | TGAGCAAGGGGTGAGTCAGGGGAAGCACAAGGTGGGCATCCTCTATTGCAAGGCCGGC | |
| | CAGAGCTCCGAGGAGGAGATGTACAACAATGAGGAGGCCGGCCCCGCCTTTGAGGAGT | |
| | TCCTCTCCCTCATCGGCGAGAAGGTCTGCCTGAAGGGCTTCACCAAGTACGCTGCCCA | |
| | GCTGGACGTCAAGACCGACTCCACGGGAACCCACTCCCTCTACACGATGTACCAGGAC | |
| | TACGAGATCATGTTCCATGTCTCCACCCTGCTCCCTTACACCCCCAACAACAGGCAGC | |
| | AGCTGCTACGGAAGAGGCACATAGGAAATGACATCGTGACGATCATCTTCCAGGAGCC | |
| | TGGCGCGCTACCGTTCACCCCCAAGAACATCCGCTCCCACTTCCAGCACGTCTTCATC | |
| | ATTGTCCGAGTCCACAACCCCTGCACTGATAACGTCTGTTACAGTATGGCTGTGACCC | |
| | GATCCAAAGACGCTCCTCCTTTCGGCCCCCCCATCCCCAGTGGAACCACATTCCGCAA | |
| | ATCCGACGTCTTCAGAGACTTCTTGCTGGCCAAGGTGATTAACGCTGAGAACGCCGCG | |
| | CACAAGTCCGACAAGTTCCACACCATGGCCACCAGGACCCGCCAGGAGTATCTCAAGG | |
| | ACCTGGCCGAAAACTGTGTCTCCAACACCCCCATCGACTCCACCGGCAAATTCAACCT | |
| | CATCTCCCTGACCTCCAAGAAGAAGGAAAAGACAAAAGCACGGGCTGGCGCTGAGCAG | |
| | CACAGTGCAGGGGCCATCGCCTGGAGGGTGGTGGCCCAGGACTACGCCCAGGGGGTGG | |
| | CAAGGAGGTGGTGTTCAACTGCTACTGCGGGGATGTCATTGGCTGGACTCCAGACTCC | |
| | TCCACACTCAAAATCTTCTATGGACGAGGAGACCACATCTTCCTACAGGCGACAGAGG | |
| | GTTCTGTGGAGGACATAAGGGAGATAGTGCAGAGACTGAAGGTGATGACCAGTGGCTG | |
| | GGAGACGGTGGACATGACGCTTCGGCGGAACGGGCTCGGGCAGCTGGGCTTCCACGTG | |
| | AAGTACGACGGCACGGTGGCCGAGGTTGAGGACTATGGGTTCGCCTGGCAGGCCGGCC | |
| | TCCGGCAGGGCAGCCGACTAGTGGAGATCTGCAAGGTGGCCGTGGTCACACTGACCCA | |
| | CGACCAGATGATCGACCTGCTGCGCACCTCTGTCACTGTGAAGGTGGTCATCATCCCG | |
| | CCTTTTGAGGACGGCACTCCCCGGAGGGGTTGGCCGGAGACCTACGACATGAATACCT | |
| | CGGAGCCCAAGACGGAGCAGGAAAGCATCACTCCTGGGGGCCGGCCCCCCTACCGCAG | |
| | CAATGCTCCCTGGCAGTGGAGCGGGCCCGCATCCCATAACTCTCTACCAGCCTCCAAG | |
| | TGGGCCACTCCAACCACTCCCGGCCATGCCCAGTCCCTGAGCCGGCCCCTGAAGCAGA | |
| | CCCCCATAGTCCCCTTCCGGGAGTCCCAGCCACTGCACAGCAAGAGGCCTGTCAGCTT | |
| | CCCAGAAACCCCTTACACAGTATCACCAGCAGGGGCCGACAGAGTCCCTCCCTACCGA | |
| | CAGCCTTCTGGGAGCTTCTCCACCCCCGGTTCGGCCACCTACGTGAGATACAAGCCAT | |
| | CCCCAGAAAGGTACACGGCTGCCCCACACCCCCTGCTATCTCTTGATCCCCACTTCAG | |
| | CCACGATGGGACGTCCAGCGGCGACTCCTCTTCCGGCGGCCTGACCAGCCAGGAGAGC | |
| | ACCATGGAACGCCAGAAGCCAGAGCCTTTGTGGCATGTGCCTGCCCAGGCCAGGCTCT | |
| | CAGCCATAGCCGGAAGCAGCGGGAACAAGCACCCGTCCAGGCAGGATGCAGCAGGCAA | |
| | AGATTCTCCCAACAGGCATTCCAAAGGAGAACCTCAATACTCAAGTCATTCCAGCAGC | |
| | AACACCCTCTCCAGCAACGCATCCAGCAGCCACAGCGACGACCGCTGGTTCGACCCCC | |
| | TGGACCCCCTGGAGCCAGAGCAAGACCCCCTCTCCAAGGGTGGCTCTAGTGACAGCGG | |
| | CATCGACACCACCCTCTACACCTCCAGCCCTAGCTGCATGTCCCTGGCCAAGGCTCCA | |
| | CGGCCCGCCAAGCCACACAAGCCCCCTGGAAGTATGGGCCTTTGTGGCGGGGGTCGCG | |
| | AGGCCGCTGGGAGGTCCCACCACGCAGACAGGCGGCGGGAGGTCTCCCCTGCCCCCGC | |
| | AGTTGCCGGCCAAAGCAAGGGCTACCGACCGAAGCTGTACTCCTCCGGCTCCAGCACC | |
| | CCCACGGGACTGGCGGGGGGCAGCCGAGACCCACCGAGGCAGCCCAGTGACATGGGCT | |
| | CGAGGGTTGGCTACCCCGCTCAGGTTTACAAAACTGCCAGTGCAGAGACTCCTCGGCC | |
| | CTCCCAGCTGGCCCAGCCCAGCCCCTTTCAGCTCTCCGCCTCCGTCCCCAAGTCCTTC | |
| | TTCTCCAAGCAGCCTGTACGCAATAAGCACCCAACAGGGTGGAAGAGAACGGAGGAGC | |
| | CCCCACCACGGCCACTCCCCTTCAGTGACCCAAAGAAGCAGGTGGACACGAACACCAA | |
| | AAATGTCTTTGGGCAACCGAGGTTGAGGGCATCCCTCCGAGACCTCCCGTCACCACGG | |
| | AAGAACTACAAATCCACCATCGAGGATGACCTGAAGAAACTCATCATCATGGACAACC | |
| | TGGGGCCAGAGCAGGAGAGAGACACGGGACAGTCACCGCAGAAGGGCCTGCAGCGGAC | |
| | GCTGTCGGACGAGAGCCTGTGCAGCGGGCGCCGGGAGCCCAGCTTCGCCAGCCCCGCT | |
| | GGCCTAGAGCCAGGGCTGCCCAGCGACGTGCTCTTCACCAGCACCTGCGCCTTCCCGT | |
| | CCAGCACGCTGCCTGCACGCCGCCAGCACCAGCACCCCCACCCGCCCGTCGGCCCCGG | |
| | TGCCACCCCTGCCGCCGGCAGCGGCTTTCCCGAGAAGAAATCCACCATCTCAGCCTCG | |
| | GAGCTCTCGCTGGCTGATGGGCGGGACCGCCCCCTGCGGCGCCTGGACCCTGGGCTGA | |
| | TGCCCCTGCCTGACACAGCTGCTGGCCTCGAGTGGTCCAGCCTGGTGAACGCAGCCAA | |
| | GGCATACGAAGTGCAAAGAGCCGTCTCACTCTTCTCTCTGAACGACCCGGCCCTGAGC | |
| | CCGGACATCCCGCCTGCACACAGTCCTGTCCACAGCCACCTGAGCCTGGAGAGGGGAC | |
| | CCCCGACCCCCAGGACCACCCCTACCATGAGCGAGGAGCCACCCCTGGATCTGACAGG | |
| | CAAGGTGTACCAGCTGGAGGTGATGCTGAAACAGCTGCACACTGACCTGCAGAAGGAG | |
| | AAGCAGGACAAGGTGGTGCTCCAGTCAGAGGTGGCCAGCCTGCGGCAGAACAACCAGC | |
| | GGCTGCAGGAGGAGTCGCAGGCCGCCAGCGAGCAGCTGCGCAAGTTTGCGGAGATCTT | |
| | CTGCAGGGAGAAGAAGGAGCTCTGAGGTGGGAGGCCGCCGCCCGCCTTCGCTCCTTCC | |
| | CCTCAGGCCGTGGCCCTGCTGCCTCTCTCCCTCCACTCAGCTCCCAGCTGCCG | |
| | ORF Start: ATG at 13 | ORF Stop: TGA at 5359 |
| | SEQ ID NO:56 | 1782 aa MW at 194606.5 kD |

| | |
|---|---|
| NOV23, | MTTYRAIPSDGVDLAASCGARVGDVLPGPHTGDYAPLGFWAQNGSMSQPLGESPATAT |
| CG59719-01 | ATATATTRPSPTTPAMPKMGVRARVADWPPKREALREHSNPSPSQDTDGTKATKMAHS |
| Protein | MRSIQNGQPPTSTPASSGSKAFHRLSRRRSKDVEFQDGWPRSPGRAFLPLRHRSSSEI |
| Sequence | TLSECDAEDAGEPRGARHTGALPLFREYGSTSSIDVQGMPEQSFFDILNEFRSEQPDA |
| | RGCQALTELLRADPGPHLMGGGGGAKGDSHNGQPAKDSLLPLQPTKSKEKARKKPARG |
| | LGGGDTVDSSIFRKLRSSKPEGEAGRSPGEADEGRSPPEASRPWVCQKSFAHFDVQSM |
| | LFDLNEAAANRVSVSQRRNTTTGASAASAASAMASLTASRAHSLGGLDPAFTSTEDLN |
| | CKENLEQDLGDDNSNDLLLSCPHFRNEIGGECERNVSFSRASVGSPSSGEGHLAEPAL |
| | SAYRTNASISVLEVPKEQQRTQSRPRQYSIEHVDLGARYYQDYFVGKEHANYFGVDEK |
| | LGPVAVSIKREKLEDHKEHGPQYQYRIIFRTRELITLRGSILEDATPTATKHGTGRGL |
| | PLKDALEYVIPELNIHCLRLALNTPKVTEQLLKLDEQGVSQGKHKVGILYCKAGQSSE |
| | EEMYNNEEAGPAFEEFLSLIGEKVCLKGFTKYAAQLDVKTDSTGTHSLYTMYQDYEIM |
| | FHVSTLLPYTPNNRQQLLRKRHIGNDIVTIIFQEPGALPFTPKNIRSHFQHVFIIVRV |
| | HNPCTDNVCYSMAVTRSKDAPPFGPPIPSGTTFRKSDVFRDFLLAKVINAENAAHKSD |
| | KFHTMATRTRQEYLKDLAENCVSNTPIDSTGKFNLISLTSKKKEKTKARAGAEQHSAG |
| | AIAWRVVAQDYAQGVEIDCILGISNEFVVLLDLRTKEVVFNCYCGDVIGWTPDSSTLK |
| | IFYGRGDHIFLQATEGSVEDIREIVQRLKVMTSGWETVDMTLRRNGLGQLGFHVKYDG |
| | TVAEVEDYGFAWQAGLRQGSRLVEICKVAVVTLTHDQMIDLLRTSVTVKVVIIPPFED |
| | GTPRRGWPETYDMNTSEPKTEQESITPGGRPPYRSNAPWQWSGPASHNSLPASKWATP |
| | TTPGHAQSLSRPLKQTPIVPFRESQPLHSKRPVSFPETPYTVSPAGADRVPPYRQPSG |
| | SFSTPGSATYVRYKPSPERYTAAPHPLLSLDPHFSHDGTSSGDSSSGGLTSQESTMER |
| | QKPEPLWHVPAQARLSAIAGSSGNKHPSRQDAAGKDSPNRHSKGEPQYSSHSSSNTLS |
| | SNASSSHSDDRWFDPLDPLEPEQDPLSKGGSSDSGIDTTLYTSSPSCMSLAKAPRPAK |
| | PHKPPGSMGLCGGGREAAGRSHHADRRREVSPAPAVAGQSKGYRPKLYSSGSSTPTGL |
| | AGGSRDPPRQPSDMGSRVGYPAQVYKTASAETPRPSQLAQPSPFQLSASVPKSFFSKQ |
| | PVRNKHPTGWKRTEEPPPRPLPFSDPKKQVDTNTKNVFGQPRLRASLRDLRSPRKNYK |
| | STIEDDLKKLIIMDNLGPEQERDTGQSPQKGLQRTLSDESLCSGRREPSFASPAGLEP |
| | GLPSDVLFTSTCAFPSSTLPARRQHQHPHPPVGPGATPAAGSGFPEKKSTISASELSL |
| | ADGRDRPLRRLDPGLMPLPDTAAGLEWSSLVNAAKAYEVQRAVSLFSLNDPALSPDIP |
| | PANSPVHSHLSLERGPPTPRTTPTMSEEPPLDLTGKVYQLEVMLKQLHTDLQKEKQDK |
| | VVLQSEVASLRQNNQRLQEESQAASEQLRKFAEIFCREKKEL |

Further analysis of the NOV23 protein yielded the following properties shown in Table 23B.

TABLE 23B
Protein Sequence Properties NOV23

| | |
|---|---|
| PSort | 0.6000 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV23 protein against the Geneseq database, a proprietary database that contains sequences published in patents and patent publication, yielded several homologous proteins shown in Table 23C.

TABLE 23C
Geneseq Results for NOV23

| | | NOV23 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAM89182 | Human immune/haematopoietic antigen | 153 . . . 536 | 382/384 (99%) | 0.0 |
| | SEQ ID NO:16775 - Homo sapiens, 384 aa. | 1 . . . 384 | 382/384 (99%) | |
| | [WO200157182-A2, 09-AUG-2001] | | | |
| AAW19765 | Human interleukin-1 receptor interacting | 318 . . . 1136 | 374/859 (43%) | e−175 |
| | protein - Homo sapiens, 1042 aa. | 99 . . . 884 | 490/859 (56%) | |
| | [WO9725347-A1, 17-JUL-1997] | | | |
| AAW93489 | Human SPA-1 protein - Homo sapiens, | 318 . . . 1136 | 374/859 (43%) | e−175 |
| | 1042 aa. [WO9910380-A1, 04-MAR-1999] | 99 . . . 884 | 487/859 (56%) | |
| AAW19766 | Mouse interleukin-1 receptor interacting | 318 . . . 1062 | 347/757 (45%) | e−171 |
| | protein - Mus musculus, 1038 aa. | 97 . . . 778 | 450/757 (58%) | |
| | [WO9725347-A1, 17-JUL-1997] | | | |
| AAW15140 | Mammalian cell cycle regulatory protein | 641 . . . 1062 | 216/434 (49%) | e−116 |
| | SPA-1 - Mus sp, 693 aa. [JP08217797-A, | 1 . . . 433 | 288/434 (65%) | |
| | 27-AUG-1996] | | | |

In a BLAST search of public sequence databases, the NOV23 protein was found to have homology to the proteins shown in the BLASTP data in Table 23D.

TABLE 23D
Public BLASTP Results for NOV23

| | | NOV23 | | |
|---|---|---|---|---|
| Protein | | Residues/ | Identities/ | |
| Accession | | Match | Similarities for the | Expect |
| Number | Protein/Organism/Length | Residues | Matched Portion | Value |
| O60292 | KIAA0545 PROTEIN - Homo sapiens | 654 . . . 1782 | 1129/1129 (100%) | 0.0 |
| | (Human), 1129 aa (fragment). | 1 . . . 1129 | 1129/1129 (100%) | |
| O95321 | HIGH-RISK HUMAN PAPILLOMA | 14 . . . 1773 | 876/1823 (48%) | 0.0 |
| | VIRUSES E6 ONCOPROTEINS | 13 . . . 1774 | 1131/1823 (61%) | |
| | TARGETED PROTEIN E6TP1 ALPHA - | | | |
| | Homo sapiens (Human), 1783 aa. | | | |
| Q9UNU4 | HIGH-RISK HUMAN PAPILLOMA | 14 . . . 1773 | 876/1842 (47%) | 0.0 |
| | VIRUSES E6 ONCOPROTEINS | 13 . . . 1795 | 1132/1842 (60%) | |
| | TARGETED PROTEIN E6TP1 BETA - | | | |
| | Homo sapiens (Human), 1804 aa. | | | |
| O35412 | SPA-1 LIKE PROTEIN P1294 - Rattus | 3 . . . 1773 | 874/1881 (46%) | 0.0 |
| | norvegicus (Rat), 1822 aa. | 9 . . . 1813 | 1120/1881 (59%) | |
| Q9P2F8 | KIAA1389 PROTEIN - Homo sapiens | 284 . . . 1775 | 751/1545 (48%) | 0.0 |
| | (Human), 1514 aa (fragment). | 66 . . . 1506 | 947/1545 (60%) | |

PFam analysis predicts that the NOV23 protein contains the domains shown in the Table 23E.

TABLE 23E
Domain Analysis of NOV23

| | | Identities/Similarities | |
|---|---|---|---|
| Pfam Domain | NOV23 Match Region | for the Matched Region | Expect Value |
| PI3K_rbd: domain 1 of 1 | 733 . . . 774 | 6/47 (13%) | 4.1 |
| | | 30/47 (64%) | |
| Rap_GAP: domain 1 of 1 | 641 . . . 829 | 116/192 (60%) | 6.2e−121 |
| | | 177/192 (92%) | |
| PDZ: domain 1 of 1 | 967 . . . 1041 | 19/83 (23%) | 0.00048 |
| | | 55/83 (66%) | |

### Example A24

The NOV24 clone was analyzed, and the nucleotide and predicted polypeptide sequences are shown in Table 24A.

TABLE 24A
NOV24 Sequence Analysis

| | SEQ ID NO:57 | 4894 bp |
|---|---|---|
| NOV24, | TTCCTCAACATCAACGAGACCTTCAAGTTAATGGAGCAGCTTGCCAACATAGCCATGA | |
| CG59777-01 | GGCAACTCTTAGACAATGAGGGATTTGAACAAGATCGAGATCTTGATGCCAGGGCAAA | |
| DNA | GAGTGAGAGATACCGTGCACTTTTCCGGCTGCCCAAAGATGAAAAATTAGATGGCCAC | |
| Sequence | ACAGACTGCACTCTCTGGACTCCATTTAACAAAATGCACATTTTGGGGCAGATGTTTG | |
| | TGTCCACAAATTACATCTGTTTTACCAGCAAGGAGGAGAACTTATGTAGCCTCATTAT | |
| | CCCGCTCCGTGAGGTAACAATTGTGGAAAAGGCAGACAGCTCCAGTGTGCTCCCCAGT | |
| | CCCTTATCCATCAGCACCCGAAACAGGATGACCTTCCTATTTGCCAACTTGAAAGATA | |
| | GAGACTTTCTAGTGCAGAGGATCTCAGATTTCCTGCAACAGACTACTTCCAAAATATA | |
| | TTCTGACAAGGAGTTTGCAGGAAGTTACAACAGTTCAGATGATGAGGTGTACTCTCGA | |
| | CCCAGCAGCCTCGTCTCCTCCAGCCCCCAGAGAAGCACGAGCTCTGATGCTGATGGAG | |
| | AGCGCCAGTTTAACCTAAATGGCAACAGCGTCCCCACAGCCACACAGACCCTGATGAC | |
| | CATGTATCGGCGGCGGTCTCCCGAGGAGTTCAACCCGAAATTGGCCAAAGAGTTTCTG | |
| | AAAGAGCAAGCCTGGAAGATTCACTTTGCTGAGTATGGGCAAGGGATCTGCATGTACC | |
| | GCACAGAGAAAACGCGGGAGCTGGTGTTGAAGGGCATCCCGGAGAGCATGCGTGGGGA | |
| | GCTCTGGCTGCTGCTGTCAGGTGCCATCAATGAGAAGGCCACACATCCTGGGTACTAT | |
| | GAAGACCTAGTGGAGAAGTCCATGGGGAAGTATAATCTCGCCACGGAGGAGATTGAGA | |
| | GGGATTTACACCGCTCCCTTCCAGAACACCCAGCTTTTCAGAATGAAATGGGCATTGC | |
| | TGCACTAAGGAGAGTCTTAACAGCTTATGCTTTTCGAAATCCCAACATAGGGTATTGC | |
| | CAGGCCATGAATATTGTCACTTCAGTGCTGCTGCTTTATGCCAAAGAGGAGGAAGCTT | |
| | TCTGGCTGCTTGTGGCTTTGTGTGAGCGCATGCTCCCAGATTACTACAACACCAGAGT | |
| | TGTGGGTGCACTGGTGGACCAAGGTGTCTTTGAGGAGCTAGCACTAGACTACGTCCCA | |
| | CAGCTGTACGACTGCATGCAAGACCTGGGCGTGATTTCCACCATCTCCCTGTCTTGGT | |
| | TCCTCACACTATTTCTCAGTGTGATGCCTTTTGAGAGTGCAGTTGTGGTTGTTGACTG | |
| | TTTCTTCTATGAAGGAATTAAAGTGATATTCCAGTTGGCCCTAGCTGTGCTGGATGCA | |
| | AATGTGGACAAACTGTTGAACTGCAAGGATGATGGGGAGGCCATGACCGTTTTGGGAA | |
| | GGTATTTAGACAGTGTGACCAATAAAGACAGCACACTGCCTCCCATTCCTCACCTCCA | |
| | CTCCTTGCTCAGCGATGATGTGGAACCTTACCCTGAGGTAGACATCTTTAGACTCATC | |
| | AGAACTTCCTACGAGAAATTCGGAACTATCCGGGCAGATTTGATTGAACAGATGAGAT | |
| | TCAAACAGAGACTGAAAGTGATCCAGACGCTGGAGGATACTACGAAACGCAACGTGGT | |
| | ACGAACCATTGTGACAGAAACTTCCTTTACCATTGATGAGCTGGAAGAACTTTATGCT | |
| | CTTTTCAAGGCAGAACATCTCACCAGCTGCTACTGGGGCGGGAGCAGCAACGCGCTGG | |
| | ACCGGCATGACCCCAGCCTGCCCTACCTGGAACAGTATCGCATTGACTTCGAGCAGTT | |
| | CAAGGGAATGTTTGCTCTTCTCTTTCCTTGGGCATGTGGAACTCACTCTGACGTTCTG | |
| | GCCTCCCGCTTGTTCCAGTTATTAGATGAAAATGGAGACTCTTTGATTAACTTCCGGG | |
| | AGTTTGTCTCTGGGCTAAGTGCTGCATGCCATGGGGACCTCACAGAGAAGCTCAAACT | |
| | CCTGTACAAAATGCACGTCTTGCCTGAGCCATCCTCTGATCAAGATGAACCAGATTCT | |
| | GCTTTTGAAGCAACTCAGTACTTCTTTGAAGATATTACCCCAGAATGTACACATGTTG | |
| | TTGGATTGGATAGCAGAAGCAAACAGGGTGCAGATGATGGCTTTGTTACGGTGAGCCT | |
| | AAAGCCAGACAAAGGGAAGAGAGCAAATTCCCAAGAAAATCGTAATTATTTGAGACTG | |
| | TGGACTCCAGAAAATAAATCTAAGTCAAAGAATGCAAAGGATTTACCCAAATTAAATC | |
| | AGGGGCAGTTCATTGAACTGTGTAAGACAATGTATAACATGTTCAGCGAAGACCCCAA | |
| | TGAGCAGGAGCTGTACCATGCCACGGCAGCAGTGACCAGCCTCCTGCTGGAGATTGGG | |
| | GAGGTCGGCAAGTTGTTCGTGGCCCAGCCTGCAAAGGAGGGCGGGAGCGGAGGCAGTG | |
| | GGCCGTCCTGCCACCAGGGCATCCCAGGCGTGCTCTTCCCCAAGAAAGGGCCAGGCCA | |
| | GCCTTACGTGGTGGAGTCTGTTGAGCCCCTGCCGGCCAGCCTGGCCCCCGACACCGAG | |
| | GAACACTCCCTTGGAGGACAAATGGAGGACATCAAGCTGGAGGACTCCTCGCCCCGGG | |
| | ACAACGGGGCCTGCTCCTCCATGCTGATCTCTGACGACGACACCAAGGACGACAGCTC | |
| | CATGTCCTCATACTCGGTGCTGAGTGCCGGCTCCCACGAGGAGGACAAGCTGCACTGC | |
| | GAGGACATCGGAGAGGACACGGTCCTGGTGCGGAGCGGCCAGGGCACGGCGGCACTGC | |
| | CCCGGAGCACCAGCCTGGACCGGGACTGGGCCATCACCTTCGAGCAGTTCCTGGCCTC | |
| | CCTCTTAACTGAGCCTGCCCTGGTCAAGTACTTTGACAAGCCCGTGTGCATGATGGCC | |
| | AGGATTACCAGTGCAAAAAACATCCGGATGATGGGCAAGCCCCTCACCTCGGCCAGTG | |
| | ACTATGAAATCTCGGCCATGTCCGGCTGACACGGGCGCCTTCCCGGGGGAGTGGGAGG | |
| | AGAGGGAGGGGAGGGATTTTTTATGTTCTTCTGTGTTGAGTTTTTTCTTTCTTTCTTT | |
| | TAAATTAAATATTTATTAGTACCTGGCTTGAAGCCTAGTGTTTTCATAATGTAATTCA | |
| | ATGAAAACTGTTGGAGAAATATTTAAACACCTCAATGTAGGTACATTACACTCTTGTT | |
| | GCGGGGAGGGGATTTACCAGAATACAGTTTATTTCGTGAATTCTAAAAAACAAAAAGA | |
| | TGAATCTGTCAGTGATATGTGTGTATTATAACTTATTAATCTTGCTGTTGAGCTGTAT | |
| | ACATGGTTTAAAAAATAGTACTGTTTAATGCTAAGTAAGGCAGCAGTCATTTGTGTAT | |
| | TCAGGCTTTTTAAATAAAATTAGAGCTGTAAGGAAAATGAAAAGCCACAAATGCAAGA | |
| | CTGTTCTTAAATGGAAGGCATAGTCAGCGAGGGTAAATCCTATACCACTTTAGGAAGT | |
| | ATTAAAAATATTTTTAAGATTTGAAATATATTTCATAGAAGTCCTCTATTCAAAATCA | |
| | TATTCCACAGATGTTCCCCTTCAAAGGGAAAACATTTGGGGTTCTAAACAGTTATGAA | |
| | AGTAAGTGATTTTTACATGATTCCAGAATAACACTTGTATTGACCAATTTAGACAGAT | |
| | ACCAGACCAATTTTGCATTTAAGAAATTGTTCTGATTATTTACGTCAACTCATTAGAA | |
| | TTCAGTGAAAAGTAACAGTCTTTTGTCACAGAGAATCTGAAAGTAGCAGCAAAGACAG | |
| | AGGGCTCATGACAGGTTTTTGCTTTTGCTTTGCTTTTGTTTTTGAAAGAGTAAAAGTA | |
| | CTGATGCTTCTGATACTGGATGTTTAGCTTCTTACTGCAAAAACATAAGTAAAACAGT | |
| | CAACTTTACCATTTCCGTATTCTCCATAGATTGAAGAAATTTATACCACATATCGCAT | |
| | ATGCTCTATTTCCTGAATGGATGTGGAAATGAAAGCTAGCGCACCTGCACTTTGAATT | |
| | CTTGCTTCTTTTTTATTACTGTTATGATTTTGCTTTTTACAGATGTTGGACGATTTTT | |
| | TCTTCTGATTGTTGAATTCATAATCATGGTCTCATTTCCTTTGCTTCTTTGGAATATT | |
| | TCTTTCAACACATTCCTTTATTTTATTATACATTGTGTCCTTTTTTTAGCTATTGCTG | |
| | CTGTTGTTTTTTATTCTATTTACAGGATGATTTTTAAACTGTCAAATGAAGTAGTGTT | |
| | AACCTCAAATAGGCTAAATGTGAACAAATAAAATACAGCAAATACTCAGATACAGCTT | |
| | TTTATCTTTGTGCTTGAGTTCCTGCCTAAGGCAATAACATTATTCTTTTGACAACTTT | |
| | TGCAGGGGAAATTATATCAGGCAACCATTTTGATTAAGTAAATAAATTTTATAGGCAA | |
| | ACATATAGAGAGATATACAATTTGTAGTATATCAATGACTATATTTATAATAAGGAAT | |
| | ATAATTGTTATCAGTTATCTAACTTAAAATGCTTATCCATAATGATCAGTGATATTCA | |
| | GCTTTTTAAAATATGCTTGTTGGTTGCATGTCTGTCTTCATATCCACATTGAGGATTC | |
| | CATCTCACACCTAGTTCATTAAATAGGGCATATTAGTTTCAGATGTTTGTCGTGGTTT | |
| | GTTTAGGTTTACTACACATATTTTCCGTTTGTGGGGAGTTGTTCCTTTGTTGCATCCA | |
| | TTGTTATTAAGGCTATGTGGGC | |
| | ORF Start: ATG at 31 | ORF Stop: TGA at 3043 |
| | SEQ ID NO:58 | 1004 aa MW at 113034.5 kD |

| | |
|---|---|
| N0V24, | MEQLANIAMRQLLDNEGFEQDRDLDARAKSERYRALFRLPKDEKLDGHTDCTLWTPFN |
| CG59777-01 | KMHILGQMFVSTNYICFTSKEENLCSLIIPLREVTIVEKADSSSVLPSPLSISTRNRM |
| Protein | TFLFANLKDRDFLVQRISDFLQQTTSKIYSDKEFAGSYNSSDDEVYSRPSSLVSSSPQ |
| Sequence | RSTSSDADGERQFNLNGNSVPTATQTLMTMYRRRSPEEFNPKLAKEFLKEQAWKIHFA |
| | EYGQGICMYRTEKTRELVLKGIPESMRGELWLLLSGAINEKATHPGYYEDLVEKSMGK |
| | YNLATEEIERDLHRSLPEHPAFQNEMGIAALRRVLTAYAFRNPNIGYCQAMNIVTSVL |
| | LLYAKEEEAFWLLVALCERNLPDYYNTRVVGALVDQGVFEELALDYVPQLYDCMQDLG |
| | VISTISLSWFLTLFLSVMPFESAVVVVDCFFYEGIKVIFQLALAVLDANVDKLLNCKD |
| | DGEAMTVLGRYLDSVTNKDSTLPPIPHLHSLLSDDVEPYPEVDIFRLIRTSYEKFGTI |
| | RADLIEQMRFKQRLKVIQTLEDTTKRNVVRTIVTETSFTIDELEELYALFKAEHLTSC |
| | YWGGSSNALDRHDPSLPYLEQYRIDFEQFKGMFALLFPWACGTHSDVLASRLFQLLDE |
| | NGDSLINFREFVSGLSAACHGDLTEKLKLLYKMHVLPEPSSDQDEPDSAFEATQYFFE |
| | DITPECTHVVGLDSRSKQGADDGFVTVSLKPDKGKRANSQENRNYLRLWTPENKSKSK |
| | NAKDLPKLNQGQFIELCKTMYNMFSEDPNEQELYHATAAVTSLLLEIGEVGKLFVAQP |
| | AKEGGSGGSGPSCHQGIPGVLFPKKGPGQPYVVESVEPLPASLAPDSEEHSLGGQMED |
| | IKLEDSSPRDNGACSSMLTSDDDTKDDSSMSSYSVLSAGSHEEDKLHCEDIGEDTVLV |
| | RSGQGTAALPRSTSLDRDWAITFEQFLASLLTEPALVKYFDKPVCMMARITSAKNIRM |
| | MGKPLTSASDYEISAMSG |

Further analysis of the NOV24 protein yielded the following properties shown in Table 24B.

TABLE 24B
Protein Sequence Properties NOV24

| | |
|---|---|
| PSort | 0.8500 probability located in endoplasmic reticulum |
| analysis: | (membrane); 0.4400 probability located in plasma membrane; |
| | 0.3000 probability located in microbody (peroxisome); 0.1000 |
| | probability located in mitochondrial inner membrane |
| SignalP | No Known Signal Sequence Predicted |
| analysis: | |

A search of the NOV24 protein against the Geneseq database, a proprietary database that contains sequences published in patents and patent publication, yielded several homologous proteins shown in Table 24C.

TABLE 24C
Geneseq Results for NOV24

| | | NOV24 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAM43565 | Human polypeptide SEQ ID NO 243 - | 218 . . . 1004 | 473/793 (59%) | 0.0 |
| | Homo sapiens, 814 aa. [WO200155308- | 54 . . . 814 | 572/793 (71%) | |
| | A2, 02-AUG-2001] | | | |
| AAM93792 | Human polypeptide, SEQ ID NO:3819 - | 402 . . . 1004 | 320/609 (52%) | e−167 |
| | Homo sapiens, 577 aa. [EP1130094-A2, | 1 . . . 577 | 406/609 (66%) | |
| | 05-SEP-2001] | | | |
| AAB92706 | Human protein sequence SEQ ID | 530 . . . 1004 | 226/481 (46%) | e−110 |
| | NO:11111 - Homo sapiens, 449 aa. | 1 . . . 449 | 295/481 (60%) | |
| | [EP1074617-A2, 07-FEB-2001] | | | |
| AAU19928 | Novel human calcium-binding protein #37 | 487 . . . 745 | 137/263 (52%) | 5e−69 |
| | - Homo sapiens, 262 aa. [WO200155304- | 9 . . . 256 | 177/263 (67%) | |
| | A2, 02-AUG-2001] | | | |
| AAM43640 | Human polypeptide SEQ ID NO 318 - | 487 . . . 745 | 137/263 (52%) | 5e−69 |
| | Homo sapiens, 262 aa. [WO200155308- | 9 . . . 256 | 177/263 (67%) | |
| | A2, 02-AUG-2001] | | | |

In a BLAST search of public sequence databases, the NOV24 protein was found to have homology to the proteins shown in the BLASTP data in Table 24D.

TABLE 24D
Public BLASTP Results for NOV24

| | | NOV24 | | |
|---|---|---|---|---|
| Protein | | Residues/ | Identities/ | |
| Accession | | Match | Similarities for the | Expect |
| Number | Protein/Organism/Length | Residues | Matched Portion | Value |
| O94958 | KIAA0882 PROTEIN - Homo sapiens | 81 . . . 1004 | 923/924 (99%) | 0.0 |
| | (Human), 924 aa (fragment). | 1 . . . 924 | 923/924 (99%) | |
| O75163 | KIAA0676 PROTEIN - Homo sapiens | 1 . . . 1004 | 612/1037 (59%) | 0.0 |
| | (Human), 1262 aa (fragment). | 253 . . . 1262 | 737/1037 (71%) | |
| Q9CUB3 | 4933431N12RIK PROTEIN - Mus | 559 . . . 1004 | 411/446 (92%) | 0.0 |
| | musculus (Mouse), 444 aa (fragment). | 1 . . . 444 | 419/446 (93%) | |
| O95759 | VASCULAR RAB-GAP/TBC- | 1 . . . 827 | 414/848 (48%) | 0.0 |
| | CONTAINING PROTEIN - Homo | 1 . . . 799 | 554/848 (64%) | |
| | sapiens (Human), 897 aa. | | | |
| Q9Z1A9 | BUB2-LIKE PROTEIN 1 (VASCULAR | 1 . . . 827 | 410/843 (48%) | 0.0 |
| | RAB-GAP/TBC-CONTAINING) - Mus | 1 . . . 798 | 551/843 (64%) | |
| | musculus (Mouse), 891 aa. | | | |

PFam analysis predicts that the NOV24 protein contains the domains shown in the Table 24E.

TABLE 24E
Domain Analysis of NOV24

| | | Identities/Similarities | |
|---|---|---|---|
| | NOV24 | for the | Expect |
| Pfam Domain | Match Region | Matched Region | Value |
| GRAM: domain 1 of 1 | 31 . . . 99 | 25/86 (29%) | 1.6e−24 |
| | | 61/86 (71%) | |
| TBC: domain 1 of 1 | 250 . . . 462 | 79/341 (23%) | 2.5e−54 |
| | | 166/341 (49%) | |
| efhand: domain 1 of 1 | 628 . . . 656 | 9/29 (31%) | 0.037 |
| | | 22/29 (76%) | |

### Example A25

The NOV25 clone was analyzed, and the nucleotide and predicted polypeptide sequences are shown in Table 25A.

TABLE 25A
NOV25 Sequence Analysis

| | SEQ ID NO:59 | 1916 bp |
|---|---|---|
| N0V25, | AAGCAAGTCCTTGGATTTACTGGGTTTTACTTGGCTTTAATGGGAGGAGACAGTAAGA | |
| CG59658-01 | AAAATACAGAACTGAGACCATTTCAGATAGAGATAAGCACTATGAGGGAAGCAAAGCT | |
| DNA | GCGTGTGTGCGGCAGGGGGCGTGGGCGCGGCGGCGCGGGCGGAGCGGCGCGGCCCGGG | |
| Sequence | CGCGGCGTGGGTAGAGCCGAGGCGGCGGCGGCGGCCGGCCTCTATCAGGATATCTACC | |
| | CTGCAGTACACACACTCACACAGGCACAAACACACACCACCCTCTGTCTCTCACATAC | |
| | ACTCACTCTCACACACACACACCCTCCCCCACACACTCTCACACACACACTTTCTCAC | |
| | ACACTCACACACACACTCTCACACACACTCTCTCACCCCCACACACTCACACACACTC | |
| | ACCCCACACACTCTCACACTCACTCTCACACACACTCACCCTACACTCCCGACTCCCC | |
| | TCAGACCCCCCCTCCTCTGGAGGAGACCCACATCTCATGCCTGTTCCCGGAGCTGCTG | |
| | GCCATGATCTTCGGCTACCTGGACGTCCGGGACAAGGGGCGCGCGGCGCAGGTGTGCA | |
| | CCGCCTGGCGGGACGCCGCCTACCACAAGTCGGTGTGGCGGGGGGTGGAGGCCAAGCT | |
| | GCACCTGCGCCGGGCCAACCCGTCGCTGTTCCCCAGCCTGCAGGCCCGGGGCATCCGC | |
| | CGGGTGCAGATCCTGAGCCTCCGCCGCAGCCTCAGCTACGTGATCCAGGGCATGGCCA | |
| | ACATCGAGAGCCTCAACCTCAGCGGCTGCTACAACCTCACCGACAACGGGCTGGGCCA | |
| | CGCGTTTGTGCAGGAGATCGGCTCCCTGCGCGCTCTCAACCTGAGCCTCTGCAAGCAG | |
| | ATCACTGACAGCAGCCTGGGCCGCATAGCCCAGTACCTCAAGGGCCTGGAGGTGCTGG | |
| | AGCTGGGAGGTTGCAGCAACATCACCAACACTGGCCTTCTGCTCATCGCCTGGGGTCT | |
| | GCAGCGCCTCAAGAGCCTTAACCTCCGCAGCTGCCGCCACCTTTCGGATGTGGGCATC | |
| | GGGCACCTGGCCGGCATGACGCGCAGCGCGGCGGAGGGCTGCCTGGGCCTGGAGCAGC | |
| | TCACGCTACAGGACTGCCAGAAGCTCACAGATCTTTCTCTAAAGCACATCTCCCGAGG | |
| | GCTGACGGGCCTGAGGCTCCTCAACCTCAGCTTCTGTGGGGGAATCTCGGACGCTGGC | |
| | CTCCTGCACCTGTCGCACATGGGCAGCCTGCGCAGCCTCAACCTGCGCTCCTGTGACA | |
| | ACATCAGTGACACGGGCATCATGCATCTGGCCATGGGCAGCCTGCGCCTCTCGGGGCT | |
| | GGATGTTTCGTTCTGTGACAAGGTGGGAGACCAGAGTCTGGCTTACATAGCCCAGGGG | |
| | CTGGATGGCCTCAAGTCTCTCTCCCTCTGCTCCTGCCACATCAGTGATGATGGCATCA | |
| | ACCGCATGGTGCGGCAGATGCACGGGCTGCGCACGCTCAACATTGGACAGTGTGTGCG | |
| | CATCACGGACAAGGGCCTGGAGCTGATCGCTGAGCACCTGAGCCAACTCACCGGCATA | |
| | GACCTGTACGGCTGCACCCGAATCACCAAGCGCGGCCTGGAGCGCATCACGCAGCTGC | |
| | CGTGCCTCAAGGTACTCAACCTGGGACTCTGGCAGATGACGGACAGTGAGAAGGTCAG | |
| | GTGAGGGCGGCAGCACCAGCTCCCCTTGTCCCGCCCTGTTCATCCTCCCATTACCACC | |
| | GCCCCCACACACTCACACGCACACTTACGCACAGATCATTGCAGCGGATGAGATGGGG | |
| | CTATGACAGAAGCCTCAGGCTCGTTTCCTCCTCCCTCCTCCAGCCCCCTCCCGGCTTC | |
| | CACTTAGATCTGCAGCCCTACCCACAACCACCTAAGCCTACTACCAGCTCCTTTTACA | |
| | CG | |
| | ORF Start: ATG at 40 | ORF Stop: TGA at 1684 |
| | SEQ ID NO:60 | 548 aa MW at 59767.0 kD |

| | |
|---|---|
| NOV25, | MGGDSKKNTELRPFQIEISTMREAKLRVCGRGRGRGGAGGAARPGRGVGRAEAAAAAG |
| CG59658-01 | LYQDIYPAVHTLTQAQTHTTLCLSHTLTLTHTHPPPHTLTHTLSHTLTHTLSHTLSHP |
| Protein | HTLTHTHPTHSHTHSHTHSPYTPDSPQTPPPLEETHISCLFPELLAMIFGYLDVRDKG |
| Sequence | RAAQVCTAWRDAAYHKSVWRGVEAKLHLRRANPSLFPSLQARGIRRVQILSLRRSLSY |
| | VIQGMANIESLNLSGCYNLTDNGLGHAFVQEIGSLRALNLSLCKQITDSSLGRIAQYL |
| | KGLEVLELGGCSNITNTGLLLIAWGLQRLKSLNLRSCRHLSDVGIGHLAGMTRSAAEG |
| | CLGLEQLTLQDCQKLTDLSLKHISRGLTGLRLLNLSFCGGISDAGLLHLSHMGSLRSL |
| | NLRSCDNISDTGIMHLAMGSLRLSGLDVSFCDKVGDQSLAYIAQGLDGLKSLSLCSCH |
| | ISDDGINRMVRQMHGLRTLNIGQCVRITDKGLELIAEHLSQLTGIDLYGCTRITKRGL |
| | ERITQLPCLKVLNLGLWQMTDSEKVR |

Further analysis of the NOV25 protein yielded the following properties shown in Table 25B.

TABLE 25B
Protein Sequence Properties NOV25

| | |
|---|---|
| PSort | 0.4500 probability located in cytoplasm; 0.3000 probability |
| analysis: | located in microbody (peroxisome); 0.2360 probability |
| | located in lysosome (lumen); 0.1000 probability located in |
| | mitochondrial matrix space |
| SignalP: | No Known Signal Sequence Predicted |
| analysis: | |

A search of the NOV25 protein against the Geneseq database, a proprietary database that contains sequences published in patents and patent publication, yielded several homologous proteins shown in Table 25C.

TABLE 25C
Geneseq Results for NOV25

| | | NOV25 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB42628 | Human ORFX ORF2392 polypeptide | 237 . . . 448 | 212/212 (100%) | e−118 |
| | sequence SEQ ID NO:4784 - Homo | 1 . . . 212 | 212/212 (100%) | |
| | sapiens, 212 aa. [WO200058473-A2, 05- | | | |
| | OCT-2000] | | | |
| AAB92791 | Human protein sequence SEQ ID | 159 . . . 533 | 126/388 (32%) | 6e−42 |
| | NO:11288 - Homo sapiens, 423 aa. | 18 . . . 396 | 190/388 (48%) | |
| | [EP1074617-A2, 07-FEB-2001] | | | |
| AAB92961 | Human protein sequence SEQ ID | 159 . . . 533 | 126/388 (32%) | 8e−42 |
| | NO:11652 - Homo sapiens, 423 aa. | 18 . . . 396 | 190/388 (48%) | |
| | [EP1074617-A2, 07-FEB-2001] | | | |
| AAB48290 | Human ZF1 protein - Homo sapiens, 466 | 159 . . . 533 | 126/388 (32%) | 1e−41 |
| | aa. [WO200075184-A1, 14-DEC-2000] | 18 . . . 396 | 190/388 (48%) | |
| AAY83090 | F-box protein FBP-22 - Homo sapiens, 437 | 159 . . . 533 | 125/388 (32%) | 1e−41 |
| | aa. [WO200012679-A1, 09-MAR-2000] | 32 . . . 410 | 188/388 (48%) | |

In a BLAST search of public sequence databases, the NOV25 protein was found to have homology to the proteins shown in the BLASTP data in Table 25D.

TABLE 25D
Public BLASTP Results for NOV25

| | | NOV25 | | |
|---|---|---|---|---|
| Protein | | Residues/ | Identities/ | |
| Accession | | Match | Similarities for the | Expect |
| Number | Protein/Organism/Length | Residues | Matched Portion | Value |
| AAH21329 | HYPOTHETICAL 43.9 KDA PROTEIN - | 150 . . . 548 | 398/399 (99%) | 0.0 |
| | Mus musculus (Mouse), 400 aa. | 2 . . . 400 | 398/399 (99%) | |
| Q922N5 | UNKNOWN (PROTEIN FOR | 222 . . . 548 | 327/327 (100%) | 0.0 |
| | IMAGE:3601186) - Mus musculus (Mouse), | 1 . . . 327 | 327/327 (100%) | |
| | 327 aa (fragment). | | | |
| Q9W214 | CG9952 PROTEIN (PARTNER OF | 8 . . . 539 | 298/535 (55%) | e−158 |
| | PAIRED) - Drosophila melanogaster (Fruit | 4 . . . 535 | 368/535 (68%) | |
| | fly), 538 aa. | | | |
| Q9PTL7 | F-BOX LEUCINE-RICH REPEAT | 150 . . . 418 | 143/291 (49%) | 2e−56 |
| | PROTEIN 13 - Xenopus laevis (African | 41 . . . 309 | 161/291 (55%) | |
| | clawed frog), 374 aa (fragment). | | | |
| Q9NVQ8 | CDNA FLJ10576 FIS, CLONE | 159 . . . 533 | 126/388 (32%) | 3e−41 |
| | NT2RP2003329, WEAKLY SIMILAR TO | 18 . . . 396 | 190/388 (48%) | |
| | PUTATIVE ADENYLATE CYCLASE | | | |
| | REGULATORY PROTEIN - Homo sapiens | | | |
| | (Human), 423 aa. | | | |

PFam analysis predicts that the NOV25 protein contains the domains shown in the Table 25E.

TABLE 25E
Domain Analysis of NOV25

| | | Identities/Similarities | |
|---|---|---|---|
| | NOV25 | for the | Expect |
| Pfam Domain | Match Region | Matched Region | Value |
| F-box: domain 1 of 1 | 151 . . . 198 | 8/48 (17%) | 0.0003 |
| | | 35/48 (73%) | |
| bac_dnaA: domain | 274 . . . 292 | 7/19 (37%) | 3.9 |
| 1 of 1 | | 17/19 (89%) | |
| LRR: domain 1 of 6 | 266 . . . 294 | 6/30 (20%) | 1.2e+02 |
| | | 22/30 (73%) | |
| LRR: domain 2 of 6 | 318 . . . 342 | 5/26 (19%) | 53 |
| | | 22/26 (85%) | |
| LRR: domain 3 of 6 | 351 . . . 379 | 6/30 (20%) | 4.1e+02 |
| | | 21/30 (70%) | |
| LRR: domain 4 of 6 | 402 . . . 428 | 8/28 (29%) | 1.4e+02 |
| | | 23/28 (82%) | |
| LRR: domain 5 of 6 | 454 . . . 478 | 4/26 (15%) | 3.6e+02 |
| | | 18/26 (69%) | |
| LRR: domain 6 of 6 | 479 . . . 507 | 5/29 (17%) | 2.8e+02 |
| | | 22/29 (76%) | |

### Example A26

The NOV26 clone was analyzed, and the nucleotide and predicted polypeptide sequences are shown in Table 26A.

TABLE 26A
NOV26 Sequence Analysis

| | SEQ ID NO:61 | 1752 bp |
|---|---|---|
| N0V26, | TGTAACCTTTAGACAATTTTGTCTCACAGGATGGACGTGGTAGAGGTCGCGGGTAGTT | |
| CG59907-01 | GGTGGGCACAAGAGCGAGAGGACATCATTATGAAATACGAAAAGGGACACCGAGCTGG | |
| DNA | GCTGCCAGAGGACAAGCGGCCTAAGTCTTTTGGAAGCTACAACAACAACGTCGATCAT | |
| Sequence | TTGGGGATTGTACAGGAGACGGAGCTGCCTCCTCTGACTGCGCGGGAGGTGAAGCAAA | |
| | TTCGGCGGGAGATCAGCCGAAAGAGCAAGTGGGTGAAAATGCTGGGAGAATGGGACAC | |
| | CTACAAAAACAGCAGAAAGCTCATAGATCGAGCGTACCAGGGAATTCCCATGAACATC | |
| | CGGGGCCCGATGTGGTCAGTCCTCCTGAACATTGAGGAAATCAAGTTGAAAAACCCCG | |
| | GAAGATACCAGATCATGAAGGAGAAGGGCAAGAGGTCATCTGAACACATCCAGCAGAT | |
| | GGACCTGGACGTAAGCGGGACATTAAGGAGGCATATATTCTTCAGGGATCGATACGGA | |
| | ACCAAGCAGCGGGAACTACTTTACATCCTCCTGGCGTATGAGGAGTATAACCCGGAGG | |
| | TGGGCTACTGCAGGGACCTGAGCCACATCGCCGCCTTGTTCCTCCTTTATCTTCCTGA | |
| | GGAGGATGCATTCTGGGCACTGGTGCAGCTGCTGGCCAGTGAGAGGCACTCCCTGCAG | |
| | GGTTTTCACAGCCCAAATGGCGGGACCGTCCAGGGGCTCCAAGACCAACAGGAGCATG | |
| | TGGTAGCCACGTCACTACCCAACACCATGTGGCATCAGGACAAGAAAGATCTATGTGG | |
| | GCAGTGTTCGTCCTTAGGCTGCCTCATCCGGATATTGATTGACGGGATCTCTCTCGGG | |
| | CTCACCCTGCGCCTGTGGGACGTGTATCTGGTAGAAGGCGAACAGGCGTTGATGCCGA | |
| | TAACAAGAATCGCCTTTAAGGTTCAGCTAGAGCGCCTCACGAAGACGTCCAGGTGTGG | |
| | CCCGTGGGCACGTTTTTGGAACCGGTTCGTTGATGCCTGGGCCAGGGATGATGACACT | |
| | GTGCTCAAGCATCTTAGGGCCTCTATGAAGAAACTAACAAGAAAGCAGGGGGACCTGC | |
| | CACCCCCAGCCAAACCCGAGCAAGGGTCGTCGGCATCCAGGCCTGTGCCAGCTTCACG | |
| | TGGCGGGAAGACCCTCTGCAAGGGGGACAGGCAGGCCCCTCCAGGCCCACCAGCCCGG | |
| | TTCCCATGGCCCATTTGGTCAGCTTCCCCGCCACGGGCACCTCGTTCTTCCACACCCT | |
| | GTCCTGGTGGGGCTGTCCGGGAAGACACCTACCCTGTGGGCACTCAGGGTGTGCCCAG | |
| | CCCGGCCCTGGCTCAGGGAGGACCTCAGGGTTCCTGGAGATTCCTGCAGTGGAACTCC | |
| | ATGCCCCGCCTCCCAACGGACCTGGACGTAGGGGACCCTTGGTTCCGCCGTTATGATT | |
| | TCAGACAGAGCTGCTGGGTCCGTGCCATATCCCAGGAGGACCAGCCGGCCACCTGCTG | |
| | GCAGGCTGAACACCCTGCGGAGCGGGTGAGATCGGCTTTCAGTGCGCCCAGCACTGAT | |
| | TCCGACCAGGGCACCCCCTTCAGAGCTAGGGACGAACAGCAGTGTGCTCCCACCTCAG | |
| | GACCTTGCCTCTGCGGCCTCCACTTGGAAAGTTCTCAGTTCCCTCCAGGCTTCTAGAA | |
| | GCATCTGGGCCAGGGCTCATGGCTGGATAATTTCCCTAGGCTTAACAACCCAAGCAAG | |
| | CTTCGCCTCCTC | |
| | ORF Start: ATG at 31 | ORF Stop: TAG at 1678 |
| | SEQ ID NO:62 | 549 aa MW at 62240.2 kD |

| | |
|---|---|
| NOV26, | MDVVEVAGSWWAQEREDIIMKYEKGHRAGLPEDKGPKSFGSYNNNVDHLGIVQETELP |
| CG59907-01 | PLTAREVKQIRREISRKSKWVKMLGEWDTYKNSRKLIDRAYQGIPMNIRGPMWSVLLN |
| Protein | IEEIKLKNPGRYQIMKEKGKRSSEHIQQMDLDVSGTLRRHIFFRDRYGTKQRELLYIL |
| Sequence | LAYEEYNPEVGYCRDLSHIAALFLLYLPEEDAFWALVQLLASERHSLQGFHSPNGGTV |
| | VEGEQALMPITRIAFKVQLERLTKTSRCGPWARFWNRFVDAWARDDDTVLKHLRASMK |
| | KLTRKQGDLPPPAKPEQGSSASRPVPASRGGKTLCKGDRQAPPGPPARFPWPIWSASP |
| | PRAPRSSTPCPGGAVREDTYPVGTQGVPSPALAQGGPQGSWRFLQWNSMPRLPTDLDV |
| | GDPWFRRYDFRQSCWVRAISQEDQPATCWQAEHPAERVRSAFSAPSTDSDQGTPFRAR |
| | DEQQCAPTSGPCLCGLHLESSQFPPGF |

Further analysis of the NOV26 protein yielded the following properties shown in Table 26B.

TABLE 26B
Protein Sequence Properties NOV26

| | |
|---|---|
| PSort | 0.4500 probability located in cytoplasm; 0.3866 probability |
| analysis: | located in microbody (peroxisome); 0.1564 probability |
| | located in lysosome (lumen); 0.1000 probability located in |
| | mitochondrial matrix space |
| SignalP | No Known Signal Sequence Predicted |
| analysis: | |

A search of the NOV26 protein against the Geneseq database, a proprietary database that contains sequences published in patents and patent publication, yielded several homologous proteins shown in Table 26C.

TABLE 26C
Geneseq Results for NOV26

| | | NOV26 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAY84901 | A human proliferation and apoptosis | 1 . . . 549 | 514/549 (93%) | 0.0 |
| | related protein - Homo sapiens, 549 aa. | 1 . . . 549 | 527/549 (95%) | |
| | [WO200023589-A2, 27-APR-2000] | | | |
| AAR90541 | pJG4-5-CDK-BP clone #118 derived | 1 . . . 366 | 289/366 (78%) | e−172 |
| | CDK4 binding protein - Synthetic, 376 aa. | 1 . . . 365 | 318/366 (85%) | |
| | [WO9533819-A2, 14-DEC-1995] | | | |
| AAM38692 | Human polypeptide SEQ ID NO 1837 - | 352 . . . 530 | 126/179 (70%) | 3e−66 |
| | Homo sapiens, 1085 aa. [WO200153312- | 34 . . . 210 | 134/179 (74%) | |
| | A1, 26-JUL-2001] | | | |
| AAM38691 | Human polypeptide SEQ ID NO 1836 - | 352 . . . 530 | 126/179 (70%) | 3e−66 |
| | Homo sapiens, 1089 aa. [WO200153312- | 34 . . . 210 | 134/179 (74%) | |
| | A1, 26-JUL-2001] | | | |
| AAW82396 | Human UBP protein #2 - Homo sapiens, | 352 . . . 530 | 126/179 (70%) | 3e−66 |
| | 1089 aa. [WO9848020-A2, 29-OCT-1998] | 34 . . . 210 | 134/179 (74%) | |

In a BLAST search of public sequence databases, the NOV26 protein was found to have homology to the proteins shown in the BLASTP data in Table 26D.

TABLE 26D
Public BLASTP Results for NOV26

| | | NOV26 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H0B9 | HYPOTHETICAL 62.2 KDA PROTEIN - | 1 . . . 549 | 513/549 (93%) | 0.0 |
| | Homo sapiens (Human), 549 aa. | 1 . . . 549 | 528/549 (95%) | |
| Q15634 | ONCOGENE - Homo sapiens (Human), 786 aa. | 1 . . . 530 | 412/530 (77%) | 0.0 |
| | | 1 . . . 527 | 449/530 (83%) | |
| S57867 | oncogene 1 - human, 376 aa. | 1 . . . 374 | 291/374 (77%) | e−172 |
| | | 1 . . . 373 | 320/374 (84%) | |
| Q15635 | ONCOGENE - Homo sapiens (Human), 376 aa. | 1 . . . 366 | 289/366 (78%) | e−171 |
| | | 1 . . . 365 | 318/366 (85%) | |
| P35125 | Ubiquitin carboxyl-terminal hydrolase 6 (EC | 352 . . . 530 | 126/179 (70%) | 1e−65 |
| | 3.1.2.15) (Ubiquitin thiolesterase 6) (Ubiquitin- | 34 . . . 210 | 134/179 (74%) | |
| | specific processing protease 6) | | | |
| | (Deubiquitinating enzyme 6) (Proto-oncogene | | | |
| | TRE-2) - Homo sapiens (Human), 1089 aa. | | | |

PFam analysis predicts that the NOV26 protein contains the domains shown in the Table 26E.

TABLE 26E
Domain Analysis of NOV26

| | NOV26 | Identities/Similarities | Expect |
|---|---|---|---|
| Pfam Domain | Match Region | for the Matched Region | Value |
| TBC: domain 1 of 1 | 98 . . . 315 | 60/343 (17%) | 2.3e−09 |
| | | 133/343 (39%) | |

### Example A27

The NOV27 clone was analyzed, and the nucleotide and predicted polypeptide sequences are shown in Table 27A.

TABLE 27A
NOV27 Sequence Analysis

| | SEQ ID NO:63 | 5973 bp |
|---|---|---|
| NOV27, | TGGACGGCATCTTTTTGAACCAAATTATGCTGCAAATAGATCCCAGGCCCACAAATCA | |
| CG59903-01 | ACGCATCAATAAGCACGTCAACAATGATGTGAACCTTCGCATTCAGAATTTGACCATC | |
| DNA | TTGGTGAGAAACATTAAGACCTACTACCAGGAAGTTCTCCAGCAGCTGATTGTAATGA | |
| Sequence | ATTTGCCCAATGTTTTGATGATTGGCAGAGACCCACTGTCTGGTGAGAGCATGGAGGA | |
| | AATCAAGAAGGTGCTGCTGCTGGTGCTGGGCTGTGCTGTCCAGTGTGAGAGGAAAGAG | |
| | GAGTTCATTGAAAGAATCAAACAGCTGGACATTGAGACCCAGGCTGGCATCGTGGCCC | |
| | ATATCCAGGAGGTAACTCACAACCAAGAGAACGTGTTTGACCTGCAGTGGCTGGAGCT | |
| | GCCCGACGTGGCTCCGGAGGAGCTGGAGGCCCTGTCGAGGAGCATGGTGCTCCACCTG | |
| | CGGAGGCTCATCGACCAGCGGGACGAGTGCACCGAGCTGATCGTGGACCTCACTCAGG | |
| | AACGGGACTACCTGCAGGCACAGCATCCACCCAGCCCCATCAAGTCCTCCAGCGCCGA | |
| | CTCCACTCCCAGCCCCACCAGCAGCCTCTCTAGCGAAGACAAGCAGCACCTGGCCGTA | |
| | GAGCTGGCCGACACCAAGGCCAGGCTGCGGCGCGTCAGGCAGGAGCTGGAGGATAAGA | |
| | CAGAGCAGCTTGTGGACACCAGACATGAGGTGGACCAGCTGGTGCTGGAACTGCAGAA | |
| | AGTTAAGCAGGAGAACATCCAGCTAGCGGCAGACGCCCGGTCTGCTCGTGCCTATCGA | |
| | TGACCCGCTGCAAGGAGAAGCTGCACGACGTGGACTTCTACAAGGCCCGCATGGAGGA | |
| | GCTGAGAGAAGATAATATCATTTTAATTGAAACCAAGGCCATGCTGGAGGAACAGCTG | |
| | ACTGCTGCTCGGGCCCGGGGCGATAAAGTCCATGAGCTGGAAAAGGAGAACCTGCAGC | |
| | TGAAATCCAAGCTTCACGACCTGGAATTGGACCGGGACACAGATAAGAAACGAATTGA | |
| | GGAGCTGCTGGAAGAAAACATGGTCCTTGAGATTGCACAGAAGCAGAGCATGAACGAA | |
| | TCTGCCCACCTTGGCTGGGAGCTGGAGCAGCTGTCCAAGAACGCAGACTTGTCATCAG | |
| | CCTCCAGGAAGTCGTTTGTGTTTGAGCTGAACGAATGTGCGTCCAGCCGCATCCTGAA | |
| | GCTGGAGAAGGAGAATCAGAGCCTCCAGAGCACCATCCAGGGGCTGCGGGACGCGTCC | |
| | CTGGTGTTGGAGGAGAGCGGCCTCAAGTGCGGGGAGCTGGAGAAGGAGAACCACCAGC | |
| | TCAGCAAGAAGGTAGAAAAGTTACAAACCCAGCTGGAGAGAGAAAAGCAGAGCAACCA | |
| | AGATCTGGAGACCCTCAGTGAGGAGCTGATCAGAGAGAAGGAGCAGCTGCAGAGTGAC | |
| | ATGGAGACCCTGAAGGCTGACAAAGCCAGGCAGATCAAGGACCTTGAGCAGGAAAAGG | |
| | ACCACCTCAACCGAGCCATGTGGTCGCTGCGGGAGAGGTCGCAGGTCAGCAGTGAGGC | |
| | CCGCATGAAAGACGTGGAGAAGGAGAACAAAGCCCTCCACCAGACGGTGACGGAGGCC | |
| | AATGGCAAGCTCAGCCAGTTGGAGTTTGAGAAGCGGCAGCTGCACAGGGACTTGGAGC | |
| | AGGCCAAGGAGAAGGGGGAGCGGGCAGAGAAGCTGGAGAGGGAGCTACAGCGACTCCA | |
| | GGAGGAGAACGGGAGGCTGGCCAGGAAGGTGACCTCCCTGGAGACAGCCACCGAGAAA | |
| | GTCGAGGCCCTGGAGCATGAGAGCCAGGGCCTGCAGCTGGAGAACCGGACTCTGAGGA | |
| | AGTCTCTGGACACCTTGCAGAACGTGTCCCTGCAGCTTGAGGGCCTGGAGCGTGACAA | |
| | CAAGCAGCTGGACGCAGAGAACCTGGAGCTGCGCAGGCTGGTGGAGACCATGCGCTTC | |
| | ACCAGCACCAAGCTGGCACAGATGGAGAGGGAGAACCAGCAGCTGGAGCGTGAGAAGG | |
| | AGGAGCTGAGGAAGAACGTGGATCTGCTCAAGGCGCTGGGCAAGAAGTCAGAGCGCCT | |
| | GGAGCTCAGCTACCAGAGCGTGAGCGCTGAGAACCTCCGGCTGCAGCAGAGCCTGGAG | |
| | AGCAGCAGCCACAAGACGCAGACCTTGGAGAGTGAGCTGGGCGAGCTGGAGGCTGAGC | |
| | GCCAGGCGCTGCGGCGGGACCTGGAGGCCCTCCGGCTGGCCAATGCACAGTTGGAGGG | |
| | GGCCGAGAAGGACAGGAAGGCCCTGGAGCAGGAGGTGGCCCAGCTCGAGAAGGATAAG | |
| | AAGCTGCTGGAGAAGGAGGCCAAGCGGCTGTGGCAGCAGGTGGAGCTCAAGGATGCAG | |
| | TCTTGGACGATAGCACTGCCAAACTGTCCGCCGTTGAGAAGGAGAGCCGCGCGCTGGA | |
| | CAAGGAGCTGGCCCGCTGCAGGGACGCAGCCGGCAAGCTGAAGGAGCTGGAGAAGGAC | |
| | AACCGGGACCTCACCAAGCAAGTCACCGTGCATGCAAGGACACTGACAACTCTGAGGG | |
| | AGGACCTGGTGCTCGAGAAGCTGAAGAGCCAGCAGCTCAGCAGTGAGCTGGACAAGCT | |
| | GAGCCAGGAACTGGAGAAGGTCGGCCTCAACAGGGAGCTGCTGTTGCAGGAGGACGAC | |
| | CAACACTAGCCATGAAAGAAGAAAAGATTGTGCTCTTAGAAGCACAGATGGAAGAGAA | |
| | AGCGAGCCTAAATCGCCAGTTAGAGAGTGAGCTGCAGATGCTAAAGAAGGAGTGTGAG | |
| | ACCCTCAGGCAGAACCAGGGAGAGGGGCAGCACTTGCAGAACTCTTTCAAGCACCCTG | |
| | CGGGGAAGACAGCCGCCAGTCACCAGGGGAAGGAGGCCTGGGGGCCCGGCCATAAGGA | |
| | AGCCACCATGGAGCTTCTCCGAGTGAAGGACCGGGCCATCGAGCTGGAGCGGAATAAT | |
| | GCAGCTCTGCAGGCTGAGAAGCAGCTGCTAAAGGAACAGCTGCAGCACCTGGAGACCC | |
| | AGAACGTGACCTTCAGCAGCCAGATCTTGACACTGCAGAAACAGAGCGCCTTCCTGCA | |
| | GGAGCACAACACCACACTGCAGACCCAGACCGCCAAGCTGCAGGTGGAGAACTCCACG | |
| | CTGAGTTCCCAGAGCGCAGCGCTCACCGCGCAGTACACGCTGCTGCAGAACCACCACA | |
| | CGGCCAAGGAGACGGAGAACGAAAGCCTGCAGAGGCAGCAGGAGCAACTTACAGCGGC | |
| | CTACGAGGCCCTGCTGCAGGACCACGAGCACCTGGGCACGCTGCACGAGCGGCAATCG | |
| | GCCGAGTACGAGGCCCTCATCCGCCAGCACAGCTGCCTAAAGACACTGCATCGGAATC | |
| | TGGAGCTGGAGCACAAGGAGCTCGGGGAGAGGCACGGTGACATGCTGAAGCGCAAGGC | |
| | GGAGCTGGAGGAGCGGGAGAAGGTCTTGACCACTGAGCGAGAGGCGCTGCAGCAGGAG | |
| | CAGAGGACAAACGCCCTCGCCATGGGCGAGAACCAGAGGCTGCGGGGCGAGCTGGACA | |
| | GGGTCAATTTCCTGCACCACCAGCTGAAGGGGGAGTACGAGGAGCTGCACGCCCACAC | |
| | CAAGGAGCTGAAAACCTCACTGAACAACGCGCAGCTGGAGCTCAACCGCTGGCAGGCC | |
| | CGCTTCGACGAGCTGAAGGAGCAGCACCAGACCATGGACATCTCGCTGACCAAGCTGG | |
| | ACAACCACTGTGAGCTGCTCTCCCGTCTCAAGGGGAACTTGGAGGAAGAAAATCATCA | |
| | CCTCCTGAGCCAGATCCAGCTGTTGAGCCAGCAGAACCAGATGCTTCTGGAGCAGAAC | |
| | ATGGAGAACAAGGAGCAGTACCATGAGGAGCAGAAGCAGTACATAGACAAATTAAATG | |
| | CCTTACGAAGACATAAGGAAAAGCTGGAAGAAAAAATCATGGATCAATACAAGTTCTA | |
| | TGATCCTCCTCCAAAGAAGAAGAACCACTGGATTGGAGCCAAAGCCTTAGTCAAACTC | |
| | ATCAAACCAAAGAAAGAGGGTTCGAGGGAACGCTTAAAATCCACCGTGGACAGCCCTC | |
| | CCTGGCAGCTGGAGTCCTCAGACCCCGCCTCGCCGGCGGCCTCTCAGCCGCTCAGATC | |
| | ACAGGCCGCGAACCCCGACACCCCCGCACTGGGCTCCAACTGTGCAGAAGAGCGCGAC | |
| | GCCCACAACGGGTCTGTGGGGAAAGGCCCTGGGGATCTAAAACCAAAGCGAGGCTCCC | |
| | CACACAGAGGCAGCCTTGACCGCACAGATGCCTCCACCGATCTGGCCATGAGGTCCTG | |
| | GCCCTCGGAGCTGGGCTCCCGGACTTGCTCAACTTCAGCCACCACTACAGCCCCTTCC | |
| | AACTCCACCCCCATCGCCCGGCACCCAGGCCGCACCAAAGGCTATAACTCAGATGACA | |
| | ACCTCTGTGAGCCATCCCTGGAGTTTGAGGTCCCCAACCACAGGCAGTACGTGTCGCG | |
| | GCCAAGTAGCTTAGAGAGCAGTAGAAACACATCCAGCAACAGCTCACCTCTTAACCTA | |
| | AAAGGCTCCTCCGAGCAGCTCCATGGCCGGTCTGAGAGCTTCAGCAGCGAAGACCTGA | |
| | TCCCCAGCAGGGACCTGGCCACTTTGCCCCGGGAAGCCAGCACACCGGGACGCAACGC | |
| | CCTCGGCCGCCACGAGTACCCCTTGCCTCGGAACGGGCCTCTCCCACAGGAGGGTGCC | |
| | CAGAAGAGGGGCACAGCCCCTCCCTACGTCGGAGTGCGGCCCTGCTCGGCCTCCCCCA | |
| | GCAGTGAGATGGTCACCTTGGAGGAGTTCCTGGAGGAGAGCAACCGCAGCTCCCCCAC | |
| | CCATGACACTCCCAGTTGCCGGGATGACCTGCTGAGTGACTACTTCCGAAAGGCCAGC | |
| | GATCCCCCAGCCATCGGAGGCCAACCAGGACCACCTGCCAAGAAAGAAGGGGCCAAGA | |
| | TGCCCACCAACTTTGTGGCCCCCACCGTCAAAATGGCCGCCCCCACCTCGGAGGGGAG | |
| | GCCGCTGAAGCCCGGGCAGTACGTAAAGCCAAACTTCAGACTCACTCAGGCCGAGGCC | |
| | CCACCCAGCGTGGCCCCGAGACAGGCCCAGCCTCCCCAGAGCCTGTCTCTGGGCAGAC | |
| | CCCGGCAGGCTCCGGTGCCCCCAGCTTCCCATGCACCTGCCAGCCGCAGTGCCTCCTT | |
| | GAGCCGGGCCTTCAGCCTGGCCTCAGCTGACCTTCTCCGGGCCAGCGGGCCAGAGGCC | |
| | TGCAAACAGGAGTCCCCTCAGAAGCTGGGGGCTCCTGAGGCCTTAGGGGGCAGAGAGA | |
| | CAGGCAGCCACACCCTGCAAAGCCCCGCACCCCCCAGCTCCCATAGCCTGGCCCGGGA | |
| | GCGGACCCCACTTGTGGGAAAGGCTGGCAGCTCCTGTCAGGGCCCAGGTCCCCGCAGC | |
| | CGGCCGCTGGACACGAGGCGCTTCTCCCTGGCTCCCCCAAAGGAGGAGAGGCTGGCCC | |
| | CCCTGCATCAGTCTGCCACAGCCCCCGCCATTGCCACTGCAGGTGCTGGTGCTGCTGC | |
| | TGCTGGCAGTGGCAGCAACTCCCAGCTCCTGCACTTCTCACCTGCTGCAGCCCCGGCT | |
| | GCCAGGACCAAGCCCAAGGCGCCCCCACGCTCAGGGGAGGTGGCCACCATCACCCCTG | |
| | TCCGGGCAGGGCTCAGCCTCTCAGAGGGAGACGGGGTCCCGGGGCAGGGCTGCAGTGA | |
| | GCCCTGGAGGACTGCAGTCGAGGGAGCGTCTCAAAGAGCAGTCCGGCCTCCCCGGAGC | |
| | CCGGCGGGGATCCGCAGACCGTGTGGTATGAGTACGGCTGTGTGTGACTGTCTCGTG | |
| | ORF Start: ATG at 27 | ORF Stop: TGA at 5961 |
| | SEQ ID NO:64 | 1978 aa MW at 222593.2 kD |

| | |
|---|---|
| NOV27, | MLQIDPRPTNQRINKHVNNDVNLRIQNLTILVRNIKTYYQEVLQQLIVMNLPNVLMIG |
| CG59903-01 | RDPLSGESMEEIKKVLLLVLGCAVQCERKEEFIERIKQLDIETQAGIVAHIQEVTHNQ |
| Protein | ENVFDLQWLELPDVAPEELEALSRSMVLHLRRLIDQRDECTELIVDLTQERDYLQAQH |
| Sequence | PPSPIKSSSADSTPSPTSSLSSEDKQHLAVELADTKARLRRVRQELEDKTEQLVDTRH |
| | EVDQLVLELQKVKQENIQLAADARSARAYRDELDSLREKANRVERLELELTRCKEKLH |
| | DVDFYKARMEELREDNIILIETKAMLEEQLTAARARGDKVHELEKENLQLKSKLHDLE |
| | LDRDTDKKRIEELLEENMVLEIAQKQSMNESAHLGWELEQLSKNADLSSASRKSFVFE |
| | LNECASSRILKLEKENQSLQSTIQGLRDASLVLEESGLKCGELEKENHQLSKKVEKLQ |
| | TQLEREKQSNQDLETLSEELIREKEQLQSDMETLKADKARQIKDLEQEKDHLNRAMWS |
| | LRERSQVSSEARMKDVEKENKALHQTVTEANGKLSQLEFEKRQLHRDLEQAKEKGERA |
| | EKLERELQRLQEENGRLARKVTSLETATEKVEALEHESQGLQLENRTLRKSLDTLQNV |
| | SLQLEGLERDNKQLDAENLELRRLVETMRFTSTKLAQMERENQQLEREKEELRKNVDL |
| | LKALGKKSERLELSYQSVSAENLRLQQSLESSSHKTQTLESELGELEAERQALRRDLE |
| | ALRLANAQLEGAEKDRKALEQEVAQLEKDKKLLEKEAKRLWQQVRLKDAVLDDSTAKL |
| | SAVEKESRALDKELARCRDAAGKLKELEKDNRDLTKQVTVHARTLTTLREDLVLEKLK |
| | SQQLSSELEKLSQELEKVGLNRELLLQEDDSGSDTKYKILEGRNESALKTTLAMKEEK |
| | IVLLEAQMEEKASLNRQLESELQMLKKECETLRQNQGEGQHLQNSFKHPAGKTAASHQ |
| | GKEAWGPGHKKATMELLRVKDRAIELERNNAALQAEKQLLKEQLQHLETQNVTFSSQI |
| | LTLQKQSAFLQEHNTTLQTQTAKLQVENSTLSSQSAALTAQYTLLQNHHTAKETENES |
| | LQRQQEQLTAAYEALLQDHEHLGTLHERQSAEYEALIRQHSCLKTLHRNLELEHKELG |
| | ERHGLMLKRKAELEEREKVLTTEREALQQEQRTNALAMGENQRLRGELDRVNFLHHQL |
| | KGEYEELHAHTKELKTSLNNAQLELNRWQARFDELKEQHQTMDISLTKLDNHCELLSR |
| | LKGNLEEENHHLLSQIQLLSQQNQMLLEQNMENKEQYHEEQKQYIDKLNALRRHKEKL |
| | EEKIMDQYKFYDPPPKKKNHWIGAKALVKLIKPKKEGSRERLKSTVDSPPWQLESSDP |
| | ASPAASQPLRSQAENPDTPALGSNCAEERDAHNGSVGKGPGDLKPKRGSPHRGSLDRT |
| | DASTDLAMRSWPSELGSRTCSTSATTTAPSNSTPIARHPGRTKGYNSDDNLCEPSLEF |
| | EVPNHRQYVSRPSSLESSRNTSSNSSPLNLKGSSEQLHGRSESFSSEDLIPSRDLATL |
| | PREASTPGRNALGRHEYPLPRNGPLPQEGAQKRGTAPPYVGVRPCSASPSSEMVTLEE |
| | FLEESNRSSPTHDTPSCRDDLLSDYFRKASDPPAIGGQPGPPAKKEGAKMPTNFVAPT |
| | VKMAAPTSEGRPLKPGQYVKPNFRLTEAEAPPSVAPRQAQPPQSLSLGRPRQAPVPPA |
| | SHAPASRSASLSRAFSLASADLLRASGPEACKQESPQKLGAPEALGGRETGSHTLQSP |
| | APPSSHSLARERTPLVGKAGSSCQGPGPRSRPLDTRRFSLAPPKEERLAPLHQSATAP |
| | AIATAGAGAAAAGSGSNSQLLHFSPAAAPAARTKPKAPPRSGEVATITPVRAGLSLSE |
| | GDGVPGQGCSEGLPAKSPGRSPDLAPHLGRALEDCSRGSVSKSSPASPEPGGDPQTVW |
| | YEYGCV |

Further analysis of the NOV27 protein yielded the following properties shown in Table 27B.

TABLE 27B
Protein Sequence Properties NOV27

| | |
|---|---|
| PSort | 0.9600 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV27 protein against the Geneseq database, a proprietary database that contains sequences published in patents and patent publication, yielded several homologous proteins shown in Table 27C.

TABLE 27C
Geneseq Results for NOV27

| | | NOV27 | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAM78520 | Human protein SEQ ID NO 1182 - Homo | 1 . . . 956 | 544/956 (56%) | 0.0 |
| | sapiens, 990 aa. [WO200157190-A2, 09- | 39 . . . 990 | 760/956 (78%) | |
| | AUG-2001] | | | |
| AAM79504 | Human protein SEQ ID NO 3150 - Homo | 22 . . . 956 | 530/935 (56%) | 0.0 |
| | sapiens, 931 aa. [WO200157190-A2, 09- | 1 . . . 931 | 743/935 (78%) | |
| | AUG-2001] | | | |
| AAB42l96 | Human ORFX ORF1960 polypeptide | 132 . . . 556 | 423/425 (99%) | 0.0 |
| | sequence SEQ ID NO: 3920 - Homo | 2 . . . 426 | 424/425 (99%) | |
| | sapiens, 426 aa. [WO200058473-A2, 05- | | | |
| | OCT-2000] | | | |
| AAB94101 | Human protein sequence SEQ ID | 1619 . . . 1978 | 359/360 (99%) | 0.0 |
| | NO: 14322 - Homo sapiens, 360 aa. | 1 . . . 360 | 359/360 (99%) | |
| | [EP1074617-A2, 07-FEB-2001] | | | |
| AAB92772 | Human protein sequence SEQ ID | 1077 . . . 1709 | 237/647 (36%) | 1e−96 |
| | NO: 11249 - Homo sapiens, 636 aa. | 2 . . . 613 | 356/647 (54%) | |
| | [EP1074617-A2, 07-FEB-2001] | | | |

In a BLAST search of public sequence databases, the NOV27 protein was found to have homology to the proteins shown in the BLASTP data in Table 27D.

TABLE 27D
Public BLASTP Results for NOV27

| | | NOV27 | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9P219 | KIAA1509 PROTEIN - Homo sapiens | 653 . . . 1978 | 1326/1326 (100%) | 0.0 |
| | (Human), 1326 aa (fragment). | 1 . . . 1326 | 1326/1326 (100%) | |
| Q9UI01 | HYPOTHETICAL 87.3 KDA | 25 . . . 764 | 429/740 (57%) | 0.0 |
| | PROTEIN - Homo sapiens (Human), | 1 . . . 736 | 594/740 (79%) | |
| | 742 aa. | | | |
| O14997 | 3-7 GENE PRODUCT - Homo | 25 . . . 731 | 413/707 (58%) | 0.0 |
| | sapiens (Human), 709 aa (fragment). | 1 . . . 703 | 569/707 (80%) | |
| Q9H9V0 | CDNA FLJ12531 FIS, CLONE | 1619 . . . 1978 | 359/360 (99%) | 0.0 |
| | NT2RM4000199 - Homo sapiens | 1 . . . 360 | 359/360 (99%) | |
| | (Human), 360 aa. | | | |
| Q9VZT7 | CG12734 PROTEIN - Drosophila | 2 . . . 1141 | 330/1186 (27%) | e−105 |
| | melanogaster (Fruit fly), 1381 aa. | 51 . . . 1138 | 578/1186 (47%) | |

PFam analysis predicts that the NOV27 protein contains the domains shown in the Table 27E.

TABLE 27E
Domain Analysis of NOV27

| | | Identities/ | |
|---|---|---|---|
| | | Similarities | |
| | NOV27 | for the | Expect |
| Pfam Domain | Match Region | Matched Region | Value |
| Flavi_NS1: | 78 . . . 88 | 6/11 (55%) | 9.2 |
| domain 1 0f 1 | | 9/11 (82%) | |
| DUF164: domain 1 of 1 | 473 . . . 683 | 41/249 (16%) | 5.3 |
| | | 131/249 (53%) | |
| ERM: domain 1 of 1 | 644 . . . 910 | 64/406 (16%) | 3.1 |
| | | 176/406 (43%) | |
| K-box: domain 1 of 1 | 980 . . . 1067 | 26/105 (25%) | 0.27 |
| | | 52/105 (50%) | |
| Rhodopsin_C: | 1620 . . . 1765 | 35/147 (24%) | 9 |
| domain 1 of 1 | | 53/147 (36%) | |

### Example A28

The NOV28 clone was analyzed, and the nucleotide and predicted polypeptide sequences are shown in Table 28A.

TABLE 28A
NOV28 Sequence Analysis

| | SEQ ID NO:65 | 1043 bp |
|---|---|---|
| NOV28, | GAGTCCTGGGAGCATGGCTTTCCCTGAGCCAAAGCCGCGGCCTCCAGAGCTGCCGCAG | |
| GG59985-01 | AAACGGTTGAAGACGCTGGACTGCGGGCAGGGGGCAGTGCGAGCCGTACGATTTAATG | |
| DNA | TGGATGGCAATTACTGCCTGACGTGCGGCAGTGACAAGACGCTGAAGCTGTGGAACCC | |
| Sequence | GCTTCGGGGGACGCTGCTGCGGACGTACAGCGGCCACGGCTACGAGGTGCTGGATGCG | |
| | GCCGGGTCCTTTGACAACAGTAGTCTCTGCTCCGGCGGCGGGGACAAGGCGGTGGTTC | |
| | TGTGGGATGTGGCATCAGGGCAGGTCGTGCGCAAATTCCGGGGCCACGCAGGGAAGGT | |
| | GAACACGGTGCAGTTTAATGAAGAGGCCACAGTTATCCTGTCCGGTTCTATTGATTCC | |
| | AGTATCCGCTGTTGGGATTGCCGCTCACGGAGGCCTGAGCCAGTGCAGACGCTGGATG | |
| | AGGCCAGAGATGGCGTGTCCAGTGTGAAGGTGTCAGACCACGAGATCCTGGCAGGGTC | |
| | CGTGGATGGCCGCGTGAGACGCTATGACCTAAGGATGGGGCAGCTCTTCTCAGACTAC | |
| | GTGGGCAGTGAGCCCATCACCTGCACCTGCTTCAGCCGGGATGGGCAGTGCACCCTGG | |
| | TGTCCAGCCTGGACTCCACATTGCGGCTCCTGGACAAAGACACAGGGGAGCTGCTGGG | |
| | CGAGTACAAGGGCCATAAGAACCAGGAATACAAGCTGGACTGCTGCCTGAGCGAGCGT | |
| | GACACACATGTGGTCAGCTGTTCTGAGGACGGGAAGGTGTTCTTCTGGGACCTGGTGG | |
| | AGGGTGCGCTGGCTCTGGCCCTGCCTGTGGGTTCCGGTGTGGTGCAGTCGCTGGCCTA | |
| | CCACCCAACAGAGCCCTGCCTGCTGACCGCCATGGGAGGCAGCGTCCAGTGCTGGCGA | |
| | GAGGAGGCCTATGAGGCAGAGGATGGAGCAGGCTGAAGCCAGGGGACCCACCAACAGG | |
| | ACCAAGGACCGAGACACAGACATGGAAGGACTTCAGATACCATCTTATTCTAGAGAC | |
| | ORF Start ATG at 14 | ORF Stop: TGA at 962 |
| | SEQ ID NO:66 | 316 aa MW at 34471.4 kD |

| | |
|---|---|
| NOV28, | MAFPEPKPRPPELPQKRLKTLDCGQGAVRAVRFNVDGNYCLTCGSDKTLKLWNPLRGT |
| CG59985-01 | LLRTYSGHGYEVLDAAGSFDNSSLCSGGGDKAVVLWDVASGQVVRKFRGHAGKVNTVQ |
| Protein | FNEEATVILSGSIDSSIRCWDCRSRRPEPVQTLDEARDGVSSVKVSDHEILAGSVDGR |
| Sequence | VRRYDLRMGQLFSDYVGSEPITCTCFSRDGQCTLVSSLDSTLRLLDKDTGELLGEYKG |
| | HKNQEYKLDCCLSERDTHVVSCSEDGKVFFWDLVEGALALALPVGSGVVQSLAYHPTE |
| | PCLLTAMGGSVQCWREEAYEAEDGAG |

Further analysis of the NOV28 protein yielded the following properties shown in Table 28B.

TABLE 28B
Protein Sequence Properties NOV28

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV28 protein against the Geneseq database, a proprietary database that contains sequences published in patents and patent publication, yielded several homologous proteins shown in Table 28C.

TABLE 28C
Geneseq Results for NOV28

| | | NOV28 | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB68545 | Human GTP-binding associated protein # | 1 . . . 316 | 312/316 (98%) | 0.0 |
| | 45 - Homo sapiens, 315 aa. [WO200105970- | 1 . . . 315 | 314/316 (98%) | |
| | A2, 25-JAN-2001] | | | |
| AAG51154 | Arabidopsis thaliana protein fragment SEQ | 12 . . . 306 | 151/296 (51%) | 7e−87 |
| | ID NO: 64895 - Arabidopsis thaliana, 299 | 5 . . . 299 | 201/296 (67%) | |
| | aa. [EP1033405-A2, 06-SEP-2000] | | | |
| AAG51153 | Arabidopsis thaliana protein fragment SEQ | 12 . . . 306 | 151/296 (51%) | 7e−87 |
| | ID NO: 64894 - Arabidopsis thaliana, 304 | 10 . . . 304 | 201/296 (67%) | |
| | aa. [EP1033405-A2, 06-SEP-2000] | | | |
| AAG51148 | Arabidopsis thaliana protein fragment SEQ | 12 . . . 296 | 149/285 (52%) | 2e−85 |
| | ID NO: 64887 - Arabidopsis thaliana, 330 | 5 . . . 288 | 193/285 (67%) | |
| | aa. [EP1033405-A2, 06-SEP-2000] | | | |
| AAM93208 | Human polypeptide, SEQ ID NO: 2603 - | 182 . . . 316 | 134/135 (99%) | 4e−75 |
| | Homo sapiens, 134 aa. [EP1130094-A2, 05- | 1 . . . 134 | 134/135 (99%) | |
| | SEP-2001] | | | |

In a BLAST search of public sequence databases, the NOV28 protein was found to have homology to the proteins shown in the BLASTP data in Table 28D.

TABLE 28D
Public BLASTP Results for NOV28

| | | NOV28 | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9BRX9 | SIMILAR TO RIKEN CDNA | 1 . . . 316 | 315/316 (99%) | 0.0 |
| | 1500041N16 GENE - Homo sapiens | 1 . . . 315 | 315/316 (99%) | |
| | (Human), 315 aa. | | | |
| Q9DAJ4 | 1500041N16RIK PROTEIN - Mus | 1 . . . 316 | 296/316 (93%) | e−177 |
| | musculus (Mouse), 315 aa. | 1 . . . 315 | 305/316 (95%) | |
| AAH19369 | RIKEN CDNA 1500041N16 GENE - | 1 . . . 316 | 295/316 (93%) | e−177 |
| | Mus musculus (Mouse), 315 aa. | 1 . . . 315 | 304/316 (95%) | |
| Q9D235 | 1500041N16RIK PROTEIN - Mus | 1 . . . 316 | 290/316 (91%) | e−173 |
| | musculus (Mouse), 315 aa. | 1 . . . 315 | 301/316 (94%) | |
| Q94AH2 | HYPOTHETICAL 33.1 KDA | 12 . . . 306 | 151/296 (51%) | 3e−86 |
| | PROTEIN - Arabidopsis thaliana | 5 . . . 299 | 201/296 (67%) | |
| | (Mouse-ear cress), 299 aa. | | | |

PFam analysis predicts that the NOV28 protein contains the domains shown in the Table 28E.

TABLE 28E
Domain Analysis of NOV28

| | | Identities/ | |
|---|---|---|---|
| | | Similarities | |
| | NOV28 | for the | Expect |
| Pfam Domain | Match Region | Matched Region | Value |
| WD40: domain 1 of 7 | 17 . . . 53 | 11/37 (30%) | 6.4e−05 |
| | | 35/37 (95%) | |
| WD40: domain 2 of 7 | 59 . . . 95 | 15/37 (41%) | 0.0039 |
| | | 27/37 (73%) | |
| WD40: domain 3 of 7 | 101 . . . 137 | 11/37 (30%) | 0.00019 |
| | | 28/37 (76%) | |
| WD40: domain 4 of 7 | 145 . . . 179 | 11/37 (30%) | 1 |
| | | 28/37 (76%) | |
| WD40: domain 5 of 7 | 184 . . . 220 | 11/37 (30%) | 9.4 |
| | | 25/37 (68%) | |
| WD40: domain 6 of 7 | 226 . . . 264 | 11/39 (28%) | 0.95 |
| | | 31/39 (79%) | |
| WD10: domain 7 of 7 | 270 . . . 305 | 13/37 (35%) | 83 |
| | | 24/37 (65%) | |

### Example B: Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology:

The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corporation proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. Table B1 shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone:

Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C. Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (13iochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20ng/ml PMA and 1-2μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resuspended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/mi IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; patient not demented but showing sever AD-like pathology

SupTemporal Ctx=Superior Temporal Cortex

Inf Temporal Ctx=Inferior Temporal Cortex

A. CG57883-01: Ring Finger Protein

Expression of gene CG57883-01 was assessed using the primer-probe set Ag3352, described in Table AA. Results of the RTQ-PCR runs are shown in Table AB.

TABLE AA
Probe Name Ag3352

| | | SEQ ID | | |
|---|---|---|---|---|
| Primers | Sequences | NO. | Length | Start Position |
| Forward | 5′-atgggtgccatatctacacatt-3′ | 67 | 22 | 801 |
| Probe | TET-5′-atcgagattcctgtttgcgagccct-3′-TAMRA | 68 | 25 | 824 |
| Reverse | 5′-gctctgatcatcttgacttcca-3′ | 69 | 22 | 874 |

TABLE AB
General_screening_panel_v1.4

| | Rel. Exp.(%) Ag3352, Run | | Rel. Exp.(%) Ag3352, Run |
|---|---|---|---|
| Tissue Name | 219797990 | Tissue Name | 219797990 |
| Adipose | 0.9 | Renal ca. TK-10 | 0.1 |
| Melanoma* | 0.0 | Bladder | 0.4 |
| Hs688(A).T | | | |
| Melanoma* Hs688(B).T | 0.0 | Gastric ca. (liver met.) | 0.5 |
| | | NCI-N87 | |
| Melanoma* M14 | 0.2 | Gastric ca. KATO III | 0.0 |
| Melanoma* LOXIMVI | 0.8 | Colon ca. SW-948 | 0.1 |
| Melanoma* SK-MEL-5 | 0.3 | Colon ca. SW480 | 2.5 |
| Squamous cell | 0.5 | Colon ca.* (SW480 met) | 0.2 |
| carcinoma SCC-4 | | SW620 | |
| Testis Pool | 1.8 | Colon ca. HT29 | 0.1 |
| Prostate ca.* (bone met) | 0.4 | Colon ca. HCT-116 | 0.7 |
| PC-3 | | | |
| Prostate Pool | 0.0 | Colon ca. CaCo-2 | 0.4 |
| Placenta | 0.0 | Colon cancer tissue | 0.3 |
| Uterus Pool | 0.0 | Colon ca. SW1116 | 0.0 |
| Ovarian ca. OVCAR-3 | 0.2 | Colon ca. Colo-205 | 0.0 |
| Ovarian ca. SK-OV-3 | 0.8 | Colon ca. SW-48 | 0.0 |
| Ovarian ca. OVCAR-4 | 0.3 | Colon Pool | 0.2 |
| Ovarian ca. OVCAR-5 | 0.0 | Small Intestine Pool | 0.1 |
| Ovarian ca. IGROV-1 | 0.0 | Stomach Pool | 0.0 |
| Ovarian ca. OVCAR-8 | 0.8 | Bone Marrow Pool | 0.0 |
| Ovary | 0.5 | Fetal Heart | 0.0 |
| Breast ca. MCF-7 | 0.0 | Heart Pool | 0.0 |
| Breast ca. MDA-MB-231 | 0.4 | Lymph Node Pool | 0.1 |
| Breast ca. BT 549 | 0.0 | Fetal Skeletal Muscle | 0.3 |
| Breast ca. T47D | 0.0 | Skeletal Muscle Pool | 0.0 |
| Breast ca. MDA-N | 0.0 | Spleen Pool | 0.2 |
| Breast Pool | 0.0 | Thymus Pool | 0.1 |
| Trachea | 0.3 | CNS cancer (glio/astro) | 3.0 |
| | | U87-MG | |
| Lung | 0.0 | CNS cancer (glio/astro) U- | 3.9 |
| | | 118-MG | |
| Fetal Lung | 0.5 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.1 | CNS cancer (astro) SNB-75 | 0.0 |
| Lung ca. NCI-H146 | 0.0 | CNS cancer (glio) SNB-19 | 0.0 |
| Lung ca. SHP-77 | 17.1 | CNS cancer (glio) SF-295 | 0.4 |
| Lung ca. A549 | 0.0 | Brain (Amygdala) Pool | 0.2 |
| Lung ca. NCI-H526 | 0.0 | Brain (cerebellum) | 0.0 |
| Lung ca. NCI-H23 | 0.3 | Brain (fetal) | 0.0 |
| Lung ca. NCI-H460 | 0.0 | Brain (Hippocampus) Pool | 0.0 |
| Lung ca. HOP-62 | 0.3 | Cerebral Cortex Pool | 0.0 |
| Lung ca. NCI-H522 | 0.1 | Brain (Substantia nigra) | 0.0 |
| | | Pool | |
| Liver | 0.5 | Brain (Thalamus) Pool | 100.0 |
| Fetal Liver | 0.1 | Brain (whole) | 0.1 |
| Liver ca. HepG2 | 0.2 | Spinal Cord Pool | 0.0 |
| Kidney Pool | 0.5 | Adrenal Gland | 0.0 |
| Fetal Kidney | 0.0 | Pituitary gland Pool | 0.0 |
| Renal ca. 786-0 | 0.2 | Salivary Gland | 0.0 |
| Renal ca. A498 | 0.3 | Thyroid (female) | 0.0 |
| Renal ca. ACHN | 0.0 | Pancreatic ca. CAPAN2 | 0.0 |
| Renal ca. UO-31 | 1.4 | Pancreas Pool | 0.1 |

CNS_neurodegeneration_v1.0 Summary: Ag3352—Expression of CG57883-01 gene is low/undetectable (CTs>35) across all of the samples on this panel (data not shown).

General_screening_panel_v1.4 Summary: Ag3352—Highest expression of the CG57883-01 gene is seen exclusively in the thalamus (CT=27.4). Therefore, expression of this gene could be used to distinguish this sample from other samples used in this panel.

In addition, low to moderate expression of this gene is seen in lung, colon, CNS, renal, ovarian and melanoma. Therefore, therapeutic modulation of the activity of this gene or its protein product, through the use of small molecule drugs, protein therapeutics or antibodies, might be beneficial in the treatment of these cancers.

Among tissues with metabolic or endocrine function, this gene is expressed at low levels in adipose, and liver (CTs=34-34.8). Therefore, therapeutic modulation of the activity of this gene may prove useful in the treatment of endocrine/metabolically related diseases, such as obesity and diabetes.

Panel 4D Summary: Ag3352—Expression of CG57883-01 gene is low/undetectable (CTs>35) across all of the samples on this panel (data not shown).

B. CG57881-01: Ring Finger Protein

Expression of gene CG557881-01 was assessed using the primer-probe set Ag3353, described in Table BA. Results of the RTQ-PCR runs are shown in Tables BB.

TABLE BA
Probe Name Ag3353

| | | SEQ ID | | Start |
|---|---|---|---|---|
| Primers | Sequences | NO. | Length | Position |
| Forward | 5′-agggccatttccttctataatg-3′ | 70 | 22 | 773 |
| Probe | TET-5′-tcagtgataggtcacatatcttcacattca-3′-TAMRA | 71 | 30 | 795 |
| Reverse | 5′-tggctcagtagcagaaattttc-3′ | 72 | 22 | 826 |

TABLE BB
Panel 4D

| | Rel. Exp. (%) Ag3353, | | Rel. Exp. (%) Ag3353, |
|---|---|---|---|
| Tissue Name | Run 165241913 | Tissue Name | Run 165241913 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 4.4 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 14.6 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 6.3 | Lung fibroblast IL-9 | 0.0 |
| Ramos (B cell) | 100.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 2.4 | Dermal fibroblast CCD1070 | 2.5 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 1.6 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 2.1 |
| HUVEC none | 0.0 | Kidney | 0.0 |
| HUVEC starved | 0.0 | | |

CNS_neurodegeneration_v1.0 Summary: Ag3353—Expression of the CG57881-01 gene is low/undetectable (CTs>35) across all of the samples on this panel (data not shown).

General_screening_panel_v1.4 Summary: Ag3353—Results from one experiment are not included. The amp plot indicates that there were experimental difficulties with this run.

Panel 4D Summary: Ag3353—Highest expression of the CG57881-01 gene is seen in ionomycin treated Ramos B cells (CT=31.7). Interestingly, there is low/undectable expression of this gene in untreated Ramos B cells (CT=35.7). Therefore, expression of this gene can be used to distinguish ionomycin treated Ramos B cells from the untreated cells and other samples used in this panel. In addition, expression of this gene in ionomycin stimulated B cells suggests that this gene may be involved in rheumatic disease including rheumatoid arthritis, lupus, osteoarthritis, and hyperproliferative B cell disorders.

Low expression of this gene is also detected in a liver cirrhosis sample (CT=34.4). The presence of this gene in liver cirrhosis (a component of which involves liver inflammation and fibrosis) suggests that antibodies to the protein encoded by this gene could also be used for the diagnosis of liver cirrhosis. Furthermore, therapeutic agents involving this gene may be useful in reducing or inhibiting the inflammation associated with fibrotic and inflammatory diseases.

C. CG57407-01: Brain cDNA, Clone MNCB-2146

Expression of gene CG57407-01 was assessed using the primer-probe set Ag3227, described in Table CA. Results of the RTQ-PCR runs are shown in Tables CB

TABLE CA
Probe Name Ag3227

| | | SED ID | | Start |
|---|---|---|---|---|
| Primers | Sequences | NO. | Length Position | |
| Forward | 5′-gcagtgctatcagagagctgtt-3′ | 73 | 22 | 628 |
| Probe | TET-5′-cccctggcctatgttctgctgctaag-3′-TAMRA | 74 | 26 | 675 |
| Reverse | 5′-tgtgtgttcacacagacaatga-3′ | 75 | 22 | 703 |

TABLE CB
General_screening_panel_v1.4

| | Rel. Exp. (%) Ag3227, | | Rel. Exp. (%) Ag3227, |
|---|---|---|---|
| Tissue Name | Run 214436795 | Tissue Name | Run 214436795 |
| Adipose | 6.3 | Renal ca. TK-10 | 19.9 |
| Melanoma* | 0.0 | Bladder | 16.2 |
| Hs688(A).T | | | |
| Melanoma* | 0.0 | Gastric ca. (liver met.) | 100.0 |
| Hs688(B).T | | NCI-N87 | |
| Melanoma* M14 | 2.8 | Gastric ca. KATO III | 0.0 |
| Melanoma* LOXIMVI | 0.0 | Colon ca. SW-948 | 0.0 |
| Melanoma* SK-MEL-5 | 17.2 | Colon ca. SW480 | 2.0 |
| Squamous cell | 2.9 | Colon ca.* (SW480 met) | 0.0 |
| carcinoma SCC-4 | | SW620 | |
| Testis Pool | 17.3 | Colon ca. HT29 | 7.6 |
| Prostate ca.* (bone | 25.5 | Colon ca. HCT-116 | 0.0 |
| met) PC-3 | | | |
| Prostate Pool | 4.9 | Colon ca. CaCo-2 | 8.8 |
| Placenta | 0.0 | Colon cancer tissue | 0.0 |
| Uterus Pool | 0.0 | Colon ca. SW1116 | 3.8 |
| Ovarian ca. OVCAR-3 | 4.6 | Colon ca. Colo-205 | 0.0 |
| Ovarian ca. SK-OV-3 | 61.6 | Colon ca. SW-48 | 0.0 |
| Ovarian ca. OVCAR-4 | 0.0 | Colon Pool | 3.6 |
| Ovarian ca. OVCAR-5 | 0.0 | Small Intestine Pool | 46.0 |
| Ovarian ca. IGROV-1 | 25.3 | Stomach Pool | 0.0 |
| Ovarian ca. OVCAR-8 | 0.0 | Bone Marrow Pool | 3.0 |
| Ovary | 0.0 | Fetal Heart | 0.0 |
| Breast ca. MCF-7 | 0.0 | Heart Pool | 0.0 |
| Breast ca. MDA-MB- | 0.0 | Lymph Node Pool | 9.7 |
| 231 | | | |
| Breast ca. BT 549 | 35.1 | Fetal Skeletal Muscle | 0.0 |
| Breast ca. T47D | 0.0 | Skeletal Muscle Pool | 29.1 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) | 0.0 |
| | | U87-MG | |
| Lung | 3.1 | CNS cancer (glio/astro) | 0.0 |
| | | U-118-MG | |
| Fetal Lung | 18.3 | CNS cancer (neuro; met) | 14.3 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) SNB- | 3.5 |
| | | 75 | |
| Lung ca. NCI-H146 | 34.2 | CNS cancer (glio) SNB- | 20.3 |
| | | 19 | |
| Lung ca. SHP-77 | 35.6 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 4.5 | Brain (fetal) | 14.0 |
| Lung ca. NCI-H460 | 0.0 | Brain (Hippocampus) | 0.0 |
| | | Pool | |
| Lung ca. HOP-62 | 12.0 | Cerebral Cortex Pool | 7.8 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.0 | Brain (whole) | 7.5 |
| Liver ca. HepG2 | 0.0 | Spinal Cord Pool | 3.8 |
| Kidney Pool | 36.1 | Adrenal Gland | 0.0 |
| Fetal Kidney | 0.0 | Pituitary gland Pool | 10.7 |
| Renal ca. 786-0 | 1.6 | Salivary Gland | 0.0 |
| Renal ca. A498 | 0.0 | Thyroid (female) | 0.0 |
| Renal ca. ACHN | 0.0 | Pancreatic ca. CAPAN2 | 2.6 |
| Renal ca. UO-31 | 3.8 | Pancreas Pool | 0.0 |

CNS_neurodegeneration_{—l v}1.0 Summary: Ag3227—Expression of this gene is low/undetectable (CTs>35) across all of the samples on this panel (data not shown).

General_screening_panel_v1.4 Summary: Ag3227 Highest expression of this gene is detected in gastric cancer cell line NCI-N87 (CT=33.7). Significant expression of this gene is also seen small intestine (CT=34.8), and ovarian cancer cell line SK-OV-3 (CT=34.4). Therefore, expression of this gene can be used to distinguish these samples from other samples on this panel. In addition, therapeutic modulation of the activity of this gene or its protein product, through the use of small molecule drugs, protein therapeutics or antibodies, might be beneficial in the treatment of gastric cancer or ovarian cancer.

Panel 2.2 Summary: Ag3227—Results from one experiment are not included. The amp plot indicates that there were experimental difficulties with this run.

Panel 4D Summary: Ag3227—Expression of this gene is low/undetectable (CTs>35) across all of the samples on this panel (data not shown).

D. CG58651-02: SM-20

Expression of gene CG58651-02 was assessed using the primer-probe set Ag3388, described in Table DA. Results of the RTQ-PCR runs are shown in Tables DB, DC and DD.

TABLE DA
Probe Name Ag3388

| | | SEQ ID | | |
|---|---|---|---|---|
| Primers | Sequences | NO. | Length Start Position | |
| Forward | 5′-gtacgtaaggcacgttgacaat-3′ | 76 | 22 | 1156 |
| Probe | TET-5′-atgggcgctgcatcacctgtatctat-3′-TAMRA | 77 | 26 | 1188 |
| Reverse | 5′-gcactaccttaacgtcccagtt-3′ | 78 | 22 | 1126 |

TABLE DB
CNS_neurodegeneration_v1.0

| | Rel. Exp. (%) | | Rel. Exp. (%) |
|---|---|---|---|
| | Ag3388, Run | | Ag3388, Run |
| Tissue Name | 210155039 | Tissue Name | 210155039 |
| AD 1 Hippo | 16.6 | Control (Path) 3 | 8.5 |
| | | Temporal Ctx | |
| AD 2 Hippo | 36.3 | Control (Path) 4 | 25.9 |
| | | Temporal Ctx | |
| AD 3 Hippo | 16.3 | AD 1 Occipital Ctx | 23.5 |
| AD 4 Hippo | 14.7 | AD 2 Occipital Ctx | 0.0 |
| | | (Missing) | |
| AD 5 hippo | 100.0 | AD 3 Occipital Ctx | 12.9 |
| AD 6 Hippo | 54.7 | AD 4 Occipital Ctx | 20.3 |
| Control 2 Hippo | 37.4 | AD 5 Occipital Ctx | 28.1 |
| Control 4 Hippo | 12.9 | AD 6 Occipital Ctx | 47.6 |
| Control (Path) 3 Hippo | 7.9 | Control 1 Occipital Ctx | 9.1 |
| AD 1 Temporal Ctx | 23.5 | Control 2 Occipital Ctx | 75.3 |
| AD 2 Temporal Ctx | 33.7 | Control 3 Occipital Ctx | 25.5 |
| AD 3 Temporal Ctx | 12.4 | Control 4 Occipital Ctx | 9.8 |
| AD 4 Temporal Ctx | 19.2 | Control (Path) 1 | 79.0 |
| | | Occipital Ctx | |
| AD 5 Inf Temporal Ctx | 84.1 | Control (Path) 2 | 12.4 |
| | | Occipital Ctx | |
| AD 5 SupTemporal Ctx | 56.3 | Control (Path) 3 | 5.8 |
| | | Occipital Ctx | |
| AD 6 Inf Temporal Ctx | 60.3 | Control (Path) 4 | 20.4 |
| | | Occipital Ctx | |
| AD 6 Sup Temporal Ctx | 46.0 | Control 1 Parietal Ctx | 15.3 |
| Control 1 Temporal Ctx | 8.4 | Control 2 Parietal Ctx | 40.6 |
| Control 2 Temporal Ctx | 39.8 | Control 3 Parietal Ctx | 0.6 |
| Control 3 Temporal Ctx | 18.2 | Control (Path) 1 | 60.7 |
| | | Parietal Ctx | |
| Control 4 Temporal Ctx | 11.8 | Control (Path) 2 | 23.2 |
| | | Parietal Ctx | |
| Control (Path) 1 | 59.0 | Control (Path) 3 | 7.8 |
| Temporal Ctx | | Parietal Ctx | |
| Control (Path) 2 | 11.3 | Control (Path) 4 | 40.6 |
| Temporal Ctx | | Parietal Ctx | |

TABLE DC
General_screening_panel_v1.4

| | Rel. Exp. (%) | | Rel. Exp. (%) |
|---|---|---|---|
| | Ag3388, Run | | Ag3388, Run |
| Tissue Name | 216821339 | Tissue Name | 216821339 |
| Adipose | 2.3 | Renal ca. TK-10 | 12.1 |
| Melanoma* | 7.4 | Bladder | 7.5 |
| Hs688(A).T | | | |
| Melanoma* Hs688(B).T | 10.5 | Gastric ca. (liver met.) | 18.4 |
| | | NCI-N87 | |
| Melanoma* M14 | 12.2 | Gastric ca. KATO III | 22.7 |
| Melanoma* LOXIMVI | 11.5 | Colon ca. SW-948 | 7.2 |
| Melanoma* SK-MEL-5 | 11.7 | Colon ca. SW480 | 24.3 |
| Squamous cell | 4.0 | Colon ca.* (SW480 met) | 7.2 |
| carcinoma SCC-4 | | SW620 | |
| Testis Pool | 9.7 | Colon ca. HT29 | 15.4 |
| Prostate ca.* (bone met) | 6.8 | Colon ca. HCT-116 | 20.7 |
| PC-3 | | | |
| Prostate Pool | 2.4 | Colon ca. CaCo-2 | 15.9 |
| Placenta | 19.3 | Colon cancer tissue | 8.1 |
| Uterus Pool | 1.1 | Colon ca. SW1116 | 2.7 |
| Ovarian ca. OVCAR-3 | 9.3 | Colon ca. Colo-205 | 6.3 |
| Ovarian ca. SK-OV-3 | 32.8 | Colon ca. SW-48 | 3.0 |
| Ovarian ca. OVCAR-4 | 8.8 | Colon Pool | 7.9 |
| Ovarian ca. OVCAR-5 | 35.6 | Small Intestine Pool | 7.4 |
| Ovarian ca. IGROV-1 | 19.2 | Stomach Pool | 6.0 |
| Ovarian ca. OVCAR-8 | 14.5 | Bone Marrow Pool | 2.4 |
| Ovary | 6.0 | Fetal Heart | 4.0 |
| Breast ca. MCF-7 | 41.2 | Heart Pool | 3.6 |
| Breast ca. MDA-MB- | 15.3 | Lymph Node Pool | 10.1 |
| 231 | | | |
| Breast ca. BT 549 | 27.7 | Fetal Skeletal Muscle | 1.7 |
| Breast ca. T47D | 100.0 | Skeletal Muscle Pool | 3.3 |
| Breast ca. MDA-N | 6.9 | Spleen Pool | 12.4 |
| Breast Pool | 9.3 | Thymus Pool | 7.2 |
| Trachea | 9.3 | CNS cancer (glio/astro) | 18.9 |
| | | U87-MG | |
| Lung | 1.9 | CNS cancer (glio/astro) U- | 20.7 |
| | | 118-MG | |
| Fetal Lung | 24.3 | CNS cancer (neuro; met) | 14.8 |
| | | SK-N-AS | |
| Lung ca. NCI-N417 | 2.0 | CNS cancer (astro) SF-539 | 14.8 |
| Lung ca. LX-1 | 6.7 | CNS cancer (astro) SNB- | 28.5 |
| | | 75 | |
| Lung ca. NCI-H146 | 9.2 | CNS cancer (glio) SNB-19 | 20.0 |
| Lung ca. SHP-77 | 11.3 | CNS cancer (glio) SF-295 | 27.9 |
| Lung ca. A549 | 26.4 | Brain (Amygdala) Pool | 6.5 |
| Lung ca. NCI-H526 | 14.7 | Brain (cerebellum) | 17.1 |
| Lung ca. NCI-H23 | 11.0 | Brain (fetal) | 10.4 |
| Lung ca. NCI-H460 | 3.7 | Brain (Hippocampus) Pool | 6.0 |
| Lung ca. HOP-62 | 7.2 | Cerebral Cortex Pool | 7.4 |
| Lung ca. NCI-H522 | 9.5 | Brain (Substantia nigra) | 7.6 |
| | | Pool | |
| Liver | 0.9 | Brain (Thalamus) Pool | 9.3 |
| Fetal Liver | 6.3 | Brain (whole) | 7.7 |
| Liver ca. HepG2 | 5.0 | Spinal Cord Pool | 4.9 |
| Kidney Pool | 15.3 | Adrenal Gland | 8.2 |
| Fetal Kidney | 5.7 | Pituitary gland Pool | 2.0 |
| Renal ca. 786-0 | 7.2 | Salivary Gland | 3.3 |
| Renal ca. A498 | 6.0 | Thyroid (female) | 4.9 |
| Renal ca. ACHN | 4.5 | Pancreatic ca. CAPAN2 | 12.1 |
| Renal ca. UO-31 | 7.0 | Pancreas Pool | 11.4 |

TABLE DD
Panel 4D

| | Rel. Exp. (%) Ag3388, | | Rel. Exp. (%) Ag3388, |
|---|---|---|---|
| Tissue Name | Run 165296476 | Tissue Name | Run 165296476 |
| Secondary Th1 act | 25.7 | HUVEC IL-1beta | 12.7 |
| Secondary Th2 act | 26.2 | HUVEC IFN gamma | 66.9 |
| Secondary Tr1 act | 28.3 | HUVEC TNF alpha + IFN | 51.4 |
| | | gamma | |
| Secondary Th1 rest | 21.5 | HUVEC TNF alpha + IL4 | 33.7 |
| Secondary Th2 rest | 17.8 | HUVEC IL-11 | 20.9 |
| Secondary Tr1 rest | 27.0 | Lung Microvascular EC none | 50.3 |
| Primary Th1 act | 22.8 | Lung Microvascular EC | 37.9 |
| | | TNF alpha + IL-1beta | |
| Primary Th2 act | 26.6 | Microvascular Dermal EC | 52.1 |
| | | none | |
| Primary Tr1 act | 30.8 | Microsvasular Dermal EC | 43.8 |
| | | TNF alpha + IL-1beta | |
| Primary Th1 rest | 88.9 | Bronchial epithelium | 49.3 |
| | | TNF alpha + IL1beta | |
| Primary Th2 rest | 36.3 | Small airway epithelium none | 14.5 |
| Primary Tr1 rest | 29.5 | Small airway epithelium | 44.1 |
| | | TNF alpha + IL-1beta | |
| CD45RA CD4 | 26.1 | Coronery artery SMC rest | 25.7 |
| lymphocyte act | | | |
| CD45RO CD4 | 30.4 | Coronery artery SMC | 12.9 |
| lymphocyte act | | TNF alpha + IL-1beta | |
| CD8 lymphocyte act | 33.7 | Astrocytes rest | 21.2 |
| Secondary CD8 | 23.7 | Astrocytes TNF alpha + IL- | 17.3 |
| lymphocyte rest | | 1beta | |
| Secondary CD8 | 12.6 | KU-812 (Basophil) rest | 45.7 |
| lymphocyte act | | | |
| CD4 lymphocyte none | 17.6 | KU-812 (Basophil) | 94.6 |
| | | PMA/ionomycin | |
| 2ry Th1/Th2/Tr1_anti- | 28.3 | CCD1106 (Keratinocytes) none | 29.5 |
| CD95 CH11 | | | |
| LAK cells rest | 31.9 | CCD1106 (Keratinocytes) | 29.9 |
| | | TNF alpha + IL-1beta | |
| LAK cells IL-2 | 28.3 | Liver cirrhosis | 5.8 |
| LAK cells IL-2 + IL-12 | 29.9 | Lupus kidney | 3.7 |
| LAK cells IL-2 + IFN | 40.1 | NCI-H292 none | 31.6 |
| gamma | | | |
| LAK cells IL-2 + IL-18 | 48.0 | NCI-H292 IL-4 | 37.6 |
| LAK cells | 20.2 | NCI-H292 IL-9 | 37.1 |
| PMA/ionomycin | | | |
| NK Cells IL-2 rest | 33.2 | NCI-H292 IL-13 | 27.0 |
| Two Way MLR 3 day | 42.3 | NCI-H292 IFN gamma | 28.9 |
| Two Way MLR 5 day | 28.1 | HPAEC none | 33.2 |
| Two Way MLR 7 day | 24.8 | HPAEC TNF alpha + IL-1beta | 57.8 |
| PBMC rest | 22.5 | Lung fibroblast none | 13.4 |
| PBMC PWM | 80.1 | Lung fibroblast TNF alpha + | 14.5 |
| | | IL-1beta | |
| PBMC PHA-L | 42.9 | Lung fibroblast IL-4 | 31.4 |
| Ramos (B cell) none | 19.8 | Lung fibroblast IL-9 | 22.2 |
| Ramos (B cell) ionomycin | 74.2 | Lung fibroblast IL-13 | 18.3 |
| B lymphocytes PWM | 100.0 | Lung fibroblast IFN gamma | 43.2 |
| B lymphocytes CD40L | 60.7 | Dermal fibroblast CCD1070 | 42.0 |
| and IL-4 | | rest | |
| EOL-1 dbcAMP | 32.3 | Dermal fibroblast CCD1070 | 93.3 |
| | | TNF alpha | |
| EOL-1 dbcAMP | 42.3 | Dermal fibroblast CCD1070 | 30.4 |
| PMA/ionomycin | | IL-1beta | |
| Dendritic cells none | 30.8 | Dermal fibroblast IFN gamma | 32.5 |
| Dendritic cells LPS | 25.9 | Dermal fibroblast IL-4 | 46.7 |
| Dendritic cells anti-CD40 | 31.4 | IBD Colitis 2 | 1.5 |
| Monocytes rest | 31.6 | IBD Crohn's | 0.8 |
| Monocytes LPS | 43.5 | Colon | 25.2 |
| Macrophages rest | 51.1 | Lung | 28.5 |
| Macrophages LPS | 28.1 | Thymus | 29.3 |
| HUVEC none | 31.2 | Kidney | 59.5 |
| HUVEC starved | 63.3 | | |

CNS_neurodegeneration_v1.0 Summary: Ag3388 This panel confirms the expression of the CG58651-02 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3388 Highest expression of the CG58651-02 gene is seen in a breast cancer cell line T47D (CT=27.3). Therefore, expression of this gene can be used to distinguish this sample from other samples on this panel. In addition, high expression of these gene is also detected in ovarian, breast, CNS, lung, gastric and colon cancer. Therefore, therapeutic modulation of the activity of this gene or its protein product, through the use of small molecule drugs, protein therapeutics or antibodies, might be beneficial in the treatment of these cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal (CT=31) when compared to adult liver (CT=34). This observation suggests that expression of this gene can be used to distinguish fetal from adult liver.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

CG58651-02 codes for a variant of EGLN2 protein. EGLN2 is a homologue of Caenorhabditis elegans gene eg1-9. See, Online Mendelian Inheritance in Man (OMIM) accno. 606424. Recently, it has been shown that C. elegans EGL-9 and mammalian homologs define a family of dioxygenases that regulate HIF by prolyl hydroxylation.. See, e.g., Epstein et al. (2001) Cell 107(l):43-54. HIF is a transcriptional complex that plays a central role in mammalian oxygen homeostasis. It is activated in a broad array of ischemic/hypoxic and neoplastic diseases. Therefore, therapeutic modulation of the activity of the protein encoded by CG58651-02 gene may be beneficial in the treatment ischemic/hypoxic and neoplastic diseases.

Panel 4D Summary: Ag3388 Highest expresion of the CG58651-02 gene is seen in PWM treated B lymphocytes (CT=29.4). However, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

E. CG59574-01: Synaptonemal Complex Protein

Expression of gene CG59574-01 was assessed using the primer-probe set Ag3477, described in Table EA. Results of the RTQ-PCR runs are shown in Tables EB, and EC.

TABLE EA
Probe Name Ag3477

| | | | | |
|---|---|---|---|---|
| | | SEQ ID | | |
| Primers | Sequences | no. | Length | Start Position |

| | | | | |
|---|---|---|---|---|
| Forward | 5′-tgaacatgtttggaaaacacaa-3′ | 79 | 22 | 333 |
| Probe | TET-5═-caagatcaaaggcagaagcttaacca-3′-TAMRA | 80 | 26 | 355 |
| Reverse | 5′-ctaaatcccactgctgaaacaa-3′ | 81 | 22 | 406 |

TABLE EB
General_screening_panel_v1.4

| | Rel. Exp. (%) | | Rel. Exp. (%) |
|---|---|---|---|
| | Ag3477, Run | | Ag3477, Run |
| Tissue Name | 217119696 | Tissue Name | 217119696 |
| Adipose | 0.2 | Renal ca. TK-10 | 2.0 |
| Melanoma* | 0.2 | Bladder | 0.4 |
| Hs688(A).T | | | |
| Melanoma* Hs688(B).T | 0.0 | Gastric ca. (liver met.) | 1.5 |
| | | NCI-N87 | |
| Melanoma* M14 | 1.4 | Gastric ca. KATO III | 0.0 |
| Melanoma* LOXIMVI | 0.0 | Colon ca. SW-948 | 0.0 |
| Melanoma* SK-MEL-5 | 1.7 | Colon ca. SW480 | 0.4 |
| Squamous cell | 0.0 | Colon ca.* (SW480 met) | 0.6 |
| carcinoma SCC-4 | | SW620 | |
| Testis Pool | 100.0 | Colon ca. HT29 | 0.0 |
| Prostate ca.* (bone met) | 0.2 | Colon ca. HCT-116 | 0.5 |
| PC-3 | | | |
| Prostate Pool | 0.7 | Colon ca. CaCo-2 | 0.4 |
| Placenta | 0.0 | Colon cancer tissue | 0.2 |
| Uterus Pool | 0.0 | Colon ca. SW1116 | 0.0 |
| Ovarian ca. OVCAR-3 | 0.7 | Colon ca. Colo-205 | 0.0 |
| Ovarian ca. SK-OV-3 | 1.4 | Colon ca. SW-48 | 0.7 |
| Ovarian ca. OVCAR-4 | 0.0 | Colon Pool | 0.2 |
| Ovarian ca. OVCAR-5 | 0.9 | Small Intestine Pool | 0.0 |
| Ovarian ca. IGROV-1 | 0.3 | Stomach Pool | 0.0 |
| Ovarian ca. OVCAR-8 | 0.4 | Bone Marrow Pool | 0.0 |
| Ovary | 0.0 | Fetal Heart | 0.0 |
| Breast ca. MCF-7 | 1.6 | Heart Pool | 0.0 |
| Breast ca. MDA-MB- | 0.7 | Lymph Node Pool | 0.6 |
| 231 | | | |
| Breast ca. BT 549 | 0.3 | Fetal Skeletal Muscle | 0.2 |
| Breast ca. T47D | 0.4 | Skeletal Muscle Pool | 1.0 |
| Breast ca. MDA-N | 1.3 | Spleen Pool | 1.0 |
| Breast Pool | 0.0 | Thymus Pool | 1.0 |
| Trachea | 0.5 | CNS cancer (glio/astro) | 0.7 |
| | | U87-MG | |
| Lung | 0.3 | CNS cancer (glio/astro) U- | 0.2 |
| | | 118-MG | |
| Fetal Lung | 0.0 | CNS cancer (neuro; met) | 1.2 |
| | | SK-N-AS | |
| Lung ca. NCI-N417 | 0.7 | CNS cancer (astro) SF-539 | 0.0 |
| Lung ca. LX-1 | 1.4 | CNS cancer (astro) SNB- | 0.0 |
| | | 75 | |
| Lung ca. NCI-H146 | 0.8 | CNS cancer (glio) SNB-19 | 0.4 |
| Lung ca. SHP-77 | 0.0 | CNS cancer (glio) SF-295 | 0.7 |
| Lung ca. A549 | 0.0 | Brain (Amygdala) Pool | 0.2 |
| Lung ca. NCI-H526 | 0.0 | Brain (cerebellum) | 1.4 |
| Lung ca. NCI-H23 | 0.4 | Brain (fetal) | 2.0 |
| Lung ca. NCI-H460 | 0.0 | Brain (Hippocampus) Pool | 0.2 |
| Lung ca. HOP-62 | 0.2 | Cerebral Cortex Pool | 0.6 |
| Lung ca. NCI-H522 | 0.0 | Brain (Substantia nigra) | 0.0 |
| | | Pool | |
| Liver | 0.0 | Brain (Thalamus) Pool | 0.4 |
| Fetal Liver | 0.2 | Brain (whole) | 0.0 |
| Liver ca. HepG2 | 0.0 | Spinal Cord Pool | 0.4 |
| Kidney Pool | 0.2 | Adrenal Gland | 0.2 |
| Fetal Kidney | 0.0 | Pituitary gland Pool | 0.0 |
| Renal ca. 786-0 | 0.3 | Salivary Gland | 0.0 |
| Renal ca. A498 | 0.2 | Thyroid (female) | 0.0 |
| Renal ca. ACHN | 0.2 | Pancreatic ca. CAPAN2 | 0.5 |
| Renal ca UO-31 | 0.6 | Pancreas Pool | 0.3 |

TABLE ED
Panel 4D

| | Rel. Exp. (%) Ag3477, | | Rel. Exp. (%) Ag3477, |
|---|---|---|---|
| Tissue Name | Run 166441539 | Tissue Name | Run 166441539 |
| Secondary Th1 act | 25.5 | HUVEC IL-1beta | 3.0 |
| Secondary Th2 act | 5.2 | HUVEC IFN gamma | 0.0 |
| Secondary Tr1 act | 31.0 | HUVEC TNF alpha + IFN | 2.0 |
| | | gamma | |
| Secondary Th1 rest | 18.7 | HUVEC TNF alpha + IL4 | 1.6 |
| Secondary Th2 rest | 3.1 | HUVEC IL-11 | 0.0 |
| Secondary Tr1 rest | 7.6 | Lung Microvascular EC none | 1.4 |
| Primary Th1 act | 4.9 | Lung Microvascular EC | 0.0 |
| | | TNF alpha + IL-1beta | |
| Primary Th2 act | 9.6 | Microvascular Dermal EC | 0.0 |
| | | none | |
| Primary Tr1 act | 18.3 | Microvascular Dermal EC | 0.0 |
| | | TNF alpha + IL-1beta | |
| Primary Th1 rest | 41.8 | Bronchial epithelium | 6.4 |
| | | TNF alpha + IL-1beta | |
| Primary Th2 rest | 18.0 | Small airway epithelium none | 1.6 |
| Primary Tr1 rest | 16.5 | Small airway epithelium | 48.6 |
| | | TNF alpha + IL-1beta | |
| CD45RA CD4 | 7.3 | Coronery artery SMC rest | 0.0 |
| lymphocyte act | | | |
| CD45RO CD4 | 24.0 | Coronery artery SMC | 2.1 |
| lymphocyte act | | TNF alpha + IL-1beta | |
| CD8 lymphocyte act | 18.8 | Astrocytes rest | 7.2 |
| Secondary CD8 | 22.7 | Astrocytes TNF alpha + IL- | 18.7 |
| lymphocyte rest | | 1beta | |
| Secondary CD8 | 21.2 | KU-812 (Basophil) rest | 2.5 |
| lymphocyte act | | | |
| CD4 lymphocyte none | 10.5 | KU-812 (Basophil) | 37.1 |
| | | PMA/ionomycin | |
| 2ry Th1/Th2/Tr1_anti- | 15.4 | CCD1106 (Keratinocytes) none | 3.8 |
| CD95 CH11 | | | |
| LAK cells rest | 8.0 | CCD1106 (Keratinocytes) | 10.3 |
| | | TNF alpha + IL-1beta | |
| LAK cells IL-2 | 44.1 | Liver cirrhosis | 0.0 |
| LAK cells IL-2 + IL-12 | 12.1 | Lupus kidney | 2.1 |
| LAK cells IL-2 + IFN | 62.9 | NCI-H292 none | 19.8 |
| gamma | | | |
| LAK cells IL-2 + IL-18 | 38.4 | NCI-H292 IL-4 | 16.6 |
| LAK cells | 3.5 | NCI-H292 IL-9 | 31.2 |
| PMA/ionomycin | | | |
| NK Cells IL-2 rest | 52.5 | NCI-H292 IL-13 | 6.7 |
| Two Way MLR 3 day | 14.8 | NCI-H292 IFN gamma | 2.0 |
| Two Way MLR 5 day | 22.8 | HPAEC none | 1.1 |
| Two Way MLR 7 day | 7.2 | HPAEC TNF alpha + IL-1beta | 0.0 |
| PBMC rest | 7.7 | Lung fibroblast none | 6.1 |
| PBMC PWM | 23.7 | Lung fibroblast TNF alpha + | 6.2 |
| | | IL-1beta | |
| PBMC PHA-L | 8.2 | Lung fibroblast IL-4 | 4.2 |
| Ramos (B cell) none | 18.6 | Lung fibroblast IL-9 | 1.7 |
| Ramos (B cell) ionomycin | 9.2 | Lung fibroblast IL-13 | 0.0 |
| B lymphocytes PWM | 38.4 | Lung fibroblast IFN gamma | 6.1 |
| B lymphocytes CD40L | 2.0 | Dermal fibroblast CCD1070 | 0.0 |
| and IL-4 | | rest | |
| EOL-1 dbcAMP | 8.5 | Dermal fibroblast CCD1070 | 100.0 |
| | | TNF alpha | |
| EOL-1 dbcAMP | 20.2 | Dermal fibroblast CCD1070 | 1.3 |
| PMA/ionomycin | | IL-1beta | |
| Dendritic cells none | 16.4 | Dermal fibroblast IFN gamma | 4.0 |
| Dendritic cells LPS | 7.3 | Dermal fibroblast IL-4 | 3.1 |
| Dendritic cells anti-CD40 | 0.9 | IBD Colitis 2 | 0.7 |
| Monocytes rest | 13.8 | IBD Crohn's | 5.8 |
| Monocytes LPS | 41.8 | Colon | 8.5 |
| Macrophages rest | 15.6 | Lung | 1.4 |
| Macrophages LPS | 7.6 | Thymus | 12.9 |
| HUVEC none | 2.0 | Kidney | 59.0 |
| HUVEC starved | 2.8 | | |

CNS_neurodegeneration_v1.0 Summary: Ag3477 Expression of the CG59574-01 gene is low/undetectable in all samples on this panel (CTs>34.3). (Data not shown.)

General_screening_panel_v1.4 Summary: Ag3477 Expression of the CG59574-01 gene is restricted to the testis (CT=30). Thus, expression of this gene could be used to differentiate between this sample and other samples on this panel and as a marker for testicular tissue. Furthermore, therapeutic modulation of the expression or function of this gene may be effective in the treatment of male infertility and hypogonadism.

Panel 4D Summary: Ag3477 Highest expression of the CG59574-01 gene is seen in dermal fibroblasts treated with TNF alpha (CT=31.7). The significant levels of expression in this sample suggests that this gene product may be involved in skin disorders, including psoriasis. In addition, this transcript is expressed in T cells, LAK cells, macrophages, and dendritic cells. In addition to these hematopoietic cell types, expression is also seen in the PMA/ionomycin treated basophil cell line KU-812, small airway epithelium treated with TNF-alpha and IL-1 beta, and treated and untreated samples from the mucoepidermoid cell line H292. Thymus and kidney also express low levels of the transcript. Thus, this transcript or the protein it encodes could be used to design therapeutics that could be important in the regulation of the function of antigen presenting cells (macrophages and dendritic cells) or T cells and be important in the treatment of asthma, emphysema, psoriasis, arthritis, and IBD.

F. CG59536-01: Paraneoplastic Antigen

Expression of gene CG59536-01 was assessed using the primer-probe set Ag3458, described in Table FA. Results of the RTQ-PCR runs are shown in Tables FB, FC, FD, and FE.

TABLE FA
Probe Name Ag3458

| | | SEQ ID | | Start |
|---|---|---|---|---|
| Primers | Sequences | NO. | Length | Position |
| Forward | 5′-ttctgagccacagtaaggatct-3′ | 82 | 22 | 1297 |
| Probe | TET-5′-tccagccctaaatgagtccttgactg-3′-TAMRA | 83 | 26 | 1321 |
| Reverse | 5′-ccctctcctgttattcccatta-3′ | 84 | 122 | 1361 |

TABLE FB
CNS_neurodegeneration_v1.0

| | Rel. Exp. (%) | | Rel. Exp. (%) |
|---|---|---|---|
| | Ag3458, Run | | Ag3458, Run |
| Tissue Name | 210375286 | Tissue Name | 210375286 |
| AD 1 Hippo | 17.6 | Control (Path) 3 | 7.0 |
| | | Temporal Ctx | |
| AD 2 Hippo | 23.2 | Control (Path) 4 | 55.5 |
| | | Temporal Ctx | |
| AD 3 Hippo | 9.2 | AD 1 Occipital Ctx | 19.9 |
| AD 4 Hippo | 7.5 | AD 2 Occipital Ctx | 0.0 |
| | | (Missing) | |
| AD 5 hippo | 86.5 | AD 3 Occipital Ctx | 12.3 |
| AD 6 Hippo | 47.0 | AD 4 Occipital Ctx | 43.8 |
| Control 2 Hippo | 25.3 | AD 5 Occipital Ctx | 37.4 |
| Control 4 Hippo | 9.7 | AD 6 Occipital Ctx | 32.5 |
| Control (Path) 3 Hippo | 10.0 | Control 1 Occipital Ctx | 7.9 |
| AD 1 Temporal Ctx | 23.8 | Control 2 Occipital Ctx | 40.1 |
| AD 2 Temporal Ctx | 66.0 | Control 3 Occipital Ctx | 11.1 |
| AD 3 Temporal Ctx | 14.9 | Control 4 Occipital Ctx | 10.2 |
| AD 4 Temporal Ctx | 40.3 | Control (Path) 1 | 81.2 |
| | | Occipital Ctx | |
| AD 5 Inf Temporal Ctx | 100.0 | Control (Path) 2 | 10.8 |
| | | Occipital Ctx | |
| AD 5 SupTemporal Ctx | 49.3 | Control (Path) 3 | 3.1 |
| | | Occipital Ctx | |
| AD 6 Inf Temporal Ctx | 70.7 | Control (Path) 4 | 10.7 |
| | | Occipital Ctx | |
| AD 6 Sup Temporal Ctx | 89.5 | Control 1 Parietal Ctx | 9.7 |
| Control 1 Temporal Ctx | 8.3 | Control 2 Parietal Ctx | 52.9 |
| Control 2 Temporal Ctx | 33.4 | Control 3 Parietal Ctx | 12.3 |
| Control 3 Temporal Ctx | 9.4 | Control (Path) 1 | 46.3 |
| | | Parietal Ctx | |
| Control 4 Temporal Ctx | 23.0 | Control (Path) 2 | 16.5 |
| | | Parietal Ctx | |
| Control (Path) 1 | 67.8 | Control (Path) 3 | 2.2 |
| Temporal Ctx | | Parietal Ctx | |
| Control (Path) 2 | 36.9 | Control (Path) 4 | 34.9 |
| Temporal Ctx | | Parietal Ctx | |

TABLE FC
General_screening_panel_v1.4

| | Rel. Exp. (%) | | Rel. Exp. (%) |
|---|---|---|---|
| | Ag3458, Run | | Ag3458, Run |
| Tissue Name | 213390552 | Tissue Name | 213390552 |
| Adipose | 1.0 | Renal ca. TK-10 | 7.2 |
| Melanoma* | 4.7 | Bladder | 3.9 |
| Hs688(A).T | | | |
| Melanoma* Hs688(B).T | 3.6 | Gastric ca. (liver met.) | 3.1 |
| | | NCI-N87 | |
| Melanoma* M14 | 8.0 | Gastric ca. KATO III | 2.5 |
| Melanoma* LOXIMVI | 1.7 | Colon ca. SW-948 | 1.0 |
| Melanoma* SK-MEL-5 | 1.5 | Colon ca. SW480 | 9.8 |
| Squamous cell | 1.0 | Colon ca.* (SW480 met) | 37.9 |
| carcinoma SCC-4 | | SW620 | |
| Testis Pool | 7.9 | Colon ca. HT29 | 0.3 |
| Prostate ca.* (bone met) | 4.5 | Colon ca. HCT-116 | 1.4 |
| PC-3 | | | |
| Prostate Pool | 2.5 | Colon ca. CaCo-2 | 1.6 |
| Placenta | 0.2 | Colon cancer tissue | 0.2 |
| Uterus Pool | 2.7 | Colon ca. SW1116 | 0.3 |
| Ovarian ca. OVCAR-3 | 9.7 | Colon ca. Colo-205 | 0.3 |
| Ovarian ca. SK-OV-3 | 4.7 | Colon ca. SW-48 | 0.4 |
| Ovarian ca. OVCAR-4 | 1.1 | Colon Pool | 8.2 |
| Ovarian ca. OVCAR-5 | 2.0 | Small Intestine Pool | 12.2 |
| Ovarian ca. IGROV-1 | 4.2 | Stomach Pool | 6.6 |
| Ovarian ca. OVCAR-8 | 19.8 | Bone Marrow Pool | 3.4 |
| Ovary | 10.2 | Fetal Heart | 100.0 |
| Breast ca. MCF-7 | 6.4 | Heart Pool | 3.4 |
| Breast ca. MDA-MB- | 9.3 | Lymph Node Pool | 18.4 |
| 231 | | | |
| Breast ca. BT 549 | 21.9 | Fetal Skeletal Muscle | 3.0 |
| Breast ca. T47D | 3.4 | Skeletal Muscle Pool | 1.8 |
| Breast ca. MDA-N | 8.3 | Spleen Pool | 11.3 |
| Breast Pool | 10.5 | Thymus Pool | 5.8 |
| Trachea | 3.2 | CNS cancer (glio/astro) | 3.3 |
| | | U87-MG | |
| Lung | 25.9 | CNS cancer (glio/astro) U- | 8.9 |
| | | 118-MG | |
| Fetal Lung | 13.6 | CNS cancer (neuro; met) | 16.5 |
| | | SK-N-AS | |
| Lung ca. NCI-N417 | 3.1 | CNS cancer (astro) SF-539 | 1.8 |
| Lung ca. LX-1 | 7.5 | CNS cancer (astro) SNB- | 11.7 |
| | | 75 | |
| Lung ca. NCI-H146 | 8.4 | CNS cancer (glio) SNB-19 | 7.1 |
| Lung ca. SHP-77 | 9.0 | CNS cancer (glio) SF-295 | 15.4 |
| Lung ca. A549 | 2.6 | Brain (Amygdala) Pool | 11.2 |
| Lung ca. NCI-H526 | 1.8 | Brain (cerebellum) | 26.4 |
| Lung ca. NCI-H23 | 5.0 | Brain (fetal) | 54.3 |
| Lung ca. NCI-H460 | 3.7 | Brain (Hippocampus) Pool | 15.2 |
| Lung ca. HOP-62 | 5.9 | Cerebral Cortex Pool | 14.2 |
| Lung ca. NCI-H522 | 52.9 | Brain (Substantia nigra) | 7.1 |
| | | Pool | |
| Liver | 0.0 | Brain (Thalamus) Pool | 13.4 |
| Fetal Liver | 1.7 | Brain (whole) | 4.6 |
| Liver ca. HepG2 | 0.9 | Spinal Cord Pool | 13.1 |
| Kidney Pool | 20.9 | Adrenal Gland | 4.0 |
| Fetal Kidney | 6.4 | Pituitary gland Pool | 4.0 |
| Renal ca. 786-0 | 5.6 | Salivary Gland | 0.0 |
| Renal ca. A498 | 0.0 | Thyroid (female) | 0.4 |
| Renal ca. ACHN | 4.8 | Pancreatic ca. CAPAN2 | 0.4 |
| Renal ca. UO-31 | 8.9 | Pancreas Pool | 14.9 |

TABLE FD
Panel 2.2

| | Rel. Exp. (%) Ag3458, | | Rel. Exp. (%) Ag3458, |
|---|---|---|---|
| Tissue Name | Run 173761865 | Tissue Name | Run 173761865 |
| Normal Colon | 0.1 | Kidney Margin (OD04348) | 0.1 |
| Colon cancer (OD06064) | 0.0 | Kidney malignant cancer | 0.1 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 0.1 |
| | | 01) | |
| Colon Margin (OD06159) | 0.0 | Kidney Margin (OD04450- | 0.1 |
| | | 03) | |
| Colon cancer (OD06297-04) | 0.0 | Kidney Cancer 8120613 | 0.0 |
| Colon Margin (OD06297- | 0.1 | Kidney Margin 8120614 | 0.0 |
| 05) | | | |
| CC Gr.2 ascend colon | 0.0 | Kidney Cancer 9010320 | 0.0 |
| (ODO3921) | | | |
| CC Margin (ODO3921) | 0.0 | Kidney Margin 9010321 | 0.1 |
| Colon cancer metastasis | 0.0 | Kidney Cancer 8120607 | 0.0 |
| (OD06104) | | | |
| Lung Margin (OD06104) | 0.0 | Kidney Margin 8120608 | 0.0 |
| Colon mets to lung | 0.0 | Normal Uterus | 0.2 |
| (OD04451-01) | | | |
| Lung Margin (OD04451-02) | 0.1 | Uterine Cancer 064011 | 0.0 |
| Normal Prostate | 0.0 | Normal Thyroid | 0.0 |
| Prostate Cancer (OD04410) | 0.1 | Thyroid Cancer 064010 | 0.1 |
| Prostate Margin (OD04410) | 0.2 | Thyroid Cancer A30152 | 0.1 |
| Normal Ovary | 0.2 | Thyroid Margin A302153 | 0.0 |
| Ovarian cancer (OD06283- | 0.1 | Normal Breast | 0.1 |
| 03) | | | |
| Ovarian Margin (OD06283- | 0.1 | Breast Cancer (OD04566) | 0.0 |
| 07) | | | |
| Ovarian Cancer 064008 | 1.5 | Breast Cancer 1024 | 0.0 |
| Ovarian cancer (OD06145) | 0.1 | Breast Cancer (OD04590- | 0.0 |
| | | 01) | |
| Ovarian Margin (OD06145) | 0.0 | Breast Cancer Mets | 0.1 |
| | | (OD04590-03) | |
| Ovarian cancer (OD06455- | 0.0 | Breast Cancer Metastasis | 0.1 |
| 03) | | (OD04655-05) | |
| Ovarian Margin (OD06455- | 0.1 | Breast Cancer 064006 | 0.0 |
| 07) | | | |
| Normal Lung | 0.0 | Breast Cancer 9100266 | 0.0 |
| Invasive poor diff. lung | 0.0 | Breast Margin 9100265 | 0.0 |
| adeno (ODO4945-01 | | | |
| Lung Margin (ODO4945- | 0.0 | Breast Cancer A209073 | 0.1 |
| 03) | | | |
| Lung Malignant Cancer | 0.0 | Breast Margin A2090734 | 0.2 |
| (OD03126) | | | |
| Lung Margin (OD03126) | 100.0 | Breast cancer (OD06083) | 0.3 |
| Lung Cancer (OD05014A) | 0.0 | Breast cancer node | 0.2 |
| | | metastasis (OD06083) | |
| Lung Margin (OD05014B) | 0.1 | Normal Liver | 0.0 |
| Lung cancer (OD06081) | 0.0 | Liver Cancer 1026 | 0.0 |
| Lung Margin (OD06081) | 0.1 | Liver Cancer 1025 | 0.0 |
| Lung Cancer (OD04237-01) | 0.1 | Liver Cancer 6004-T | 0.0 |
| Lung Margin (OD04237-02) | 0.0 | Liver Tissue 6004-N | 0.1 |
| Ocular Melanoma | 0.1 | Liver Cancer 6005-T | 0.0 |
| Metastasis | | | |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 0.0 | Normal Bladder | 0.2 |
| Normal Kidney | 0.0 | Bladder Cancer 1023 | 0.0 |
| Kidney Ca, Nuclear grade 2 | 0.1 | Bladder Cancer A302173 | 0.1 |
| (OD04338) | | | |
| Kidney Margin (OD04338) | 0.0 | Normal Stomach | 0.4 |
| Kidney Ca Nuclear grade | 0.1 | Gastric Cancer 9060397 | 0.0 |
| 1/2 (OD04339) | | | |
| Kidney Margin (OD04339) | 0.0 | Stomach Margin 9060396 | 0.1 |
| Kidney Ca, Clear cell type | 0.1 | Gastric Cancer 9060395 | 0.7 |
| (OD04340) | | | |
| Kidney Margin (OD04340) | 0.1 | Stomach Margin 9060394 | 0.0 |
| Kidney Ca, Nuclear grade 3 | 0.0 | Gastric Cancer 064005 | 0.1 |
| (OD04348) | | | |

TABLE FE
Panel 4D

| | Rel. Exp. (%) Ag3458, | | Rel. Exp. (%) Ag3458, |
|---|---|---|---|
| Tissue Name | Run 166417096 | Tissue Name | Run 166417096 |
| Secondary Th1 act | 7.7 | HUVEC IL-1beta | 7.1 |
| Secondary Th2 act | 5.2 | HUVEC IFN gamma | 15.7 |
| Secondary Tr1 act | 12.3 | HUVEC TNF alpha + IFN | 7.7 |
| | | gamma | |
| Secondary Th1 rest | 6.9 | HUVEC TNF alpha + IL4 | 15.3 |
| Secondary Th2 rest | 4.6 | HUVEC IL-11 | 23.5 |
| Secondary Tr1 rest | 8.4 | Lung Microvascular EC none | 13.3 |
| Primary Th1 act | 7.1 | Lung Microvascular EC | 23.2 |
| | | TNF alpha + IL-1beta | |
| Primary Th2 act | 14.6 | Microvascular Dermal EC | 16.0 |
| | | none | |
| Primary Tr1 act | 11.7 | Microsvasular Dermal EC | 13.0 |
| | | TNF alpha + IL-1beta | |
| Primary Th1 rest | 23.5 | Bronchial epithelium | 0.0 |
| | | TNF alpha + IL1beta | |
| Primary Th2 rest | 22.8 | Small airway epithelium none | 0.0 |
| Primary Tr1 rest | 7.9 | Small airway epithelium | 3.8 |
| | | TNF alpha + IL-1beta | |
| CD45RA CD4 | 19.5 | Coronery artery SMC rest | 5.8 |
| lymphocyte act | | | |
| CD45RO CD4 | 19.3 | Coronery artery SMC | 1.1 |
| lymphocyte act | | TNF alpha + IL-1beta | |
| CD8 lymphocyte act | 39.2 | Astrocytes rest | 24.1 |
| Secondary CD8 | 11.0 | Astrocytes TNF alpha + IL- | 35.8 |
| lymphocyte rest | | 1beta | |
| Secondary CD8 | 27.5 | KU-812 (Basophil) rest | 11.5 |
| lymphocyte act | | | |
| CD4 lymphocyte none | 27.7 | KU-812 (Basophil) | 12.1 |
| | | PMA/ionomycin | |
| 2ry Th1/Th2/Tr1_anti- | 13.7 | CCD1106 (Keratinocytes) none | 7.9 |
| CD95 CH11 | | | |
| LAK cells rest | 19.8 | CCD1106 (Keratinocytes) | 18.7 |
| | | TNF alpha + IL-1beta | |
| LAK cells IL-2 | 82.4 | Liver cirrhosis | 11.2 |
| LAK cells IL-2 + IL-12 | 22.7 | Lupus kidney | 4.1 |
| LAK cells IL-2 + IFN | 57.4 | NCI-H292 none | 3.4 |
| gamma | | | |
| LAK cells IL-2 + IL-18 | 24.8 | NCI-H292 IL-4 | 5.0 |
| LAK cells | 2.4 | NCI-H292 IL-9 | 8.4 |
| PMA/ionomycin | | | |
| NK Cells IL-2 rest | 59.9 | NCI-H292 IL-13 | 1.2 |
| Two Way MLR 3 day | 44.1 | NCI-H292 IFN gamma | 0.9 |
| Two Way MLR 5 day | 31.9 | HPAEC none | 21.6 |
| Two Way MLR 7 day | 22.4 | HPAEC TNF alpha + IL-1Beta | 18.6 |
| PBMC rest | 14.9 | Lung fibroblast none | 20.4 |
| PBMC PWM | 21.5 | Lung fibroblast TNF alpha + | 11.0 |
| | | IL-1Beta | |
| PBMC PHA-L | 7.4 | Lung fibroblast IL-4 | 10.9 |
| Ramos (B cell) none | 67.8 | Lung fibroblast IL-9 | 5.5 |
| Ramos (B cell) ionomycin | 30.4 | Lung fibroblast IL-13 | 6.4 |
| B lymphocytes PWM | 34.2 | Lung fibroblast IFN gamma | 13.4 |
| B lymphocytes CD40L | 23.7 | Dermal fibroblast CCD1070 | 20.7 |
| and IL-4 | | rest | |
| EOL-1 dbcAMP | 2.1 | Dermal fibroblast CCD1070 | 44.4 |
| | | TNF alpha | |
| EOL-1 dbcAMP | 3.5 | Dermal fibroblast CCD1070 | 2.0 |
| PMA/ionomycin | | IL-1Beta | |
| Dendritic cells none | 6.7 | Dermal fibroblast IFN gamma | 0.7 |
| Dendritic cells LPS | 3.6 | Dermal fibroblast IL-4 | 2.6 |
| Dendritic cells anti-CD40 | 2.6 | IBD Colitis 2 | 6.3 |
| Monocytes rest | 4.3 | IBD Crohn's | 3.4 |
| Monocytes LPS | 6.5 | Colon | 100.0 |
| Macrophages rest | 20.2 | Lung | 9.9 |
| Macrophages LPS | 2.0 | Thymus | 7.4 |
| HUVEC none | 39.5 | Kidney | 25.0 |
| HUVEC starved | 57.8 | | |

CNS_neurodegeneration_v1.0 Summary: Ag3458 This panel confirms the expression of CG59536-01 gene at significant levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3458 Expression of the CG59536-01 gene is widespread throughout this panel, with highest expression in the fetal heart (CT=29.6). In addition, expression of this gene appears to be higher in fetal heart when compared to expression in the adult heart (CT=34.5). Thus, expression of this gene could be used to differentiate between adult and fetal heart.

This gene is also expressed at moderate levels in samples derived from colon cancer and lung cancer cell lines. Thus, expression of this gene could be used to differentiate between these samples and other samples on this panel and as a marker to detect the presence of colon and lung cancer. Furthermore, therapeutic modulation of the expression or function of this gene may be effective in the treatment of colon and lung cancer.

Among tissues with metabolic or endocrine function, this gene is expressed at low to moderate levels in pancreas, adrenal gland, pituitary gland, fetal skeletal muscle, heart, and the gastrointestinal tract. Therefore, therapeutic modulation of the activity of this gene may prove useful in the treatment of endocrine/metabolically related diseases, such as obesity and diabetes.

In addition, this gene is expressed at high to moderate levels (CTs=30-33) in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

CG59536-01 codes for protein similar to paraneoplastic cancer-testis-brain antigen. Proteins belonging to paraneoplastic cancer-testis-brain antigen are known to be associated with paraneoplastic syndrome, an immune-mediated disorder. The tumor expression of these proteins normally restricted to neurons (or other immunoprivileged sites, such as testis) but ectopically expressed in some cancers results in an immunological response characterized by high titers of antibodies targeting the “onconeuronal” antigen. A T-cell response is also elicited in some paraneoplastic syndromes and may be the cause of neuronal destruction. In some individuals with cancer but no paraneoplastic syndrome, low titers of antibody can be identified in the serum. Low titers of antibody are associated with a better prognosis of the cancer. Experimental animals immunized against a paraneoplastic antigen are partially protected against tumors that express that antigen. See, e.g., Posner et al., (2000) Clin Chem Lab Med 38(2):117-22.

Panel 2.2 Summary: Ag3458 Expression of the CG59536-01 gene is restricted to a sample derived from normal lung (CT=28.4). Thus, expression of this gene could be used to differentiate between this sample and other samples on this panel and as a marker to detect the presence of lung cancer. Furthermore, therapeutic modulation of the expression or function of this gene may be effective in the treatment of lung cancer.

Panel 4D Summary: Ag3458 Highest expression of the CG59536-01 gene is seen in the colon (CT=31.1). Therefore, expression of this gene could potentially be used to distinguish colon from the other tissues on this panel. Furthermore, expression of this gene is decreased in colon samples from patients with IBD colitis and Crohn's disease relative to normal colon. Therefore, therapeutic modulation of the activity of the protein encoded by this gene may be useful in the treatment of inflammatory bowel disease.

In addition, this gene is expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin. This widespread pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel CNS_{—}1 Summary: Ag3458 Results from one experiment with the CG59536-01 gene are not included. The amp plot indicates that there were experimental difficulties with this run.

G. CG59299-01: Q9UJI0 C380A1.1B (Novel Protein)

Expression of gene CG59299-01 was assessed using the primer-probe set Ag3535, described in Table GA. Results of the RTQ-PCR runs are shown in Tables GB, GC and GD.

TABLE GA
Probe Name Ag3535

| Primers | Sequences | SEQ ID NO. | Length | Start Position |
|---|---|---|---|---|
| Forward | 5′-tctccaagaagggtgtcaaag-3′ | 85 | 21 | 665 |
| Probe | TET-5′-atgagcctcaagcgctccaccat-3′-TAMRA | 86 | 23 | 688 |
| Reverse | 5′-ctgcatctgttcaagcataacc-3′ | 87 | 22 | 729 |

TABLE GB
CNS_neurodegeneration_v1.0

| | Rel. Exp. (%) | | Rel. Exp. (%) |
|---|---|---|---|
| | Ag3535, Run | | Ag3535, Run |
| Tissue Name | 210631620 | Tissue Name | 210631620 |
| AD 1 Hippo | 16.2 | Control (Path) 3 | 6.3 |
| | | Temporal Ctx | |
| AD 2 Hippo | 34.2 | Control (Path) 4 | 39.2 |
| | | Temporal Ctx | |
| AD 3 Hippo | 14.8 | AD 1 Occipital Ctx | 12.3 |
| AD 4 Hippo | 11.4 | AD 2 Occipital Ctx | 0.0 |
| | | (Missing) | |
| AD 5 Hippo | 73.2 | AD 3 Occipital Ctx | 6.3 |
| AD 6 Hippo | 42.3 | AD 4 Occipital Ctx | 25.9 |
| Control 2 Hippo | 46.3 | AD 5 Occipital Ctx | 45.7 |
| Control 4 Hippo | 7.4 | AD 6 Occipital Ctx | 12.8 |
| Control (Path) 3 Hippo | 7.5 | Control 1 Occipital Ctx | 3.4 |
| AD 1 Temporal Ctx | 16.3 | Control 2 Occipital Ctx | 76.8 |
| AD 2 Temporal Ctx | 37.4 | Control 3 Occipital Ctx | 23.5 |
| AD 3 Temporal Ctx | 10.5 | Control 4 Occipital Ctx | 5.7 |
| AD 4 Temporal Ctx | 30.6 | Control (Path) 1 | 97.9 |
| | | Occipital Ctx | |
| AD 5 Inf Temporal Ctx | 83.5 | Control (Path) 2 | 12.7 |
| | | Occipital Ctx | |
| AD 5 Sup Temporal | 40.1 | Control (Path) 3 | 2.6 |
| Ctx | | Occipital Ctx | |
| AD 6 Inf Temporal Ctx | 38.7 | Control (Path) 4 | 17.8 |
| | | Occipital Ctx | |
| AD 6 Sup Temporal | 43.8 | Control 1 Parietal Ctx | 7.2 |
| Ctx | | | |
| Control 1 Temporal | 7.4 | Control 2 Parietal Ctx | 35.6 |
| Ctx | | | |
| Control 2 Temporal | 72.7 | Control 3 Parietal Ctx | 22.5 |
| Ctx | | | |
| Control 3 Temporal | 34.2 | Control (Path) 1 | 100.0 |
| Ctx | | Parietal Ctx | |
| Control 3 Temporal | 14.8 | Control (Path) 2 | 25.7 |
| Ctx | | Parietal Ctx | |
| Control (Path) 1 | 88.9 | Control (Path) 3 | 4.7 |
| Temporal Ctx | | Parietal Ctx | |
| Control (Path) 2 | 50.7 | Control (Path) 4 | 47.0 |
| Temporal Ctx | | Parietal Ctx | |

TABLE GC
General_screening_panel_v1.4

| | Rel. Exp. (%) | | Rel. Exp. (%) |
|---|---|---|---|
| | Ag3535, Run | | Ag3535, Run |
| Tissue Name | 217044649 | Tissue Name | 217044649 |
| Adipose | 0.0 | Renal ca. TK-10 | 0.0 |
| Melanoma* | 0.0 | Bladder | 0.3 |
| Hs688(A).T | | | |
| Melanoma* Hs688(B).T | 0.0 | Gastric ca. (liver met.) | 2.6 |
| | | NCI-N87 | |
| Melanoma* M14 | 0.1 | Gastric ca. KATO III | 5.7 |
| Melanoma* LOXIMVI | 0.6 | Colon ca. SW-948 | 0.0 |
| Melanoma* SK-MEL-5 | 7.5 | Colon ca. SW480 | 12.2 |
| Squamous cell | 0.0 | Colon ca.* (SW480 met) | 4.9 |
| carcinoma SCC-4 | | SW620 | |
| Testis Pool | 1.8 | Colon ca. HT29 | 0.0 |
| Prostate ca.* (bone met) | 2.1 | Colon ca. HCT-116 | 23.5 |
| PC-3 | | | |
| Prostate Pool | 0.1 | Colon ca. CaCo-2 | 0.5 |
| Placenta | 0.0 | Colon cancer tissue | 0.1 |
| Uterus Pool | 0.0 | Colon ca. SW1116 | 5.4 |
| Ovarian ca. OVCAR-3 | 0.3 | Colon ca. Colo-205 | 0.1 |
| Ovarian ca. SK-OV-3 | 2.3 | Colon ca. SW-48 | 0.0 |
| Ovarian ca. OVCAR-4 | 0.3 | Colon Pool | 0.1 |
| Ovarian ca. OVCAR-5 | 16.7 | Small Intestine Pool | 0.0 |
| Ovarian ca. IGROV-1 | 0.1 | Stomach Pool | 0.0 |
| Ovarian ca. OVCAR-8 | 0.2 | Bone Marrow Pool | 0.0 |
| Ovary | 0.0 | Fetal Heart | 0.0 |
| Breast ca. MCF-7 | 8.8 | Heart Pool | 0.0 |
| Breast ca. MDA-MB- | 0.4 | Lymph Node Pool | 0.0 |
| 231 | | | |
| Breast ca. BT 549 | 3.6 | Fetal Skeletal Muscle | 0.0 |
| Breast ca. T47D | 27.9 | Skeletal Muscle Pool | 0.0 |
| Breast ca. MDA-N | 0.0 | Spleen Pool | 0.1 |
| Breast Pool | 0.0 | Thymus Pool | 0.0 |
| Trachea | 0.3 | CNS cancer (glio/astro) | 1.5 |
| | | U87-MG | |
| Lung | 0.0 | CNS cancer (glio/astro) U- | 2.5 |
| | | 118-MG | |
| Fetal Lung | 0.0 | CNS cancer (neuro; met) | 6.2 |
| | | SK-N-AS | |
| Lung ca. NCI-N417 | 4.2 | CNS cancer (astro) SF-539 | 0.0 |
| Lung ca. LX-1 | 22.8 | CNS cancer (astro) SNB- | 22.4 |
| | | 75 | |
| Lung ca. NCI-H146 | 9.0 | CNS cancer (glio) SNB-19 | 0.1 |
| Lung ca. SHP-77 | 9.0 | CNS cancer (glio) SF-295 | 2.0 |
| Lung ca. A549 | 0.1 | Brain (Amygdala) Pool | 65.1 |
| Lung ca. NCI-H526 | 3.5 | Brain (cerebellum) | 62.9 |
| Lung ca. NCI-H23 | 0.4 | Brain (fetal) | 24.7 |
| Lung ca. NCI-H460 | 0.9 | Brain (Hippocampus) Pool | 65.1 |
| Lung ca. HOP-62 | 0.0 | Cerebral Cortex Pool | 86.5 |
| Lung ca. NCI-H522 | 14.2 | Brain (Substantia nigra) | 94.6 |
| | | Pool | |
| Liver | 0.0 | Brain (Thalamus) Pool | 100.0 |
| Fetal Liver | 0.1 | Brain (whole) | 92.7 |
| Liver ca. HepG2 | 0.0 | Spinal Cord Pool | 6.3 |
| Kidney Pool | 0.0 | Adrenal Gland | 0.9 |
| Fetal Kidney | 0.0 | Pituitary gland Pool | 2.6 |
| Renal ca. 786-0 | 0.1 | Salivary Gland | 1.4 |
| Renal ca. A498 | 2.3 | Thyroid (female) | 0.0 |
| Renal ca. ACHN | 1.0 | Pancreatic ca. CAPAN2 | 0.0 |
| Renal ca. UO-31 | 0.0 | Pancreas Pool | 0.2 |

TABLE GD
Panel 4.1D

| | Rel. Exp. (%) Ag3535, | | Rel. Exp. (%) Ag3535, |
|---|---|---|---|
| Tissue Name | Run 169840817 | Tissue Name | Run 169840817 |
| Secondary Th1 act | 0.5 | HUVEC IL-1beta | 0.0 |
| Secondary Th2 act | 1.7 | HUVEC IFN gamma | 0.0 |
| Secondary Tr1 act | 0.0 | HUVEC TNF alpha + IFN | 0.0 |
| | | gamma | |
| Secondary Th1 rest | 2.7 | HUVEC TNF alpha + IL4 | 0.0 |
| Secondary Th2 rest | 4.3 | HUVEC IL-11 | 0.0 |
| Secondary Tr1 rest | 1.6 | Lung Microvascular EC none | 0.4 |
| Primary Th1 act | 0.0 | Lung Microvascular EC | 0.6 |
| | | TNF alpha + IL-1beta | |
| Primary Th2 act | 0.8 | Microvascular Dermal EC | 0.0 |
| | | none | |
| Primary Tr1 act | 1.0 | Microvasular Dermal EC | 0.8 |
| | | TNF alpha + IL-1beta | |
| Primary Th1 rest | 0.0 | Bronchial epithelium | 42.3 |
| | | TNF alpha + IL1beta | |
| Primary Th2 rest | 0.0 | Small airway epithelium none | 18.0 |
| Primary Tr1 rest | 0.0 | Small airway epithelium | 12.2 |
| | | TNF alpha + IL-1beta | |
| CD45RA CD4 | 0.0 | Coronery artery SMC rest | 2.5 |
| lymphocyte act | | | |
| CD45RO CD4 | 0.0 | Coronery artery SMC | 4.1 |
| lymphocyte act | | TNF alpha + IL-1beta | |
| CD8 lymphocyte act | 0.0 | Astrocytes rest | 70.7 |
| Secondary CD8 | 0.2 | Astrocytes TNF alpha + IL- | 100.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 1.7 | CCD1106 (Keratinocytes) none | 44.8 |
| CD95 CH11 | | | |
| LAK cells rest | 0.0 | CCD1106 (Keratinocytes) | 47.3 |
| | | TNF alpha + IL-1beta | |
| LAK cells IL-2 | 0.0 | Liver cirrhosis | 0.2 |
| LAK cells IL-2 + IL-12 | 0.0 | NCI-H292 none | 3.2 |
| LAK cells IL-2 + IFN | 0.0 | NCI-H292 IL-4 | 6.0 |
| gamma | | | |
| LAK cells IL-2 + IL-18 | 0.0 | NCI-H292 IL-9 | 4.9 |
| LAK cells | 0.0 | NCI-H292 IL-13 | 12.0 |
| PMA/ionomycin | | | |
| NK Cells IL-2 rest | 0.9 | NCI-H292 IFN gamma | 5.5 |
| Two Way MLR 3 day | 0.0 | HPAEC none | 0.0 |
| Two Way MLR 5 day | 0.0 | HPAEC TNF alpha + IL-1Beta | 1.0 |
| Two Way MLR 7 day | 0.0 | Lung fibroblast none | 2.9 |
| PBMC rest | 0.0 | Lung fibroblast TNF alpha + | 0.0 |
| | | IL-1Beta | |
| PBMC PWM | 0.0 | Lung fibroblast IL-4 | 2.1 |
| PBMC PHA-L | 0.0 | Lung fibroblast IL-9 | 3.7 |
| Ramos (B cell) none | 0.0 | Lung fibroblast IL-13 | 3.8 |
| Ramos (B cell) ionomycin | 0.0 | Lung fibroblast IFN gamma | 1.4 |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1Beta | |
| EOL-1 dbcAMP | 0.0 | Dermal fibroblast IFN gamma | 0.8 |
| PMA/ionomycin | | | |
| Dendritic cells none | 0.0 | Dermal fibroblast IL-4 | 1.6 |
| Dendritic cells LPS | 0.0 | Dermal Fibroblasts rest | 4.1 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.4 |
| Macrophages rest | 0.0 | Lung | 0.0 |
| Macrophages LPS | 0.0 | Thymus | 2.8 |
| HUVEC none | 2.9 | Kidney | 0.0 |
| HUVEC starved | 1.6 | | |

CNS_neurodegeneration_v1.0 Summary: Ag3535 This panel confirms the expression of the CG59299-01 gene at high levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3535 High expression of the CG59299-01 is detected throughout the CNS, including in amygdala, substantia nigra, thalamus, cerebellum, cerebral cortex, spinal cord, and CNS cancer samples (CTs=24.9-27). Therefore, this gene may play a role in central nervous system disorders such as Alzheimer's disease, Parkinson's disease, epilepsy, multiple sclerosis, schizophrenia and depression.

Significant expression of this gene is also detected in testis, prostate, one melenoma cell line, prostate cancer, ovarian cancer, breast cancer, lung cancer, renal cancer, gastric cancer and colon cancer (CTs=26.8-28). Therefore, therapeutic modulation of the activity of this gene or its protein product, through the use of small molecule drugs, protein therapeutics or antibodies, might be beneficial in the treatment of these cancers.

Panel 4.1D Summary: Ag3535 Highest expression of the CG59299-01 gene is detected in TNFalpha+IL1beta treated astrocytes (CT=30). Significant expression of this gene is also detected in resting secondary Th2, TNFalpha+IL1beta treated bronchial epithelium, astrocytes, small airway epithelium, CCD1106 (keratinocytes), IL-9 and IL-13 treated lung fibroblast, IL treated NCI-H292, and dermal fibroblast. Therefore, therapeutic modulation of this gene or its protein product may be beneficial in the treatment of general autoimmunity, asthma and parasitic disease, psoriasis and emphysema.

H. CG59632-01 and CG59632-01: Novel Expressed Mitochondrial Protein

Expression of gene CG59632-01 was assessed using the primer-probe set Ag3426, described in Table HA. Please note that CG59632-02 represents a full-length physical clone of the CG59632-01 gene, validating the prediction of the gene sequence.

TABLE HA
Probe Name Ag3426

| | | SEQ ID | | |
|---|---|---|---|---|
| Primers | Sequences | NO. | Length | Start Position |
| Forward | 5′-tgagtaagatggcgtccaag-3′ | 88 | 20 | 78 |
| Probe | TET-5′-gtgctggtggatgacaccagcagt-3′-TAMRA | 89 | 24 | 110 |
| Reverse | 5′-tacagctcatccagcacctc-3′ | 90 | 20 | 134 |

CNS_neurodegeneration_v1.0 Summary: Ag3426 Expression of the CG59632-01 gene is low/undetectable (CTs>34.5) across all of the samples on this panel (data not shown).

General_screening_panel_v1.4 Summary: Ag3426 Results from one experiment with the CG59632-01 gene are not included. The amp plot indicates that there were experimental difficulties with this run.

Panel 4.1D Summary: Ag3426 Expression of the CG59632-01 gene is low/undetectable (CTs>34.5) across all of the samples on this panel (data not shown).

I. CG59303-01: CAC15523 DJ697K14.9.1 (Novel Protein)

Expression of gene CG59303-01 was assessed using the primer-probe set Ag3537, described in Table IA. Results of the RTQ-PCR runs are shown in Tables IB, IC and ID.

TABLE IA
Probe Name Ag3537

| | | SEQ ID | | Start |
|---|---|---|---|---|
| Primers | Sequences | NO. | Length | Position |
| Foward | 5′-accttagggacctcaagaagag-3′ | 91 | 22 | 647 |
| Probe | TET-5′-ttcccctaaatctgtacttattgcagg-3′-TAMRA | 92 | 27 | 674 |
| Reverse | 5′-aaagacttgccatcagactttg-3′ | 93 | 22 | 701 |

TABLE IB
CNS_neurodegeneration_v1.0

| | Rel. Exp. (%) | | Rel. Exp. (%) |
|---|---|---|---|
| | Ag3537, Run | | Ag3537, Run |
| Tissue Name | 210629737 | Tissue Name | 210629737 |
| AD 1 Hippo | 15.8 | Control (Path) 3 | 8.3 |
| | | Temporal Ctx | |
| AD 2 Hippo | 29.7 | Control (Path) 4 | 29.3 |
| | | Temporal Ctx | |
| AD 3 Hippo | 11.7 | AD 1 Occipital Ctx | 39.2 |
| AD 4 Hippo | 3.8 | AD 2 Occipital Ctx | 0.0 |
| | | (Missing) | |
| AD 5 Hippo | 34.2 | AD 3 Occipital Ctx | 21.3 |
| AD 6 Hippo | 63.3 | AD 4 Occipital Ctx | 11.6 |
| Control 2 Hippo | 21.2 | AD 5 Occipital Ctx | 22.5 |
| Control 4 Hippo | 13.9 | AD 6 Occipital Ctx | 13.3 |
| Control (Path) 3 Hippo | 3.8 | Control 1 Occipital Ctx | 12.9 |
| AD 1 Temporal Ctx | 31.4 | Control 2 Occipital Ctx | 48.0 |
| AD 2 Temporal Ctx | 32.1 | Control 3 Occipital Ctx | 25.3 |
| AD 3 Temporal Ctx | 10.4 | Control 4 Occipital Ctx | 5.5 |
| AD 4 Temporal Ctx | 16.0 | Control (Path) 1 | 87.1 |
| | | Occipital Ctx | |
| AD 5 Inf Temporal Ctx | 100.0 | Control (Path) 2 | 11.5 |
| | | Occipital Ctx | |
| AD 5 Sup Temporal | 51.4 | Control (Path) 3 | 5.9 |
| Ctx | | Occipital Ctx | |
| AD 6 Inf Temporal Ctx | 58.6 | Control (Path) 4 | 16.0 |
| | | Occipital Ctx | |
| AD 6 Sup Temporal | 51.8 | Control 1 Parietal Ctx | 11.3 |
| Ctx | | | |
| Control 1 Temporal | 8.9 | Control 2 Parietal Ctx | 39.8 |
| Ctx | | | |
| Control 2 Temporal | 19.6 | Control 3 Parietal Ctx | 24.8 |
| Ctx | | | |
| Control 3 Temporal | 28.3 | Control (Path) 1 | 67.8 |
| Ctx | | Parietal Ctx | |
| Control 3 Temporal | 8.7 | Control (Path) 2 | 27.0 |
| Ctx | | Parietal Ctx | |
| Control (Path) 1 | 38.7 | Control (Path) 3 | 2.3 |
| Temporal Ctx | | Parietal Ctx | |
| Control (Path) 2 | 26.1 | Control (Path) 4 | 26.4 |
| Temporal Ctx | | Parietal Ctx | |

TABLE IC
General_screening_panel_v1.4

| | Rel. Exp. (%) | | Rel. Exp. (%) |
|---|---|---|---|
| | Ag3537, Run | | Ag3537, Run |
| Tissue Name | 217044717 | Tissue Name | 217044717 |
| Adipose | 3.6 | Renal ca. TK-10 | 38.2 |
| Melanoma* | 4.7 | Bladder | 11.2 |
| Hs688(A).T | | | |
| Melanoma* Hs688(B).T | 7.0 | Gastric ca. (liver met.) | 85.9 |
| | | NCI-N87 | |
| Melanoma* M14 | 9.8 | Gastric ca. KATO III | 53.6 |
| Melanoma* LOXIMVI | 1.6 | Colon ca. SW-948 | 2.8 |
| Melanoma* SK-MEL-5 | 5.3 | Colon ca. SW480 | 67.8 |
| Squamous cell | 24.0 | Colon ca.* (SW480 met) | 28.5 |
| carcinoma SCC-4 | | SW620 | |
| Testis Pool | 7.0 | Colon ca. HT29 | 29.1 |
| Prostate ca.* (bone met) | 28.3 | Colon ca. HCT-116 | 14.7 |
| PC-3 | | | |
| Prostate Pool | 8.4 | Colon ca. CaCo-2 | 25.3 |
| Placenta | 0.8 | Colon cancer tissue | 10.9 |
| Uterus Pool | 6.3 | Colon ca. SW1116 | 4.2 |
| Ovarian ca. OVCAR-3 | 32.5 | Colon ca. Colo-205 | 8.5 |
| Ovarian ca. SK-OV-3 | 15.1 | Colon ca. SW-48 | 9.3 |
| Ovarian ca. OVCAR-4 | 3.8 | Colon Pool | 31.2 |
| Ovarian ca. OVCAR-5 | 78.5 | Small Intestine Pool | 34.6 |
| Ovarian ca. IGROV-1 | 16.6 | Stomach Pool | 25.3 |
| Ovarian ca. OVCAR-8 | 5.8 | Bone Marrow Pool | 19.3 |
| Ovary | 10.6 | Fetal Heart | 10.3 |
| Breast ca. MCF-7 | 70.7 | Heart Pool | 13.1 |
| Breast ca. MDA-MB- | 27.2 | Lymph Node Pool | 34.2 |
| 231 | | | |
| Breast Ca. BT 549 | 22.1 | Fetal Skeletal Muscle | 10.0 |
| Breast ca. T47D | 100.0 | Skeletal Muscle Pool | 5.0 |
| Breast ca. MDA-N | 11.8 | Spleen Pool | 9.3 |
| Breast Pool | 29.1 | Thymus Pool | 18.8 |
| Trachea | 13.1 | CNS cancer (glio/astro) | 9.0 |
| | | U87-MG | |
| Lung | 45.4 | CNS cancer (glio/astro) U- | 20.7 |
| | | 118-MG | |
| Fetal Lung | 38.7 | CNS cancer (neuro; met) | 4.5 |
| | | SK-N-AS | |
| Lung ca. NCI-N417 | 1.3 | CNS cancer (astro) SF-539 | 6.9 |
| Lung ca. LX-1 | 59.9 | CNS cancer (astro) SNB- | 28.9 |
| | | 75 | |
| Lung ca. NCI-H146 | 7.7 | CNS cancer (glio) SNB-19 | 20.2 |
| Lung ca. SHP-77 | 11.4 | CNS cancer (glio) SF-295 | 45.1 |
| Lung ca. A549 | 12.3 | Brain (Amygdala) Pool | 13.2 |
| Lung ca. NCI-H526 | 5.6 | Brain (cerebellum) | 39.8 |
| Lung ca. NCI-H23 | 66.0 | Brain (fetal) | 34.4 |
| Lung ca. NCI-H460 | 33.7 | Brain (Hippocampus) Pool | 14.0 |
| Lung ca. HOP-62 | 14.0 | Cerebral Cortex Pool | 8.7 |
| Lung ca. NCI-H522 | 45.1 | Brain (Substantia nigra) | 17.6 |
| | | Pool | |
| Liver | 0.4 | Brain (Thalamus) Pool | 24.3 |
| Fetal Liver | 3.7 | Brain (whole) | 12.6 |
| Liver ca. HepG2 | 4.9 | Spinal Cord Pool | 20.0 |
| Kidney Pool | 71.2 | Adrenal Gland | 1.6 |
| Fetal Kidney | 22.8 | Pituitary gland Pool | 1.7 |
| Renal ca. 786-0 | 21.9 | Salivary Gland | 9.1 |
| Renal ca. A498 | 6.9 | Thyroid (female) | 4.3 |
| Renal ca. ACHN | 9.0 | Pancreatic ca. CAPAN2 | 47.3 |
| Renal ca. UO-31 | 4.0 | Pancreas Pool | 27.2 |

TABLE ID
Panel 4D

| | Rel. Exp. (%) Ag3537, | | Rel. Exp. (%) Ag3537, |
|---|---|---|---|
| Tissue Name | Run 166444752 | Tissue Name | Run 166444752 |
| Secondary Th1 act | 7.4 | HUVEC IL-1beta | 2.8 |
| Secondary Th2 act | 5.0 | HUVEC IFN gamma | 1.5 |
| Secondary Tr1 act | 15.1 | HUVEC TNF alpha + IFN | 1.6 |
| | | gamma | |
| Secondary Th1 rest | 6.7 | HUVEC TNF alpha + IL4 | 2.1 |
| Secondary Th2 rest | 4.2 | HUVEC IL-11 | 0.0 |
| Secondary Tr1 rest | 6.0 | Lung Microvascular EC none | 4.0 |
| Primary Th1 act | 5.8 | Lung Microvascular EC | 2.5 |
| | | TNF alpha + IL-1beta | |
| Primary Th2 act | 11.5 | Microvascular Dermal EC | 8.5 |
| | | none | |
| Primary Tr1 act | 12.9 | Microsvasular Dermal EC | 1.2 |
| | | TNF alpha + IL-1beta | |
| Primary Th1 rest | 19.9 | Bronchial epithelium | 6.2 |
| | | TNF alpha + IL 1beta | |
| Primary Th2 rest | 8.8 | Small airway epithelium none | 3.5 |
| Primary Tr1 rest | 12.2 | Small airway epithelium | 16.7 |
| | | TNF alpha + IL-1beta | |
| CD45RA CD4 | 5.0 | Coronery artery SMC rest | 9.9 |
| lymphocyte act | | | |
| CD45RO CD4 | 8.0 | Coronery artery SMC | 2.2 |
| lymphocyte act | | TNF alpha + IL-1beta | |
| CD8 lymphocyte act | 8.5 | Astrocytes rest | 7.5 |
| Secondary CD8 | 5.2 | Astrocytes TNF alpha + IL- | 4.0 |
| lymphocyte rest | | 1beta | |
| Secondary CD8 | 4.7 | KU-812 (Basophil) rest | 1.1 |
| lymphocyte act | | | |
| CD4 lymphocyte none | 30.6 | KU-812 (Basophil) | 0.5 |
| | | PMA/ionomycin | |
| 2ry Th1/Th2/Tr1_anti- | 2.3 | CCD1106 (Keratinocytes) none | 14.0 |
| CD95 CH11 | | | |
| LAK cells rest | 4.3 | CCD1106 (Keratinocytes) | 31.4 |
| | | TNF alpha + IL-1beta | |
| LAK cells IL-2 | 8.1 | Liver cirrhosis | 17.9 |
| LAK cells IL-2 + IL-12 | 15.6 | Lupus kidney | 8.5 |
| LAK cells IL-2 + IFN | 20.6 | NCI-H292 none | 28.1 |
| gamma | | | |
| LAK cells IL-2 + IL-18 | 15.9 | NCI-H292 IL-4 | 29.7 |
| LAK cells | 2.2 | NCI-H292 IL-9 | 26.8 |
| PMA/ionomycin | | | |
| NK Cells IL-2 rest | 7.5 | NCI-H292 IL-13 | 15.8 |
| Two Way MLR 3 day | 15.2 | NCI-H292 IFN gamma | 13.3 |
| Two Way MLR 5 day | 6.7 | HPAEC none | 0.9 |
| Two Way MLR 7 day | 2.5 | HPAEC TNF alpha + IL-1Beta | 0.8 |
| PBMC rest | 16.0 | Lung fibroblast none | 7.6 |
| PBMC PWM | 3.6 | Lung fibroblast TNF alpha + | 6.6 |
| | | IL-1Beta | |
| PBMC PHA-L | 1.8 | Lung fibroblast IL-4 | 2.8 |
| Ramos (B cell) none | 0.6 | Lung fibroblast IL-9 | 5.0 |
| Ramos (B cell) ionomycin | 0.9 | Lung fibroblast IL-13 | 2.1 |
| B lymphocytes PWM | 6.5 | Lung fibroblast IFN gamma | 3.3 |
| B lymphocytes CD40L | 17.4 | Dermal fibroblast CCD1070 | 8.1 |
| and IL-4 | | rest | |
| EOL-1 dbcAMP | 2.2 | Dermal fibroblast CCD1070 | 13.2 |
| | | TNF alpha | |
| EOL-1 dbcAMP | 5.6 | Dermal fibroblast CCD1070 | 4.4 |
| PMA/ionomycin | | IL-1Beta | |
| Dendritic cells none | 3.1 | Dermal fibroblast IFN gamma | 1.7 |
| Dendritic cells LPS | 1.0 | Dermal fibroblast IL-4 | 4.1 |
| Dendritic cells anti-CD40 | 3.6 | IBD Colitis 2 | 4.0 |
| Monocytes rest | 12.9 | IBD Crohn's | 6.2 |
| Monocytes LPS | 1.4 | Colon | 100.0 |
| Macrophages rest | 3.4 | Lung | 7.6 |
| Macrophages LPS | 0.5 | Thymus | 16.4 |
| HUVEC none | 4.9 | Kidney | 32.8 |
| HUVEC starved | 14.2 | | |

CNS_neurodegeneration_v1.0 Summary: Ag3537 This panel confirms the expression of the CG59303-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3537 Highest expession of the CG59303-01 gene is detected in sample derived from the breast cancer cell line T47D (CT=29.3). However, similar high expression of this gene is also seen in a cluster of lung, ovarian, breast, colon, gastric and CNS cancer cell lines. Therefore, therapeutic modulation of the activity of this gene or its protein product, through the use of small molecule drugs, protein therapeutics or antibodies, might be beneficial in the treatment of these cancers.

Among tissues with metabolic or endocrine function, this gene is expressed at low to moderate levels in pancreas, adipose, thyroid, skeletal muscle, heart, and the gastrointestinal tract. Therefore, therapeutic modulation of the activity of this gene may prove useful in the treatment of endocrine/metabolically related diseases, such as obesity and diabetes.

Low expression is also detected in fetal liver. Interestingly, this gene is expressed at much higher levels in fetal (CT=34) when compared to adult liver (CT=37). This observation suggests that expression of this gene can be used to distinguish fetal from adult liver.

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4D Summary: Ag3537 Highest expression of the CG59303-01 gene is detected in samples derived from colon (CT=29.5). Thus expression of this gene can be used to distinguish this sample from other samples in the panel. Furthermore, expression of this gene is decreased in colon samples from patients with IBD colitis and Crohn's disease relative to normal colon. Therefore, therapeutic modulation of the activity of the protein encoded by this gene may be useful in the treatment of inflammatory bowel disease.

In addition, this gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

J. CG59719-01: Novel GAP Protein

Expression of gene CG59719-01 was assessed using the primer-probe set Ag3654, described in Table JA. Results of the RTQ-PCR runs are shown in Tables JB, JC and JD.

TABLE JA
Probe Name Ag3654

| | | SEQ ID | | |
|---|---|---|---|---|
| Primers | Sequences | NO. | Length | Start Position |
| Forward | 5′-aggtggtgttcaactgctactg-3′ | 194 | 22 | 2732 |
| Probe | TET-5′-ctggactccagactcctccacactca-3′-TAMRA | 95 | 26 | 2769 |
| Reverse | 5′-ggtctcctcgtccatagaagat-3′ | 96 | 22 | 2797 |

TABLE JB
CNS_neurodegeneration_v1.0

| | Rel. Exp. (%) | | Rel. Exp. (%) |
|---|---|---|---|
| | Ag3654, Run | | Ag3654, Run |
| Tissue Name | 224337929 | Tissue Name | 224337929 |
| AD 1 Hippo | 32.1 | Control (Path) 3 Temporal Ctx | 10.3 |
| AD 2 Hippo | 33.0 | Control (Path) 4 Temporal Ctx | 35.4 |
| AD 3 Hippo | 20.6 | AD 1 Occipital Ctx | 17.6 |
| AD 4 Hippo | 14.7 | AD 2 Occipital Ctx (Missing) | 0.0 |
| AD 5 Hippo | 75.3 | AD 3 Occipital Ctx | 11.9 |
| AD 6 Hippo | 100.0 | AD 4 Occipital Ctx | 25.0 |
| Control 2 Hippo | 75.3 | AD 5 Occipital Ctx | 50.0 |
| Control 4 Hippo | 14.7 | AD 6 Occipital Ctx | 17.0 |
| Control (Path) 3 Hippo | 16.7 | Control 1 Occipital Ctx | 7.1 |
| AD 1 Temporal Ctx | 30.1 | Control 2 Occipital Ctx | 51.4 |
| AD 2 Temporal Ctx | 29.7 | Control 3 Occipital Ctx | 16.4 |
| AD 3 Temporal Ctx | 13.4 | Control 4 Occipital Ctx | 8.8 |
| AD 4 Temporal Ctx | 26.1 | Control (Path) 1 Occipital Ctx | 71.7 |
| AD 5 Inf Temporal Ctx | 80.1 | Control (Path) 2 Occipital Ctx | 9.0 |
| AD 5 Sup Temporal Ctx | 71.7 | Control (Path) 3 Occipital Ctx | 5.2 |
| AD 6 Inf Temporal Ctx | 63.3 | Control (Path) 4 Occipital Ctx | 18.0 |
| AD 6 Sup Temporal Ctx | 56.3 | Control 1 Parietal Ctx | 10.8 |
| Control 1 Temporal Ctx | 8.2 | Control 2 Parietal Ctx | 42.6 |
| Control 2 Temporal Ctx | 37.1 | Control 3 Parietal Ctx | 17.8 |
| Control 3 Temporal Ctx | 20.7 | Control (Path) 1 Parietal Ctx | 63.7 |
| Control 3 Temporal Ctx | 15.4 | Control (Path) 2 Parietal Ctx | 17.8 |
| Control (Path) 1 Temporal Ctx | 61.6 | Control (Path) 3 Parietal Ctx | 5.7 |
| Control (Path) 2 Temporal Ctx | 19.5 | Control (Path) 4 Parietal Ctx | 43.2 |

TABLE JC
General_screening_panel_v1.4

| | Rel. Exp. (%) | | Rel. Exp. (%) |
|---|---|---|---|
| | Ag3654, Run | | Ag3654, Run |
| Tissue Name | 218951398 | Tissue Name | 218951398 |
| Adipose | 10.9 | Renal ca. TK-10 | 53.6 |
| Melanoma* Hs688(A).T | 13.7 | Bladder | 23.5 |
| Melanoma* Hs688(B).T | 15.4 | Gastric ca. (liver met.) NCI-N87 | 62.9 |
| Melanoma* M14 | 12.3 | Gastric ca. KATO III | 63.3 |
| Melanoma* LOXIMVI | 29.5 | Colon ca. SW-948 | 8.7 |
| Melanoma* SK-MEL-5 | 11.3 | Colon ca. SW480 | 42.6 |
| Squamous cell carcinoma SCC-4 | 12.1 | Colon ca.* (SW480 met) SW620 | 33.9 |
| Testis Pool | 6.9 | Colon ca. HT29 | 34.4 |
| Prostate ca.* (bone met) PC-3 | 39.5 | Colon ca. HCT-116 | 34.9 |
| Prostate Pool | 10.1 | Colon ca. CaCo-2 | 100.0 |
| Placenta | 3.8 | Colon cancer tissue | 12.9 |
| Uterus Pool | 2.1 | Colon ca. SW1116 | 7.1 |
| Ovarian ca. OVCAR-3 | 66.9 | Colon ca. Colo-205 | 8.1 |
| Ovarian ca. SK-OV-3 | 55.5 | Colon ca. SW-48 | 4.2 |
| Ovarian ca. OVCAR-4 | 27.7 | Colon Pool | 8.5 |
| Ovarian ca. OVCAR-5 | 62.0 | Small Intestine Pool | 7.1 |
| Ovarian ca. IGROV-1 | 19.2 | Stomach Pool | 4.9 |
| Ovarian ca. OVCAR-8 | 7.6 | Bone Marrow Pool | 4.0 |
| Ovary | 7.2 | Fetal Heart | 5.9 |
| Breast ca. MCF-7 | 25.3 | Heart Pool | 3.4 |
| Breast ca. MDA-MB-231 | 54.7 | Lymph Node Pool | 10.4 |
| Breast ca. BT 549 | 74.7 | Fetal Skeletal Muscle | 5.1 |
| Breast ca. T47D | 93.3 | Skeletal Muscle Pool | 4.2 |
| Breast ca. MDA-N | 6.8 | Spleen Pool | 9.8 |
| Breast Pool | 8.9 | Thymus Pool | 10.2 |
| Trachea | 22.5 | CNS cancer (glio/astro) U87-MG | 18.0 |
| Lung | 2.1 | CNS cancer (glio/astro) U-118-MG | 93.3 |
| Fetal Lung | 45.4 | CNS cancer (neuro; met) SK-N-AS | 22.5 |
| Lung ca. NCI-N417 | 8.9 | CNS cancer (astro) SF-539 | 20.4 |
| Lung ca. LX-1 | 21.6 | CNS cancer (astro) SNB-75 | 32.3 |
| Lung ca. NCI-H146 | 17.2 | CNS cancer (glio) SNB-19 | 22.2 |
| Lung ca. SHP-77 | 55.9 | CNS cancer (glio) SF-295 | 28.1 |
| Lung ca. A549 | 91.4 | Brain (Amygdala) Pool | 4.4 |
| Lung ca. NCI-H526 | 7.5 | Brain (cerebellum) | 74.7 |
| Lung ca. NCI-H23 | 11.4 | Brain (fetal) | 28.5 |
| Lung ca. NCI-H460 | 5.4 | Brain (Hippocampus) Pool | 7.8 |
| Lung ca. HOP-62 | 15.5 | Cerebral Cortex Pool | 10.4 |
| Lung ca. NCI-H522 | 21.6 | Brain (Substantia nigra) Pool | 6.7 |
| Liver | 1.3 | Brain (Thalamus) Pool | 11.3 |
| Fetal Liver | 11.0 | Brain (whole) | 13.1 |
| Liver ca. HepG2 | 14.4 | Spinal Cord Pool | 5.8 |
| Kidney Pool | 10.3 | Adrenal Gland | 7.1 |
| Fetal Kidney | 15.7 | Pituitary gland Pool | 3.1 |
| Renal ca. 786-0 | 18.3 | Salivary Gland | 14.6 |
| Renal ca. A498 | 21.3 | Thyroid (female) | 7.1 |
| Renal ca. ACHN | 43.8 | Pancreatic ca. CAPAN2 | 43.2 |
| Renal ca. UO-31 | 56.3 | Pancreas Pool | 15.7 |

TABLE JD
Panel 4.1D

| | Rel. Exp. (%) | | Rel. Exp. (%) |
|---|---|---|---|
| | Ag3654, Run | | Ag3654, Run |
| Tissue Name | 169976062 | Tissue Name | 169976062 |
| Secondary Th1 act | 18.9 | HUVEC IL-1beta | 58.2 |
| Secondary Th2 act | 23.3 | HUVEC IFN gamma | 40.3 |
| Secondary Tr1 act | 27.0 | HUVEC TNF alpha + IFN gamma | 45.4 |
| Secondary Th1 rest | 9.7 | HUVEC TNF alpha + IL4 | 79.6 |
| Secondary Th2 rest | 12.5 | HUVEC IL-11 | 27.0 |
| Secondary Tr1 rest | 12.4 | Lung Microvascular EC none | 70.7 |
| Primary Th1 act | 17.9 | Lung Microvascular EC TNF alpha + | 58.6 |
| | | IL-1beta | |
| Primary Th2 act | 23.5 | Microvascular Dermal EC none | 63.7 |
| Primary Tr1 act | 20.7 | Microsvasular Dermal EC | 50.0 |
| | | TNF alpha + IL-1beta | |
| Primary Th1 rest | 12.7 | Bronchial epithelium TNF alpha + | 46.3 |
| | | IL1beta | |
| Primary Th2 rest | 15.2 | Small airway epithelium none | 20.6 |
| Primary Tr1 rest | 25.7 | Small airway epithelium TNF alpha + | 37.1 |
| | | IL-1beta | |
| CD45RA CD4 lymphocyte act | 40.1 | Coronery artery SMC rest | 46.7 |
| CD45RO CD4 lymphocyte act | 27.7 | Coronery artery SMC TNF alpha + | 40.3 |
| | | IL-1beta | |
| CD8 lymphocyte act | 21.8 | Astrocytes rest | 16.5 |
| Secondary CD8 lymphocyte rest | 22.5 | Astrocytes TNF alpha + IL-1beta | 11.8 |
| Secondary CD8 lymphocyte act | 11.9 | KU-812 (Basophil) rest | 25.2 |
| CD4 lymphocyte none | 19.2 | KU-812 (Basophil) | 25.9 |
| | | PMA/ionomycin | |
| 2ry Th1/Th2/Tr1_anti-CD95 | 20.3 | CCD1106 (Keratinocytes) none | 90.1 |
| CH11 | | | |
| LAK cells rest | 33.9 | CCD1106 (Keratinocytes) | 78.5 |
| | | TNF alpha + IL-1beta | |
| LAK cells IL-2 | 24.7 | Liver cirrhosis | 18.2 |
| LAK cells IL-2 + IL-12 | 21.5 | NCI-H292 none | 43.8 |
| LAK cells IL-2 + IFN gamma | 24.5 | NCI-H292 IL-4 | 66.0 |
| LAK cells IL-2 + IL-18 | 27.0 | NCI-H292 IL-9 | 79.0 |
| LAK cells PMA/ionomycin | 9.5 | NCI-H292 IL-13 | 72.2 |
| NK Cells IL-2 rest | 21.8 | NCI-H292 IFN gamma | 61.6 |
| Two Way MLR 3 day | 54.3 | HPAEC none | 44.8 |
| Two Way MLR 5 day | 26.8 | HPAEC TNF alpha + IL-1Beta | 95.3 |
| Two Way MLR 7 day | 13.1 | Lung fibroblast none | 38.7 |
| PBMC rest | 28.1 | Lung fibroblast TNF alpha + IL-1 | 26.6 |
| | | beta | |
| PBMC PWM | 24.0 | Lung fibroblast IL-4 | 55.5 |
| PBMC PHA-L | 24.8 | Lung fibroblast IL-9 | 79.0 |
| Ramos (B cell) none | 72.7 | Lung fibroblast IL-13 | 55.1 |
| Ramos (B cell) ionomycin | 84.7 | Lung fibroblast IFN gamma | 37.1 |
| B lymphocytes PWM | 29.3 | Dermal fibroblast CCD1070 rest | 85.9 |
| B lymphocytes CD40L and IL-4 | 100.0 | Dermal fibroblast CCD1070 TNF | 83.5 |
| | | alpha | |
| EOL-1 dbcAMP | 44.8 | Dermal fibroblast CCD1070 IL-1 | 48.0 |
| | | beta | |
| EOL-1 dbcAMP | 35.6 | Dermal fibroblast IFN gamma | 28.3 |
| PMA/ionomycin | | | |
| Dendritic cells none | 25.3 | Dermal fibroblast IL-4 | 73.7 |
| Dendritic cells LPS | 18.8 | Dermal Fibroblasts rest | 20.3 |
| Dendritic cells anti-CD40 | 27.9 | Neutrophils TNFa + LPS | 3.5 |
| Monocytes rest | 44.8 | Neutrophils rest | 12.1 |
| Monocytes LPS | 22.2 | Colon | 55.1 |
| Macrophages rest | 27.5 | Lung | 41.5 |
| Macrophages LPS | 10.7 | Thymus | 57.0 |
| HUVEC none | 46.3 | Kidney | 57.0 |
| HUVEC starved | 53.6 | | |

CNS_neurodegeneration_v1.0 Summary: Ag3654 This panel confirms the expression of the CG59719-01 gene at low to moderate levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3654 Highest expression of the CG59719-01 gene is detected in a sample derived from one of the colon cancer cell line (CT=26.5 1). Thus, expression of this gene can be used to distinguish this sample from other samples in the panel. In addition, low levels of expression of this gene is also associated with colon cancer, ovarian cancer, breast cancer, and CNS cancer cell lines. Therefore, therapeutic modulation of the activity of this gene or its protein product, through the use of small molecule drugs, protein therapeutics or antibodies, might be beneficial in the treatment of these cancers.

Interestingly, this gene is expressed at much higher levels in fetal (CT=27.7) when compared to adult lung (CT=32). This observation suggests that expression of this gene can be used to distinguish fetal from adult lung.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at significant levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

CG59719-01 codes for a protein with Ran-GAP domain. The Rap/ran-GAP domain is found in the GTPase activating protein (GAP) responsible for the activation of nuclear Ras-related regulatory proteins Rap1, Rsr1 and Ran in vitro converting it to the putatively inactive GDP-bound state. See, e.g., Rubinfeld et al. (1991) Cell 65(6): 1033-1042; Hattori et al. (1995) Mol. Cell. Biol. 15(1): 552-560.

Panel 4.1D Summary: Ag3654 The CG59719-01 gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

K. CG59777-01: GTPase Activator Protein

Expression of gene CG59777-01 was assessed using the primer-probe set Ag3582, described in Table KA. Results of the RTQ-PCR runs are shown in Tables KB, and KC.

TABLE KA
Probe Name Ag3582

| | | SEQ ID | | Start |
|---|---|---|---|---|
| Primers | Sequences | NO. | Length | Position |
| Forward | 5′agcgatgatgtggaaccttac-3′ | 97 | 21 | 1519 |
| Probe | TET-5′-cctgaggtagacatctttagactcatcaga-3′-TAMRA | 98 | 30 | 1540 |
| Reverse | 5′-agttccgaatttctcgtaggaa-3′ | 99 | 22 | 1572 |

TABLE KB
CNS_neurodegeneration_v1.0

| | Rel. Exp. (%) | | Rel. Exp. (%) |
|---|---|---|---|
| | Ag3582, Run | | Ag3582, Run |
| Tissue Name | 211006253 | Tissue Name | 211006253 |
| AD 1 Hippo | 7.4 | Control (Path) 3 | 3.2 |
| | | Temporal Ctx | |
| AD 2 Hippo | 31.6 | Control (Path) 4 | 31.9 |
| | | Temporal Ctx | |
| AD 3 Hippo | 3.6 | AD 1 Occipital Ctx | 10.2 |
| AD 4 Hippo | 4.5 | AD 2 Occipital Ctx | 0.0 |
| | | (Missing) | |
| AD 5 Hippo | 100.0 | AD 3 Occipital Ctx | 1.8 |
| AD 6 Hippo | 40.9 | AD 4 Occipital Ctx | 16.4 |
| Control 2 Hippo | 33.2 | AD 5 Occipital Ctx | 47.0 |
| Control 4 Hippo | 7.2 | AD 6 Occipital Ctx | 21.6 |
| Control (Path) 3 Hippo | 7.0 | Control 1 Occipital Ctx | 2.6 |
| AD 1 Temporal Ctx | 5.4 | Control 2 Occipital Ctx | 72.7 |
| AD 2 Temporal Ctx | 27.7 | Control 3 Occipital Ctx | 13.6 |
| AD 3 Temporal Ctx | 2.5 | Control 4 Occipital Ctx | 3.8 |
| AD 4 Temporal Ctx | 9.8 | Control (Path) 1 | 46.7 |
| | | Occipital Ctx | |
| AD 5 Inf Temporal Ctx | 94.6 | Control (Path) 2 | 7.7 |
| | | Occipital Ctx | |
| AD 5 Sup Temporal | 34.9 | Control (Path) 3 | 2.5 |
| Ctx | | Occipital Ctx | |
| AD 6 Inf Temporal Ctx | 39.0 | Contral (Path) 4 | 21.6 |
| | | Occipital Ctx | |
| AD 6 Sup Temporal | 39.2 | Control 1 Parietal Ctx | 3.2 |
| Ctx | | | |
| Control 1 Temporal | 2.8 | Control 2 Parietal Ctx | 30.1 |
| Ctx | | | |
| Control 2 Temporal | 43.5 | Control 3 Parietal Ctx | 16.5 |
| Ctx | | | |
| Control 3 Temporal | 0.3 | Control (Path) 1 | 39.8 |
| Ctx | | Parietal Ctx | |
| Control 3 Temporal | 4.7 | Control (Path) 2 | 19.5 |
| Ctx | | Parietal Ctx | |
| Control (Path) 1 | 32.8 | Control (Path) 3 | 2.7 |
| Temporal Ctx | | Parietal Ctx | |
| Control (Path) 2 | 37.9 | Control (Path) 4 | 44.8 |
| Temporal Ctx | | Parietal Ctx | |

TABLE KC
Panel 4.1D

| | Rel. Exp. (%) Ag3582 | | Rel. Exp. (%) Ag3582, |
|---|---|---|---|
| Tissue Name | Run 169910467 | Tissue Name | Run 169910467 |
| Secondary Th1 act | 0.0 | HUVEC IL-1beta | 14.3 |
| Secondary Th2 act | 0.1 | HUVEC IFN gamma | 17.2 |
| Secondary Tr1 act | 0.0 | HUVEC TNF alpha + IFN | 16.0 |
| | | gamma | |
| Secondary Th1 rest | 0.0 | HUVEC TNF alpha + IL4 | 10.9 |
| Secondary Th2 rest | 0.0 | HUVEC IL-11 | 4.6 |
| Secondary Tr1 rest | 0.0 | Lung Microvascular EC none | 15.9 |
| Primary Th1 act | 0.0 | Lung Microvascular EC | 16.8 |
| | | TNF alpha + IL-1beta | |
| Primary Th2 act | 0.0 | Microvascular Dermal EC | 6.3 |
| | | none | |
| Primary Tr1 act | 0.0 | Microsvasular Dermal EC | 10.0 |
| | | TNF alpha + IL-1beta | |
| Primary Th1 rest | 0.0 | Bronchial epithelium | 10.4 |
| | | TNF alpha + IL1beta | |
| Primary Th2 rest | 0.0 | Small airway epithelium none | 2.5 |
| Primary Tr1 rest | 0.0 | Small airway epithelium | 4.6 |
| | | TNF alpha + IL-1beta | |
| CD45RA CD4 | 3.2 | Coronery artery SMC rest | 8.2 |
| lymphocyte act | | | |
| CD45RO CD4 | 0.3 | Coronery artery SMC | 6.7 |
| lymphocyte act | | TNF alpha + IL-1beta | |
| CD8 lymphocyte act | 0.0 | Astrocytes rest | 5.0 |
| Secondary CD8 | 0.1 | Astrocytes TNF alpha + IL- | 5.0 |
| lymphocyte rest | | 1beta | |
| Secondary CD8 | 0.0 | KU-812 (Basophil) rest | 0.0 |
| lymphocyte act | | | |
| CD4 lymphocyte none | 0.2 | KU-812 (Basophil) | 0.0 |
| | | PMA/ionomycin | |
| 2ry Th1/Th2/Tr1_anti- | 0.0 | CCD1106 (Keratinocytes) none | 3.5 |
| CD95 CH11 | | | |
| LAK cells rest | 7.9 | CCD1106 (Keratinocytes) | 6.2 |
| | | TNF alpha + IL-1beta | |
| LAK cells IL-2 | 0.4 | Liver cirrhosis | 1.8 |
| LAK cells IL-2 + IL-12 | 0.5 | NCI-H292 none | 3.5 |
| LAK cells IL-2 + IFN | 0.4 | NCI-H292 IL-4 | 4.5 |
| gamma | | | |
| LAK cells IL-2 + IL-18 | 0.6 | NCI-H292 IL-9 | 7.0 |
| LAK cells | 4.7 | NCI-H292 IL-13 | 4.3 |
| PMA/ionomycin | | | |
| NK Cells IL-2 rest | 0.2 | NCI-H292 IFN gamma | 5.0 |
| Two Way MLR 3 day | 7.7 | HPAEC none | 6.9 |
| Two Way MLR 5 day | 4.2 | HPAEC TNF alpha + IL-1Beta | 25.3 |
| Two Way MLR 7 day | 0.8 | Lung fibroblast none | 3.6 |
| PBMC rest | 1.5 | Lung fibroblast TNF alpha + | 6.7 |
| | | IL-1Beta | |
| PBMC PWM | 8.5 | Lung fibroblast IL-4 | 4.3 |
| PBMC PHA-L | 4.1 | Lung fibroblast IL-9 | 5.4 |
| Ramos (B cell) none | 0.0 | Lung fibroblast IL-13 | 3.5 |
| Ramos (B cell) ionomycin | 0.0 | Lung fibroblast IFN gamma | 6.7 |
| B lymphocytes PWM | 0.5 | Dermal fibroblast CCD1070 | 4.5 |
| | | rest | |
| B lymphocytes CD40L | 1.4 | Dermal fibroblast CCD1070 | 6.0 |
| and IL-4 | | TNF alpha | |
| EOL-1 dbcAMP | 0.5 | Dermal fibroblast CCD1070 | 6.0 |
| | | IL-1Beta | |
| EOL-1 dbcAMP | 0.0 | Dermal fibroblast IFN gamma | 6.1 |
| PMA/ionomycin | | | |
| Dendritic cells none | 8.5 | Dermal fibroblast IL-4 | 6.6 |
| Dendritic cells LPS | 22.1 | Dermal Fibroblasts rest | 4.2 |
| Dendritic cells anti-CD40 | 6.1 | Neutrophils TNFa + LPS | 0.1 |
| Monocytes rest | 20.9 | Neutrophils rest | 0.1 |
| Monocytes LPS | 100.0 | Colon | 2.4 |
| Macrophages rest | 14.5 | Lung | 6.9 |
| Macrophages LPS | 24.0 | Thymus | 2.2 |
| HUVEC none | 5.8 | Kidney | 6.6 |
| HUVEC starved | 10.8 | | |

CNS_neurodegeneration_v1.0 Summary: Ag3582 This panel demonstrates the expression of the CG59777-01 gene at moderate levels in the brains of several individuals. However, no differential expression of this gene was detected between Alzheimer's diseased postmortem brains and those of non-demented controls in this experiment. The CG59777-01 gene encodes a protein with homology to vascular Rab-GAP/TBC domain-containing protein (VRP), a protein identified in a screen for angiogenesis-related proteins. See, e.g., Yonekura (2001) Ann N Y Acad Sci 947:382-6.

General_screening_panel_v1.4 Summary: Ag3582 Results from one experiment with the CG59777-01 gene are not included. The amp plot indicates that there were experimental difficulties with this run.

Panel 4.1D Summary: Ag3582 Expression of the CG59777-01 gene is up regulated in LPS-stimulated monocytes (CT=26.2), compared to resting monocytes (CT=28.4). Thus, expression of this gene may be used as a marker for activated monocytes. The putative GTPase activating protein encoded by this gene may therefore be involved in the activation of monocytes in their function as antigen-presenting cells. This suggests that inhibition of the activity of this gene or its protein product may be useful as anti-inflammatory therapeutics for the treatment of autoimmune and inflammatory diseases.

This gene is also expressed at moderate levels in a number of other samples on this panel, including dermal fibroblasts, lung fibroblasts, keratinocytes, astrocytes, bronchial and small airway epithelium, dendritic cells, and macrophages as well as normal tissues represented by colon, lung, thymus and kidney. This ubiquitous pattern of expression suggests that this gene product may be involved in homeostatic processes for these and other cell types and tissues. Furthermore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

L. CG59658-01: F-Box

Expression of gene CG59658-01 was assessed using the primer-probe sets Ag3359 and Ag3765, described in Tables LA and LB. Results of the RTQ-PCR runs are shown in Tables LC, LD, and LE.

TABLE LA
Probe Name Ag3359

| | | SEQ ID | | |
|---|---|---|---|---|
| Primers | Sequences | NO. | Length | Start Position |
| Forward | 5′-gccagaagctcacagatcttt-3′ | 100 | 21 | 1118 |
| Probe | TET-5′-ctctaaagcacatctcccgagggct-3′-TAMRA | 101 | 25 | 1139 |
| Reverse | 5′-ccacagaagctgaggttgag-3′ | 102 | 20 | 1180 |

TABLE LB

PROBE NAME Ag3765

| | | | | |
|---|---|---|---|---|
| | | | | Start |
| Primers | Sequences | SEQ ID NO. | Length | Position |

| | | | | |
|---|---|---|---|---|
| Forward | 5′-accaccctctgtctctcacata-3′ | 103 | 22 | 268 |
| Probe | TET-5′-ccccacacactctcacacacacactt-3′- | 104 | 26 | 317 |
| | TAMRA | | | |
| Reverse | 5′-gagtgtgtgtgtgagtgtgtga-3′ | 105 | 22 | 345 |

TABLE LC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3359, Run | Ag3765, Run | | Ag3359, Run | Ag3765, Run |
| Tissue Name | 210142854 | 211175972 | Tissue Name | 210142854 | 211175972 |
| AD 1 Hippo | 12.9 | 22.5 | Control (Path) 3 | 6.1 | 63.3 |
| | | | Temporal Ctx | | |
| AD 2 Hippo | 28.7 | 29.5 | Control (Path) 4 | 27.7 | 50.3 |
| | | | Temporal Ctx | | |
| AD 3 Hippo | 13.3 | 19.8 | AD 1 Occipital Ctx | 21.3 | 27.7 |
| AD 4 Hippo | 7.9 | 25.5 | AD 2 Occipital Ctx | 0.0 | 48.6 |
| | | | (Missing) | | |
| AD 5 Hippo | 100.0 | 52.5 | AD 3 Occipital Ctx | 11.8 | 21.6 |
| AD 6 Hippo | 41.8 | 46.3 | AD 4 Occipital Ctx | 13.1 | 16.6 |
| Control 2 Hippo | 26.4 | 46.3 | AD 5 Occipital Ctx | 42.9 | 24.5 |
| Control 4 Hippo | 10.7 | 5.5 | AD 6 Occipital Ctx | 14.4 | 52.9 |
| Control (Path) 3 | 3.9 | 26.1 | Control 1 Occipital | 10.7 | 29.7 |
| Hippo | | | Ctx | | |
| AD 1 Temporal Ctx | 23.7 | 62.0 | Control 2 Occipital | 70.7 | 15.2 |
| | | | Ctx | | |
| AD 2 Temporal Ctx | 25.2 | 7.7 | Control 3 Occipital | 17.3 | 32.8 |
| | | | Ctx | | |
| AD 3 Temporal Ctx | 14.0 | 24.1 | Control 4 Occipital | 9.1 | 23.3 |
| | | | Ctx | | |
| AD 4 Temporal Ctx | 15.6 | 25.5 | Control (Path) 1 | 90.1 | 8.8 |
| | | | Occipital Ctx | | |
| AD 5 Inf Temporal | 91.4 | 7.4 | Control (Path) 2 | 8.0 | 8.4 |
| Ctx | | | Occipital Ctx | | |
| AD 5 Sup | 44.1 | 26.1 | Control (Path) 3 | 3.5 | 19.9 |
| Temporal Ctx | | | Occipital Ctx | | |
| AD 6 Inf Temporal | 43.8 | 13.3 | Control (Path) 4 | 17.4 | 9.0 |
| Ctx | | | Occipital Ctx | | |
| AD 6 Sup | 43.2 | 43.5 | Control 1 Parietal | 6.7 | 18.7 |
| Temporal Ctx | | | Ctx | | |
| Control 1 Temporal | 7.9 | 23.3 | Control 2 Parietal | 36.3 | 49.7 |
| Ctx | | | Ctx | | |
| Control 2 Temporal | 40.3 | 15.3 | Control 3 Parietal | 11.7 | 5.9 |
| Ctx | | | Ctx | | |
| Control 3 Temporal | 12.5 | 16.6 | Control (Path) 1 | 75.8 | 49.0 |
| Ctx | | | Parietal Ctx | | |
| Control 3 Temporal | 6.7 | 20.7 | Control (Path) 2 | 20.0 | 10.2 |
| Ctx | | | Parietal Ctx | | |
| Control (Path) 1 | 61.6 | 13.0 | Control (Path) 3 | 7.6 | 100.0 |
| Temporal Ctx | | | Parietal Ctx | | |
| Control (Path) 2 | 26.4 | 34.6 | Control (Path) 4 | 44.8 | 53.2 |
| Temporal Ctx | | | Parietal Ctx | | |

TABLE LD
General_screening_panel_v1.4

| | Rel. Exp. (%) | | Rel. Exp. (%) |
|---|---|---|---|
| | Ag3359, Run | | Ag3359, Run |
| Tissue Name | 216523479 | Tissue Name | 216523479 |
| Adipose | 5.1 | Renal ca. TK-10 | 15.7 |
| Melanoma* Hs688(A).T | 7.9 | Bladder | 10.4 |
| Melanoma* Hs688(B).T | 6.7 | Gastric ca. (liver met.) NCI-N87 | 48.6 |
| Melanoma* M14 | 12.9 | Gastric ca. KATO III | 94.0 |
| Melanoma* LOXIMVI | 29.3 | Colon ca. SW-948 | 15.8 |
| Melanoma* SK-MEL-5 | 21.2 | Colon ca. SW480 | 57.4 |
| Squamous cell carcinoma SCC-4 | 15.0 | Colon ca.* (SW480 met) SW620 | 47.3 |
| Testis Pool | 12.0 | Colon ca. HT29 | 13.6 |
| Prostate ca.* (bone met) PC-3 | 18.6 | Colon ca. HCT-116 | 43.2 |
| Prostate Pool | 5.4 | Colon ca. CaCo-2 | 41.2 |
| Placenta | 8.0 | Colon cancer tissue | 21.5 |
| Uterus Pool | 3.1 | Colon ca. SW1116 | 9.9 |
| Ovarian ca. OVCAR-3 | 40.6 | Colon ca. Colo-205 | 14.7 |
| Ovarian ca. SK-OV-3 | 23.3 | Colon ca. SW-48 | 11.0 |
| Ovarian ca. OVCAR-4 | 10.2 | Colon Pool | 9.2 |
| Ovarian ca. OVCAR-5 | 28.3 | Small Intestine Pool | 11.8 |
| Ovarian ca. IGROV-1 | 12.5 | Stomach Pool | 7.1 |
| Ovarian ca. OVCAR-8 | 17.6 | Bone Marrow Pool | 4.9 |
| Ovary | 7.2 | Fetal Heart | 5.0 |
| Breast ca. MCF-7 | 19.2 | Heart Pool | 3.4 |
| Breast ca. MDA-MB-231 | 21.0 | Lymph Node Pool | 8.0 |
| Breast ca. BT 549 | 31.0 | Fetal Skeletal Muscle | 3.2 |
| Breast ca. T47D | 51.8 | Skeletal Muscle Pool | 3.8 |
| Breast ca. MDA-N | 12.4 | Spleen Pool | 6.3 |
| Breast Pool | 11.0 | Thymus Pool | 9.5 |
| Trachea | 24.0 | CNS cancer (glio/astro) U87-MG | 36.3 |
| Lung | 4.4 | CNS cancer (glio/astro) U-118-MG | 100.0 |
| Fetal Lung | 17.6 | CNS cancer (neuro; met) SK-N-AS | 27.0 |
| Lung ca. NCI-N417 | 6.3 | CNS cancer (astro) SF-539 | 10.9 |
| Lung ca. LX-1 | 37.4 | CNS cancer (astro) SNB-75 | 24.1 |
| Lung ca. NCI-H146 | 21.2 | CNS cancer (glio) SNB-19 | 15.2 |
| Lung ca. SHP-77 | 13.2 | CNS cancer (glio) SF-295 | 45.7 |
| Lung ca. A549 | 25.0 | Brain (Amygdala) Pool | 12.7 |
| Lung ca. NCI-H526 | 29.7 | Brain (cerebellum) | 45.7 |
| Lung ca. NCI-H23 | 51.1 | Brain (fetal) | 23.8 |
| Lung ca. NCI-H460 | 21.8 | Brain (Hippocampus) Pool | 12.5 |
| Lung ca. HOP-62 | 18.4 | Cerebral Cortex Pool | 13.8 |
| Lung ca. NCI-H522 | 24.1 | Brain (Substantia nigra) Pool | 16.4 |
| Liver | 1.0 | Brain (Thalamus) Pool | 17.6 |
| Fetal Liver | 8.7 | Brain (whole) | 22.4 |
| Liver ca. HepG2 | 6.0 | Spinal Cord Pool | 12.9 |
| Kidney Pool | 14.4 | Adrenal Gland | 9.3 |
| Fetal Kidney | 11.3 | Pituitary gland Pool | 8.8 |
| Renal ca. 786-0 | 13.5 | Salivary Gland | 7.2 |
| Renal ca. A498 | 7.2 | Thyroid (female) | 10.1 |
| Renal ca. ACHN | 20.9 | Pancreatic ca. CAPAN2 | 37.1 |
| Renal ca. UO-31 | 21.6 | Pancreas Pool | 14.4 |

TABLE LE
Panel 4D

| | Rel. Exp. (%) | | Rel. Exp. (%) |
|---|---|---|---|
| | Ag3359, Run | | Ag3359, Run |
| Tissue Name | 165231211 | Tissue Name | 165231211 |
| Secondary Th1 act | 15.9 | HUVEC IL-1beta | 5.3 |
| Secondary Th2 act | 16.2 | HUVEC IFN gamma | 8.8 |
| Secondary Tr1 act | 21.5 | HUVEC TNF alpha + IFN gamma | 9.3 |
| Secondary Th1 rest | 8.1 | HUVEC TNF alpha + IL4 | 5.1 |
| Secondary Th2 rest | 8.1 | HUVEC IL-11 | 2.9 |
| Secondary Tr1 rest | 9.9 | Lung Microvascular EC none | 8.2 |
| Primary Th1 act | 9.5 | Lung Microvascular EC TNF alpha + | 4.7 |
| | | IL-1beta | |
| Primary Th2 act | 10.0 | Microvascular Dermal EC none | 9.2 |
| Primary Tr1 act | 17.8 | Microsvasular Dermal EC TNF alpha + | 8.2 |
| | | IL-1beta | |
| Primary Th1 rest | 35.6 | Bronchial epithelium TNF alpha + | 7.5 |
| | | IL1beta | |
| Primary Th2 rest | 19.3 | Small airway epithelium none | 5.1 |
| Primary Tr1 rest | 23.3 | Small airway epithelium TNF alpha + | 20.4 |
| | | IL-1beta | |
| CD45RA CD4 lymphocyte act | 9.5 | Coronery artery SMC rest | 8.4 |
| CD45RO CD4 lymphocyte act | 20.4 | Coronery artery SMC TNF alpha + | 3.8 |
| | | IL-1beta | |
| CD8 lymphocyte act | 20.2 | Astrocytes rest | 6.4 |
| Secondary CD8 lymphocyte rest | 15.8 | Astrocytes TNF alpha + IL-1beta | 4.1 |
| Secondary CD8 lymphocyte act | 10.6 | KU-812 (Basophil) rest | 2.8 |
| CD4 lymphocyte none | 13.0 | KU-812 (Basophil) PMA/ionomycin | 8.6 |
| 2ry Th1/Th2/Tr1_anti-CD95 | 12.5 | CCD1106 (Keratinocytes) none | 6.3 |
| CH11 | | | |
| LAK cells rest | 12.2 | CCD1106 (Keratinocytes) TNF alpha + | 4.4 |
| | | IL-1beta | |
| LAK cells IL-2 | 21.9 | Liver cirrhosis | 2.0 |
| LAK cells IL-2 + IL-12 | 17.0 | Lupus kidney | 3.3 |
| LAK cells IL-2 + IFN gamma | 28.3 | NCI-H292 none | 17.8 |
| LAK cells IL-2 + IL-18 | 19.8 | NCI-H292 IL-4 | 31.2 |
| LAK cells PMA/ionomycin | 4.8 | NCI-H292 IL-9 | 38.7 |
| NK Cells IL-2 rest | 13.7 | NCI-H292 IL-13 | 16.6 |
| Two Way MLR 3 day | 18.2 | NCI-H292 IFN gamma | 20.3 |
| Two Way MLR 5 day | 9.2 | HPAEC none | 6.2 |
| Two Way MLR 7 day | 7.7 | HPAEC TNF alpha + IL-1beta | 6.8 |
| PBMC rest | 9.3 | Lung fibroblast none | 9.3 |
| PBMC PWM | 44.1 | Lung fibroblast TNF alpha + IL-1 | 7.7 |
| | | beta | |
| PBMC PHA-L | 21.5 | Lung fibroblast IL-4 | 15.9 |
| Ramos (B cell) none | 32.8 | Lung fibroblast IL-9 | 15.2 |
| Ramos (B cell) ionomycin | 100.0 | Lung fibroblast IL-13 | 12.1 |
| B lymphocytes PWM | 64.6 | Lung fibroblast IFN gamma | 16.5 |
| B lymphocytes CD40L and IL-4 | 36.3 | Dermal fibroblast CCD1070 rest | 16.5 |
| EOL-1 dbcAMP | 7.9 | Dermal fibroblast CCD1070 TNF | 42.6 |
| | | alpha | |
| EOL-1 dbcAMP | 7.3 | Dermal fibroblast CCD1070 IL-1 | 9.8 |
| PMA/ionomycin | | beta | |
| Dendritic cells none | 8.9 | Dermal fibroblast IFN gamma | 10.6 |
| Dendritic cells LPS | 7.9 | Dermal fibroblast IL-4 | 11.8 |
| Dendritic cells anti-CD40 | 8.4 | IBD Colitis 2 | 0.2 |
| Monocytes rest | 10.4 | IBD Crohn's | 0.6 |
| Monocytes LPS | 11.0 | Colon | 12.7 |
| Macrophages rest | 9.2 | Lung | 10.7 |
| Macrophages LPS | 6.9 | Thymus | 16.7 |
| HUVEC none | 8.7 | Kidney | 35.8 |
| HUVEC starved | 4.8 | | |

CNS_neurodegeneration_v1.0 Summary: Ag3359/Ag3765 This panel confirms the expression of the CG59658-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3359 Highest expression of the CG59658-01 gene is seen in one of the CNS cancer (glio/astro) U-118-MG cell line (CT=28). In addition, expression of this gene is up-regulated in lung cancer, CNS cancer, colon cancer, gastric cancer, pancreatic cancer, ovarian cancer, breast cancer and melanoma cell-lines. Thus, expression of this gene can be used to distinguish cancer cells from normal tissue used in this panel. Furthermore, therapeutic modulation of the activity of the protein encoded by this gene may be beneficial in the treatment of these cancers.

Among tissues with metabolic or endocrine function, this gene is expressed at low to moderate levels in pancreas, adipose, adrenal gland, thyroid, pancrease, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag3765 Results from one experiment with the CG59658-01 gene are not included. The amp plot indicates that there were experimental difficulties with this run.

Panel 4D Summary: Ag3359 Highest expression of the CG59658-01 gene is seen in ionomycin treated Ramos (B cell) sample (CT=27). Lower but still significant levels of expression of this gene are seen in untreated Ramos B cells. In addition, expression of this gene is up-regulated in PWM treated—PBMC and B lymphocytes (CTs=28). B cells represent a principle component of immunity and contribute to the immune response in a number of important functional roles, including antibody production. Production of antibodies against self-antigens is a major component in autoimmune disorders. Since B cells play an important role in autoimmunity, inflammatory processes and inflammatory cascades, therapeutic modulation of this gene product may reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, systemic lupus erythematosus and other autoimmune disorders.

Also, this gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

M. CG59907-01: Novel GAP Protein

Expression of gene CG59907-01 was assessed using the primer-probe set Ag3629, described in Table MA. Results of the RTQ-PCR runs are shown in Tables MB, MC and MD.

TABLE MA

PROBE NAME Ag3629

| | | | | |
|---|---|---|---|---|
| | | SEQ ID | | |
| Primers | Sequences | NO. | Length | Start Position |

| | | | | |
|---|---|---|---|---|
| Forward | 5′-cttcagagctagggacgaaca-3′ | 106 | 21 | 1584 |
| Probe | TET-5′-agtgtgctcccacctcaggacctt-3′- | 107 | 24 | 1607 |
| | TAMRA | | | |
| Reverse | 5′-tctagaagcctggagggaact-3′ | 108 | 21 | 1661 |

TABLE MB
CNS_neurodegeneration_v1.0

| | Rel. Exp. (%) | | Rel. Exp. (%) |
|---|---|---|---|
| | Ag3629, Run | | Ag3629, Run |
| Tissue Name | 211020446 | Tissue Name | 211020446 |
| AD 1 Hippo | 13.4 | Control (Path) 3 Temporal Ctx | 2.8 |
| AD 2 Hippo | 28.3 | Control (Path) 4 Temporal Ctx | 20.2 |
| AD 3 Hippo | 17.7 | AD 1 Occipital Ctx | 19.5 |
| AD 4 Hippo | 8.4 | AD 2 Occipital Ctx (Missing) | 0.0 |
| AD 5 Hippo | 45.4 | AD 3 Occipital Ctx | 16.6 |
| AD 6 Hippo | 51.1 | AD 4 Occipital Ctx | 17.0 |
| Control 2 Hippo | 21.2 | AD 5 Occipital Ctx | 25.5 |
| Control 4 Hippo | 4.5 | AD 6 Occipital Ctx | 14.7 |
| Control (Path) 3 Hippo | 1.8 | Control 1 Occipital Ctx | 1.5 |
| AD 1 Temporal Ctx | 26.8 | Control 2 Occipital Ctx | 41.2 |
| AD 2 Temporal Ctx | 47.0 | Control 3 Occipital Ctx | 32.3 |
| AD 3 Temporal Ctx | 18.3 | Control 4 Occipital Ctx | 4.1 |
| AD 4 Temporal Ctx | 25.0 | Control (Path) 1 Occipital Ctx | 77.4 |
| AD 5 Inf Temporal Ctx | 33.2 | Control (Path) 2 Occipital Ctx | 22.1 |
| AD 5 Sup Temporal Ctx | 21.6 | Control (Path) 3 Occipital Ctx | 1.0 |
| AD 6 Inf Temporal Ctx | 80.1 | Control (Path) 4 Occipital Ctx | 23.7 |
| AD 6 Sup Temporal Ctx | 100.0 | Control 1 Parietal Ctx | 4.3 |
| Control 1 Temporal Ctx | 3.6 | Control 2 Parietal Ctx | 25.0 |
| Control 2 Temporal Ctx | 17.4 | Control 3 Parietal Ctx | 11.8 |
| Control 3 Temporal Ctx | 26.6 | Control (Path) 1 Parietal Ctx | 39.8 |
| Control 3 Temporal Ctx | 6.6 | Control (Path) 2 Parietal Ctx | 26.8 |
| Control (Path) 1 Temporal Ctx | 47.0 | Control (Path) 3 Parietal Ctx | 1.6 |
| Control (Path) 2 Temporal Ctx | 40.1 | Control (Path) 4 Parietal Ctx | 26.6 |

TABLE MC
General_screening_panel_v1.4

| | Rel. Exp. (%) | | Rel. Exp. (%) |
|---|---|---|---|
| | Ag3629, Run | | Ag3629, Run |
| Tissue Name | 218212064 | Tissue Name | 218212064 |
| Adipose | 0.8 | Renal ca. TK-10 | 9.8 |
| Melanoma* Hs688(A).T | 1.3 | Bladder | 3.0 |
| Melanoma* Hs688(B).T | 1.4 | Gastric ca. (liver met.) NCI-N87 | 19.9 |
| Melanoma* M14 | 1.9 | Gastric ca. KATO III | 3.8 |
| Melanoma* LOXIMVI | 0.5 | Colon ca. SW-948 | 1.1 |
| Melanoma* SK-MEL-5 | 1.3 | Colon ca. SW480 | 6.9 |
| Squamous cell carcinoma | 2.0 | Colon ca.* (SW480 met) SW620 | 2.8 |
| SCC-4 | | | |
| Testis Pool | 22.1 | Colon ca. HT29 | 7.2 |
| Prostate ca.* (bone met) PC-3 | 1.7 | Colon ca. HCT-116 | 4.6 |
| Prostate Pool | 0.0 | Colon ca. CaCo-2 | 6.8 |
| Placenta | 0.7 | Colon cancer tissue | 3.5 |
| Uterus Pool | 1.4 | Colon ca. SW1116 | 1.7 |
| Ovarian ca. OVCAR-3 | 2.8 | Colon ca. Colo-205 | 0.6 |
| Ovarian ca. SK-OV-3 | 15.1 | Colon ca. SW-48 | 0.4 |
| Ovarian ca. OVCAR-4 | 0.8 | Colon Pool | 3.6 |
| Ovarian ca. OVCAR-5 | 70.7 | Small Intestine Pool | 4.7 |
| Ovarian ca. IGROV-1 | 3.1 | Stomach Pool | 2.9 |
| Ovarian ca. OVCAR-8 | 2.0 | Bone Marrow Pool | 1.9 |
| Ovary | 2.0 | Fetal Heart | 1.6 |
| Breast ca. MCF-7 | 2.8 | Heart Pool | 1.4 |
| Breast ca. MDA-MB-231 | 4.7 | Lymph Node Pool | 6.4 |
| Breast ca. BT 549 | 14.3 | Fetal Skeletal Muscle | 2.5 |
| Breast ca. T47D | 100.0 | Skeletal Muscle Pool | 5.3 |
| Breast ca. MDA-N | 1.3 | Spleen Pool | 3.3 |
| Breast Pool | 5.7 | Thymus Pool | 6.1 |
| Trachea | 3.1 | CNS cancer (glio/astro) U87-MG | 2.7 |
| Lung | 1.7 | CNS cancer (glio/astro) U-118- | 7.5 |
| | | MG | |
| Fetal Lung | 15.2 | CNS cancer (neuro; met) SK-N- | 7.8 |
| | | AS | |
| Lung ca. NCI-N417 | 0.2 | CNS cancer (astro) SF-539 | 2.2 |
| Lung ca. LX-1 | 4.4 | CNS cancer (astro) SNB-75 | 7.0 |
| Lung ca. NCI-H146 | 1.1 | CNS cancer (glio) SNB-19 | 3.8 |
| Lung ca. SHP-77 | 1.3 | CNS cancer (glio) SF-295 | 15.1 |
| Lung ca. A549 | 3.8 | Brain (Amygdala) Pool | 1.4 |
| Lung ca. NCI-H526 | 0.5 | Brain (cerebellum) | 5.0 |
| Lung ca. NCI-H23 | 15.1 | Brain (fetal) | 9.3 |
| Lung ca. NCI-H460 | 3.7 | Brain (Hippocampus) Pool | 1.5 |
| Lung ca. HOP-62 | 6.3 | Cerebral Cortex Pool | 1.9 |
| Lung ca. NCI-H522 | 7.6 | Brain (Substantia nigra) Pool | 1.4 |
| Liver | 0.0 | Brain (Thalamus) Pool | 3.9 |
| Fetal Liver | 2.5 | Brain (whole) | 1.8 |
| Liver ca. HepG2 | 1.0 | Spinal Cord Pool | 3.0 |
| Kidney Pool | 6.1 | Adrenal Gland | 2.6 |
| Fetal Kidney | 10.9 | Pituitary gland Pool | 1.8 |
| Renal ca. 786-0 | 11.8 | Salivary Gland | 1.1 |
| Renal ca. A498 | 3.1 | Thyroid (female) | 0.3 |
| Renal ca. ACHN | 1.3 | Pancreatic ca. CAPAN2 | 30.6 |
| Renal ca. UO-31 | 3.7 | Pancreas Pool | 6.6 |

TABLE MD
Panel 4.1D

| | Rel. Exp. (%) | | Rel. Exp. (%) |
|---|---|---|---|
| | Ag3629, Run | | Ag3629, Run |
| Tissue Name | 169960601 | Tissue Name | 169960601 |
| Secondary Th1 act | 32.5 | HUVEC IL-1beta | 29.3 |
| Secondary Th2 act | 47.3 | HUVEC IFN gamma | 37.4 |
| Secondary Tr1 act | 57.0 | HUVEC TNF alpha + IFN gamma | 28.7 |
| Secondary Th1 rest | 24.8 | HUVEC TNF alpha + IL4 | 32.8 |
| Secondary Th2 rest | 47.0 | HUVEC IL-11 | 35.4 |
| Secondary Tr1 rest | 41.5 | Lung Microvascular EC none | 42.0 |
| Primary Th1 act | 30.6 | Lung Microvascular EC TNF alpha + | 33.0 |
| | | IL-1beta | |
| Primary Th2 act | 32.5 | Microvascular Dermal EC none | 44.8 |
| Primary Tr1 act | 38.4 | Microsvasular Dermal EC | 38.2 |
| | | TNF alpha + IL-1beta | |
| Primary Th1 rest | 62.9 | Bronchial epithelium TNF alpha + | 85.3 |
| | | IL1beta | |
| Primary Th2 rest | 42.0 | Small airway epithelium none | 14.8 |
| Primary Tr1 rest | 47.6 | Small airway epithelium TNF alpha + | 23.8 |
| | | IL-1beta | |
| CD45RA CD4 lymphocyte act | 31.2 | Coronery artery SMC rest | 8.2 |
| CD45RO CD4 lymphocyte act | 59.9 | Coronery artery SMC TNF alpha + | 8.4 |
| | | IL-1beta | |
| CD8 lymphocyte act | 49.3 | Astrocytes rest | 27.0 |
| Secondary CD8 lymphocyte | 62.4 | Astrocytes TNF alpha + IL-1beta | 24.0 |
| rest | | | |
| Secondary CD8 lymphocyte | 36.3 | KU-812 (Basophil) rest | 28.5 |
| act | | | |
| CD4 lymphocyte none | 32.5 | KU-812 (Basophil) | 29.5 |
| | | PMA/ionomycin | |
| 2ry Th1/Th2/Tr1_anti-CD95 | 48.0 | CCD1106 (Keratinocytes) none | 11.7 |
| CH11 | | | |
| LAK cells rest | 77.4 | CCD1106 (Keratinocytes) | 17.0 |
| | | TNF alpha + IL-1beta | |
| LAK cells IL-2 | 77.9 | Liver cirrhosis | 21.2 |
| LAK cells IL-2 + IL-12 | 62.4 | NCI-H292 none | 68.8 |
| LAK cells IL-2 + IFN gamma | 62.4 | NCI-H292 IL-4 | 82.4 |
| LAK cells IL-2 + IL-18 | 57.0 | NCI-H292 IL-9 | 70.7 |
| LAK cells PMA/ionomycin | 31.0 | NCI-H292 IL-13 | 63.7 |
| NK Cells IL-2 rest | 62.9 | NCI-H292 IFN gamma | 66.0 |
| Two Way MLR 3 day | 76.3 | HPAEC none | 17.7 |
| Two Way MLR 5 day | 34.4 | HPAEC TNF alpha + IL-1Beta | 31.4 |
| Two Way MLR 7 day | 31.9 | Lung fibroblast none | 44.4 |
| PBMC rest | 25.3 | Lung fibroblast TNF alpha + IL-1 | 23.3 |
| | | beta | |
| PBMC PWM | 26.4 | Lung fibroblast IL-4 | 20.3 |
| PBMC PHA-L | 59.0 | Lung fibroblast IL-9 | 27.9 |
| Ramos (B cell) none | 84.7 | Lung fibroblast IL-13 | 26.8 |
| Ramos (B cell) ionomycin | 73.7 | Lung fibroblast IFN gamma | 25.3 |
| B lymphocytes PWM | 28.9 | Dermal fibroblast CCD1070 rest | 25.0 |
| B lymphocytes CD40L and IL-4 | 60.7 | Dermal fibroblast CCD1070 TNF | 74.2 |
| | | alpha | |
| EOL-1 dbcAMP | 32.5 | Dermal fibroblast CCD1070 IL-1 | 14.1 |
| | | beta | |
| EOL-1 dbcAMP | 60.3 | Dermal fibroblast IFN gamma | 13.0 |
| PMA/ionomycin | | | |
| Dendritic cells none | 43.5 | Dermal fibroblast IL-4 | 21.8 |
| Dendritic cells LPS | 49.3 | Dermal Fibroblasts rest | 10.6 |
| Dendritic cells anti-CD40 | 51.8 | Neutrophils TNFa + LPS | 9.5 |
| Monocytes rest | 67.4 | Neutrophils rest | 50.7 |
| Monocytes LPS | 84.1 | Colon | 9.1 |
| Macrophages rest | 50.3 | Lung | 3.6 |
| Macrophages LPS | 51.1 | Thymus | 100.0 |
| HUVEC none | 32.3 | Kidney | 22.1 |
| HUVEC starved | 52.5 | | |

CNS_neurodegeneration_v1.0 Summary: Ag3629 This panel confirms the expression of the CG59907-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3629 Highest expression of the CG59907-01 gene is seen in a breast cancer cell line (CT=26.4), with expression widespread throughout this panel. Significant levels of expression are also seen in samples derived from an ovarian cancer cell line and a pancreatic cancer cell line. Thus, expression of this gene could be used to differentiate between these samples and other samples on this panel and as a marker to detect the presence of breast, ovarian, and pancreatic cancers. Furthermore, therapeutic modulation of the expression or function of this gene may be effective in the treatment of breast, ovarian, and pancreatic cancers.

In addition, this gene is expressed at significant levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic function, this gene is expressed at moderate to low levels in pituitary, adipose, adrenal gland, pancreas, thyroid, fetal liver and adult and fetal skeletal muscle, and heart. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal lung and liver tissue (CTs=29-32) when compared to expression in the adult counterpart (CTs=32-38). Thus, expression of this gene may be used to differentiate between the fetal and adult source of these tissue.

Panel 4.1D Summary: Ag3629 Expression of the CG59907-01 gene is ubiquitous in this panel, with highest expression in the thymus (CT=31.7). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

N. CG59903-01: Novel Nuclear Protein

Expression of gene CG59903-01 was assessed using the primer-probe set Ag3628, described in Table NA. Results of the RTQ-PCR runs are shown in Tables NB, NC and ND.

TABLE NA

PROBE NAME Ag3628

| | | | | |
|---|---|---|---|---|
| | | SEQ ID | | |
| Primers | Sequences | NO. | Length | Start Position |

| | | | | |
|---|---|---|---|---|
| Forward | 5′-tgaccatcttggtgagaaaca-3′ | 109 | 21 | 109 |
| Probe | TET-5′-ctaccaggaagttctccagcagctga-3′- | 110 | 26 | 140 |
| | TAMRA | | | |
| Reverse | 5′-aacattgggcaaattcattaca-3′ | 111 | 22 | 167 |

TABLE NB
CNS_neurodegeneration_v1.0

| | Rel. Exp. (%) | | Rel. Exp. (%) |
|---|---|---|---|
| | Ag3628, Run | | Ag3628, Run |
| Tissue Name | 211020355 | Tissue Name | 211020355 |
| AD 1 Hippo | 10.2 | Control (Path) 3 Temporal Ctx | 12.2 |
| AD 2 Hippo | 35.6 | Control (Path) 4 Temporal Ctx | 42.3 |
| AD 3 Hippo | 12.9 | AD 1 Occipital Ctx | 16.8 |
| AD 4 Hippo | 21.8 | AD 2 Occipital Ctx (Missing) | 0.0 |
| AD 5 hippo | 63.3 | AD 3 Occipital Ctx | 7.9 |
| AD 6 Hippo | 50.0 | AD 4 Occipital Ctx | 19.1 |
| Control 2 Hippo | 13.9 | AD 5 Occipital Ctx | 15.4 |
| Control 4 Hippo | 20.6 | AD 6 Occipital Ctx | 24.1 |
| Control (Path) 3 Hippo | 9.1 | Control 1 Occipital Ctx | 21.6 |
| AD 1 Temporal Ctx | 28.3 | Control 2 Occipital Ctx | 36.6 |
| AD 2 Temporal Ctx | 36.9 | Control 3 Occipital Ctx | 10.6 |
| AD 3 Temporal Ctx | 16.3 | Control 4 Occipital Ctx | 7.5 |
| AD 4 Temporal Ctx | 33.7 | Control (Path) 1 Occipital Ctx | 91.4 |
| AD 5 Inf Temporal Ctx | 97.3 | Control (Path) 2 Occipital Ctx | 14.4 |
| AD 5 SupTemporal Ctx | 36.9 | Control (Path) 3 Occipital Ctx | 6.2 |
| AD 6 Inf Temporal Ctx | 59.9 | Control (Path) 4 Occipital Ctx | 13.2 |
| AD 6 Sup Temporal Ctx | 75.3 | Control 1 Parietal Ctx | 19.1 |
| Control 1 Temporal Ctx | 29.1 | Control 2 Parietal Ctx | 49.3 |
| Control 2 Temporal Ctx | 35.1 | Control 3 Parietal Ctx | 12.5 |
| Control 3 Temporal Ctx | 12.5 | Control (Path) 1 Parietal Ctx | 100.0 |
| Control 4 Temporal Ctx | 16.5 | Control (Path) 2 Parietal Ctx | 44.8 |
| Control (Path) 1 Temporal Ctx | 87.1 | Control (Path) 3 Parietal Ctx | 8.2 |
| Control (Path) 2 Temporal Ctx | 74.2 | Control (Path) 4 Parietal Ctx | 34.9 |

TABLE NC
General_screening_panel_v1.4

| | Rel. Exp. (%) | | Rel. Exp. (%) |
|---|---|---|---|
| | Ag3628, Run | | Ag3628, Run |
| Tissue Name | 218211823 | Tissue Name | 218211823 |
| Adipose | 6.1 | Renal ca. TK-10 | 23.2 |
| Melanoma* Hs688(A).T | 0.0 | Bladder | 19.1 |
| Melanoma* Hs688(B).T | 0.1 | Gastric ca. (liver met.) NCI-N87 | 15.5 |
| Melanoma* M14 | 0.0 | Gastric ca. KATO III | 56.3 |
| Melanoma* LOXIMVI | 0.1 | Colon ca. SW-948 | 12.2 |
| Melanoma* SK-MEL-5 | 11.0 | Colon ca. SW480 | 25.3 |
| Squamous cell carcinoma SCC-4 | 28.5 | Colon ca.* (SW480 met) SW620 | 27.5 |
| Testis Pool | 7.7 | Colonl ca. HT29 | 16.7 |
| Prostate ca.* (bone met) PC-3 | 14.6 | Colon ca. HCT-116 | 23.7 |
| Prostate Pool | 0.0 | Colon ca. CaCo-2 | 21.6 |
| Placenta | 3.0 | Colon cancer tissue | 13.6 |
| Uterus Pool | 3.0 | Colon ca. SW1116 | 7.7 |
| Ovarian ca. OVCAR-3 | 19.1 | Colon ca. Colo-205 | 4.2 |
| Ovarian ca. SK-OV-3 | 13.2 | Colon ca. SW-48 | 4.0 |
| Ovarian ca. OVCAR-4 | 14.3 | Colon Pool | 8.5 |
| Ovarian ca. OVCAR-5 | 64.2 | Small Intestine Pool | 7.4 |
| Ovarian ca. IGROV-1 | 5.1 | Stomach Pool | 5.4 |
| Ovarian ca. OVCAR-8 | 2.1 | Bone Marrow Pool | 3.6 |
| Ovary | 3.3 | Fetal Heart | 3.9 |
| Breast ca. MCF-7 | 23.8 | Heart Pool | 2.2 |
| Breast ca. MDA-MB-231 | 31.9 | Lymph Node Pool | 9.0 |
| Breast ca. BT 549 | 34.9 | Fetal Skeletal Muscle | 4.7 |
| Breast ca. T47D | 100.0 | Skeletal Muscle Pool | 2.0 |
| Breast ca. MDA-N | 0.0 | Spleen Pool | 34.2 |
| Breast Pool | 11.0 | Thymus Pool | 47.3 |
| Trachea | 28.1 | CNS cancer (glio/astro) U87-MG | 0.0 |
| Lung | 0.7 | CNS cancer (glio/astro) U-118-MG | 0.0 |
| Fetal Lung | 60.3 | CNS cancer (neuro; met) SK-N-AS | 0.0 |
| Lung ca. NCI-N417 | 3.4 | CNS cancer (astro) SF-539 | 0.1 |
| Lung ca. LX-1 | 33.0 | CNS cancer (astro) SNB-75 | 0.5 |
| Lung ca. NCI-H146 | 8.7 | CNS cancer (glio) SNB-19 | 7.0 |
| Lung ca. SHP-77 | 61.1 | CNS cancer (glio) SF-295 | 7.7 |
| Lung ca. A549 | 11.6 | Brain (Amygdala) Pool | 3.6 |
| Lung ca. NCI-H526 | 7.7 | Brain (cerebellum) | 1.5 |
| Lung ca. NCI-H23 | 8.7 | Brain (fetal) | 23.8 |
| Lung ca. NCI-H460 | 9.3 | Brain (Hippocampus) Pool | 2.9 |
| Lung ca. HOP-62 | 3.5 | Cerebral Cortex Pool | 2.1 |
| Lung ca. NCI-H522 | 32.5 | Brain (Substantia nigra) Pool | 2.5 |
| Liver | 0.6 | Brain (Thalamus) Pool | 5.7 |
| Fetal Liver | 17.0 | Brain (whole) | 6.0 |
| Liver ca. HepG2 | 14.0 | Spinal Cord Pool | 4.2 |
| Kidney Pool | 14.3 | Adrenal Gland | 2.2 |
| Fetal Kidney | 18.6 | Pituitary gland Pool | 3.2 |
| Renal ca. 786-0 | 4.8 | Salivary Gland | 8.1 |
| Renal ca. A498 | 0.6 | Thyroid (female) | 1.5 |
| Renal ca. ACHN | 4.9 | Pancreatic ca. CAPAN2 | 23.7 |
| Renal ca. UO-31 | 7.4 | Pancreas Pool | 12.9 |

TABLE ND
Panel 4.1D

| | Rel. Exp.(%) | | Rel. Exp.(%) |
|---|---|---|---|
| | Ag3628, | | Ag3628, |
| | Run | | Run |
| Tissue Name | 169960545 | Tissue Name | 169960545 |
| Secondary Th1 act | 46.0 | HUVEC IL-1beta | 11.5 |
| Secondary Th2 act | 82.4 | HUVEC IFN gamma | 14.3 |
| Secondary Tr1 act | 85.3 | HUVEC TNF alpha + | 7.1 |
| | | IFN gamma | |
| Secondary Th1 rest | 37.1 | HUVEC TNF alpha + | 7.2 |
| | | IL4 | |
| Secondary Th2 rest | 77.4 | HUVEC IL-11 | 10.7 |
| Secondary Tr1 rest | 65.5 | Lung Microvascular | 10.7 |
| | | EC none | |
| Primary Th1 act | 25.0 | Lung Microvascular | 6.1 |
| | | EC TNFalpha + | |
| | | IL-1beta | |
| Primary Th2 act | 49.7 | Microvascular Dermal | 9.1 |
| | | EC none | |
| Primary Tr1 act | 36.9 | Microsvasular Dermal | 5.9 |
| | | EC TNFalpha + | |
| | | IL-1beta | |
| Primary Th1 rest | 80.7 | Bronchial epithelium | 9.7 |
| | | TNFalpha + IL1beta | |
| Primary Th2 rest | 74.2 | Small airway | 2.8 |
| | | epithelium none | |
| Primary Tr1 rest | 100.0 | Small airway | 6.3 |
| | | epithelium | |
| | | TNFalpha + IL-1beta | |
| CD45RA CD4 | 27.2 | Coronery artery SMC | 0.2 |
| lymphocyte act | | rest | |
| CD45RO CD4 | 64.2 | Coronery artery SMC | 0.5 |
| lymphocyte act | | TNFalpha + IL-1beta | |
| CD8 lymphocyte act | 55.5 | Astrocytes rest | 2.5 |
| Secondary CD8 | 52.5 | Astrocytes | 1.6 |
| lymphocyte rest | | TNFalpha + IL-1beta | |
| Secondary CD8 | 40.3 | KU-812 (Basophil) | 22.8 |
| lymphocyte act | | rest | |
| CD4 lymphocyte | 75.8 | KU-812 (Basophil) | 17.2 |
| none | | PMA/ionomycin | |
| 2ry | 70.2 | CCD1106 | 18.0 |
| Th1/Th2/Tr1_anti- | | (Keratinocytes) none | |
| CD95 CH11 | | | |
| LAK cells rest | 40.9 | CCD1106 | 21.0 |
| | | (Keratinocytes) | |
| | | TNFalpha + IL-1beta | |
| LAK cells IL-2 | 75.3 | Liver cirrhosis | 5.9 |
| LAK cells IL-2 + | 56.6 | NCI-H292 none | 7.9 |
| IL-12 | | | |
| LAK cells IL-2 + | 85.9 | NCI-H292 IL-4 | 11.9 |
| IFN gamma | | | |
| LAK cells IL-2 + | 79.6 | NCI-H292 IL-9 | 14.5 |
| IL-18 | | | |
| LAK cells | 11.7 | NCI-H292 IL-13 | 9.6 |
| PMA/ionomycin | | | |
| NK Cells IL-2 rest | 95.3 | NCI-H292 IFN gamma | 18.6 |
| Two Way MLR | 76.8 | HPAEC none | 12.8 |
| 3 day | | | |
| Two Way MLR | 29.5 | HPAEC TNF | 5.7 |
| 5 day | | alpha + IL-1 beta | |
| Two Way MLR | 28.9 | Lung fibroblast none | 0.2 |
| 7 day | | | |
| PBMC rest | 64.6 | Lung fibroblast TNF | 0.1 |
| | | alpha + IL-1 beta | |
| PBMC PWM | 24.3 | Lung fibroblast IL-4 | 0.3 |
| PBMC PHA-L | 24.5 | Lung fibroblast IL-9 | 0.3 |
| Ramos (B cell) none | 81.2 | Lung fibroblast IL-13 | 0.6 |
| Ramos (B cell) | 51.1 | Lung fibroblast | 0.4 |
| ionomycin | | IFN gamma | |
| B lymphocytes | 26.4 | Dermal fibroblast | 0.0 |
| PWM | | CCD1070 rest | |
| B lymphocytes | 65.5 | Dermal fibroblast | 63.3 |
| CD40L and IL-4 | | CCD1070 TNF alpha | |
| EOL-1 dbcAMP | 17.6 | Dermal fibroblast | 0.0 |
| | | CCD1070 IL-1 beta | |
| EOL-1 dbcAMP | 42.3 | Dermal fibroblast | 0.0 |
| PMA/ionomycin | | IFN gamma | |
| Dendritic cells none | 7.3 | Dermal fibroblast IL-4 | 0.0 |
| Dendritic cells LPS | 0.6 | Dermal Fibroblasts | 0.0 |
| | | rest | |
| Dendritic cells | 1.6 | Neutrophils | 2.0 |
| anti-CD40 | | TNFa + LPS | |
| Monocytes rest | 57.4 | Neutrophils rest | 6.9 |
| Monocytes LPS | 8.3 | Colon | 7.4 |
| Macrophages rest | 12.4 | Lung | 15.9 |
| Macrophages LPS | 2.8 | Thymus | 73.7 |
| HUVEC none | 8.6 | Kidney | 12.5 |
| HUVEC starved | 15.4 | | |

CNS_neurodegeneration_v1.0 Summary: Ag3628 This panel does not show differential expression of the CG59903-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3628 Highest expression of the CG59903-01 gene is seen in a breast cancer cell line (CT=25.5). Significant levels of expression are also seen in cell lines derived from gastric, lung, ovarian, and colon cancers. Thus, expression of this gene could be used as a marker to detect the presence of these cancers. Furthermore, therapeutic modulation of the expression or function of this gene may be effective in the treatment of breast, gastric, lung, ovarian, and colon cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is also expressed at moderate levels in the CNS and may be a small molecule target for the treatment of neurologic diseases.

In addition, expression of this gene is higher in fetal lung and liver (CTs=26-28) when compared to expression in the corresponding adult tissues (CTs=32). Thus, expression of this gene may be used to differentiate between the fetal and adult sources of these tissue.

Panel 4.1D Summary: Ag3628 Highest expression of the CG59903-01 gene is seen in a sample derived from primary resting T cells (CT=27), with expression widespread throughout this panel.

Significant levels of expression are also found in other T cells including activated primary Th1, Th2 and Tr1 cells, resting primary Th2 and Tr1 cells, CD45RO CD4 lymphocytes, resting secondary CD8 lymphocytes, and IL2+IL12 and IL2+IL18 stimulated lymphokine activated killer (LAK) cells. In additional, expression is detected in peripheral blood mononuclear cells (PBMC), polkweed mitogen stimulated B lymphocytes, normal thymus, and stimulated dermal fibroblasts. Since eosinophils B cells and T cells play an important role in lung pathology, inflammatory bowel disease and autoimmune disorders, therapeutic modulation of the protein encoded by this gene could block or inhibit inflammation or tissue damage due to lung conditions including asthma, allergies, hypersensitivity reactions, inflammatory bowel disease, viral infections and autoimmune diseases, such as rheumatoid arthritis, systemic lupus erythematosus. In addition, the expression of this gene in T cells, B cells, and PBMCs also suggests that therapeutic modulation of this gene product may ameliorate symptoms associated with synovitis associated with osteoarthritis. Furthermore, detection of this gene in LAK cells suggests that modulation of the function of this gene product may also lead to improvement of symptoms associated with tumor immunology and tumor cell clearance, as well as removal of virally and bacterial infected cells.

O. CG59985-01: WD Repeat Protein—Isoform1, Submitted to Study DDNPAT on Mar. 28, 2001 by cpena; Clone Status=FIS; Novelty=Novel; ORF Start=14, ORF Stop=962, Frame=2; 1043 bp.

Expression of gene CG59985-01 was assessed using the primer-probe set Ag3642, described in Table OA. Results of the RTQ-PCR runs are shown in Tables OB, and OC.

TABLE OA

PROBE NAME Ag3642

| | | | | |
|---|---|---|---|---|
| Primers | Sequences | SEQ ID NO. | Length | Start Position |

| | | | | |
|---|---|---|---|---|
| Forward | 5′-ggcctatgaggcagaggat-3′ | 112 | 19 | 934 |
| Probe | TET-5′-caccaacaggaccaaggaccgag-3′-TAMRA | 113 | 23 | 977 |
| Reverse | 5′-tgaagtccttccatgtctgtgt-3′ | 114 | 22 | 1000 |

TABLE OB
General_screening_panel_v1.4

| | Rel. Exp.(%) | | Rel. Exp.(%) |
|---|---|---|---|
| | Ag3642, | | Ag3642, |
| | Run | | Run |
| Tissue Name | 218306351 | Tissue Name | 218306351 |
| Adipose | 2.7 | Renal ca. TK-10 | 15.6 |
| Melanoma* Hs688(A).T | 3.1 | Bladder | 11.0 |
| Melanoma* Hs688(B).T | 3.4 | Gastric ca. (liver | 25.3 |
| | | met.) NCI-N87 | |
| Melanoma* M14 | 25.2 | Gastric ca. | 15.1 |
| | | KATO III | |
| Melanoma* LOXIMVI | 6.7 | Colon ca. | 8.2 |
| | | SW-948 | |
| Melanoma* SK-MEL-5 | 8.0 | Colon ca. SW480 | 24.7 |
| Squamous cell | 4.9 | Colon ca.* | 22.4 |
| carcinoma SCC-4 | | (SW480 met) | |
| | | SW620 | |
| Testis Pool | 6.6 | Colon ca. HT29 | 6.8 |
| Prostate ca.* | 31.2 | Colon ca. | 37.1 |
| (bone met) PC-3 | | HCT-116 | |
| Prostate Pool | 5.1 | Colon ca. CaCo-2 | 14.5 |
| Placenta | 3.8 | Colon cancer | 6.6 |
| | | tissue | |
| Uterus Pool | 3.6 | Colon ca. | 7.9 |
| | | SW1116 | |
| Ovarian ca. OVCAR-3 | 11.3 | Colon ca. | 7.9 |
| | | Colo-205 | |
| Ovarian ca. SK-OV-3 | 27.7 | Colon ca. SW-48 | 4.9 |
| Ovarian ca. OVCAR-4 | 11.5 | Colon Pool | 9.7 |
| Ovarian ca. OVCAR-5 | 50.7 | Small Intestine | 10.9 |
| | | Pool | |
| Ovarian ca. IGROV-1 | 19.3 | Stomach Pool | 6.8 |
| Ovarian ca. OVCAR-8 | 17.0 | Bone Marrow | 2.0 |
| | | Pool | |
| Ovary | 4.6 | Fetal Heart | 6.6 |
| Breast ca. MCF-7 | 35.6 | Heart Pool | 4.4 |
| Breast ca. MDA-MB-231 | 33.4 | Lymph Node | 9.0 |
| | | Pool | |
| Breast ca. BT 549 | 24.5 | Fetal Skeletal | 4.4 |
| | | Muscle | |
| Breast ca. T47D | 100.0 | Skeletal Muscle | 4.4 |
| | | Pool | |
| Breast ca. MDA-N | 23.7 | Spleen Pool | 5.3 |
| Breast Pool | 9.1 | Thymus Pool | 13.3 |
| Trachea | 5.9 | CNS cancer | 25.9 |
| | | (glio/astro) | |
| | | U87-MG | |
| Lung | 4.0 | CNS cancer | 32.1 |
| | | (glio/astro) | |
| | | U-118-MG | |
| Fetal Lung | 20.4 | CNS cancer | 33.0 |
| | | (neuro;met) | |
| | | SK-N-AS | |
| Lung ca. NCI-N417 | 5.1 | CNS cancer | 4.8 |
| | | (astro) SF-539 | |
| Lung ca. LX-1 | 29.7 | CNS cancer | 31.2 |
| | | (astro) SNB-75 | |
| Lung ca. NCI-H146 | 19.9 | CNS cancer | 15.9 |
| | | (glio) SNB-19 | |
| Lung ca. SHP-77 | 24.5 | CNS cancer | 63.7 |
| | | (glio) SF-295 | |
| Lung ca. A549 | 11.2 | Brain (Amygdala) | 8.4 |
| | | Pool | |
| Lung ca. NCI-H526 | 9.9 | Brain | 27.7 |
| | | (cerebellum) | |
| Lung ca. NCI-H23 | 19.1 | Brain (fetal) | 13.9 |
| Lung ca. NCI-H460 | 8.8 | Brain | 8.1 |
| | | (Hippocampus) | |
| | | Pool | |
| Lung ca. HOP-62 | 8.7 | Cerebral Cortex | 7.9 |
| | | Pool | |
| Lung ca. NCI-H522 | 25.9 | Brain (Substantia | 9.7 |
| | | nigra) Pool | |
| Liver | 0.1 | Brain (Thalamus) | 10.7 |
| | | Pool | |
| Fetal Liver | 5.6 | Brain (whole) | 5.9 |
| Liver ca. HepG2 | 10.1 | Spinal Cord Pool | 6.9 |
| Kidney Pool | 19.3 | Adrenal Gland | 4.5 |
| Fetal Kidney | 15.5 | Pituitary gland | 4.2 |
| | | Pool | |
| Renal ca. 786-0 | 9.8 | Salivary Gland | 2.5 |
| Renal ca. A498 | 5.6 | Thyroid (female) | 3.8 |
| Renal ca. ACHN | 9.7 | Pancreatic ca. | 9.2 |
| | | CAPAN2 | |
| Renal ca. UO-31 | 7.1 | Pancreas Pool | 11.1 |

TABLE OD
Panel 4.1D

| | Rel. Exp.(%) | | Rel. Exp.(%) |
|---|---|---|---|
| | Ag3642, | | Ag3642, |
| | Run | | Run |
| Tissue Name | 169975143 | Tissue Name | 169975143 |
| Secondary Th1 act | 63.3 | HUVEC IL-1beta | 30.1 |
| Secondary Th2 act | 74.2 | HUVEC IFN gamma | 25.2 |
| Secondary Tr1 act | 81.2 | HUVEC TNF alpha + | 18.4 |
| | | IFN gamma | |
| Secondary Th1 rest | 15.6 | HUVEC TNF | 12.1 |
| | | alpha + IL4 | |
| Secondary Th2 rest | 33.0 | HUVEC IL-11 | 18.6 |
| Secondary Tr1 rest | 18.2 | Lung Microvascular | 33.0 |
| | | EC none | |
| Primary Th1 act | 96.6 | Lung Microvascular | 36.6 |
| | | EC TNFalpha + | |
| | | IL-1beta | |
| Primary Th2 act | 100.0 | Microvascular Dermal | 18.3 |
| | | EC none | |
| Primary Tr1 act | 84.1 | Microsvasular Dermal | 15.6 |
| | | EC TNFalpha + | |
| | | IL-1beta | |
| Primary Th1 rest | 19.8 | Bronchial epithelium | 19.8 |
| | | TNFalpha + IL1beta | |
| Primary Th2 rest | 16.8 | Small airway | 10.7 |
| | | epithelium none | |
| Primary Tr1 rest | 47.6 | Small airway | 15.1 |
| | | epithelium | |
| | | TNFalpha + IL-1beta | |
| CD45RA CD4 | 27.7 | Coronery artery | 11.2 |
| lymphocyte act | | SMC rest | |
| CD45RO CD4 | 59.0 | Coronery artery SMC | 5.3 |
| lymphocyte act | | TNFalpha + IL-1beta | |
| CD8 lymphocyte act | 73.7 | Astrocytes rest | 18.8 |
| Secondary CD8 | 38.4 | Astrocytes | 11.9 |
| lymphocyte rest | | TNFalpha + IL-1beta | |
| Secondary CD8 | 23.8 | KU-812 (Basophil) | 25.0 |
| lymphocyte act | | rest | |
| CD4 lymphocyte | 35.6 | KU-812 (Basophil) | 32.1 |
| none | | PMA/ionomycin | |
| 2ry | 21.8 | CCD1106 | 27.4 |
| Th1/Th2/Tr1_anti- | | (Keratinocytes) | |
| CD95 CH11 | | none | |
| LAK cells rest | 33.0 | CCD1106 | 22.2 |
| | | (Keratinocytes) | |
| | | TNFalpha + IL-1beta | |
| LAK cells IL-2 | 25.5 | Liver cirrhosis | 6.0 |
| LAK cells IL-2 + | 21.9 | NCI-H292 none | 20.3 |
| IL-12 | | | |
| LAK cells IL-2 + | 28.7 | NCI-H292 IL-4 | 33.4 |
| IFN gamma | | | |
| LAK cells IL-2 + | 27.5 | NCI-H292 IL-9 | 42.6 |
| IL-18 | | | |
| LAK cells | 27.4 | NCI-H292 IL-13 | 30.1 |
| PMA/ionomycin | | | |
| NK Cells IL-2 rest | 69.7 | NCI-H292 IFN gamma | 39.5 |
| Two Way MLR | 67.8 | HPAEC none | 11.1 |
| 3 day | | | |
| Two Way MLR | 39.2 | HPAEC TNF alpha + | 27.5 |
| 5 day | | IL-1 beta | |
| Two Way MLR | 20.4 | Lung fibroblast none | 19.8 |
| 7 day | | | |
| PBMC rest | 29.5 | Lung fibroblast | 11.7 |
| | | TNF alpha + IL-1 beta | |
| PBMC PWM | 75.8 | Lung fibroblast IL-4 | 13.6 |
| PBMC PHA-L | 37.4 | Lung fibroblast IL-9 | 23.7 |
| Ramos (B cell) none | 37.9 | Lung fibroblast IL-13 | 24.3 |
| Ramos (B cell) | 61.1 | Lung fibroblast | 17.6 |
| ionomycin | | IFN gamma | |
| B lymphocytes | 34.4 | Dermal fibroblast | 32.5 |
| PWM | | CCD1070 rest | |
| B lymphocytes | 38.4 | Dermal fibroblast | 45.1 |
| CD40L and IL-4 | | CCD1070 TNF alpha | |
| EOL-1 dbcAMP | 97.9 | Dermal fibroblast | 17.9 |
| | | CCD1070 IL-1 beta | |
| EOL-1 dbcAMP | 70.7 | Dermal fibroblast | 7.3 |
| PMA/ionomycin | | IFN gamma | |
| Dendritic cells none | 24.7 | Dermal fibroblast IL-4 | 25.9 |
| Dendritic cells LPS | 24.3 | Dermal Fibroblasts | 14.5 |
| | | rest | |
| Dendritic cells | 27.5 | Neutrophils | 0.5 |
| anti-CD40 | | TNFa + LPS | |
| Monocytes rest | 41.5 | Neutrophils rest | 19.2 |
| Monocytes LPS | 30.8 | Colon | 5.5 |
| Macrophages rest | 38.4 | Lung | 6.7 |
| Macrophages LPS | 14.4 | Thymus | 57.4 |
| HUVEC none | 17.6 | Kidney | 28.9 |
| HUVEC starved | 16.7 | | |

CNS_neurodegeneration_v1.0 Summary: Ag3642 Results from one experiment with the CG59985-01 gene are not included. The amp plot indicates that there were experimental difficulties with this run.

General_screening_panel_v1.4 Summary: Ag3642 Expression of the CG59985-01 gene is ubiquitous in this panel, with highest expression in a breast cancer cell line (CT=26.6). Overall, expression of this gene appears to be higher in samples derived from cancer cell lines than in normal tissues. This widespread expression suggests that this gene product is involved in cell growth and proliferation. Thus, expression of this gene could be used as a marker to detect the presence of cancer. Furthermore, therapeutic modulation of the expression or function of this gene may be useful in the treatment of cancer.

Interestingly, this gene is expressed at much higher levels in fetal lung and liver (CTs=29-30) when compared to expression in the adult counterpart (CTs=33). Thus, expression of this gene may be used to differentiate between the fetal and adult sources of these tissue.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at low to moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag3642 The CG59985-01gene is expressed at high to moderate levels in a wide range of cell types of significance in the immune response in health and disease. Highest expression of the gene is seen in activated primary T cells (CT=30.7). Significant levels of expression are also seen in members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### Example D. Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV3 SNP Data:

NOV3 has three SNP variants, whose variant positions for its nucleotide and amino acid sequences is numbered according to SEQ ID NOs:7 and 8, respectively. The nucleotide sequence of the NOV3 variant differs as shown in Table SNP1.

TABLE SNP1
NOV3 variants.

| | |
|---|---|
| Nucleotides | Amino Acids |

| Variant | Position | Initial | Modified | Position | Initial | Modified |
|---|---|---|---|---|---|---|
| 13377522 | 280 | C | T | 81 | Pro | Ser |
| 13377521 | 457 | C | T | 140 | Gln | End |
| 13377520 | 762 | T | C | 241 | His | His |

NOV6a SNP Data:

NOV6a has two SNP variants, whose variant positions for its nucleotide and amino acid sequences is numbered according to SEQ ID NOs:13 and 14, respectively. The nucleotide sequence of the NOV6a variant differs as shown in Table SNP2.

TABLE SNP2
NOV6a variants.

| | |
|---|---|
| Nucleotides | Amino Acids |

| Variant | Position | Initial | Modified | Position | Initial | Modified |
|---|---|---|---|---|---|---|
| 13377525 | 297 | C | T | 90 | Ala | Val |
| 13377523 | 1327 | T | C | 0 | | No change |

NOV8 SNP Data:

NOV8 has two SNP variants, whose variant positions for its nucleotide and amino acid sequences is numbered according to SEQ ID NOs:19 and 20, respectively. The nucleotide sequence of the NOV8 variant differs as shown in Table SNP3.

TABLE SNP3
NOV8 variants.

| | |
|---|---|
| Nucleotides | Amino Acids |

| Variant | Position | Initial | Modified | Position | Initial | Modified |
|---|---|---|---|---|---|---|
| 13377527 | 2601 | A | G | 839 | Arg | Arg |
| 13377526 | 2607 | T | C | 841 | Arg | Arg |

NOV12 SNP Data:

NOV12 has 2 SNP variants, whose variant positions for its nucleotide and amino acid sequences is numbered according to SEQ ID NOs:29 and 30, respectively. The nucleotide sequence of the NOV12 variant differs as shown in Table SNP4.

TABLE SNP4
NOV12 variants.

| | |
|---|---|
| Nucleotides | Amino Acids |

| Variant | Position | Initial | Modified | Position | Initial | Modified |
|---|---|---|---|---|---|---|
| 13377529 | 437 | A | G | 139 | Asp | Gly |
| 13377530 | 444 | T | C | 141 | Asp | Asp |

NOV13a SNP Data:

NOV13a has two SNP variants, whose variant positions for its nucleotide and amino acid sequences is numbered according to SEQ ID NOs:31 and 32, respectively. The nucleotide sequence of the NOV13a variant differs as shown in Table SNP5.

TABLE SNP5
NOV13a variants.

| | |
|---|---|
| Nucleotides | Amino Acids |

| Variant | Position | Initial | Modified | Position | Initial | Modified |
|---|---|---|---|---|---|---|
| 13377532 | 201 | C | A | 64 | Pro | Thr |
| 13377533 | 1075 | A | G | 355 | Tyr | Cys |

NOV15 SNP Data:

NOV15 has one SNP variant, whose variant position for its nucleotide and amino acid sequences is numbered according to SEQ ID NOs:37 and 38, respectively. The nucleotide sequence of the NOV15 variant differs as shown in Table SNP6.

TABLE SNP6
NOV15 variants.

| | |
|---|---|
| Nucleotides | Amino Acids |

| Variant | Position | Initial | Modified | Position | Initial | Modified |
|---|---|---|---|---|---|---|
| 13377534 | 89 | T | C | 0 | | No change |

NOV21 SNP Data:

NOV21 has two SNP variants, whose variant positions for its nucleotide and amino acid sequences is numbered according to SEQ ID NOs:51 and 52, respectively. The nucleotide sequence of the NOV21 variant differs as shown in Table SNP7.

TABLE SNP7
NOV21 variants.

| | |
|---|---|
| Nucleotides | Amino Acids |

| Variant | Position | Initial | Modified | Position | Initial | Modified |
|---|---|---|---|---|---|---|
| 13377535 | 2435 | C | G | 419 | Asp | Glu |
| 13377536 | 2487 | C | T | 437 | Leu | Leu |

NOV22 SNP Data:

NOV22 has one SNP variant, whose variant position for its nucleotide and amino acid sequences is numbered according to SEQ ID NOs:53 and 54, respectively. The nucleotide sequence of the NOV22 variant differs as shown in Table SNP8.

TABLE SNP8
NOV22 variants.

| | |
|---|---|
| Nucleotides | Amino Acids |

| Variant | Position | Initial | Modified | Position | Initial | Modified |
|---|---|---|---|---|---|---|
| 13377538 | 1042 | C | T | 294 | His | His |

NOV24 SNP Data:

NOV24 has one SNP variant, whose variant position for its nucleotide and amino acid sequences is numbered according to SEQ ID NOs:57 and 58, respectively. The nucleotide sequence of the NOV24 variant differs as shown in Table SNP9.

TABLE SNP9
NOV24 variant.

| | |
|---|---|
| Nucleotides | Amino Acids |

| Variant | Position | Initial | Modified | Position | Initial | Modified |
|---|---|---|---|---|---|---|
| 13377539 | 225 | G | A | 65 | Gln | Gln |

NOV25 SNP Data:

NOV25 has one SNP variant, whose variant position for its nucleotide and amino acid sequences is numbered according to SEQ ID NOs:59 and 60, respectively. The nucleotide sequence of the NOV25 variant differs as shown in Table SNP10.

TABLE SNP10
NOV25 variant.

| | |
|---|---|
| Nucleotides | Amino Acids |

| Variant | Position | Initial | Modified | Position | Initial | Modified |
|---|---|---|---|---|---|---|
| 13377540 | 753 | C | G | 238 | Ala | Ala |

NOV27 SNP Data:

NOV27 has two SNP variants, whose variant positions for its nucleotide and amino acid sequences is numbered according to SEQ ID NOs:63 and 64, respectively. The nucleotide sequence of the NOV27 variant differs as shown in Table SNP11.

TABLE SNP11
NOV27 variants.

| | Nucleotides | | | Amino Acids | | |
|---|---|---|---|---|---|---|
| Variant | Position | Initial | Modified | Position | Initial | Modified |
| 13377541 | 1649 | G | A | 541 | Glu | Glu |
| 13377542 | 2308 | C | A | 761 | Ala | Glu |

Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

The claims presented are representative of the inventions disclosed herein. Other, unclaimed inventions are also contemplated. Applicants reserve the right to pursue such inventions in later claims.

What is claimed is:

1. An isolated polypeptide comprising an amino acid sequence selected from the group consisting of:
a) a mature form of the amino acid sequence selected from the group consisting of SEQ ID NO: 2n, wherein n is an integer between 1 and 33;
b) a variant of a mature form of the amino acid sequence selected from the group consisting of SEQ ID NO: 2n, wherein n is an integer between 1 and 33, wherein any amino acid in the mature form is changed to a different amino acid, provided that no more than 15% of the amino acid residues in the sequence of the mature form are so changed;
c) the amino acid sequence selected from the group consisting of SEQ ID NO: 2n, wherein n is an integer between 1 and 33;
d) a variant of the amino acid sequence selected from the group consisting of SEQ ID NO: 2n, wherein n is an integer between 1 and 33, wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence selected from the group consisting of SEQ ID NO: 2n, wherein n is an integer between 1 and 33.

3. The polypeptide of claim 2, wherein the allelic variant comprises an amino acid sequence that is the translation of a nucleic acid sequence differing by a single nucleotide from a nucleic acid sequence selected from the group consisting of SEQ ID NOS: 2n, wherein n is an integer between 1 and 33.

4. The polypeptide of claim 1 that is a variant polypeptide described therein, wherein any amino acid specified in the chosen sequence is changed to provide a conservative substitution.

5. A pharmaceutical composition comprising the polypeptide of claim 1 and a pharmaceutically acceptable carrier.

6. A kit comprising in one or more containers, the pharmaceutical composition of claim 5.

7. The use of a therapeutic in the manufacture of a medicament for treating a syndrome associated with a human disease, the disease selected from a pathology associated with the polypeptide of claim 1, wherein the therapeutic is the polypeptide of claim 1.

8. A method for determining the presence or amount of the polypeptide of claim 1 in a sample, the method comprising:
(a) providing the sample;
(b) introducing the sample to an antibody that binds immunospecifically to the polypeptide; and
(c) determining the presence or amount of antibody bound to the polypeptide, thereby determining the presence or amount of polypeptide in the sample.

9. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of the polypeptide in the sample of step (a) to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

10. A method of identifying an agent that binds to the polypeptide of claim 1, the method comprising:
(a) introducing the polypeptide to the agent; and
(b) determining whether the agent binds to the polypeptide.

11. The method of claim 10 wherein the agent is a cellular receptor or a downstream effector.

12. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

13. A method for screening for a modulator of activity or of latency or predisposition to a pathology associated with the polypeptide of claim 1, the method comprising:
a) administering a test compound to a test animal at increased risk for a pathology associated with the polypeptide of claim 1, wherein the test animal recombinantly expresses the polypeptide of claim 1;
b) measuring the activity of the polypeptide in the test animal after administering the compound of step (a); and
c) comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of claim 1.

14. The method of claim 13, wherein the test animal is a recombinant test animal that expresses a test protein transgene or expresses the transgene under the control of a promoter at an increased level relative to a wild-type test animal, and wherein the promoter is not the native gene promoter of the transgene.

15. A method for modulating the activity of the polypeptide of claim 1, the method comprising introducing a cell sample expressing the polypeptide of the claim with a compound that binds to the polypeptide in an amount sufficient to modulate the activity of the polypeptide.

16. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

17. The method of claim 16, wherein the subject is a human.

18. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2n, wherein n is an integer between 1 and 33, or a biologically active fragment thereof.

19. An isolated nucleic acid molecule comprising a nucleic acid sequence encoding a polypeptide comprising an amino acid sequence selected from the group consisting of:
a) a mature form of the amino acid sequence given SEQ ID NO: 2n, wherein n is an integer between 1 and 33;
b) a variant of a mature form of the amino acid sequence selected from the group consisting of SEQ ID NO: 2n, wherein n is an integer between 1 and 33, wherein any amino acid in the mature form of the chosen sequence is changed to a different amino acid, provided that no more than 15% of the amino acid residues in the sequence of the mature form are so changed;
c) the amino acid sequence selected from the group consisting of SEQ ID NO: 2n, wherein n is an integer between 1 and 33;
d) a variant of the amino acid sequence selected from the group consisting of SEQ ID NO: 2n, wherein n is an integer between 1 and 33, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence selected from the group consisting of SEQ ID NO: 2n, wherein n is an integer between 1 and 33, or any variant of the polypeptide wherein any amino acid of the chosen sequence is changed to a different amino acid, provided that no more than 10% of the amino acid residues in the sequence are so changed; and
f) the complement of any of the nucleic acid molecules.

20. The nucleic acid molecule of claim 19, wherein the nucleic acid molecule comprises the nucleotide sequence of a naturally occurring allelic nucleic acid variant.

21. The nucleic acid molecule of claim 19 that encodes a variant polypeptide, wherein the variant polypeptide has the polypeptide sequence of a naturally occurring polypeptide variant.

22. The nucleic acid molecule of claim 19, wherein the nucleic acid molecule differs by a single nucleotide from a nucleic acid sequence selected from the group consisting of SEQ ID NOS: 2n-1, wherein n is an integer between 1 and 33.

23. The nucleic acid molecule of claim 19, wherein the nucleic acid molecule comprises a nucleotide sequence selected from the group consisting of
a) the nucleotide sequence selected from the group consisting of SEQ ID NO: 2n-1, wherein n is an integer between 1 and 33;
b) a nucleotide sequence wherein one or more nucleotides in the nucleotide sequence selected from the group consisting of SEQ ID NO: 2n-1, wherein n is an integer between 1 and 33, is changed from that selected from the group consisting of the chosen sequence to a different nucleotide provided that no more than 15% of the nucleotides are so changed;
c) a nucleic acid fragment of the sequence selected from the group consisting of SEQ ID NO: 2n-1, wherein n is an integer between 1 and 33; and
d) a nucleic acid fragment wherein one or more nucleotides in the nucleotide sequence selected from the group consisting of SEQ ID NO: 2n-1, wherein n is an integer between 1 and 33, is changed from that selected from the group consisting of the chosen sequence to a different nucleotide provided that no more than 15% of the nucleotides are so changed.

24. The nucleic acid molecule of claim 19, wherein the nucleic acid molecule hybridizes under stringent conditions to the nucleotide sequence selected from the group consisting of SEQ ID NO: 2n-1, wherein n is an integer between 1 and 33, or a complement of the nucleotide sequence.

25. The nucleic acid molecule of claim 19, wherein the nucleic acid molecule comprises a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

26. A vector comprising the nucleic acid molecule of claim 9.

27. The vector of claim 26, further comprising a promoter operably linked to the nucleic acid molecule.

28. A cell comprising the vector of claim 27.

29. A method for determining the presence or amount of the nucleic acid molecule of claim 19 in a sample, the method comprising:
(a) providing the sample;
(b) introducing the sample to a probe that binds to the nucleic acid molecule; and
(c) determining the presence or amount of the probe bound to the nucleic acid molecule,
thereby determining the presence or amount of the nucleic acid molecule in the sample.

30. The method of claim 29 wherein presence or amount of the nucleic acid molecule is used as a marker for cell or tissue type.

31. The method of claim 30 wherein the cell or tissue type is cancerous.

32. A method for determining the presence of or predisposition to a disease associated with altered levels of the nucleic acid molecule of claim 19 in a first mammalian subject, the method comprising:
a) measuring the amount of the nucleic acid in a sample from the first mammalian subject; and
b) comparing the amount of the nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.
